SEQUENCE LISTING (473 sequences)

SEQ ID NO: 1 - DNA, 6730 bp, Homo sapiens
gcagatctaattcactggttacaatctgcaaaagaccggctagaattttggactcagcaatctgtgacagtcccacaagagctggaaatggtccgtgatcatctaaatgctttcctggagttttctaaagaagtggatgcccaatcttccctgaaatcatctgttctgagtactggaaatcagctccttcgactaaaaaaggtggacacagccacgctgcgctctgagctgtcgcgcattgatagccagtggactgacctgctaaccaatatcccagccgtccaggagaagctccaccagctccagatggataaactgccttcccgccatgccatttctgaagtcatgagttggatttctctaatggaaaatgttattcagaaggatgaagataatattaaaaattccataggttacaaggcaattcatgaataccttcagaaatataagggttttaagatagacattaactgtaaacagctgacagtggattttgtgaaccagtccgtgctacaaatcagcagtcaggatgtggaaagtaagcgtagtgataagactgattttgctgagcaacttggagcaatgaataaaagttggcaaattctgcaaggtctagtaactgagaagatccagctgttggaaggcttattggaatcttggtcagaatatgaaaataatgtacaatgtctgaaaacatggtttgaaacccaggaaaagagactaaaacaacagcatcgaattggagatcaggcttctgttcaaaatgcactgaaagactgtcaggatctggaagatttgattaaagcaaaagaaaaagaagtagagaaaattgagcagaatggacttgctttgattcagaacaagaaagaagacgtctctagcattgtcatgagcacactgcgagagctcggccaaacctgggcaaatttagatcacatggttggacaattaaagatactgctgaaatcagtgcttgaccaatggagtagtcacaaagtggcctttgacaagataaacagttacctcatggaggccagatactctctttcccgattccgtctgctgactggctccttagaagctgtgcaagttcaggtggacaatcttcagaatctccaagatgatctggaaaaacaggaaaggagcttacagaaatttggctctatcaccaaccaattattaaaagagtgtcacccacccgtgacagaaactcttaccaatacactgaaagaagtcaacatgagatggaataacttgctggaagagattgctgagcagctacagtccagcaaggccctacttcagctttggcaaagatacaaggactactccaaacagtgtgcttcgacagttcagcagcaggaggatcgaaccaatgagctgttgaaggcagccacaaacaaggacattgccgatgatgaggttgccacatggattcaagattgcaacgacctcctcaaaggactgggcacagttaaagattccctcttttttctccatgagctgggagagcaactgaagcaacaagtggatgcttccgcagcatcagctattcaatcggatcaactctctttgagtcaacacttgtgtgccctggagcaagctctctgcaaacagcagacttcattacaggctggagttcttgattatgaaacctttgccaagagtttagaagctttggaggcctggatagtggaagctgaagaaatactacaagggcaggaccctagccactcatctgacctctccacaatccaggaaaggatggaagaacttaagggacagatgttaaaattcagcagcatggctccagatttagaccgtctaaatgagcttggatataggttacccttgaatgataaggaaatcaaaagaatgcagaatctgaaccgccattggtctctgatctcctctcagactacagaaagattcagcaagttgcagtcatttttgctacaacatcagactttcttggaaaaatgtgaaacatggatggaattcctagttcagacagaacaaaagttagcagtagagatttcaggaaattatcagcaccttttggaacagcagagagcacacgagttgtttcaagccgagatgttcagtcgtcagcagattttgcactcaatcattattgatgggcaacgtcttctagaacaaggtcaagttgatgacagggatgaattcaacctgaaattgacactcctcagtaatcaatggcagggagtgattcgcagggcccagcagaggcgggggatcattgacagccagattcgccagtggcagcgctatagggagatggcagaaaagcttcgtaaatggttggttgaagtgtcctacctccccatgagtggtctcggaagtgttcctataccactgcaacaagcaaggaccctctttgatgaagtgcagttcaaagaaaaagtgtttctgcggcaacaaggcagctacatcctgactgtggaggctggcaagcaactccttctctcggcggacagtggcgctgaggccgccttgcaggccgaactcgctgaaatccaagagaaatggaaatcagccagcatgcggctggaagaacagaagaaaaaactagccttcttgttgaaagactgggaaaaatgtgagaaaggaatagcagattccctggagaaactacgaactttcaaaaagaagctttcgcagtctctcccggatcaccatgaagagctccatgcagaacaaatgcgttgcaaggaattagaaaatgcagttgggagctggacagatgacttgacccagttgagcctgctgaaggacaccctctctgcctatatcagtgctgatgatatctccattcttaatgaacgcgtagagcttctgcaaaggcagtgggaagaactatgccaccagctctccttaaggcggcagcaaataggtgaaagattgaatgaatgggcagtcttcagtgaaaagaacaaggaactctgtgagtggttgactcaaatggaaagcaaagtttctcagaatggagacattctcattgaagaaatgatagagaagctcaagaaggattatcaagaggaaattgctattgctcaagagaacaaaatacagctccaacaaatgggagaacgacttgctaaagccagccatgaaagcaaagcatctgagattgaatacaagctgggaaaggtcaacgaccggtggcagcatctcctggacctcattgcagccagggtgaagaagctgaaggagaccctggtagccgtgcagcagcttgataagaacatgagcagcctgaggacctggctcgctcacatcgagtcagagctggccaagccaatagtctacgattcctgtaactcggaagaaatacagagaaagcttaatgagcagcaggagcttcagagagacatagagaagcacagtacaggtgttgcatctgtcctcaacctgtgtgaagtcctgctgcacgactgtgacgcctgtgccactgatgccgagtgtgactctatacagcaggctacgagaaacctggaccggcggtggagaaacatttgtgctatgtccatggaaaggaggctgaaaatcgaagagacgtggcgattgtggcagaaatttctggatgactattcacgttttgaagattggctgaagtcttcagaaaggacagctgcttttcccagctcttctggggtgatctatacagttgccaaggaagaactaaagaaatttgaggctttccagcgacaggtccacgagtgcctgacgcagctggaactgatcaacaagcagtaccgccgcctggccagggagaaccgcactgattcagcatgtagcctcaaacagatggttcacgaaggcaaccagagatgggacaacctgcaaaagcgtgtcacctccatcttgcgcagactcaagcattttattggccagcgtgaggagtttgagactgcgcgggacagcattctggtctggctcacagagatggatctgcagctcactaatattgaacatttttctgagtgtgatgttcaagctaaaataaagcaactcaaggccttccagcaggaaatttcactgaaccacaataagattgagcagataattgcccaaggagaacagctgatagaaaagagtgagcccttggatgcagcgatcatcgaggaggaactagatgagctccgacggtactgccaggaggtcttcgggcgtgtggaaagataccataagaaactgatccgcctgcctctcccagacgatgagcacgacctctcagacagggagctggagctggaagactctgcagctctgtcggacctgcactggcacgaccgctctgcagacagcctgctttctccacagccttcctccaatctctccctctcgctcgctcagcccctccggagcgagcggtcaggacgagacaccccagctagtgtggactccatccccctggagtgggatcacgactatgacctcagtcgggacctggagtctgcaatgtccagagctctgccctctgaggatgaagaaggtcaggatgacaaagatttctacctccggggagctgttgccttatcaggggaccacagtgccctagagtcacagatccgacaactgggcaaagccctggatgatagccgctttcagatacagcaaaccgaaaatatcattcgcagcaaaactcccacggggccggagctagacaccagctacaaaggctacatgaaactgctgggcgaatgcagtagcagtatagactccgtgaagagactggagcacaaactgaaggaggaagaggagagccttcctggctttgttaacctgcatagtaccgaaacccaaacggctggtgtgattgaccgatgggagcttctccaggcccaggcattgagcaaggagttgaggatgaagcagaacctccagaagtggcagcagtttaactcagacttgaacagcatctgggcctggctgggggacacggaggaggagttggaacagctccagcgtctggaactcagcactgacatccagaccatcgagctccagatcaaaaagctcaaggagctccagaaagctgtggaccaccgcaaagccatcatcctctccatcaatctctgcagccctgagttcacccaggctgacagcaaggagagccgggacctgcaggatcgcttgtcgcagatgaatgggcgctgggaccgagtgtgctctctgctggaggagtggcggggcctgctgcaggatgccctgatgcagtgccagggtttccatgaaatgagccatggtttgcttcttatgctggagaacattgacagaaggaaaaatgaaattgtccctattgattctaaccttgatgcagagatacttcaggaccatcacaaacagcttatgcaaataaagcatgagctgttggaatcccaactcagagtagcctctttgcaagacatgtcttgccaactactggtgaatgctgaaggaacagactgtttagaagccaaagaaaaagtccatgttattggaaatcggctcaaacttctcttgaaggaggtcagtcgtcatatcaaggaactggagaagttattagacgtgtcaagtagtcagcaggatttgtcttcctggtcttctgctgatgaactggacacctcagggtctgtgagtcccacatcaggaaggagcaccccaaacagacagaaaacgccacgaggcaagtgtagtctctcacagcctggaccctctgtcagcagtccacatagcaggtccacaaaaggtggctccgattcctccctttctgagccagggccaggtcggtccggccgcggcttcctgttcagagtcctccgagcagctcttccccttcagcttctcctgctcctcctcatcgggcttgcctgccttgtaccaatgtcagaggaagactacagctgtgccctctccaacaactttgcccggtcattccaccccatgctcagatacacgaatggccctcctccactctgaactaagcagatgccatctgcagaagtgctggtagcataaggaggatcgggtcataagcaatcccaaactaccaacaagaggaccttgatcttggcgaaagccctcggtgtggcagctttagccctcctccagatcacatgtgtgcaaattatggcttcagaggtggaagataaacagtgacgggggaacaaacagacaacaagaaggtttggaagaaatctggtttgagactctgaaccttagcactaaggagattgagtaaggacctccaaagttccccggactcatgaattctgggcccttggcccattctgtgcacagccaaggacttcagtagaccatctgggcagctttcccatggtgctgctccaaccatcagataaatgaccctcccaagcaccatgtcagtgtcgtacaatctaccaaccaaccagtgctgaagagattttagaaccttgtaacatacaatttttaagagcttatatggcagcttcctttttaccttgttttcctttggggcatgatgttttaacctttgctttagaagcacaagctgtaaatctaaaaggcacttttttttagaggtataaagaaaaactagatgtaataaataagatcatggaaggctttatgtgaaaaaagttgaatgttatagtaaaaaaaaaaagatatttatgtatgtacagtttgctaaagccaagttttgtttgtattgatttctttgcatttattatagatattataaaat

SEQ ID NO: 2 - protein, 1993 aa, Homo sapiens
ADLIHWLQSAKDRLEFWTQQSVTVPQELEMVRDHLNAFLEFSKEVDAQSSLKSSVLSTGNQLLRLKKVDTATLRSELSRIDSQWTDLLTNIPAVQEKLHQLQMDKLPSRHAISEVMSWISLMENVIQKDEDNIKNSIGYKAIHEYLQKYKGFKIDINCKQLTVDFVNQSVLQISSQDVESKRSDKTDFAEQLGAMNKSWQILQGLVTEKIQLLEGLLESWSEYENNVQCLKTWFETQEKRLKQQHRIGDQASVQNALKDCQDLEDLIKAKEKEVEKIEQNGLALIQNKKEDVSSIVMSTLRELGQTWANLDHMVGQLKILLKSVLDQWSSHKVAFDKINSYLMEARYSLSRFRLLTGSLEAVQVQVDNLQNLQDDLEKQERSLQKFGSITNQLLKECHPPVTETLTNTLKEVNMRWNNLLEEIAEQLQSSKALLQLWQRYKDYSKQCASTVQQQEDRTNELLKAATNKDIADDEVATWIQDCNDLLKGLGTVKDSLFFLHELGEQLKQQVDASAASAIQSDQLSLSQHLCALEQALCKQQTSLQAGVLDYETFAKSLEALEAWIVEAEEILQGQDPSHSSDLSTIQERMEELKGQMLKFSSMAPDLDRLNELGYRLPLNDKEIKRMQNLNRHWSLISSQTTERFSKLQSFLLQHQTFLEKCETWMEFLVQTEQKLAVEISGNYQHLLEQQRAHELFQAEMFSRQQILHSIIIDGQRLLEQGQVDDRDEFNLKLTLLSNQWQGVIRRAQQRRGIIDSQIRQWQRYREMAEKLRKWLVEVSYLPMSGLGSVPIPLQQARTLFDEVQFKEKVFLRQQGSYILTVEAGKQLLLSADSGAEAALQAELAEIQEKWKSASMRLEEQKKKLAFLLKDWEKCEKGIADSLEKLRTFKKKLSQSLPDHHEELHAEQMRCKELENAVGSWTDDLTQLSLLKDTLSAYISADDISILNERVELLQRQWEELCHQLSLRRQQIGERLNEWAVFSEKNKELCEWLTQMESKVSQNGDILIEEMIEKLKKDYQEEIAIAQENKIQLQQMGERLAKASHESKASEIEYKLGKVNDRWQHLLDLIAARVKKLKETLVAVQQLDKNMSSLRTWLAHIESELAKPIVYDSCNSEEIQRKLNEQQELQRDIEKHSTGVASVLNLCEVLLHDCDACATDAECDSIQQATRNLDRRWRNICAMSMERRLKIEETWRLWQKFLDDYSRFEDWLKSSERTAAFPSSSGVIYTVAKEELKKFEAFQRQVHECLTQLELINKQYRRLARENRTDSACSLKQMVHEGNQRWDNLQKRVTSILRRLKHFIGQREEFETARDSILVWLTEMDLQLTNIEHFSECDVQAKIKQLKAFQQEISLNHNKIEQIIAQGEQLIEKSEPLDAAIIEEELDELRRYCQEVFGRVERYHKKLIRLPLPDDEHDLSDRELELEDSAALSDLHWHDRSADSLLSPQPSSNLSLSLAQPLRSERSGRDTPASVDSIPLEWDHDYDLSRDLESAMSRALPSEDEEGQDDKDFYLRGAVALSGDHSALESQIRQLGKALDDSRFQIQQTENIIRSKTPTGPELDTSYKGYMKLLGECSSSIDSVKRLEHKLKEEEESLPGFVNLHSTETQTAGVIDRWELLQAQALSKELRMKQNLQKWQQFNSDLNSIWAWLGDTEEELEQLQRLELSTDIQTIELQIKKLKELQKAVDHRKAIILSINLCSPEFTQADSKESRDLQDRLSQMNGRWDRVCSLLEEWRGLLQDALMQCQGFHEMSHGLLLMLENIDRRKNEIVPIDSNLDAEILQDHHKQLMQIKHELLESQLRVASLQDMSCQLLVNAEGTDCLEAKEKVHVIGNRLKLLLKEVSRHIKELEKLLDVSSSQQDLSSWSSADELDTSGSVSPTSGRSTPNRQKTPRGKCSLSQPGPSVSSPHSRSTKGGSDSSLSEPGPGRSGRGFLFRVLRAALPLQLLLLLLIGLACLVPMSEEDYSCALSNNFARSFHPMLRYTNGPPPL

SEQ ID NO: 3 - DNA, 10741 bp, Homo sapiens
caaaaatcagtctgatctcgggaaacctggagaaatttattttctgtactctaatgttctttcattttggtgaccatcaaggtgctgggagaggaattagatggctgtaattcaaagttaatggaattagatgcagcagtacagaaattcttggaacagaatggccaactgggtaagccactggccaagaagataggaaaactgactgaacttcaccagcagaccattagacaagctgagaatcggctctccaagctcaatcaggcaacatcacatttagaagaatacaatgaaatgcttgaattaattttgaagtggattgaaaaagctaaagtcttggctcatggaactattgcatggaattctgcaagccagcttcggaaacaatatattttgcatcagaccctgctagaagaatccaaagaaattgacagtgagctggaagcaatgactgagaaattacagtacctcactagcgtgtactgtacagaaaaaatgtctcagcaagtggcagaactgggacgggagactgaggagttgcgacagatgatcaaaattcgtttgcagaacctccaagatgcagctaaggatatgaaaaaatttgaagcagagttgaaaaagttacaagctgccttggagcaagcccaggcaacactgacttctccagaagttggacgtctcagtctcaaggagcagctctctcatcggcagcatttgttgtctgagatggagtcactgaagccgaaggtgcaagcagtgcagctctgccagagtgccctccggatccccgaggatgtggttgccagcttacctctctgtcatgctgctctgcggctgcaggaagaggccagccggctgcagcacaccgccatccagcagtgtaacatcatgcaggaagctgtggtacaatatgaacaatatgagcaagaaatgaaacatctccagcaactgatagaaggagctcacagagagattgaggataaacctgttgccaccagtaacatacaggagctgcaggctcagatttctcggcatgaggagctggcgcagaaaattaagggctaccaggagcagatcgcttctttgaattccaagtgcaagatgctgacgatgaaagccaagcacgccaccatgctgctgacggtgaccgaggtcgaggggctggcggaagggacagaggacctggatgggagctcctccccacgccttcggcccacccctctgtggtcatgatgactgcaggtcgctgtcacactttgctgtcaccggtcactgaggagtctggggaggagggaaccaacagtgagatttcctctccacctgcctgtcgctccccttcacctgtggctaatacagatgcttctgttaaccaggacattgcatattaccaagccttgtctgctgagaggttgcagacagatgctgcaaaaattcaccccagcacatccgcatcccaggagttctatgaaccgggattggagccatccgctactgccaaactgggtgatttgcagcgttcttgggaaaccttaaagaatgtgatcagtgagaagcagcgcacactctatgaagctttggagcgccagcagaagtaccaggactccctccagtccatctctacgaagatggaggccattgagctgaaactcagtgagagcccagagcctggcaggagtccagaaagccagatggctgaacatcaggcattgatggatgagattctcatgctccaggatgaaatcaatgagctccagtcctctctcgcagaggagctggtatccgagtcttgtgaggccgaccctgcggagcagctggccttgcagtccacgctcactgtcttagccgagcgaatgtccaccatcaggatgaaagcctcggggaaacggcagcttttggaggagaagttgaatgatcagctggaggaacaaaggcaggaacaggccctgcagaggtatcgctgtgaagccgatgagctggacagctggctcttgagtaccaaggccactctggacactgcgctgagtccacccaaggagcccatggacatggaggcccagcttatggactgccagaatatgctggtggaaatagagcagaaggtggtggctttatcagaactgtcagtccacaatgagaacctgctgctggagggcaaagctcacaccaaggacgaggccgagcagctggctggaaagctgagaaggctcaaggggagcctgctggagctgcagagagccctgcatgataagcagctcaacatgcagggaacagcacaggagaaggaggagagcgatgttgacctaacagccacgcagagccccggcgtccaggaatggctggcccaagctcgcaccacatggacccagcagcggcagagcagtctccagcaacaaaaagagttagaacaggaattagccgagcagaagagtctccttcgctcagtagccagtcgtggagaggagattctaattcaacattcggcggcagagacctctggtgatgctggcgaaaaacctgatgtgttatcccaggagttggggatggaaggggagaaatcatccgctgaagaccagatgagaatgaaatgggaaagcctacatcaagaatttagtaccaagcagaaactactacagaatgttctggaacaggaacaagagcaagtgctttatagcaggccaaatcgactcttgtctggtgtgccactgtacaaaggggacgtgccaacccaagataaatctgcagttacatctttgctggatggactgaaccaagccttcgaggaggtttcatcccagagtggaggggcaaagaggcagagtatacacttggagcagaagttgtatgatggagtctcagccacctctacttggttggatgacgttgaagaacgtttatttgttgccacagcacttttaccagaagaaacagagacttgtctcttcaaccaagagattcttgccaaagacattaaggaaatgtctgaagaaatggataagaacaaaaacttgttttcccaagcttttccagagaatggtgataatcgagatgttattgaagatactttgggttgtcttttgggcaggttatccttgctagactcagtagtgaatcaacgatgtcatcagatgaaagaaagacttcagcaaatactaaatttccagaatgatctgaaagtgctgtttacatcactggctgacaacaaatacatcattctgcaaaaactggcaaatgtgtttgaacagcccgtagcagaacaaatagaggcaatacaacaggctgaagatggactcaaagaatttgatgcaggaatcattgaattaaagaggcgtggtgacgagctacaggtcgagcagccgtccatgcaagaactctccaagctccaggacatgtatgatgagctgatgatgatcattggctcccggaggagtggtctgaatcagaaccttacactcaagagtcagtatgagagggccctacaagatctggctgacctgctagaaactggtcaggagaagatggcaggagaccagaaaatcatcgtgtcttccaaagaggaaatccagcaaccacttgacaaacataaggaatactttcagggcctggaatctcatatgatcttgactgtaacactcttcagaaagataatcagctttgcagtccaaaaggaaacccagttccatacagagctgatggctcaggcttctgctgtactgaaacgggctcacaagaggggtgtggagctggagtacattctagagacgtggtcccatctggatgaggaccagcaggagctcagcagacagctggaggtggtggaaagcagcatcccaagcgtgggtctggtggaggagaacgaggacaggcttattgaccgcataacactctaccagcatttaaaatctagccttaatgaataccagcccaaattatatcaagtattagatgatgggaaacgacttctgatatccatcagctgctcagatctagaaagccaactaaatcaacttggagagtgctggctaagtaacaccaataaaatgtctaaggaacttcacagactggaaacaatattgaaacactggaccagatatcaaagtgaatctgcagatctaattcactggttacaatctgcaaaagaccggctagaattttggactcagcaatctgtgacagtcccacaagagctggaaatggtccgtgatcatctaaatgctttcctggagttttctaaagaagtggatgcccaatcttccctgaaatcatctgttctgagtactggaaatcagctccttcgactaaaaaaggtggacacagccacgctgcgctctgagttgtcgcgcattgatagccagtggactgacctgctaaccaatatcccagccgtccaggagaagctccaccagctccagatggataaactgccttcccgccatgccatttctgaagtcatgagttggacttctctaatggaaaatgctattcagaaggatgaagataatattaaaaattccataggttacaaggcaattcatgaataccttcagaaatataagggttttaagatagacattaactgtaaacagctgacagtggattttgtgaaccagtccgtgctacaaatcagcagtcaggatgtggaaagtaagcgtagtgataagactgattttgctgagcaacttggagcaatgaataaaagttggcaaattctgcaaggtctagtaactgagaagatccagctgttggaaggcttattggaatcttggtcagaatatgaaaataatgtacaatgtctgaaaacatggtttgaaacccaggaaaagagactaaaacaacagcatcgaattggagatcaggcttctgttcaaaatgcactgaaagactgtcaggatctggaagatctgattaaagcaaaagataaagaagtagagaaaattgagcagaatggacttgctttgattcagaccaagaaagaagacgtctctagcattgtcatgagcacactgcgagagctcggccaaacctgggcaaatttagatcacatggttggacaattaaagatactgctgaaatcagtgcttgaccaatggagtagtcacaaagtggcctttgacaagataaacagttacctcatggaggccagatactctctttcccgattccgtctgctgactggctccttagaagctgtgcaagttcaggtggacaatcttcagaatctccaagatgatctggaaaaacaggaaaggagcttacagaaatttggctctatcaccaaccaattattaaaagagtgtcacccacccgtgacagaaactcttaccaatacactgaaagaagtcaacatgagatggaataacttgctggaagagattgctgagcagctacagtccagcaaggccctacttcagctttggcaaagatacaaggactactccaaacagtgtgcttcgacagttcagcagcaggaggatcgaaccaatgagctgttgaaggcagccacaaacaaggacattgccgatgatgaggttgccacatggattcaagattgcaacgacctcctcaaaggactgggcacagttaaagattccctctttgttctccatgagctgggagagcaactgaagcaacaagtggatgcttccgcagcatcagctattcaatcggatcaactctctttgagtcaacacttgtgtgccctggagcaagctctctgcaaacagcagacttcattacaggctggagttcttgattatgaaacctttgccaagagtttagaagctttggaggcctggatagtggaagctgaagaaatactacaagggcaggaccctagccactcatctgacctctccacaatccaggaaaggatggaagaacttaagggacagatgttaaaattcagcagcatggctccagatttagaccgtctaaatgagcttggatataggttacccttgaatgataaggaaatcaaaagaatgcagaatctgaaccgccattggtctctgatctcctctcagactacagaaagattcagcaagttgcagtcatttttgctacaacatcagactttcttggaaaaatgtgaaacatggatggaattcctagttcagacagaacaaaagttagcagtagagatttcaggaaattatcagcaccttttggaacagcagagagcacacgagttgtttcaagccgagatgttcagtcgtcagcagattttgcactcaatcattattgatgggcaacgtcttctagaacaaggtcaagttgatgacagggatgaattcaacctgaaattgacactcctcagtaatcaatggcagggagtgattcgcagggcccagcagaggcgggggatcattgacagccagattcgccagtggcagcgctatagggagatggcagaaaagcttcgtaaatggttggttgaagtgtcctacctccccatgagtggtctcggaagtgttcctataccactgcaacaagcaaggaccctctttgatgaagtgcagttcaaagaaaaagtgtttctgcggcaacaaggcagctacatcctgactgtggaggctggcaagcaactccttctctcggcggacagtggcgctgaggccgccttgcaggccgaactcgctgaaatccaagagaaatggaaatcagccagcatgcggctggaagaacagaagaaaaaactagccttcttgttgaaagactgggaaaaatgtgagaaaggaatagcagattccctggagaaactacgaactttcaaaaagaagctttcgcagtctctcccggatcaccatgaagagctccatgcagaacaaatgcgttgcaaggaattagaaaatgcagttgggagctggacagatgacttgacccagttgagcctgctgaaggacaccctctctgcctatatcagtgctgatgatatctccattcttaatgaacgcgtagagcttctgcaaaggcagtgggaagaactatgccaccagctctccttaaggcggcagcaaataggtgaaagattgaatgaatgggcagtcttcagtgaaaagaacaaggaactctgtgagtggttgactcaaatggaaagcaaagtttctcagaatggagacattctcattgaagaaatgatagagaagctcaagaaggattatcaagaggaaattgctattgctcaagagaacaaaatacagctccaacaaatgggagaacgacttgctaaagccagccatgaaagcaaagcatctgagattgaatacaagctgggaaaggtcaacgaccggtggcagcatctcctggacctcattgcagccagggtgaagaagctgaaggagaccctggtagccgtgcagcagcttgataagaacatgagcagcctgaggacctggctcgctcacatcgagtcagagctggccaagccaatagtctacgattcctgtaactcggaagaaatacagagaaagcttaatgagcagcaggagcttcagagagacatagagaagcacagtacaggtgttgcatctgtcctcaacctgtgtgaagtcctgctgcacgactgtgacgcctgtgccactgatgccgagtgtgactctatacagcaggctacgagaaacctggaccggcggtggagaaacatttgtgctatgtccatggaaaggaggctgaaaatcgaagagacgtggcgattgtggcagaaatttctggatgactattcacgttttgaagattggctgaagtcttcagaaaggacagctgcttttcccagctcttctggggtgatctatacagttgccaaggaagaactaaagaaatttgaggctttccagcgacaggtccacgagtgcctgacgcagctggaactgatcaacaagcagtaccgccgcctggccagggagaaccgcactgattcagcatgtagcctcaaacagatggttcacgaaggcaaccagagatgggacaacctgcaaaagcgtgtcacctccatcttgcgcagactcaagcattttattggccagcgtgaggagtttgagactgcgcgggacagcattctggtctggctcacagagatggatctgcagctcactaatattgaacatttttctgagtgtgatgttcaagctaaaataaagcaactcaaggccttccagcaggaaatttcactgaaccacaataagattgagcagataattgcccaaggagaacagctgatagaaaagagtgagcccttggatgcagcgatcatcgaggaggaactagatgagctccgacggtactgccaggaggtcttcgggcgtgtggaaagataccataagaaactgatccgcctgcctctcccagacgatgagcacgacctctcagacagggagctggagctggaagactctgcagctctgtcggacctgcactggcacgaccgctctgcagacagcctgctttctccacagccttcctccaatctctccctctcgctcgctcagcccctccggagcgagcggtcaggacgagacaccccagctagtgtggactccatccccctggagtgggatcacgactatgacctcagtcgggacctggagtctgcaatgtccagagctctgccctctgaggatgaagaaggtcaggatgacaaagatttctacctccggggagctgttgccttatcaggggaccacagtgccctagagtcacagatccgacaactgggcaaagccctggatgatagccgctttcagatacagcaaaccgaaaatatcattcgcagcaaaactcccacggggccggagctagacaccagctacaaaggctacatgaaactgctgggcgaatgcagtagcagtatagactccgtgaagagactggagcacaaactgaaggaggaagaggagagccttcctggctttgttaacctgcatagtaccgaaacccaaacggctggtgtgattgaccgatgggagcttctccaggcccaggcattgagcaaggagttgaggatgaagcagaacctccagaagtggcagcagtttaactcagacttgaacagcatctgggcctggctgggggacacggaggaggagttggaacagctccagcgtctggaactcagcactgacatccagaccatcgagctccagatcaaaaagctcaaggagctccagaaagctgtggaccaccgcaaagccatcatcctctccatcaatctctgcagccctgagttcacccaggctgacagcaaggagagccgggacctgcaggatcgcttgtcgcagatgaatgggcgctgggaccgagtgtgctctctgctggaggagtggcggggcctgctgcaggatgccctgatgcagtgccagggtttccatgaaatgagccatggtttgcttcttatgctggagaacattgacagaaggaaaaatgaaattgtccctattgattctaaccttgatgcagagatacttcaggaccatcacaaacagcttatgcaaataaagcatgagctgttggaatcccaactcagagtagcctctttgcaagacatgtcttgccaactactggtgaatgctgaaggaacagactgtttagaagccaaagaaaaagtccatgttattggaaatcggctcaaacttctcttgaaggaggtcagtcgtcatatcaaggaactggagaagttattagacgtgtcaagtagtcagcaggatttgtcttcctggtcttctgctgatgaactggacacctcagggtctgtgagtcccacatcaggaaggagcaccccaaacagacagaaaacgccacgaggcaagtgtagtctctcacagcctggaccctctgtcagcagtccacatagcaggtccacaaaaggtggctccgattcctccctttctgagccagggccaggtcggtccggccgcggcttcctgttcagagtcctccgagcagctcttccccttcagcttctcctgctcctcctcatcgggcttgcctgccttgtaccaatgtcagaggaagactacagctgtgccctctccaacaactttgcccggtcattccaccccatgctcagatacacgaatggccctcctccactctgaactaagcagatgccatctgcagaagtgctggtagcataaggaggatcgggtcataagcaatcccaaactaccaacaagaggaccttgatcttggcgaaagccctcggtgtggcagctttagccctcctccagatcacatgtgtgcaaattatggcttcagaggtggaagataaacagtgacgggggaacaaacagacaacaagaaggtttggaagaaatctggtttgagactctgaaccttagcactaaggagattgagtaaggacctccaaagttccccggactcatgaattctgggcccttggcccattctgtgcacagccaaggacttcagtagaccatctgggcagctttcccatggtgctgctccaaccatcagataaatgaccctcccaagcaccatgtcagtgtcgtacaatctaccaaccaaccagtgctgaagagattttagaaccttgtaacatacaatttttaagagcttatatggcagcttcctttttaccttgttttcctttggggcatgatgttttaacctttgctttagaagcacaagctgtaaatctaaaaggcacttttttttagaggtataaagaaaaactagatgtaataaataagatcatggaaggctttatgtgaaaaaagttgaatgttatagt

SEQ ID NO: 4 - protein, 3321 aa, Homo sapiens
MELDAAVQKFLEQNGQLGKPLAKKIGKLTELHQQTIRQAENRLSKLNQATSHLEEYNEMLELILKWIEKAKVLAHGTIAWNSASQLRKQYILHQTLLEESKEIDSELEAMTEKLQYLTSVYCTEKMSQQVAELGRETEELRQMIKIRLQNLQDAAKDMKKFEAELKKLQAALEQAQATLTSPEVGRLSLKEQLSHRQHLLSEMESLKPKVQAVQLCQSALRIPEDVVASLPLCHAALRLQEEASRLQHTAIQQCNIMQEAVVQYEQYEQEMKHLQQLIEGAHREIEDKPVATSNIQELQAQISRHEELAQKIKGYQEQIASLNSKCKMLTMKAKHATMLLTVTEVEGLAEGTEDLDGELLPTPSAHPSVVMMTAGRCHTLLSPVTEESGEEGTNSEISSPPACRSPSPVANTDASVNQDIAYYQALSAERLQTDAAKIHPSTSASQEFYEPGLEPSATAKLGDLQRSWETLKNVISEKQRTLYEALERQQKYQDSLQSISTKMEAIELKLSESPEPGRSPESQMAEHQALMDEILMLQDEINELQSSLAEELVSESCEADPAEQLALQSTLTVLAERMSTIRMKASGKRQLLEEKLNDQLEEQRQEQALQRYRCEADELDSWLLSTKATLDTALSPPKEPMDMEAQLMDCQNMLVEIEQKVVALSELSVHNENLLLEGKAHTKDEAEQLAGKLRRLKGSLLELQRALHDKQLNMQGTAQEKEESDVDLTATQSPGVQEWLAQARTTWTQQRQSSLQQQKELEQELAEQKSLLRSVASRGEEILIQHSAAETSGDAGEKPDVLSQELGMEGEKSSAEDQMRMKWESLHQEFSTKQKLLQNVLEQEQEQVLYSRPNRLLSGVPLYKGDVPTQDKSAVTSLLDGLNQAFEEVSSQSGGAKRQSIHLEQKLYDGVSATSTWLDDVEERLFVATALLPEETETCLFNQEILAKDIKEMSEEMDKNKNLFSQAFPENGDNRDVIEDTLGCLLGRLSLLDSVVNQRCHQMKERLQQILNFQNDLKVLFTSLADNKYIILQKLANVFEQPVAEQIEAIQQAEDGLKEFDAGIIELKRRGDELQVEQPSMQELSKLQDMYDELMMIIGSRRSGLNQNLTLKSQYERALQDLADLLETGQEKMAGDQKIIVSSKEEIQQPLDKHKEYFQGLESHMILTVTLFRKIISFAVQKETQFHTELMAQASAVLKRAHKRGVELEYILETWSHLDEDQQELSRQLEVVESSIPSVGLVEENEDRLIDRITLYQHLKSSLNEYQPKLYQVLDDGKRLLISISCSDLESQLNQLGECWLSNTNKMSKELHRLETILKHWTRYQSESADLIHWLQSAKDRLEFWTQQSVTVPQELEMVRDHLNAFLEFSKEVDAQSSLKSSVLSTGNQLLRLKKVDTATLRSELSRIDSQWTDLLTNIPAVQEKLHQLQMDKLPSRHAISEVMSWTSLMENAIQKDEDNIKNSIGYKAIHEYLQKYKGFKIDINCKQLTVDFVNQSVLQISSQDVESKRSDKTDFAEQLGAMNKSWQILQGLVTEKIQLLEGLLESWSEYENNVQCLKTWFETQEKRLKQQHRIGDQASVQNALKDCQDLEDLIKAKDKEVEKIEQNGLALIQTKKEDVSSIVMSTLRELGQTWANLDHMVGQLKILLKSVLDQWSSHKVAFDKINSYLMEARYSLSRFRLLTGSLEAVQVQVDNLQNLQDDLEKQERSLQKFGSITNQLLKECHPPVTETLTNTLKEVNMRWNNLLEEIAEQLQSSKALLQLWQRYKDYSKQCASTVQQQEDRTNELLKAATNKDIADDEVATWIQDCNDLLKGLGTVKDSLFVLHELGEQLKQQVDASAASAIQSDQLSLSQHLCALEQALCKQQTSLQAGVLDYETFAKSLEALEAWIVEAEEILQGQDPSHSSDLSTIQERMEELKGQMLKFSSMAPDLDRLNELGYRLPLNDKEIKRMQNLNRHWSLISSQTTERFSKLQSFLLQHQTFLEKCETWMEFLVQTEQKLAVEISGNYQHLLEQQRAHELFQAEMFSRQQILHSIIIDGQRLLEQGQVDDRDEFNLKLTLLSNQWQGVIRRAQQRRGIIDSQIRQWQRYREMAEKLRKWLVEVSYLPMSGLGSVPIPLQQARTLFDEVQFKEKVFLRQQGSYILTVEAGKQLLLSADSGAEAALQAELAEIQEKWKSASMRLEEQKKKLAFLLKDWEKCEKGIADSLEKLRTFKKKLSQSLPDHHEELHAEQMRCKELENAVGSWTDDLTQLSLLKDTLSAYISADDISILNERVELLQRQWEELCHQLSLRRQQIGERLNEWAVFSEKNKELCEWLTQMESKVSQNGDILIEEMIEKLKKDYQEEIAIAQENKIQLQQMGERLAKASHESKASEIEYKLGKVNDRWQHLLDLIAARVKKLKETLVAVQQLDKNMSSLRTWLAHIESELAKPIVYDSCNSEEIQRKLNEQQELQRDIEKHSTGVASVLNLCEVLLHDCDACATDAECDSIQQATRNLDRRWRNICAMSMERRLKIEETWRLWQKFLDDYSRFEDWLKSSERTAAFPSSSGVIYTVAKEELKKFEAFQRQVHECLTQLELINKQYRRLARENRTDSACSLKQMVHEGNQRWDNLQKRVTSILRRLKHFIGQREEFETARDSILVWLTEMDLQLTNIEHFSECDVQAKIKQLKAFQQEISLNHNKIEQIIAQGEQLIEKSEPLDAAIIEEELDELRRYCQEVFGRVERYHKKLIRLPLPDDEHDLSDRELELEDSAALSDLHWHDRSADSLLSPQPSSNLSLSLAQPLRSERSGRDTPASVDSIPLEWDHDYDLSRDLESAMSRALPSEDEEGQDDKDFYLRGAVALSGDHSALESQIRQLGKALDDSRFQIQQTENIIRSKTPTGPELDTSYKGYMKLLGECSSSIDSVKRLEHKLKEEEESLPGFVNLHSTETQTAGVIDRWELLQAQALSKELRMKQNLQKWQQFNSDLNSIWAWLGDTEEELEQLQRLELSTDIQTIELQIKKLKELQKAVDHRKAIILSINLCSPEFTQADSKESRDLQDRLSQMNGRWDRVCSLLEEWRGLLQDALMQCQGFHEMSHGLLLMLENIDRRKNEIVPIDSNLDAEILQDHHKQLMQIKHELLESQLRVASLQDMSCQLLVNAEGTDCLEAKEKVHVIGNRLKLLLKEVSRHIKELEKLLDVSSSQQDLSSWSSADELDTSGSVSPTSGRSTPNRQKTPRGKCSLSQPGPSVSSPHSRSTKGGSDSSLSEPGPGRSGRGFLFRVLRAALPLQLLLLLLIGLACLVPMSEEDYSCALSNNFARSFHPMLRYTNGPPPL

SEQ ID NO: 5 - DNA, 1464 bp, Homo sapiens
agcgggctgggtgcgtgcgcggcgactgcgacgggcagtggcagtcatggcggctcagtgcgtgaggttggcgcggcgcagtcttcctgctttggcgttgtctctcaggccatctccccggttgttgtgcacagccacgaaacaaaagaacagtggccagaacctggaagaggacatgggtcagagtgaacagaaggcagatcctcctgctacagagaagaccctcctggaagagaaggtcaagttggaggaacagctgaaggagactgtggaaaaatataaacgagctttggcagacactgagaacttacggcagaggagccagaaattggtggaggaggcaaaattatacggcattcaagccttctgcaaggacttgttggaggtggcagacgttctggagaaggcaacacagtgtgttccaaaagaagaaattaaagacgataaccctcacctgaagaacctctatgaggggctggtcatgactgaagtccagatccagaaggtgttcacaaagcatggcttgctcaagttgaaccctgtcggagccaagttcgacccttatgaacatgaggccttgttccacacaccggttgaggggaaggagccaggcacagtggccctagttagcaaagtggggtacaagctgcatgggcgcactctgagacccgccctggtgggggtggtgaaggaagcttagctgctgttgatggggtgggtgtttttaaactcacttgatgtaactctcaaggctggttcattgtttctcatctatgagtacgtgtgaccttttcccaaaccttattggaaaccttaagtaaccagtggctaaacagaaaagccggttgcccaactgcattaatgaactctaattcgggagtctgttcccttttagtgccacgcgttgaatagttccacatactttcagaagagctcagcagggccctgcctggtctcccgagcatcatgagtaacgtgtctgctcagactctgctgacaccaaagtattttaaacaaataagagagttgaaacatttttcttacttaaaaaaaatgatctttgtgaagaacatagtgagtgcggcgcatgagtgactggcgtatttagcccgtcacatttcattcgctgaaggaaaggcatcgtttgtcttcagtcaacagcggctgaaactgaccactgagaaatgggtgtgggcactgacagttctcccccattatttggccaggaattgagcttggcttggcaaagttccttttaccctgttctgttcatctaaatgcagacatatttaaatcatattcaactagttactaatgacctcaagttgtattccctggcaaaatggactttctcaaaataggactgcacgcttggtgtactttaaatgttaatgtttaatttaaaatttttatttaagaggattaaagccctaatgtttattttcctacaaaaaaaaaaaaaaa

SEQ ID NO: 6 - protein, 217 aa, Homo sapiens
MAAQCVRLARRSLPALALSLRPSPRLLCTATKQKNSGQNLEEDMGQSEQKADPPATEKTLLEEKVKLEEQLKETVEKYKRALADTENLRQRSQKLVEEAKLYGIQAFCKDLLEVADVLEKATQCVPKEEIKDDNPHLKNLYEGLVMTEVQIQKVFTKHGLLKLNPVGAKFDPYEHEALFHTPVEGKEPGTVALVSKVGYKLHGRTLRPALVGVVKEA

SEQ ID NO: 7 - DNA, 1519 bp, Homo sapiens
gctgggtgcgtgcgcggcgactgcgacgggcagtggcagtcatggcggctcagtgcgtgaggttggcgcggcgcagtcttcctgctttggcgttgtctctcaggccatctccccggttgttgtgcacagccacgaaacaaaagaacagtggccagaacctggaagaggacatgggtcagagtgaacagaaggcagatcctcctgctacagagaagaccctcctggaagagaaggtcaagttggaggaacagctgaaggagactgtggaaaaatataaacgagctttggcagacactgagaacttacggcagaggagccagaaattggtggaggaggcaaaattatacggcattcaagccttctgcaaggacttgttggaggtggcagacgttctggagaaggcaacacagtgtgttccaaaagaagaaattaaagacgataaccctcacctgaagaacctctatgaggggctggtcatgactgaagtccagatccagaaggtgttcacaaagcatggcttgctcaagttgaaccctgtcggagccaagttcgacccttatgaacatgaggccttgttccacacaccggttgaggggaaggagccaggcacagtggccctagttagcaaagtggggtacaagctgcatgggcgcactctgagacccgccctggtgggggtggtgaaggaagcttagctgctgttgatggggtgggtgtttttaaactcacttgatgtaactctcaaggctggttcattgtttctcatctatgagtacgtgtgaccttttcccaaaccttattggaaaccttaagtaaccagtggctaaacagaaaagccggttgcccaactgcattaatgaactctaattcgggagtctgttcccttttagtgccacgcgttgaatagttccacatactttcagaagagctcagcagggccctgcctggtctcccgagcatcatgagtaacgtgtctgctcagactctgctgacaccaaagtattttaaacaaataaaaggtcttggggaattctgtttggctacctgggcacgccagtctgcaccatgtgtccctgcggcgcatgagtgactggcgtatttagcccgtcacatttcattcgctgaaggaaaggcaagagagttgaaacatttttcttacttaaaaaaaatgatctttgtgaagaacatagtgagttcgtttgtcttcagtcaacagcggctgaaactgaccactgagaaatgggtgtgggcactgacagttctcccccattatttggccaggaattgagcttggcttggcaaagttccttttaccctgttctgttcatctaaatgcagacatatttaaatcatattcaactagttactaatgacctcaagttgtattccctggcaaaatggactttctcaaaataggactgcacgcttggtgtactttaaatgttaatgtttaatttaaaatttttatttaagaggattaaagccctaatgtttattttcctacaaaaaaaaaaaaaaa

SEQ ID NO: 8 - protein, 217 aa, Homo sapiens; identical to SEQ ID NO: 6

SEQ ID NO: 9 - DNA, 20 bp, Homo sapiens
catgactgaggttctcctga

SEQ ID NO: 10 - DNA, 20 bp, Homo sapiens
gcggcgcccgccatggctcc

SEQ ID NO: 11 - DNA, 20 bp, Homo sapiens
ggcgcccgccatggctccga

SEQ ID NO: 12 - DNA, 20 bp, Homo sapiens
cgcccgccatggctccgacg

SEQ ID NO: 13 - DNA, 20 bp, Homo sapiens
tcggtggccgccatctcggc

SEQ ID NO: 14 - DNA, 20 bp, Homo sapiens
ggtggccgccatctcggctg

SEQ ID NO: 15 - DNA, 20 bp, Homo sapiens
tggccgccatctcggctgcg

SEQ ID NO: 16 - DNA, 20 bp, Homo sapiens
gcaaaattgtccatggtgca

SEQ ID NO: 17 - DNA, 20 bp, Homo sapiens
aaaattgtccatggtgcaca

SEQ ID NO: 18 - DNA, 20 bp, Homo sapiens
aattgtccatggtgcacaag

SEQ ID NO: 19 - DNA, 20 bp, Homo sapiens
tttctgtcagccatggccaa

SEQ ID NO: 20 - DNA, 20 bp, Homo sapiens
tctgtcagccatggccaaag

SEQ ID NO: 21 - DNA, 20 bp, Homo sapiens
tgtcagccatggccaaagtt

SEQ ID NO: 22 - DNA, 20 bp, Homo sapiens
aagagcgagttcatggtggc

SEQ ID NO: 23 - DNA, 20 bp, Homo sapiens
gagcgagttcatggtggcgc

SEQ ID NO: 24 - DNA, 20 bp, Homo sapiens
gcgagttcatggtggcgctc

SEQ ID NO: 25 - DNA, 20 bp, Homo sapiens
acaaaccaagacatgatggc

SEQ ID NO: 26 - DNA, 20 bp, Homo sapiens
aaaccaagacatgatggcag

SEQ ID NO: 27 - DNA, 20 bp, Homo sapiens
accaagacatgatggcagca

SEQ ID NO: 28 - DNA, 20 bp, Homo sapiens
agccacagtaccatgaacag

SEQ ID NO: 29 - DNA, 20 bp, Homo sapiens
ccacagtaccatgaacagtc

SEQ ID NO: 30 - DNA, 20 bp, Homo sapiens
acagtaccatgaacagtctc

SEQ ID NO: 31 - DNA, 20 bp, Homo sapiens
ggtggccgggacatggcgag

SEQ ID NO: 32 - DNA, 20 bp, Homo sapiens
tggccgggacatggcgagcg

SEQ ID NO: 33 - DNA, 20 bp, Homo sapiens
gccgggacatggcgagcgcg

SEQ ID NO: 34 - DNA, 20 bp, Homo sapiens
tgggcgagcggcatctgccg

SEQ ID NO: 35 - DNA, 20 bp, Homo sapiens
ggcgagcggcatctgccgcc

SEQ ID NO: 36 - DNA, 20 bp, Homo sapiens
cgagcggcatctgccgccgc

SEQ ID NO: 37 - DNA, 20 bp, Homo sapiens
tttctgtcagccatggccaa

SEQ ID NO: 38 - DNA, 20 bp, Homo sapiens
tctgtcagccatggccaaag

SEQ ID NO: 39 - DNA, 20 bp, Homo sapiens
tgtcagccatggccaaagtt

SEQ ID NO: 40 - DNA, 20 bp, Homo sapiens
cccagctgcgccatggccgc

SEQ ID NO: 41 - DNA, 20 bp, Homo sapiens
cagctgcgccatggccgcgc

SEQ ID NO: 42 - DNA, 20 bp, Homo sapiens
gctgcgccatggccgcgcca

SEQ ID NO: 43 - DNA, 20 bp, Homo sapiens
tcataaaactccatggtact

SEQ ID NO: 44 - DNA, 20 bp, Homo sapiens
ataaaactccatggtacttt

SEQ ID NO: 45 - DNA, 20 bp, Homo sapiens
aaaactccatggtacttttg

SEQ ID NO: 46 - DNA, 20 bp, Homo sapiens
agccaaagtggccttaccca

SEQ ID NO: 47 - DNA, 20 bp, Homo sapiens
gtgagacttgtggggccact

SEQ ID NO: 48 - DNA, 20 bp, Homo sapiens
tcaagccttctagactgaaa

SEQ ID NO: 49 - DNA, 20 bp, Homo sapiens
ttgaggccggccatggcgcg

SEQ ID NO: 50 - DNA, 20 bp, Homo sapiens
gaggccggccatggcgcgga

SEQ ID NO: 51 - DNA, 20 bp, Homo sapiens
ggccggccatggcgcggagc

SEQ ID NO: 52 - DNA, 20 bp, Homo sapiens
cagcaccaaatttgccctgc

SEQ ID NO: 53 - DNA, 20 bp, Homo sapiens
tccatggccagcagctgcct

SEQ ID NO: 54 - DNA, 20 bp, Homo sapiens
ccagggaagaggtactgttg

SEQ ID NO: 55 - DNA, 20 bp, Homo sapiens
caggcccgcagcatctcggt

SEQ ID NO: 56 - DNA, 20 bp, Homo sapiens
ggcccgcagcatctcggtca

SEQ ID NO: 57 - DNA, 20 bp, Homo sapiens
cccgcagcatctcggtcaag

SEQ ID NO: 58 - DNA, 20 bp, Homo sapiens
gtttccgccgccatctttcg

SEQ ID NO: 59 - DNA, 20 bp, Homo sapiens
ttccgccgccatctttcgtt

SEQ ID NO: 60 - DNA, 20 bp, Homo sapiens
ccgccgccatctttcgttcc

SEQ ID NO: 61 - DNA, 20 bp, Homo sapiens
aaactgtccccagtcgacat

SEQ ID NO: 62 - DNA, 20 bp, Homo sapiens
ctgggcttctgcatggcggt

SEQ ID NO: 63 - DNA, 20 bp, Homo sapiens
gggcttctgcatggcggtgc

SEQ ID NO: 64 - DNA, 20 bp, Homo sapiens
gcttctgcatggcggtgcca

SEQ ID NO: 65 - DNA, 20 bp, Homo sapiens
tcctcacccgacatggcctc

SEQ ID NO: 66 - DNA, 20 bp, Homo sapiens
ctcacccgacatggcctcct

SEQ ID NO: 67 - DNA, 20 bp, Homo sapiens
cacccgacatggcctcctgg

SEQ ID NO: 68 - DNA, 20 bp, Homo sapiens
gcatagcttgtcaaactttt

SEQ ID NO: 69 - DNA, 20 bp, Homo sapiens
tcttcttcttttccaaaagt

SEQ ID NO: 70 - DNA, 20 bp, Homo sapiens
ggaacgactgagcaggcctc

SEQ ID NO: 71 - DNA, 20 bp, Homo sapiens
tcgatccgagacataatcgg

SEQ ID NO: 72 - DNA, 20 bp, Homo sapiens
gatccgagacataatcggac

SEQ ID NO: 73 - DNA, 20 bp, Homo sapiens
tccgagacataatcggacgg

SEQ ID NO: 74 - DNA, 20 bp, Homo sapiens
cactgcagactcatcctgtt

SEQ ID NO: 75 - DNA, 20 bp, Homo sapiens
ctgcagactcatcctgttgc

SEQ ID NO: 76 - DNA, 20 bp, Homo sapiens
gcagactcatcctgttgcta

SEQ ID NO: 77 - DNA, 20 bp, Homo sapiens
acctcgttgcccatggcggg

SEQ ID NO: 78 - DNA, 20 bp, Homo sapiens
ctcgttgcccatggcgggca

SEQ ID NO: 79 - DNA, 20 bp, Homo sapiens
cgttgcccatggcgggcagg

SEQ ID NO: 80 - DNA, 20 bp, Homo sapiens
tgctttatgtacatgattcc

SEQ ID NO: 81 - DNA, 20 bp, Homo sapiens
ctttatgtacatgattccgg

SEQ ID NO: 82 - DNA, 20 bp, Homo sapiens
ttatgtacatgattccgggc

SEQ ID NO: 83 - DNA, 20 bp, Homo sapiens
aacggaatcatcatatgtga

SEQ ID NO: 84 - DNA, 20 bp, Homo sapiens
cggaatcatcatatgtgaca

SEQ ID NO: 85 - DNA, 20 bp, Homo sapiens
gaatcatcatatgtgacaga

SEQ ID NO: 86 - DNA, 20 bp, Homo sapiens
atttttcgagacatttttat

SEQ ID NO: 87 - DNA, 20 bp, Homo sapiens
ttttcgagacatttttatta

SEQ ID NO: 88 - DNA, 20 bp, Homo sapiens
ttcgagacatttttattatt

SEQ ID NO: 89 - DNA, 20 bp, Homo sapiens
agtccaccattccgccgcct

SEQ ID NO: 90 - DNA, 20 bp, Homo sapiens
gtagtccaccattccgccgc

SEQ ID NO: 91 - DNA, 20 bp, Homo sapiens
tggtagtccaccattccgcc

SEQ ID NO: 92 - DNA, 20 bp, Homo sapiens
ggaggggggaccattccagg

SEQ ID NO: 93 - DNA, 20 bp, Homo sapiens
aggggggaccattccaggct

SEQ ID NO: 94 - DNA, 20 bp, Homo sapiens
gggggaccattccaggcttc

SEQ ID NO: 95 - DNA, 20 bp, Homo sapiens
ttgccaaacgccatggtagc

SEQ ID NO: 96 - DNA, 20 bp, Homo sapiens
gccaaacgccatggtagcgc

SEQ ID NO: 97 - DNA, 20 bp, Homo sapiens
caaacgccatggtagcgcca

SEQ ID NO: 98 - DNA, 20 bp, Homo sapiens
cctggaaccgacattttaac

SEQ ID NO: 99 - DNA, 20 bp, Homo sapiens
tggaaccgacattttaaccg

SEQ ID NO: 100 - DNA, 20 bp, Homo sapiens
gaaccgacattttaaccgcc

SEQ ID NO: 101 - DNA, 20 bp, Homo sapiens
tgatcacggaccatttggag

SEQ ID NO: 102 - DNA, 20 bp, Homo sapiens
atcacggaccatttggagct

SEQ ID NO: 103 - DNA, 20 bp, Homo sapiens
cacggaccatttggagctct

SEQ ID NO: 104 - DNA, 20 bp, Homo sapiens
cggtattgaagcatccactg

SEQ ID NO: 105 - DNA, 20 bp, Homo sapiens
gtattgaagcatccactgtg

SEQ ID NO: 106 - DNA, 20 bp, Homo sapiens
attgaagcatccactgtggg

SEQ ID NO: 107 - DNA, 20 bp, Homo sapiens
cattcccgtgtggatgctgt

SEQ ID NO: 108 - DNA, 20 bp, Homo sapiens
ggcctgagaagccagtgcct

SEQ ID NO: 109 - DNA, 20 bp, Homo sapiens
acctcctgcacaatctcagc

SEQ ID NO: 110 - DNA, 20 bp, Homo sapiens
cttcctgggtcctctgcttt

SEQ ID NO: 111 - DNA, 20 bp, Homo sapiens
tcagcctctttatttcttca

SEQ ID NO: 112 - DNA, 20 bp, Homo sapiens
cagatctcgattttcccttg

SEQ ID NO: 113 - DNA, 20 bp, Homo sapiens
ggtatgacattctgacccca

SEQ ID NO: 114 - DNA, 20 bp, Homo sapiens
tgggtatgacattctgaccc

SEQ ID NO: 115 - DNA, 20 bp, Homo sapiens
cctgggtatgacattctgac

SEQ ID NO: 116 - DNA, 20 bp, Homo sapiens
ctaaccgactccattaccag

SEQ ID NO: 117 - DNA, 20 bp, Homo sapiens
aaccgactccattaccagcc

SEQ ID NO: 118 - DNA, 20 bp, Homo sapiens
ccgactccattaccagcccc

SEQ ID NO: 119 - DNA, 20 bp, Homo sapiens
tttagcatactcattttctt

SEQ ID NO: 120 - DNA, 20 bp, Homo sapiens
tagcatactcattttctttg

SEQ ID NO: 121 - DNA, 20 bp, Homo sapiens
gcatactcattttctttgta

SEQ ID NO: 122 - DNA, 20 bp, Homo sapiens
gaggtgtcagagacatgcgt

SEQ ID NO: 123 - DNA, 20 bp, Homo sapiens
ttgcggttgttgtccatgga

SEQ ID NO: 124 - DNA, 20 bp, Homo sapiens
gaggtggtggtgtacttgat

SEQ ID NO: 125 - DNA, 20 bp, Homo sapiens
tcatgatgtaaaatcagagg

SEQ ID NO: 126 - DNA, 20 bp, Homo sapiens
gcaattgcaaagagtgaaga

SEQ ID NO: 127 - DNA, 20 bp, Homo sapiens
tccagctctaccttgttcat

SEQ ID NO: 128 - DNA, 20 bp, Homo sapiens
ttgaggccggccatggcgcg

SEQ ID NO: 129 - DNA, 20 bp, Homo sapiens
gaggccggccatggcgcgga

SEQ ID NO: 130 - DNA, 20 bp, Homo sapiens
ggccggccatggcgcggagc

SEQ ID NO: 131 - DNA, 20 bp, Homo sapiens
atcctccttaacatggttta

SEQ ID NO: 132 - DNA, 20 bp, Homo sapiens
cctccttaacatggtttaac

SEQ ID NO: 133 - DNA, 20 bp, Homo sapiens
tccttaacatggtttaacca

SEQ ID NO: 134 - DNA, 20 bp, Homo sapiens
aggcggaggaggacttgggg

SEQ ID NO: 135 - DNA, 20 bp, Homo sapiens
agaatgtttcatgtgataca

SEQ ID NO: 136 - DNA, 20 bp, Homo sapiens
aaagcccagctcattttgtt

SEQ ID NO: 137 - DNA, 20 bp, Homo sapiens
agcccagctcattttgttct

SEQ ID NO: 138 - DNA, 20 bp, Homo sapiens
cccagctcattttgttctgc

SEQ ID NO: 139 - DNA, 20 bp, Homo sapiens
gcgtcctctcccatggctag

SEQ ID NO: 140 - DNA, 20 bp, Homo sapiens
gtcctctcccatggctaggc

SEQ ID NO: 141 - DNA, 20 bp, Homo sapiens
cctctcccatggctaggcag

SEQ ID NO: 142 - DNA, 20 bp, Homo sapiens
ttcactcgccacatcttcaa

SEQ ID NO: 143 - DNA, 20 bp, Homo sapiens
cactcgccacatcttcaacc

SEQ ID NO: 144 - DNA, 20 bp, Homo sapiens
ctcgccacatcttcaaccag

SEQ ID NO: 145 - DNA, 20 bp, Homo sapiens
agagaatgtttcatgtgata

SEQ ID NO: 146 - DNA, 20 bp, Homo sapiens
agaatgtttcatgtgataca

SEQ ID NO: 147 - DNA, 20 bp, Homo sapiens
aatgtttcatgtgatacagg

SEQ ID NO: 148 - DNA, 20 bp, Homo sapiens
cttctggaagacatctctga

SEQ ID NO: 149 - DNA, 20 bp, Homo sapiens
tctggaagacatctctgaaa

SEQ ID NO: 150 - DNA, 20 bp, Homo sapiens
tggaagacatctctgaaatg

SEQ ID NO: 151 - DNA, 20 bp, Homo sapiens
atcccatgaaacatcctcta

SEQ ID NO: 152 - DNA, 20 bp, Homo sapiens
cccatgaaacatcctctagg

SEQ ID NO: 153 - DNA, 20 bp, Homo sapiens
catgaaacatcctctaggct

SEQ ID NO: 154 - DNA, 20 bp, Homo sapiens
ttgtccacgctcatggctcc

SEQ ID NO: 155 - DNA, 20 bp, Homo sapiens
gtccacgctcatggctcccg

SEQ ID NO: 156 - DNA, 20 bp, Homo sapiens
ccacgctcatggctcccgat

SEQ ID NO: 157 - DNA, 20 bp, Homo sapiens
ggaaactgagccatggttct

SEQ ID NO: 158 - DNA, 20 bp, Homo sapiens
aaactgagccatggttctgt

SEQ ID NO: 159 - DNA, 20 bp, Homo sapiens
actgagccatggttctgtta

SEQ ID NO: 160 - DNA, 20 bp, Homo sapiens
ccagggacactcatgtccct

SEQ ID NO: 161 - DNA, 20 bp, Homo sapiens
agggacactcatgtccctcc

SEQ ID NO: 162 - DNA, 20 bp, Homo sapiens
ggacactcatgtccctccag

SEQ ID NO: 163 - DNA, 20 bp, Homo sapiens
ctccagctctcgggtcagcc

SEQ ID NO: 164 - DNA, 20 bp, Homo sapiens
agtcaggatctcatatctct

SEQ ID NO: 165 - DNA, 20 bp, Homo sapiens
tcaggatctcatatctctgc

SEQ ID NO: 166 - DNA, 20 bp, Homo sapiens
aggatctcatatctctgccg

SEQ ID NO: 167 - DNA, 20 bp, Homo sapiens
tcataatggtccatgatggt

SEQ ID NO: 168 - DNA, 20 bp, Homo sapiens
ataatggtccatgatggtgc

SEQ ID NO: 169 - DNA, 20 bp, Homo sapiens
aatggtccatgatggtgcgc

SEQ ID NO: 170 - DNA, 20 bp, Homo sapiens
cactgagccgccatgactgc

SEQ ID NO: 171 - DNA, 20 bp, Homo sapiens
ctgagccgccatgactgcca

SEQ ID NO: 172 - DNA, 20 bp, Homo sapiens
gagccgccatgactgccact

SEQ ID NO: 173 - DNA, 20 bp, Homo sapiens
ccttgatgctacattcgttg

SEQ ID NO: 174 - DNA, 20 bp, Homo sapiens
ttgatgctacattcgttgtt

SEQ ID NO: 175 - DNA, 20 bp, Homo sapiens
gatgctacattcgttgttgg

SEQ ID NO: 176 - DNA, 20 bp, Homo sapiens
tctgaggaagacatggccca

SEQ ID NO: 177 - DNA, 20 bp, Homo sapiens
tgaggaagacatggcccagg

SEQ ID NO: 178 - DNA, 20 bp, Homo sapiens
tctgaggaagacatggccca

SEQ ID NO: 179 - DNA, 20 bp, Homo sapiens
tttcggtcgcacatggttac

SEQ ID NO: 180 - DNA, 20 bp, Homo sapiens
tcggtcgcacatggttaccg

SEQ ID NO: 181 - DNA, 20 bp, Homo sapiens
ggtcgcacatggttaccgtg

SEQ ID NO: 182 - DNA, 20 bp, Homo sapiens
ggcgatatcatcatccatgg

SEQ ID NO: 183 - DNA, 20 bp, Homo sapiens
cgatatcatcatccatggtg

SEQ ID NO: 184 - DNA, 20 bp, Homo sapiens
atatcatcatccatggtgag

SEQ ID NO: 185 - DNA, 20 bp, Homo sapiens
tcccaccctgacatgttggc

SEQ ID NO: 186 - DNA, 20 bp, Homo sapiens
ccaccctgacatgttggcta

SEQ ID NO: 187 - DNA, 20 bp, Homo sapiens
accctgacatgttggctact

SEQ ID NO: 188 - DNA, 20 bp, Homo sapiens
agtcgcagaggcatggtgcg

SEQ ID NO: 189 - DNA, 20 bp, Homo sapiens
tcgcagaggcatggtgcgtc

SEQ ID NO: 190 - DNA, 20 bp, Homo sapiens
gcagaggcatggtgcgtcgg

SEQ ID NO: 191 - DNA, 20 bp, Homo sapiens
actgcaattctcatggtagt

SEQ ID NO: 192 - DNA, 20 bp, Homo sapiens
tgcaattctcatggtagtga

SEQ ID NO: 193 - DNA, 20 bp, Homo sapiens
caattctcatggtagtgagt

SEQ ID NO: 194 - DNA, 20 bp, Homo sapiens
cgcatgttgtacatgacact

SEQ ID NO: 195 - DNA, 20 bp, Homo sapiens
catgttgtacatgacacttc

SEQ ID NO: 196 - DNA, 20 bp, Homo sapiens
tgttgtacatgacacttcca

SEQ ID NO: 197 - DNA, 20 bp, Homo sapiens
gttgccgaagtcatgctccc

SEQ ID NO: 198 - DNA, 20 bp, Homo sapiens
tgccgaagtcatgctccctc

SEQ ID NO: 199 - DNA, 20 bp, Homo sapiens
ccgaagtcatgctccctctg

SEQ ID NO: 200 - DNA, 20 bp, Homo sapiens
cggtagataatcatgatggc

SEQ ID NO: 201 - DNA, 20 bp, Homo sapiens
gtagataatcatgatggcga

SEQ ID NO: 202 - DNA, 20 bp, Homo sapiens
agataatcatgatggcgact

SEQ ID NO: 203 - DNA, 20 bp, Homo sapiens
aaccgcattttcattctgga

SEQ ID NO: 204 - DNA, 20 bp, Homo sapiens
ccgcattttcattctggagc

SEQ ID NO: 205 - DNA, 20 bp, Homo sapiens
gcattttcattctggagctg

SEQ ID NO: 206 - DNA, 20 bp, Homo sapiens
ctcggcgggtccatcgccag

SEQ ID NO: 207 - DNA, 20 bp, Homo sapiens
cggcgggtccatcgccagct

SEQ ID NO: 208 - DNA, 20 bp, Homo sapiens
gcgggtccatcgccagctgc

SEQ ID NO: 209 - DNA, 20 bp, Homo sapiens
agccggtcccgcatccccgc

SEQ ID NO: 210 - DNA, 20 bp, Homo sapiens
ccggtcccgcatccccgccg

SEQ ID NO: 211 - DNA, 20 bp, Homo sapiens
ggtcccgcatccccgccggc

SEQ ID NO: 212 - DNA, 20 bp, Homo sapiens
ttctgcgctgccatgtctgc

SEQ ID NO: 213 - DNA, 20 bp, Homo sapiens
ctgcgctgccatgtctgcgt

SEQ ID NO: 214 - DNA, 20 bp, Homo sapiens
gcgctgccatgtctgcgtcg

SEQ ID NO: 215 - DNA, 20 bp, Homo sapiens
ccgagtgaggccatccttca

SEQ ID NO: 216 - DNA, 20 bp, Homo sapiens
gagtgaggccatccttcagc

SEQ ID NO: 217 - DNA, 20 bp, Homo sapiens
gtgaggccatccttcagctg

SEQ ID NO: 218 - DNA, 20 bp, Homo sapiens
accgcagctttcatcctgaa

SEQ ID NO: 219 - DNA, 20 bp, Homo sapiens
cgcagctttcatcctgaagg

SEQ ID NO: 220 - DNA, 20 bp, Homo sapiens
cagctttcatcctgaagggc

SEQ ID NO: 221 - DNA, 20 bp, Homo sapiens
ataatcggctcagcatttct

SEQ ID NO: 222 - DNA, 20 bp, Homo sapiens
aatcggctcagcatttctag

SEQ ID NO: 223 - DNA, 20 bp, Homo sapiens
tcggctcagcatttctagaa

SEQ ID NO: 224 - DNA, 20 bp, Homo sapiens
gctccgttctccatcgcctc

SEQ ID NO: 225 - DNA, 20 bp, Homo sapiens
tccgttctccatcgcctcgg

SEQ ID NO: 226 - DNA, 20 bp, Homo sapiens
cgttctccatcgcctcgggc

SEQ ID NO: 227 - DNA, 20 bp, Homo sapiens
ccactttggtccatggtgcc

SEQ ID NO: 228 - DNA, 20 bp, Homo sapiens
actttggtccatggtgcccc

SEQ ID NO: 229 - DNA, 20 bp, Homo sapiens
tttggtccatggtgccccca

SEQ ID NO: 230 - DNA, 20 bp, Homo sapiens
gttagcacctgcatggttgc

SEQ ID NO: 231 - DNA, 20 bp, Homo sapiens
tagcacctgcatggttgctt

SEQ ID NO: 232 - DNA, 20 bp, Homo sapiens
gcacctgcatggttgcttcc

SEQ ID NO: 233 - DNA, 20 bp, Homo sapiens
cgggccgctgtggcctgtga

SEQ ID NO: 234 - DNA, 20 bp, Homo sapiens
ttgggggtggctacacagcc

SEQ ID NO: 235 - DNA, 20 bp, Homo sapiens
tgggtacacccctctaacca

SEQ ID NO: 236 - protein, 20 aa, Homo sapiens
NEFCASCPTFLRMDNNAAAI

SEQ ID NO: 237 - protein, 20 aa, Homo sapiens
FQVKLETRQITWSRGADKIE

SEQ ID NO: 238 - protein, 50 aa, Homo sapiens
GCGPGAPSDAEVLHLCRSLEVGTVMTLFYSKKSQRPERKTFQVKLETRQI

SEQ ID NO: 239 - protein, 20 aa, Homo sapiens
YTVWHFRRVLLKSLQKDLHE

SEQ ID NO: 240 - protein, 20 aa, Homo sapiens
QYTLEMIKLVPHNESAWNYL

SEQ ID NO: 241 - protein, 50 aa, Homo sapiens
LIAFLVDIYEDMLENQCDNKEDILNKALELCEILAKEKDTIRKEYWRYIG

SEQ ID NO: 242 - protein, 20 aa, Homo sapiens
MLGEYDWALQANIKAQKLCK

SEQ ID NO: 243 - protein, 20 aa, Homo sapiens
WPMLSIFFTEYKYHITKIVM

SEQ ID NO: 244 - protein, 50 aa, Homo sapiens
EFSKERFDIAIIYYTRAIEYRPENYLLYGNRALCFLRTGQFRNALGDGKR

SEQ ID NO: 245 - protein, 20 aa, Homo sapiens
VNKAGNDLIESSAGEEASNL

SEQ ID NO: 246 - protein, 20 aa, Homo sapiens
EKYVAADTLYSQIKEDVKKR

SEQ ID NO: 247 - protein, 50 aa, Homo sapiens
AWDSLNKAWKDRIDKLEEAMQAAVQYQDGLQAVFDWVDIAGGKLASMSPI

SEQ ID NO: 248 - protein, 20 aa, Homo sapiens
LEIDELQKRLGSVAVKREQR

SEQ ID NO: 249 - protein, 20 aa, Homo sapiens
AYEHFHRGHDHVLQFLVSIP

SEQ ID NO: 250 - protein, 50 aa, Homo sapiens
LIERCDLEIYQLKKEIQALKDTKPQVQTKEVVQEILQFQEDPQTKEEVAS

SEQ ID NO: 251 - protein, 20 aa, Homo sapiens
GKVRKAASSIDQFSIRLGIF

SEQ ID NO: 252 - protein, 20 aa, Homo sapiens
LRRYPIARVFVIIYMALLHL

SEQ ID NO: 253 - protein, 50 aa, Homo sapiens
EKNSLVFQLERLEQQMNSASGSSSNGSSINMSGIDNGEGTRLRNVPVLFN

SEQ ID NO: 254 - protein, 20 aa, Homo sapiens
ESNTAGLDIFAKFSAYIKNS

SEQ ID NO: 255 - protein, 20 aa, Homo sapiens
NAYAREEFASTCPDDEEIEL

SEQ ID NO: 256 - protein, 50 aa, Homo sapiens
VDETSAEDEGVSQRKFLDGNELTLADCNLLPKLHIVQVVCKKYRGFTIPE

SEQ ID NO: 257 - protein, 20 aa, Homo sapiens
VDAFNQAWHLVAHECPNYFR

SEQ ID NO: 258 - protein, 20 aa, Homo sapiens
LQCPQVDLFYLHTPDHGTPV

SEQ ID NO: 259 - protein, 50 aa, Homo sapiens
ELFPCLRHFGLRFYAYNPLAGGLLTGKYKYEDKDGKQPVGRFFGNSWAET

SEQ ID NO: 260 - protein, 20 aa, Homo sapiens
HAWQHRQWVIQEFKLWDNEL

SEQ ID NO: 261 - protein, 20 aa, Homo sapiens
DMLENQCDNKEDILNKALEL

SEQ ID NO: 262 - protein, 50 aa, Homo sapiens
AQGGEPGQPAQPPPQPHPPPPQQQHKEEMAAEAGEAVASPMDDGFVSLDS

SEQ ID NO: 263 - protein, 20 aa, Homo sapiens
EKYVAADTLYSQIKEDVKKR

SEQ ID NO: 264 - protein, 20 aa, Homo sapiens
LALGQFQHALDELLAWLTHT

SEQ ID NO: 265 - protein, 50 aa, Homo sapiens
QQEAAETIREEIDGLQEELDIVINLGSELIAACGEPDKPIVKKSIDELNS

SEQ ID NO: 266 - protein, 20 aa, Homo sapiens
EILPEERMNGQVVCAVPHNL

SEQ ID NO: 267 - protein, 20 aa, Homo sapiens
VIFEDISIEHFEGTVTKVIP

SEQ ID NO: 268 - protein, 50 aa, Homo sapiens
QRNHAIRIKKLPKGTVSFHSHSDHRFLGTVEKEATFSNPKTTSPNKGKEK

SEQ ID NO: 269 - protein, 20 aa, Homo sapiens
VQELQNLLKGKEEQMNTMKA

SEQ ID NO: 270 - protein, 20 aa, Homo sapiens
LFKSQIEQLKQQNYQQASSF

SEQ ID NO: 271 - protein, 50 aa, Homo sapiens
EENESLKAHVQEVAQHNLKEASSASQFEELEIVLKEKGNELKRLEAMLKE

SEQ ID NO: 272 - protein, 20 aa, Homo sapiens
KAAGALLTEGEACHMSLSSP

SEQ ID NO: 273 - protein, 20 aa, Homo sapiens
AEEAQIDDEAHPVLLQPVAK

SEQ ID NO: 274 - protein, 50 aa, Homo sapiens
DILIPNVLLSQEKNAVLGLPVALQDKAVTDPQGVGTPEMIPLDWEKGKLE

SEQ ID NO: 275 - protein, 20 aa, Homo sapiens
EKAADESERGMKVIENRAMK

SEQ ID NO: 276 - protein, 20 aa, Homo sapiens
EADRKYEEVARKLVILEGEL

SEQ ID NO: 277 - protein, 20 aa, Homo sapiens
EVCAQRDAYQQKLVQLQEKC

SEQ ID NO: 278 - protein, 20 aa, Homo sapiens
LALQAQITALTKQNEQHIKE

SEQ ID NO: 279 - protein, 50 aa, Homo sapiens
KSYQEELDKLRQLLKKTRVSTDQAAAEQLSLVQAELQTQWEAKCEHLLAS

SEQ ID NO: 280 - protein, 20 aa, Homo sapiens
PAVQPEESLKTDHPEIGEGK

SEQ ID NO: 281 - protein, 20 aa, Homo sapiens
NAKKKEVAGAKPHITAAEGK

SEQ ID NO: 282 - protein, 50 aa, Homo sapiens
PTPALSEEASSSSIRERPPEEVAARLAQQEKQEQVKIESLAKSLEDALRQ

SEQ ID NO: 283 - protein, 20 aa, Homo sapiens
TIRTPRLCPHPLLRPPPSAA

SEQ ID NO: 284 - protein, 20 aa, Homo sapiens
IKRLEEKQSPELVKKHKKKR

SEQ ID NO: 285 - protein, 50 aa, Homo sapiens
PQAILCHPSLQPEEYMAYVQRQADSKQYGDKIIEELQDLGPQVWSETKSG

SEQ ID NO: 286 - protein, 20 aa, Homo sapiens
KDYYQLQAILEAERRDRGHD

SEQ ID NO: 287 - protein, 20 aa, Homo sapiens
AVRLRKSHTEMSKSISQLES

SEQ ID NO: 288 - protein, 50 aa, Homo sapiens
VSQIEKEKMLLQHRINEYQRKAEQENEKRRNVENEVSTLKDQLEDLKKVS

SEQ ID NO: 289 - protein, 20 aa, Homo sapiens
ASDHQKSLEDLKATLNSGPG

SEQ ID NO: 290 - protein, 20 aa, Homo sapiens
RHSPGPERDLSREVHKAEWR

SEQ ID NO: 291 - protein, 50 aa, Homo sapiens
ESLREKLLVAENRLQAVEALCSSQHTHMIESNDISEETIRTKETVEGLQD

SEQ ID NO: 292 - protein, 20 aa, Homo sapiens
KVQSGSESVIQEYVDLRTHY

SEQ ID NO: 293 - protein, 20 aa, Homo sapiens
QLAEAHAQAKAQAEREAKEL

SEQ ID NO: 294 - protein, 50 aa, Homo sapiens
QLRQSSEAEIQAKARQAEAAERSRLRIEEEIRVVRLQLEATERQRGGAEG

SEQ ID NO: 295 - protein, 20 aa, Homo sapiens
GSRGAPQHYPKTAGNSEFLG

SEQ ID NO: 296 - protein, 20 aa, Homo sapiens
KALEEPKYSSLYAQLCLRLA

SEQ ID NO: 297 - protein, 50 aa, Homo sapiens
NSAANNSANEKERHDAIFRKVRGILNKLTPEKFDKLCLELLNVGVESKLI

SEQ ID NO: 298 - protein, 20 aa, Homo sapiens
GPGVELLLTPREPALPLEPD

SEQ ID NO: 299 - protein, 20 aa, Homo sapiens
AALGQTEQLPAEAPWARRPV

SEQ ID NO: 300 - protein, 50 aa, Homo sapiens
LRSPQEEALQRLVNLYGLLHGLQAAVAQQDTLMEARFPEGPERREKLCRA

SEQ ID NO: 301 - protein, 20 aa, Homo sapiens
LEEHAKLQAAVDGPMDKKEE

SEQ ID NO: 302 - protein, 50 aa, Homo sapiens
LKADLQKLKDELASTKQKLEKAENQVLAMRKQSEGLTKEYDRLLEEHAKL

SEQ ID NO: 303 - protein, 20 aa, Homo sapiens
EAAGAPAPAPLAGQKPPADA

SEQ ID NO: 304 - protein, 20 aa, Homo sapiens
LVQRELQELQTIKHHVLQQQ

SEQ ID NO: 305 - protein, 50 aa, Homo sapiens
APGGGSGALSRPGFEKEEASQEERQRKQQEQLLQLERERVELEKLRQLRL

SEQ ID NO: 306 - protein, 20 aa, Homo sapiens
RALEYTIYNQELNETRAKLD

SEQ ID NO: 307 - protein, 20 aa, Homo sapiens
QKLLEKIEEKQKELAETEPK

SEQ ID NO: 308 - protein, 50 aa, Homo sapiens
QRTKLELKAKDLQDELAGNSEQRKRLLKERQKLLEKIEEKQKELAETEPK

SEQ ID NO: 309 - protein, 20 aa, Homo sapiens
SFNPEQLKKLQDKIEKCKQD

SEQ ID NO: 310 - protein, 20 aa, Homo sapiens
TLSRREKKKATDGFTLTGIN

SEQ ID NO: 311 - protein, 50 aa, Homo sapiens
VQKHLNLSNVAGYKAIYHDLEQSIRAADAVEDLRWFRANHGPGMAMNWPQ

SEQ ID NO: 312 - protein, 20 aa, Homo sapiens
DEAERVLFSYGYGANVPTTA

SEQ ID NO: 313 - protein, 20 aa, Homo sapiens
EDNIFTPKPTLFTKKEAPEW

SEQ ID NO: 314 - protein, 50 aa, Homo sapiens
KERAVMAEKDALEKHDQLLDRYRELQLSTESKVTEFLHQSKLKSFESERV

SEQ ID NO: 315 - protein, 20 aa, Homo sapiens
NELDYYDSHNVNTRCQKICD

SEQ ID NO: 316 - protein, 20 aa, Homo sapiens
SFKVLAGDKNFITAEELRRE

SEQ ID NO: 317 - protein, 50 aa, Homo sapiens
VGPWIQTKMEEIGRISIEMNGTLEDQLSHLKQYERSIVDYKPNLDLLEQQ

SEQ ID NO: 318 - protein, 20 aa, Homo sapiens
AHMSVRKSTGDSQNLGSSSP

SEQ ID NO: 319 - protein, 20 aa, Homo sapiens
LRKQLASRYASVEKARKALT

SEQ ID NO: 320 - protein, 50 aa, Homo sapiens
LAKLSQNSLASQEEGSLGEAWAQVKKSLADEAEVHLKFSAKLHSEVEKPL

SEQ ID NO: 321 - protein, 20 aa, Homo sapiens
AILMENTPGSENHEDIELLQ

SEQ ID NO: 322 - protein, 20 aa, Homo sapiens
EVYLDLVKKGVQFPPSEAEA

SEQ ID NO: 323 - protein, 50 aa, Homo sapiens
LTLSLIDMCVQNCGPSFQSLIVKKEFVKENLVKLLNPRYNLPLDIQNRIL

SEQ ID NO: 324 - protein, 20 aa, Homo sapiens
MSVPGPYQAATGPSSAPSAP

SEQ ID NO: 325 - protein, 20 aa, Homo sapiens
SYYPTPPAPMPGPTTGLVTG

SEQ ID NO: 326 - protein, 50 aa, Homo sapiens
YYTQPAPIPNNNPITVQTVYVQHPITFLDRPIQMCCPSCNKMIVSQLSYN

SEQ ID NO: 327 - protein, 20 aa, Homo sapiens
CNIMQEAVVQYEQYEQEMKH

SEQ ID NO: 328 - protein, 50 aa, Homo sapiens
HEELAQKIKGYQEQIASLNSKCKMLTMKAKHATMLLTVTEVEGLAEGTED

SEQ ID NO: 329 - protein, 20 aa, Homo sapiens
NLPLPIPTVDASIPTSQNGF

SEQ ID NO: 330 - protein, 50 aa, Homo sapiens
VPDAFPELSELSVSQLTDMNEQEEVLLEQFLTLPQLKQIITDKDDLVKSI

SEQ ID NO: 331 - protein, 20 aa, Homo sapiens
DPPQPNHSIHTGMVPQGTKV

SEQ ID NO: 332 - protein, 20 aa, Homo sapiens
SKGTPGRFDPRRELEAEIYR

SEQ ID NO: 333 - protein, 50 aa, Homo sapiens
PENRDISEDQSSAEQTQALASQASQFLAKVESFERLIQAGRLMPQDQVKG

SEQ ID NO: 334 - protein, 20 aa, Homo sapiens
MISLKNGVGTSSSMGSGVSD

SEQ ID NO: 335 - protein, 20 aa, Homo sapiens
AEAVKEKWLPYEAGQRFLEF

SEQ ID NO: 336 - protein, 50 aa, Homo sapiens
KISITEGIERGIVDSITGQRLLEAQACTGGIIHPTTGQKLSLQDAVSQGV

SEQ ID NO: 337 - protein, 20 aa, Homo sapiens
QYPYPSGFPPMGGGAYPQVP

SEQ ID NO: 338 - protein, 20 aa, Homo sapiens
LVSMCQGNRDENQSINHQMA

SEQ ID NO: 339 - protein, 50 aa, Homo sapiens
ANRSNDQRQKIKAAFKTSYGKDLIKDLKSELSGNMEELILALFMPPTYYD

SEQ ID NO: 340 - protein, 20 aa, Homo sapiens
QMSEQVHTLREEKECSMSRV

SEQ ID NO: 341 - protein, 20 aa, Homo sapiens
TTISRALSQNRELKEQLAEL

SEQ ID NO: 342 - protein, 50 aa, Homo sapiens
EPPAGPSEVEQQLQAEAEHLRKELEGLAGQLQAQVQDNEGLSRLNREQEE

SEQ ID NO: 343 - protein, 20 aa, Homo sapiens
AKFREKDEREEQLIKAKEKL

SEQ ID NO: 344 - protein, 50 aa, Homo sapiens
DALRKASSEGKSEMKKLRQQLEAAEKQIKHLEIEKNAESSKASSITRELQ

SEQ ID NO: 345 - protein, 20 aa, Homo sapiens
PSVSRTSFTSVSRSGGGGGG

SEQ ID NO: 346 - protein, 20 aa, Homo sapiens
INKRTTAENEFVMLKKDVDA

SEQ ID NO: 347 - protein, 50 aa, Homo sapiens
MFFDAELSQMQTHVSDTSVVLSMDNNRNLDLDSIIAEVKAQYEEIANRSR

SEQ ID NO: 348 - protein, 20 aa, Homo sapiens
IAEINRMIQRLRSEIDHVKK

SEQ ID NO: 349 - protein, 20 aa, Homo sapiens
DAEQRGEMALKDAKNKLEGL

SEQ ID NO: 350 - protein, 20 aa, Homo sapiens
ELEAALQRAKQDMARQLREY

SEQ ID NO: 351 - protein, 20 aa, Homo sapiens
TSSSRAVVVKKIETRDGKLV

SEQ ID NO: 352 - protein, 50 aa, Homo sapiens
LEGEESRLESGMQNMSIHTKTTGGYAGGLSSAYGGLASPGLSYSLGSSFG

SEQ ID NO: 353 - protein, 20 aa, Homo sapiens
DVPGDENAEMDARTILLNTK

SEQ ID NO: 354 - protein, 20 aa, Homo sapiens
SLKYTAARLHEKGVLLEIED

SEQ ID NO: 355 - protein, 50 aa, Homo sapiens
RNQRRYRQRRKAELVKLQQTYAALNSKATFYGEQVDYYKSYIKTCLDNLA

SEQ ID NO: 356 - protein, 20 aa, Homo sapiens
QKGKAAILDLEKTCKELKHQ

SEQ ID NO: 357 - protein, 20 aa, Homo sapiens
TQAAQELAAEKEKISVLQNN

SEQ ID NO: 358 - protein, 50 aa, Homo sapiens
EQKELKKSLEKEKEASHQLKLELNSMQEQLIQAQNTLKQNEKEEQQLQGN

SEQ ID NO: 359 - protein, 20 aa, Homo sapiens
VEHQLEEEKKANNEKQKAER

SEQ ID NO: 360 - protein, 20 aa, Homo sapiens
REKRLQQLAEPQSDLEELKH

SEQ ID NO: 361 - protein, 50 aa, Homo sapiens
LERSLSRLKNQMAEPLPPDAPAVSSEVELQDLRKELERVAGELQAQVENN

SEQ ID NO: 362 - protein, 20 aa, Homo sapiens
CKSELERSQQAAQSADVSLN

SEQ ID NO: 363 - protein, 20 aa, Homo sapiens
TMNHRDIARHQASSSVFSWQ

SEQ ID NO: 364 - protein, 50 aa, Homo sapiens
LQGHEKEMKGQVNKFQELQLQLEKAKVELIEKEKVLNKCRDELVRTTAQY

SEQ ID NO: 365 - protein, 20 aa, Homo sapiens
QRQISLEGSVKGIQNDLTKL

SEQ ID NO: 366 - protein, 20 aa, Homo sapiens
DEEALEDSAEEKVEESRAEK

SEQ ID NO: 367 - protein, 50 aa, Homo sapiens
VSAHTRAVKERMDRQCAQVKRLENNHAQLLRRNHFKVLIFQEENEIPASV

SEQ ID NO: 368 - protein, 20 aa, Homo sapiens
SQHTHCAQDLAMKDELLCQL

SEQ ID NO: 369 - protein, 20 aa, Homo sapiens
LCSLLQESKEEAIRTLQRKI

SEQ ID NO: 370 - protein, 50 aa, Homo sapiens
DTVENLTAKLASTIADNQEQDLEKTRQYSQKLGLLTEQLQSLTLFLQTKL

SEQ ID NO: 371 - protein, 20 aa, Homo sapiens
ADHSKSDRMTRGLRAQVDDL

SEQ ID NO: 372 - protein, 20 aa, Homo sapiens
EIQKLMGQIHQLRSELQDME

SEQ ID NO: 373 - protein, 50 aa, Homo sapiens
LKREQESYKQMQSEFAARLNKVEMERQNLAEAITLAERKYSDEKKRVDEL

SEQ ID NO: 374 - protein, 20 aa, Homo sapiens
GHVTQLKESLKEVQLERDQY

SEQ ID NO: 375 - protein, 20 aa, Homo sapiens
RLREQEERLQEQQERLREQE

SEQ ID NO: 376 - protein, 50 aa, Homo sapiens
HDTHRVEELERSLSRLKNQMAEPLPPDAPAVSSEVELQDLRKELERVAGE

SEQ ID NO: 377 - protein, 20 aa, Homo sapiens
SKSETTLEKLKGEIAHLKTS

SEQ ID NO: 378 - protein, 20 aa, Homo sapiens
KIFELEKKTETAAHSLPQQT

SEQ ID NO: 379 - protein, 50 aa, Homo sapiens
EKNAYQLTEKDKEIQRLRDQLKARYSTTALLEQLEETTREGERREQVLKA

SEQ ID NO: 380 - protein, 20 aa, Homo sapiens
IGAPGNKPELYEEVKLYKNA

SEQ ID NO: 381 - protein, 20 aa, Homo sapiens
VRQMLFDLESAYNAFNRFLH

SEQ ID NO: 382 - protein, 50 aa, Homo sapiens
DKGNLNRCIADVVSLFITVMDKLRLEIRAMDEIQPDLRELMETMHRMSHL

SEQ ID NO: 383 - protein, 20 aa, Homo sapiens
RQSSERAGQLEATLTSCRRR

SEQ ID NO: 384 - protein, 20 aa, Homo sapiens
TSTPVDNLAAEILPAELRET

SEQ ID NO: 385 - protein, 50 aa, Homo sapiens
RQEELQRHLEDANRARHGLETQHRLNQQQLSELRAQVEDLQKALQEQGGK

SEQ ID NO: 386 - protein, 20 aa, Homo sapiens
ALLEQQRKEQERLAQLERAE

SEQ ID NO: 387 - protein, 20 aa, Homo sapiens
REIHNKQQLQKQKSMEAERL

SEQ ID NO: 388 - protein, 50 aa, Homo sapiens
ELEALNDKKHQLEGKLQDIRCRLTTQRQEIESTNKSRELRIAEITHLQQQ

SEQ ID NO: 389 - protein, 20 aa, Homo sapiens
EAGEPTPGLSDTSPDEGLIE

SEQ ID NO: 390 - protein, 20 aa, Homo sapiens
QLAEGLLSHYLPDLQRSKQA

SEQ ID NO: 391 - protein, 20 aa, Homo sapiens
SGWENSPLQDDRKIQLHSSQ

SEQ ID NO: 392 - protein, 20 aa, Homo sapiens
YSSSRTPSISPVRVSPNNRS

SEQ ID NO: 393 - protein, 50 aa, Homo sapiens
LWHPSIVKPYLTLLSECSNPDTLEGAAGALQNLAAGSWKWSVYIRAAVRK

SEQ ID NO: 394 - protein, 20 aa, Homo sapiens
LTTIARTINEVENQILTRDA

SEQ ID NO: 395 - protein, 20 aa, Homo sapiens
IARMAPYTGPDSVPGALDYM

SEQ ID NO: 396 - protein, 50 aa, Homo sapiens
EFARIMSIVDPNRLGVVTFQAFIDFMSRETADTDTADQVMASFKILAGDK

SEQ ID NO: 397 - protein, 20 aa, Homo sapiens
QSACNLEKKQKKFDQLLAEE

SEQ ID NO: 398 - protein, 20 aa, Homo sapiens
AKLRLEVNLQAMKAQFERDL

SEQ ID NO: 399 - protein, 50 aa, Homo sapiens
ELERLNKQFRTEMEDLMSSKDDVGKSVHELEKSKRALEQQVEEMKTQLEE

SEQ ID NO: 400 - protein, 20 aa, Homo sapiens
ADPPATEKTLLEEKVKLEEQ

SEQ ID NO: 401 - protein, 50 aa, Homo sapiens
EEDMGQSEQKADPPATEKTLLEEKVKLEEQLKETVEKYKRALADTENLRQ

SEQ ID NO: 402 - protein, 20 aa, Homo sapiens
KLPVEISGAIEEEFTVARLY

SEQ ID NO: 403 - protein, 20 aa, Homo sapiens
LQMESHREAHHRQLARLRDE

SEQ ID NO: 404 - protein, 50 aa, Homo sapiens
VVKRCRQLENLQVECHRKMEVTGRELSSCQLLISQHEAKIRSLTEYMQSV

SEQ ID NO: 405 - protein, 20 aa, Homo sapiens
QDRVQNLRKDFTELQKTVKE

SEQ ID NO: 406 - protein, 20 aa, Homo sapiens
KEALAGLLVTYPNSQEAENW

SEQ ID NO: 407 - protein, 50 aa, Homo sapiens
EEVHKEANSVLQWLESKEEVLKSMDAMSSPTKTETVKAQAESNKAFLAEL

SEQ ID NO: 408 - protein, 20 aa, Homo sapiens
NFGSYVTHETKHFIYFYLGQ

SEQ ID NO: 409 - protein, 50 aa, Homo sapiens
MCDRKAVIKNADMSEEMQQDSVECATQALEKYNIEKDIAAHIKKEFDKKY

SEQ ID NO: 410 - protein, 20 aa, Homo sapiens
ANLFSDVQFKMSHKRIMLFT

SEQ ID NO: 411 - protein, 20 aa, Homo sapiens
EESLVIGSSTLFSALLIKCL

SEQ ID NO: 412 - protein, 50 aa, Homo sapiens
DLLRKVRAKETRKRALSRLKLKLNKDIVISVGIYNLVQKALKPPPIKLYR

SEQ ID NO: 413 - protein, 20 aa, Homo sapiens
AKQYPLVTPNEERNVMEEGK

SEQ ID NO: 414 - protein, 50 aa, Homo sapiens
QELDGKPASPTPVIVASHTANKEEKSLLELEVDLDNLELEDIDTTDINLD

SEQ ID NO: 415 - protein, 20 aa, Homo sapiens
MRIAVICFCLLGITCAIPVK

SEQ ID NO: 416 - protein, 50 aa, Homo sapiens
LGITCAIPVKQADSGSSEEKQLYNKYPDAVATWLNPDPSQKQNLLAPQTL

SEQ ID NO: 417 - protein, 20 aa, Homo sapiens
EVDERDQDDEEARREMARIL

SEQ ID NO: 418 - protein, 20 aa, Homo sapiens
TFEEPVTHVSESIGIMEVKV

SEQ ID NO: 419 - protein, 50 aa, Homo sapiens
RRGGDLTNTVFVDFRTEDGTANAGSDYEFTEGTVVFKPGDTQKEIRVGII

SEQ ID NO: 420 - protein, 20 aa, Homo sapiens
ESQDLKEELVAAVEDVRKQG

SEQ ID NO: 421 - protein, 20 aa, Homo sapiens
ADMADVMRLLSHLKIVEEAL

SEQ ID NO: 422 - protein, 50 aa, Homo sapiens
MTSATSPIILKWDPKSLEIRTLTVERLLEPLVTQVTTLVNTSNKGPSGKK

SEQ ID NO: 423 - protein, 20 aa, Homo sapiens
NPDGMVALLDYREDGVTPYM

SEQ ID NO: 424 - protein, 20 aa, Homo sapiens
DIVMNHHLQETSFTKEAYKK

SEQ ID NO: 425 - protein, 50 aa, Homo sapiens
DEMFSDIYKIREIADGLCLEVEGKMVSRTEGNIDDSLIGGNASAEGPEGE

SEQ ID NO: 426 - protein, 20 aa, Homo sapiens
ISYWGFPSEEYLVETEDGYI

SEQ ID NO: 427 - protein, 20 aa, Homo sapiens
TTHSPAGTSVQNMLHWSQAV

SEQ ID NO: 428 - protein, 50 aa, Homo sapiens
RGNTWSRKHKTLSVSQDEFWAFSYDEMAKYDLPASINFILNKTGQEQVYY

SEQ ID NO: 429 - protein, 20 aa, Homo sapiens
REQLQDLHDQIAGQKASKQE

SEQ ID NO: 430 - protein, 20 aa, Homo sapiens
VIIYMALLHLWVMIVLLTYT

SEQ ID NO: 431 - protein, 50 aa, Homo sapiens; identical to SEQ ID NO: 253

SEQ ID NO: 432 - protein, 20 aa, Homo sapiens
AIATKKIISDYHQQFRYKLQ

SEQ ID NO: 433 - protein, 20 aa, Homo sapiens
YELEEKIVSLIKNLLVALKD

SEQ ID NO: 434 - protein, 50 aa, Homo sapiens
KFTYLINYIQDEINTIFNDYIPYVFKLLKENLCLNLHKFNEFIQNELQEA

SEQ ID NO: 435 - protein, 20 aa, Homo sapiens
DDELEEMLESGKPSIFTSDI

SEQ ID NO: 436 - protein, 20 aa, Homo sapiens
DYVEHAKEETKKAIKYQSKA

SEQ ID NO: 437 - protein, 50 aa, Homo sapiens
ISDSQITRQALNEIESRHKDIMKLETSIRELHEMFMDMAMFVETQGEMIN

SEQ ID NO: 438 - protein, 20 aa, Homo sapiens
AEAEGLSGTTLLPKLIPSGA

SEQ ID NO: 439 - protein, 20 aa, Homo sapiens
AVFWVLGATLVVIGSHAAFH

SEQ ID NO: 440 - protein, 50 aa, Homo sapiens
VEYYQSNYVFVFLGLILYCVVTSPMLLVALAVFFGACYILYLRTLESKLV

SEQ ID NO: 441 - protein, 20 aa, Homo sapiens
PALEAHCVEFLTKHLRADNA

SEQ ID NO: 442 - protein, 50 aa, Homo sapiens
FMLLTQARLFDEPQLASLCLDTIDKSTMDAISAEGFTDIDIDTLCAVLER

SEQ ID NO: 443 - protein, 20 aa, Homo sapiens
KETEGLRQEMSKDLEEVKAK

SEQ ID NO: 444 - protein, 20 aa, Homo sapiens
GLLPVLESFKVSFLSALEEY

SEQ ID NO: 445 - protein, 50 aa, Homo sapiens
SDELRQRLAARLEALKENGGARLAEYHAKATEHLSTLSEKAKPALEDLRQ

SEQ ID NO: 446 - protein, 20 aa, Homo sapiens
LKVQYQRQASPETSASPDGS

SEQ ID NO: 447 - protein, 20 aa, Homo sapiens
QQLCNTRQEVNELRKLLEEE

SEQ ID NO: 448 - protein, 50 aa, Homo sapiens
LEHSEWDSSRTPIIGSCGTQEQALLIDLTSNSCRRTRSGVGWKRVLRSLC

SEQ ID NO: 449 - protein, 20 aa, Homo sapiens
AEAEGLSGTTLLPKLIPSGA

SEQ ID NO: 450 - protein, 20 aa, Homo sapiens
VVIGSHAAFHQIEAVDGEEL

SEQ ID NO: 451 - protein, 50 aa, Homo sapiens; identical to SEQ ID NO: 440

SEQ ID NO: 452 - protein, 20 aa, Homo sapiens
RLEQQMNSASGSSSNGSSIN

SEQ ID NO: 453 - protein, 20 aa, Homo sapiens
VIIYMALLHLWVMIVLLTYT

SEQ ID NO: 454 - protein, 50 aa, Homo sapiens
EFHYIEEDLYRTKNTLQSRIKDRDEEIQKLRNQLTNKTLSNSSQSELENR

SEQ ID NO: 455 - protein, 20 aa, Homo sapiens
TEEDPGPARGPRSGLAAYFF

SEQ ID NO: 456 - protein, 20 aa, Homo sapiens
LSLLILIVYCTPFYERVDTT

SEQ ID NO: 457 - protein, 50 aa, Homo sapiens
TSAEIAAIVFGFIASFMFLLDFITMLYEKRQESQLRKPENTTRAEALTEP

SEQ ID NO: 458 - protein, 20 aa, Homo sapiens
RLLPDHLQLKDILRKQDEYH

SEQ ID NO: 459 - protein, 20 aa, Homo sapiens
LRQRTLPQAQTDQAQSHQFP

SEQ ID NO: 460 - protein, 50 aa, Homo sapiens
PVYPAFSPLQMLWWQQMYAHQYYMQYQAAVSAQATSNVNPTQPTTSQPLN

SEQ ID NO: 461 - protein, 20 aa, Homo sapiens
QLSGPGGQAQAEAPDLYTYF

SEQ ID NO: 462 - protein, 20 aa, Homo sapiens
HHILMHLTEKAQEVTRKYQE

SEQ ID NO: 463 - protein, 50 aa, Homo sapiens
TMLKAICVDVDHGLLPREEWQAKVAGSEENGTAETEEVEDESASGELDLE

SEQ ID NO: 464 - protein, 20 aa, Homo sapiens
VKLAPDFDKIVESLSLLKDF

SEQ ID NO: 465 - protein, 20 aa, Homo sapiens
INTVTSGMAPDTKETIFSIS

SEQ ID NO: 466 - protein, 50 aa, Homo sapiens
VVGDQSAGKTSVLEMIAQARIFPRGSGEMMTRSPVKVTLSEGPHHVALFK

SEQ ID NO: 467 - protein, 20 aa, Homo sapiens
IEHGIVTNWDDMEKIWHHTF

SEQ ID NO: 468 - protein, 22 aa, Homo sapiens
QAVLSLYASGRTTGIVMDSGDG

SEQ ID NO: 469 - protein, 50 aa, Homo sapiens
GQVITIGNERFRCPEALFQPSFLGMESCGIHETTFNSIMKCDVDIRKDLY

SEQ ID NO: 470 - DNA, 897 bp, Homo sapiens
  CDS (39)..(842)
agagactgcgagaaggaggtcccccacggcccttcaggatgaaagctgcggtgctgaccttggccgtgctcttcctgacggggagccaggctcggcatttctggcagcaagatgaacccccccagagcccctgggatcgagtgaaggacctggccactgtgtacgtggatgtgctcaaagacagcggcagagactatgtgtcccagtttgaaggctccgccttgggaaaacagctaaacctaaagctccttgacaactgggacagcgtgacctccaccttcagcaagctgcgcgaacagctcggccctgtgacccaggagttctgggataacctggaaaaggagacagagggcctgaggcaggagatgagcaaggatctggaggaggtgaaggccaaggtgcagccctacctggacgacttccagaagaagtggcaggaggagatggagctctaccgccagaaggtggagccgctgcgcgcagagctccaagagggcgcgcgccagaagctgcacgagctgcaagagaagctgagcccactgggcgaggagatgcgcgaccgcgcgcgcgcccatgtggacgcgctgcgcacgcatctggccccctacagcgacgagctgcgccagcgcttggccgcgcgccttgaggctctcaaggagaacggcggcgccagactggccgagtaccacgccaaggccaccgagcatctgagcacgctcagcgagaaggccaagcccgcgctcgaggacctccgccaaggcctgctgcccgtgctggagagcttcaaggtcagcttcctgagcgctctcgaggagtacactaagaagctcaacacccagtgaggcgcccgccgccgccccccttcccggtgctcagaataaacgtttccaaagtggg

SEQ ID NO: 471 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 472 - DNA, 770 bp, Homo sapiens
  CDS (31)..(588)
cggagcagctctacccctcacgacgcagacatggcagcgcagaaggaccagcagaaagatgccgaggcggaagggctgagcggcacgaccctgctgccgaagctgattccctccggtgcaggccgggagtggctggagcggcgccgcgcgaccatccggccctggagcaccttcgtggaccagcagcgcttctcacggccccgcaacctgggagagctgtgccagcgcctcgtacgcaacgtggagtactaccagagcaactatgtgttcgtgttcctgggcctcatcctgtactgtgtggtgacgtcccctatgttgctggtggctctggctgtctttttcggcgcctgttacattctctatctgcgcaccttggagtccaagcttgtgctctttggccgagaggtgagcccagagcatcagtatgctctggctggaggcatctccttccccttcttctggctggctggtgcgggctcggccgtcttctgggtgctgggagccaccctggtggtcatcggctcccacgctgccttccaccagattgaggctgtggacggggaggagctgcagatggaacccgtgtgaggtgtcttctgggacctgccggcctcccgggccagctgccccacccctgcccatgcctgtcctgcacggctctgctgctcgggcccacagcgccgtcccatcacaagcccggggagggatcccgcctttgaaaataaagctgttatgggtgtcattcaggaaaaaaaaaaaaaaaaaaaaaa

SEQ ID NO: 473 - protein, 185 aa, Homo sapiens
MAAQKDQQKDAEAEGLSGTTLLPKLIPSGAGREWLERRRATIRPWSTFVDQQRFSRPRNLGELCQRLVRNVEYYQSNYVFVFLGLILYCVVTSPMLLVALAVFFGACYILYLRTLESKLVLFGREVSPEHQYALAGGISFPFFWLAGAGSAVFWVLGATLVVIGSHAAFHQIEAVDGEELQMEPV

(19) United States
(12) Patent Application Publication
(10) Pub. No.: US 2009/0082265 A1
(43) Pub. Date: Mar. 26, 2009

(54) COMPOSITIONS AND METHODS FOR TREATING DISEASES

(75) Inventors: Paul Bartel, Salt Lake City, UT (US); Daniel Cimbora, Salt Lake City, UT (US); Janice Sugiyama, Salt Lake City, UT (US); Daniel A. Wettstein, Salt Lake City, UT (US); Karen Heichman, Salt Lake City, UT (US)

Correspondence Address: MYRIAD GENETICS INC.;INTELLECUTAL PROPERTY DEPARTMENT, 320 WAKARA WAY, SALT LAKE CITY, UT, 84108, US

(73) Assignee: Myriad Genetics, Incorporated, Salt Lake City, UT (US)

(21) Appl. No.: 11/865,591

(22) Filed: Oct. 1, 2007

Related U.S. Application Data
(63) Continuation-in-part of application No. 11/075,234, filed on Mar. 7, 2005, now abandoned.
Application No. 11/075,234 is a continuation-in-part of application No. 10/135,802, filed on Apr. 29, 2002, now abandoned.
Application No. 10/135,802 is a continuation-in-part of application No. 10/035,344, filed on Jan. 4, 2002, now abandoned.
Application No. 10/035,344 is a continuation-in-part of application No. 10/098,979, filed on Mar. 14, 2002, now abandoned.
Application No. 10/098,979 is a continuation-in-part of application No. 10/099,924, filed on Mar. 14, 2002, now abandoned.
Application No. 10/099,924 is a continuation-in-part of application No. 10/122,573, filed on Apr. 15, 2002, now abandoned.
Application No. 10/122,573 is a continuation-in-part of application No. 10/124,550, filed on Apr. 17, 2002, now abandoned.
Application No. 10/124,550 is a continuation-in-part of application No. 10/124,767, filed on Apr. 17, 2002, now abandoned.
Application No. 10/124,767 is a continuation-in-part of application No. 10/125,639, filed on Apr. 18, 2002, now abandoned.
Application No. 10/125,639 is a continuation-in-part of application No. 10/100,503, filed on Mar. 18, 2002, now Pat. No. 7,141,401.

(51) Int. Cl. A61K 38/16; C07K 14/00; G01N 33/68; G01N 33/53; C12Q 1/68; A61P 9/00; A61P 31/12; A61P 25/00; A61P 35/00; A61K 31/7052; C12N 1/00; C40B 40/10

(52) U.S. Cl. 514/12; 530/402; 436/86; 435/7.2; 435/7.31; 435/243; 506/18; 514/44; 435/6

(57) ABSTRACT

Protein complexes are provided comprising at least one interacting pair of proteins. The protein complexes are useful in screening assays for identifying compounds effective in modulating the protein complexes, and in treating and/or preventing diseases and disorders associated with the protein complexes and/or their constituent interacting members.

### RELATED U.S. APPLICATIONS

This application is a continuation-in-part of U.S. patent application Ser. No. 11/075,234 filed on Mar. 7, 2005 which is a continuation-in-part of U.S. patent application Ser. No. 10/135,802 filed on Apr. 29, 2002, Ser. No. 10/035,344 filed on Jan. 4, 2002, Ser. No. 10/098,979 filed on Mar. 14, 2002, Ser. No. 10/099,924 filed on Mar. 14, 2002, Ser. No. 10/122,573 filed on Apr. 15, 2002, Ser. No. 10/124,550 filed on Apr. 17, 2002, Ser. No. 10/124,767 filed on Apr. 17, 2002, Ser. No. 10/125,639 filed on Apr. 18, 2002 and Ser. No. 10/100,503 filed on Mar. 18, 2002, each of which is incorporated herein by reference.

U.S. patent application Ser. No. 10/135,802 is related to U.S. provisional patent application Ser. No. 60/287,513 filed on Apr. 30, 2001. U.S. patent application Ser. No. 10/035,344 is related to U.S. provisional patent application Ser. No. 60/259,573 filed on Jan. 4, 2001 and U.S. provisional patent application Ser. No. 60/259,571 filed on Jan. 4, 2001. U.S. patent application Ser. No. 10/098,979 is related to U.S. provisional patent application Ser. No. 60/276,259 filed on Mar. 14, 2001, U.S. provisional patent application Ser. No. 60/304,101 filed on Jul. 10, 2001, U.S. provisional patent application Ser. No. 60/347,829 filed on Oct. 22, 2001 and U.S. provisional patent application 60/346,384 filed on Jan. 7, 2002. U.S. patent application Ser. No. 10/099,924 is related to U.S. provisional patent application Ser. No. 60/276,179 filed on Mar. 15, 2001, U.S. provisional patent application Ser. No. 60/307,233 filed on Jul. 23, 2001, and U.S. provisional patent application Ser. No. 60/343,818, filed on Oct. 25, 2001. U.S. patent application Ser. No. 10/122,573 is related to U.S. provisional patent application Ser. No. 60/284,095 filed on Apr. 16, 2001. U.S. patent application Ser. No. 10/124,550 is related to U.S. Provisional Application Ser. No. 60/284,404 filed on Apr. 17, 2001. U.S. patent application Ser. No. 10/124,767 is related to U.S. provisional patent application Ser. No. 60/284,220 filed on Apr. 17, 2001 and U.S. provisional patent application Ser. No. 60/354,899 filed on Feb. 6, 2002. U.S. patent application Ser. No. 10/125,639 is related to U.S. provisional patent application Ser. No. 60/285,324 filed on Apr. 19, 2001 and U.S. provisional patent application Ser. No. 60/349,843 filed on Jan. 17, 2002. U.S. patent application Ser. No. 10/100,503 is related to U.S. provisional patent application Ser. No. 60/277,013 filed on Mar. 19, 2001. All priority documents, including provisional applications and non-provisional applications provided above, are incorporated in their entirety herein by reference.

### SEQUENCE LISTING

This application is being filed with a formal Sequence Listing submitted electronically as a text file. This text file, which is named “1835-01-1X-2007-10-01-SEQ-LIST-HLL.ST25.txt”, was created on Oct. 1, 2007 and is 200,950 bytes in size. Its contents are incorporated by reference herein in their entirety.

### FIELD OF THE INVENTION

The present invention generally relates to methods and compositions for treating diseases, particularly to methods of using and modulating specific proteins and protein-protein interactions for purposes of drug screening and treatment of diseases.

### BACKGROUND OF THE INVENTION

Most drug discovery efforts today employ approaches to empirically identify small molecules that bind particular biological targets in vitro. These approaches generally involve “primary” high throughput screens designed to search vast combinatorial libraries of small molecules for “lead compounds” that often show a relatively weak affinity for the chosen target. However, once such lead compounds are identified in a “primary” high throughput screen, they can be subjected to further iterative rounds of chemical modification and testing by the process known to medicinal chemists as Structure Activity Relationship, or SAR. Generally, after several rounds of SAR-guided modification and in vitro screening, a set of optimized and related drug candidate compounds are subjected to the next phase of testing. This next phase generally involves the in vivo screening of the drug candidates in cell-based assays specifically designed to test the efficacy, toxicity and bioavailablity of the candidates. If the desired effects are obtained with reasonable dosages in these cell-based assays, animal studies are then initiated to determine whether the drug candidates have the desired activity in vivo. Only after careful study in well-defined animal models will a drug candidate be administered to humans in carefully regulated clinical trials.

The success or failure of a drug discovery program is heavily dependent on the identification and selection of druggable targets. In addition, once an appropriate drug target has been identified an efficient, preferably high throughput, screening assay needs to be established for drug screening against that particular drug target, which can be often be difficult to pragmatically achieve. The present invention provides novel drug targets for diseases such as abnormal cell proliferation (hyperproliferation or dysproliferation), keloid, liver cirrhosis, psoriasis, altered wound healing, cancer, especially cancers of the breast, ovary, colon, prostate, and lung, melanomas and cancers of hematopoietic origin, type 2 diabetes, obesity, hypertension, hypercholesterolemia, atherosclerosis, coronary artery disease, peripheral vascular disease, optic neuropathy, inflammatory response, amyloidosis, dementia, Alzheimer's disease, Tangier disease, Angelman syndrome, and specific viral infections; and discloses screening assays for identifying potential drugs that may be effective in the alleviation or treatment of diseases through modulating the drug targets.

### SUMMARY OF THE INVENTION

The present invention is based on the discovery of novel interactions between pairs of proteins described in the tables below. The specific interactions lead to the identification of desirable novel drug targets. Specifically, the interactions implicate several newly discovered interactors in the abnormal cell proliferation (hyperproliferation or dysproliferation), keloid, liver cirrhosis, psoriasis, altered wound healing, cancer, especially cancers of the breast, ovary, colon, prostate, and lung, melanomas and cancers of hematopoietic origin, type 2 diabetes, obesity, hypertension, hypercholesterolemia, atherosclerosis, coronary artery disease, peripheral vascular disease, optic neuropathy, inflammatory response, amyloidosis, dementia, Alzheimer's disease, Tangier disease, Angelman syndrome, and specific viral infections; and other disease pathways; and such interactions suggest that modulation of such interactors may lead to alleviation or treatment of the diseases. In addition, the interactions can lead to the formation of protein complexes both in vitro and in vivo. This enables novel approaches for drug screening to select not only drug candidates that modulate the well-known drug targets used as baits in the interaction discovery, but also modulators of the newly discovered interactors and protein-protein interactions. For example, screening assays can be established based on the interaction between a protein known to be involved in a disease pathway and one of its newly discovered protein interactors. Compounds that modulate or interact with the known target protein can be selected based on their ability either to compete, either competitively or noncompetitively, with a newly discovered interactor for interaction with the target protein, or to promote the interaction between the target protein and the interactor.

Thus, in accordance with a first aspect of the present invention, isolated protein complexes are provided which are formed by the protein-protein interactions provided in the tables. In addition, homologues, derivatives, or fragments of the interacting proteins may also be used in forming protein complexes. In a specific embodiment, fragments of an interacting pair of proteins described in the tables containing regions responsible for the protein-protein interaction (e.g., the interactions domains) are used in forming a protein complex of the present invention. In another embodiment, at least one interacting protein member in a protein complex of the present invention is a fusion protein containing the amino acid sequence of a protein in the tables below, or a homologue, derivative, or fragment thereof. In yet another embodiment, a protein complex is provided by way of a hybrid protein, which comprises, covalently linked together, either directly or through a linker, a pair of the interacting proteins described in the tables below, or homologues, derivatives, or fragments thereof. In addition, nucleic acids encoding such hybrid protein are also provided.

In another aspect, the present invention provides a method for making the protein complexes of the invention. The method includes the steps of providing the first protein and the second protein of the protein complexes of the present invention, and contacting said first protein with said second protein. In addition, such protein complexes can be prepared by isolation or purification from tissues and cells, or, alternatively, produced by recombinant expression of their respective protein members. The protein complexes can be incorporated into a protein microchip or microarray, which are useful in large-scale high throughput screening assays involving the protein complexes.

In accordance with yet another aspect of the invention, antibodies are provided that are immunoreactive with a protein complex of the present invention. In one embodiment, an antibody is selectively immunoreactive with a protein complex of the present invention. In another embodiment, a bifunctional antibody is provided that has two different antigen binding sites, each being specific to a different interacting protein partner in a protein complex of the present invention. The antibodies of the present invention can take various forms including polyclonal antibodies, monoclonal antibodies, chimeric antibodies, antibody fragments such as Fv fragments, single-chain Fv fragments (scFv), Fab′ fragments, and F(ab′)₂ fragments. Preferably, the antibodies are partially or fully humanized antibodies. The antibodies of the present invention can be readily prepared using procedures generally known in the art. For example, recombinant libraries such as phage display libraries and ribosome display libraries may be used to screen for antibodies with desirable specificities. In addition, various mutagenesis techniques such as site-directed mutagenesis and PCR diversification may be used in combination with the screening assays.

The present invention also provides detection methods for use in determining whether there is any aberration in a patient with respect to a protein complex formed by one or more interactions provided in accordance with this invention. In one embodiment, the method comprises detecting an aberrant concentration of the protein complexes of the present invention. Alternatively, the concentrations of one or more interacting protein members (at the protein, cDNA, or mRNA level) of a protein complex of the present invention are measured. In addition, the cellular localization, or tissue or organ distribution of a protein complex of the present invention is determined to detect any aberrant localization or distribution of the protein complex. In another embodiment, mutations in one or more interacting protein members of a protein complex of the present invention can be detected. In particular, it is desirable to determine whether the interacting protein members have any mutations that will lead to, or are associated with, changes in the functional activity of the proteins, or changes in their binding affinity to other interacting protein partners in forming a protein complex of the present invention. In yet another embodiment, the binding constant of the interacting protein members of one or more protein complexes is determined. A kit may be used for conducting the detection methods of the present invention. Typically, the kit contains reagents useful in any of the above-described embodiments of the detection methods, including, e.g., antibodies specific to a protein complex of the present invention or interacting members thereof, and oligonucleotides selectively hybridizable to the cDNAs or mRNAs encoding one or more interacting protein members of a protein complex. The detection methods may be useful in diagnosing a disease or disorder such as abnormal cell proliferation (hyperproliferation or dysproliferation), keloid, liver cirrhosis, psoriasis, altered wound healing, cancer, especially cancers of the breast, ovary, colon, prostate, and lung, melanomas and cancers of hematopoietic origin, type 2 diabetes, obesity, hypertension, hypercholesterolemia, atherosclerosis, coronary artery disease, peripheral vascular disease, optic neuropathy, inflammatory response, amyloidosis, dementia, Alzheimer's disease, Tangier disease, Angelman syndrome, and specific viral infections; and to staging the disease or disorder, or identifying a predisposition to the disease or disorder.

The present invention also provides screening methods for selecting modulators of a protein complex provided according to the present invention. Screening methods are also provided for selecting modulators of the individual interacting proteins. The compounds identified in the screening methods of the present invention can be useful in modulating the functions or activities of the individual interacting proteins, or the protein complexes of the present invention. They may also be effective in modulating the cellular processes involving the proteins and protein complexes, and in preventing or ameliorating diseases or disorders such as abnormal cell proliferation (hyperproliferation or dysproliferation), keloid, liver cirrhosis, psoriasis, altered wound healing, cancer, especially cancers of the breast, ovary, colon, prostate, and lung, melanomas and cancers of hematopoietic origin, type 2 diabetes, obesity, hypertension, hypercholesterolemia, atherosclerosis, coronary artery disease, peripheral vascular disease, optic neuropathy, inflammatory response, amyloidosis, dementia, Alzheimer's disease, Tangier disease, Angelman syndrome, and specific viral infections.

Thus, test compounds may be screened in in vitro binding assays to identify compounds capable of binding a protein complex of the present invention, or its individual interacting protein members. The assays may include the steps of contacting the protein complex with a test compound and detecting the interaction between the interacting partners. In addition, in vitro dissociation assays may also be employed to select compounds capable of dissociating or destabilizing the protein complexes identified in accordance with the present invention. For example, the assays may entail (1) contacting the interacting members of a protein complex with each other in the presence of a test compound; and (2) detecting the interaction between the interacting members. An in vitro screening assay may also be used to identify compounds that trigger or initiate the formation of, or stabilize, a protein complex of the present invention.

In preferred embodiments, in vivo assays such as yeast two-hybrid assays and various derivatives thereof, preferably reverse two-hybrid assays, are utilized in identifying compounds that interfere with or disrupt the protein-protein interactions discovered according to the present invention. In addition, systems such as yeast two-hybrid assays are also useful in selecting compounds capable of triggering or initiating, enhancing or stabilizing the protein-protein interactions provided in the tables. In a specific embodiment, the screening method includes: (a) providing in a host cell a first fusion protein having a first protein of an interacting protein pair, or a homologue, derivative, or fragment thereof, and a second fusion protein having the second protein of the pair, or a homologue, derivative, or fragment thereof, wherein a DNA binding domain is fused to one of the first and second proteins while a transcription-activating domain is fused to the other of said first and second proteins; (b) providing in the host cell a reporter gene, wherein the transcription of the reporter gene is determined by the interaction between the first protein and the second protein; (c) allowing the first and second fusion proteins to interact with each other within the host cell in the presence of a test compound; and (d) determining the presence or absence of expression of the reporter gene.

In addition, the present invention also provides a method for selecting a compound capable of modulating a protein-protein interaction in accordance with the present invention, which comprises the steps of (1) contacting a test compound with an interacting protein disclosed in the tables, or a homologue, derivative, or fragment thereof; and (2) determining whether said test compound is capable of binding said protein. In a preferred embodiment, the method further includes testing a selected test compound capable of binding said interacting protein for its ability to interfere with a protein-protein interaction according to the present invention involving said interacting protein, and optionally further testing the selected test compound for its ability to modulate cellular activities associated with said interacting protein and/or said protein-protein interaction.

The present invention also relates to a virtual screen method for providing a compound capable of modulating the interaction between the interacting members in a protein complex of the present invention. In one embodiment, the method comprises the steps of providing atomic coordinates defining a three-dimensional structure of a protein complex of the present invention, and designing or selecting, based on said atomic coordinates, compounds capable of interfering with the interaction between the interacting protein members of the protein complex. In another embodiment, the method comprises the steps of providing atomic coordinates defining a three-dimensional structure of an interacting protein described in the tables, and designing or selecting compounds capable of binding the interacting protein based on said atomic coordinates. In preferred embodiments, the method further includes testing a selected test compound for its ability to interfere with a protein-protein interaction provided in accordance with the present invention involving said interacting protein, and optionally further testing the selected test compound for its ability to modulate cellular activities associated with the interacting protein.

The present invention further provides a composition having two expression vectors. One vector contains a nucleic acid encoding a first protein of an interacting protein pair according to the present invention, or a homologue, derivative, or fragment thereof. Another vector contains the second protein of the interacting pair, or a homologue, derivative, or fragment thereof. In addition, an expression vector is also provided containing (1) a first nucleic acid encoding a first protein of an interacting protein pair of the present invention, or a homologue, derivative, or fragment thereof, and (2) a second nucleic acid encoding a second protein of the interacting pair, or a homologue, derivative, or fragment thereof.

Host cells are also provided containing the first and second nucleic acids or comprising the expression vector(s) described above. In addition, the present invention also provides a host cell having two expression cassettes. One expression cassette includes a promoter operably linked to a nucleic acid encoding a first protein of an interacting pair of the present invention, or a homologue, derivative, or fragment thereof. Another expression cassette includes a promoter operably linked to a nucleic acid encoding a second protein of the interacting pair, or a homologue, derivative, or fragment thereof. Preferably, the expression cassettes are chimeric expression cassettes with heterologous promoters operably linked to the protein coding sequences.

In specific embodiments of the host cells or expression vectors, one of the two nucleic acids is linked to a nucleic acid encoding a DNA binding domain, and the other is linked to a nucleic acid encoding a transcription-activation domain, whereby two fusion proteins can be encoded.

In accordance with yet another aspect of the present invention, methods are provided for modulating the functions and activities of a protein complex of the present invention, or the interacting protein members thereof. The methods may be used in treating or preventing diseases and disorders such as abnormal cell proliferation (hyperproliferation or dysproliferation), keloid, liver cirrhosis, psoriasis, altered wound healing, cancer, especially cancers of the breast, ovary, colon, prostate, and lung, melanomas and cancers of hematopoietic origin, type 2 diabetes, obesity, hypertension, hypercholesterolemia, atherosclerosis, coronary artery disease, peripheral vascular disease, optic neuropathy, inflammatory response, amyloidosis, dementia, Alzheimer's disease, Tangier disease, Angelman syndrome, and specific viral infections. In one embodiment, the method comprises reducing a protein complex concentration and/or inhibiting the functional activities of the protein complex. Alternatively, the concentration and/or activity of one or more interacting members of a protein complex may be reduced or inhibited. Thus, the methods may include administering to a patient an antibody specific to a protein complex or an interacting protein member thereof, or an siRNA or antisense oligo or ribozyme selectively hybridizable to a gene or mRNA encoding an interacting member of the protein complex. Also useful is a compound identified in a screening assay of the present invention capable of disrupting the interaction between two interacting members of a protein complex, or inhibiting the activities of an interacting member of the protein complex. In addition, gene therapy methods may also be used in reducing the expression of the gene(s) encoding one or more interacting protein partners of a protein complex of the present invention.

In another embodiment, the methods for modulating the functions and activities of a protein complex of the present invention, or the interacting protein members thereof, comprise increasing the protein complex concentration and/or activating the functional activities of the protein complex. Alternatively, the concentration and/or activity of one or more interacting members of a protein complex of the present invention may be increased. Thus, one or more interacting protein members of a protein complex of the present invention may be administered directly to a patient. Or, exogenous genes encoding one or more protein members of a protein complex of the present invention may be introduced into a patient by gene therapy techniques. In addition, a patient needing treatment or prevention may also be administered with compounds identified in a screening assay of the present invention capable of triggering or initiating, enhancing or stabilizing a protein-protein interaction of the present invention.

The foregoing and other advantages and features of the invention, and the manner in which the same are accomplished, will become more readily apparent upon consideration of the following detailed description of the invention taken in conjunction with the accompanying examples, which illustrate preferred and exemplary embodiments.

### BRIEF DESCRIPTION OF THE DRAWINGS

FIGS. 1-80 depict siRNA molecules designed to target the transcript encoding the identified interacting protein and induce the RNAi-mediated degradation of that transcript.

### DETAILED DESCRIPTION OF THE INVENTION

### 1. Definitions

The terms “polypeptide,” “protein,” and “peptide” are used herein interchangeably to refer to amino acid chains in which the amino acid residues are linked by peptide bonds or modified peptide bonds. The amino acid chains can be of any length of greater than two amino acids. Unless otherwise specified, the terms “polypeptide,” “protein,” and “peptide” also encompass various modified forms thereof. Such modified forms may be naturally occurring modified forms or chemically modified forms. Examples of modified forms include, but are not limited to, glycosylated forms, phosphorylated forms, myristoylated forms, palmitoylated forms, ribosylated forms, acetylated forms, ubiquitinated forms, etc. Modifications also include intra-molecular crosslinking and covalent attachment to various moieties such as lipids, flavin, biotin, polyethylene glycol or derivatives thereof, etc. In addition, modifications may also include cyclization, branching and cross-linking. Further, amino acids other than the conventional twenty amino acids encoded by the codons of genes may also be included in a polypeptide.

The term “isolated polypeptide” as used herein is defined as a polypeptide molecule that is present in a form other than that found in nature. Thus, an isolated polypeptide can be a non-naturally occurring polypeptide. For example, an “isolated polypeptide” can be a “hybrid polypeptide.” An “isolated polypeptide” can also be a polypeptide derived from a naturally occurring polypeptide by additions or deletions or substitutions of amino acids. An isolated polypeptide can also be a “purified polypeptide” which is used herein to mean a specified polypeptide in a substantially homogeneous preparation substantially free of other cellular components, other polypeptides, viral materials, or culture medium, or when the polypeptide is chemically synthesized, chemical precursors or by-products associated with the chemical synthesis. A “purified polypeptide” can be obtained from natural or recombinant host cells by standard purification techniques, or by chemical synthesis, as will be apparent to skilled artisans.

The terms “hybrid protein,” “hybrid polypeptide,” “hybrid peptide,” “fusion protein,” “fusion polypeptide,” and “fusion peptide” are used herein interchangeably to mean a non-naturally-occurring polypeptide or isolated polypeptide having a specified polypeptide molecule covalently linked to one or more other polypeptide molecules that do not link to the specified polypeptide in nature. Thus, a “hybrid protein” may be two naturally occurring proteins or fragments thereof linked together by a covalent linkage. A “hybrid protein” may also be a protein formed by covalently linking two artificial polypeptides together. Typically but not necessarily, the two or more polypeptide molecules are linked or “fused” together by a peptide bond forming a single non-branched polypeptide chain.

As used herein, the term “interacting” or “interaction” means that two protein domains, fragments or complete proteins exhibit sufficient physical affinity to each other so as to bring the two “interacting” protein domains, fragments or proteins physically close to each other. An extreme case of interaction is the formation of a chemical bond that results in continual and stable proximity of the two entities. Interactions that are based solely on physical affinities, although usually more dynamic than chemically bonded interactions, can be equally effective in co-localizing two proteins. Examples of physical affinities and chemical bonds include but are not limited to, forces caused by electrical charge differences, hydrophobicity, hydrogen bonds, van der Waals force, ionic force, covalent linkages, and combinations thereof. The state of proximity between the interaction domains, fragments, proteins or entities may be transient or permanent, reversible or irreversible. In any event, it is in contrast to and distinguishable from contact caused by natural random movement of two entities. Typically, although not necessarily, an “interaction” is exhibited by the binding between the interaction domains, fragments, proteins, or entities. Examples of interactions include specific interactions between antigen and antibody, ligand and receptor, enzyme and substrate, and the like.

An “interaction” between two protein domains, fragments or complete proteins can be determined by a number of methods. For example, an interaction is detectable by any commonly accepted approaches, including functional assays such as the two-hybrid systems. Protein-protein interactions can also be determined by various biophysical and biochemical approaches based on the affinity binding between the two interacting partners. Such biochemical methods generally known in the art include, but are not limited to, protein affinity chromatography, affinity blotting, immunoprecipitation, and the like. The binding constant for two interacting proteins, which reflects the strength or quality of the interaction, can also be determined using methods known in the art, including surface plasmon resonance and isothermal titration calorimetry binding analyses. See Phizicky and Fields, Microbiol. Rev., 59:94-123 (1995).

As used herein, the term “protein complex” means a composite unit that is a combination of two or more proteins formed by interaction between the proteins. Typically but not necessarily, a “protein complex” is formed by the binding of two or more proteins together through specific non-covalent binding affinities. However, covalent bonds may also be present between the interacting partners. For instance, the two interacting partners can be covalently crosslinked so that the protein complex becomes more stable.

The term “isolated protein complex” means a naturally occurring protein complex present in a composition or environment that is different from that found in its native or original cellular or biological environment in nature. An “isolated protein complex” may also be a protein complex that is not found in nature.

The term “protein fragment” as used herein means a polypeptide that represents a portion of a protein. When a protein fragment exhibits interactions with another protein or protein fragment, the two entities are said to interact through interaction domains that are contained within the entities. Interaction domains can be compact structures formed by amino acid residues that are close to one another in the primary sequence of a protein. Alternatively, interaction domains can be comprised of amino acid residues from portions of the polypeptide chain that are not close to one another in the primary sequence, but are brought together by the tertiary fold of the polypeptide chain.

As used herein, the term “domain” means a functional portion, segment or region of a protein, or polypeptide. “Interaction domain” refers specifically to a portion, segment or region of a protein, polypeptide or protein fragment that is responsible for the physical affinity of that protein, protein fragment or isolated domain for another protein, protein fragment or isolated domain.

The term “isolated” when used in reference to nucleic acids (which include gene sequences) of this invention is intended to mean that a nucleic acid molecule is present in a form other than that found in nature.

Thus, an isolated nucleic acid can be a non-naturally occurring nucleic acid. For example, the term “isolated nucleic acid” encompasses “recombinant nucleic acid” which is used herein to mean a hybrid nucleic acid produced by recombinant DNA technology having the specified nucleic acid molecule covalently linked to one or more nucleic acid molecules that are not the nucleic acids naturally flanking the specified nucleic acid in the naturally existing chromosome. One example of recombinant nucleic acid is a hybrid nucleic acid encoding a fusion protein. Another example is an expression vector having the specified nucleic acid inserted in a vector and operably linked to a promotor.

The term “isolated nucleic acid” also encompasses nucleic acid molecules that are present in a form other than that found in its original environment in nature with respect to its association with other molecules. In this respect, an “isolated nucleic acid” as used herein means a nucleic acid molecule having only a portion of the nucleic acid sequence in the chromosome but not one or more other portions present on the same chromosome. Thus, an isolated nucleic acid present in a form other than that found in its original environment in nature with respect to its association with other molecules typically includes no more than 10 kb of the naturally occurring nucleic acid sequences that immediately flank the gene in the naturally existing chromosome or genomic DNA. Thus, the term “isolated nucleic acid” encompasses the term “purified nucleic acid,” which means an isolated nucleic acid in a substantially homogeneous preparation substantially free of other cellular components, other nucleic acids, viral materials, or culture medium, or chemical precursors or by-products associated with chemical reactions for chemical synthesis of nucleic acids. Typically, a “purified nucleic acid” can be obtained by standard nucleic acid purification methods, as will be apparent to skilled artisans.

An isolated nucleic acid can be in a vector. However, it is noted that an “isolated nucleic acid” as used herein is distinct from a clone in a conventional library such as a genomic DNA library or a cDNA library in that the clones in a library are still in admixture with almost all the other nucleic acids from a chromosome or a cell.

The term “high stringency hybridization conditions,” when used in connection with nucleic acid hybridization, means hybridization conducted overnight at 42° C. in a solution containing 50% formamide, 5×SSC (750 mM NaCl, 75 mM sodium citrate), 50 mM sodium phosphate, pH 7.6, 5×Denhardt's solution, 10% dextran sulfate, and 20 microgram/ml denatured and sheared salmon sperm DNA, with hybridization filters washed in 0.1×SSC at about 65° C. The term “moderate stringent hybridization conditions,” when used in connection with nucleic acid hybridization, means hybridization conducted overnight at 37° C. in a solution containing 50% formamide, 5×SSC (750 mM NaCl, 75 mM sodium citrate), 50 mM sodium phosphate, pH 7.6, 5×Denhardt's solution, 10% dextran sulfate, and 20 microgram/ml denatured and sheared salmon sperm DNA, with hybridization filters washed in 1×SSC at about 50° C. It is noted that many other hybridization methods, solutions and temperatures can be used to achieve comparable stringent hybridization conditions as will be apparent to skilled artisans.

As used herein, the term “homologue,” when used in connection with a first native protein or fragment thereof, that is discovered, according to the present invention, to interact with a second native protein or fragment thereof, means a polypeptide that exhibits a sufficient amino acid sequence homology (greater than 20%) and structural resemblance to the first native interacting protein, or to one of the interacting domains or interacting fragments of the first native protein, such that it is capable of interacting with the second native protein. Typically, a protein homologue of a native protein can have an amino acid sequence that is at least about 50%, 55%, 60%, 65% or 70%, preferably at least about 75%, more preferably at least about 80%, 85%, 86%, 87%, 88% or 89%, even more preferably at least 90%, 91%, 92%, 93% or 94%, and most preferably about 95%, 96%, 97%, 98% or 99% identical to the native protein. Examples of homologues may be the orthologous proteins of other species including animals, plants, yeast, bacteria, and the like. Homologues may be native, naturally-occurring proteins or, alternatively, created by mutagenesis of a native, naturally-occurring protein. For example, homologues may be created by site-specific mutagenesis in combination with assays for detecting protein-protein interactions, e.g., the yeast two-hybrid system described below, as will be apparent to skilled artisans apprised of the present invention. Other techniques for detecting protein-protein interactions between homologous proteins include, e.g., affinity chromatography, affinity blotting, in vitro binding assays, such as “surface plasmon resonance binding analyses,” antibody “pull-down” assays, and the like.

For the purpose of comparing two different nucleic acid or polypeptide sequences, one sequence (the test sequence) may be described to be a specific “percent identical to” another sequence (the reference sequence) in the present disclosure. In this respect, the percentage identity is determined by the algorithm of Karlin and Altschul, Proc. Natl. Acad. Sci. USA, 90:5873-5877 (1993), which is incorporated into various BLAST programs. Specifically, the percentage identity is determined by the “BLAST 2 Sequences” tool, which is available from the National Center for Biotechnology Information, U.S. National Library of Medicine, Bethesda, Md. See Tatusova and Madden, FEMS Microbiol. Lett., 174(2):247-250 (1999). For pairwise DNA-DNA comparison, the BLASTN 2.1.2 program is used with default parameters (Match: 1; Mismatch: −2; Open gap: 5 penalties; extension gap: 2 penalties; gap x_dropoff: 50; expect: 10; and word size: 11, with filter). For pairwise protein-protein sequence comparison, the BLASTP 2.1.2 program is employed using default parameters (Matrix: BLOSUM62; gap open: 11; gap extension: 1; x_dropoff: 15; expect: 10.0; and wordsize: 3, with filter). Percent identity of two sequences is calculated by aligning a test sequence with a reference sequence using BLAST 2.1.2., determining the number of amino acids or nucleotides in the aligned test sequence that are identical to amino acids or nucleotides in the same position of the reference sequence, and dividing the number of identical amino acids or nucleotides by the number of amino acids or nucleotides in the reference sequence. When BLAST 2.1.2 is used to compare two sequences, it aligns the sequences and yields the percent identity over defined, aligned regions. If the two sequences are aligned across their entire length, the percent identity yielded by the BLAST 2.1.1 is the percent identity of the two sequences. If BLAST 2.1.2 does not align the two sequences over their entire length, then the number of identical amino acids or nucleotides in the unaligned regions of the test sequence and reference sequence is considered to be zero and the percent identity is calculated by adding the number of identical amino acids or nucleotides in the aligned regions and dividing that number by the length of the reference sequence.

The term “derivative,” when used in connection with a first native protein (or fragment thereof) that is discovered, according to the present invention, to interact with a second native protein (or fragment thereof), means a modified form of the first native protein prepared by modifying the side chain groups of the first native protein without changing the amino acid sequence of the first native protein. The modified form, i.e., the derivative should be capable of interacting with the second native protein. Examples of modified forms include glycosylated forms, phosphorylated forms, myristylated forms, ribosylated forms, ubiquitinated forms, and the like. Derivatives also include hybrid or fusion proteins containing a native protein or a fragment thereof. Methods for preparing such derivative forms should be apparent to skilled artisans. The prepared derivatives can be easily tested for their ability to interact with the native interacting partner using techniques known in the art, e.g., protein affinity chromatography, affinity blotting, in vitro binding assays, yeast two-hybrid assays, and the like.

The term “antibody” as used herein encompasses both monoclonal and polyclonal antibodies that fall within any antibody classes, e.g., IgG, IgM, IgA, IgE, or derivatives thereof. The term “antibody” also includes antibody fragments including, but not limited to, Fab, F(ab′)₂, and conjugates of such fragments, and single-chain antibodies comprising an antigen recognition epitope. In addition, the term “antibody” also means humanized antibodies, including partially or fully humanized antibodies. An antibody may be obtained from an animal, or from a hybridoma cell line producing a monoclonal antibody, or obtained from cells or libraries recombinantly expressing a gene encoding a particular antibody.

The term “selectively immunoreactive” as used herein means that an antibody is reactive thus binds to a specific protein or protein complex, but not other similar proteins or fragments or components thereof.

The term “activity” when used in connection with proteins or protein complexes means any physiological or biochemical activities displayed by, or associated with, a particular protein or protein complex including, but not limited to, activities exhibited in biological processes and cellular functions, ability to interact with or bind another molecule or a moiety thereof, binding affinity or specificity to certain molecules, in vitro or in vivo stability (e.g., protein degradation rate, or in the case of protein complexes, the ability to maintain the form of a protein complex), antigenicity and immunogenicity, enzymatic activities, etc. Such activities may be detected or assayed by any of a variety of suitable methods as will be apparent to skilled artisans.

The term “compound” as used herein encompasses all types of organic or inorganic molecules, including but not limited proteins, peptides, polysaccharides, lipids, nucleic acids, small organic molecules, inorganic compounds, and derivatives thereof.

As used herein, the term “interaction antagonist” means a compound that interferes with, blocks, disrupts or destabilizes a protein-protein interaction; blocks or interferes with the formation of a protein complex; or destabilizes, disrupts or dissociates an existing protein complex.

The term “interaction agonist” as used herein means a compound that triggers, initiates, propagates, nucleates, or otherwise enhances the formation of a protein-protein interaction; triggers, initiates, propagates, nucleates, or otherwise enhances the formation of a protein complex; or stabilizes an existing protein complex.

Unless otherwise specified, the names of interacting proteins, as used herein, and when referring to the protein complexes of the present invention, are meant to refer to full-length proteins, as well as any fragments thereof that are capable of interacting with a specified interacting partner protein as disclosed in the tables below. Additionally, while the names of interacting proteins, as used herein, generally refer to the human form of the named protein, they may also refer to homologous proteins from other species that interact in a manner analogous to the human protein. Preferably, such homologous proteins are at least about 80%, 85%, 90%, 92%, 93%, 94%, 95%, 96%, 97%, 98%, 99% or more, identical to the human form of the protein. Also, preferably, such homologous proteins interact with the human form of the corresponding interacting partner protein disclosed in the tables. Finally, the names of interacting proteins, as used herein, are also meant to refer to fragments of such human proteins, or their homologues, that interact to form complexes with the interacting partner proteins, as disclosed in the tables. For example, the term “TSG101” refers not only to the full length human tumor susceptibility gene 101 protein (TSG101), but also refers to fragments of human TSG101, homologues of human TSG101, and fragments of these homologues of human TSG101, that retain the ability to interact with a TSG101 partner or prey protein specified in the tables.

### 2. Protein Complexes

Novel protein-protein interactions have been discovered. The protein-protein interactions are provided in the Tables below. Specific fragments capable of conferring interacting properties on the interacting proteins have also been identified. The GenBank® reference numbers for the cDNA sequences encoding the interacting proteins are also noted in the tables.

### Tables

TABLE 1
FMS-Related Tyrosine Kinase 4 and Its Interacting Partners

| Bait Protein | | | Prey Proteins | | | |
|---|---|---|---|---|---|---|
| Name and | Amino Acid | | | GenBank | Amino Acid | |
| GenBank ® | Coordinates | | | Accession | Coordinates | |
| Accession No | Start | Stop | Names | Nos. | Start | Stop |
| FMS-Related | 791 | 177 | phospholipase C | M34667 | 445 | 757 |
| Tyrosine Kinase | | | gamma 1 (PLCG1) | | 505 | 772 |
| 4 (FLT4) | | | E6-AP ubiquitin- | L07557 | 636 | 861 |
| (GenBank | | | protein ligase | | 705 | 861 |
| Accession No: | | | (UBE3A) | | 720 | 861 |
| X68203) | | | | | | |

TABLE 2
AKT1 and Its Interacting Partners

| Bait Protein | | | Prey Proteins | | | |
|---|---|---|---|---|---|---|
| Name and | Amino Acid | | | GenBank ® | Amino Acid | |
| GenBank ® | Coordinates | | | Accession | Coordinates | |
| Accession No | Start | Stop | Names | Nos. | Start | Stop |
| v-akt murine | 1 | 150 | farnesyltransferase, | L00634 | 189 | 328 |
| thymoma viral | | | CAAX box, alpha | | | |
| oncogene | | | (FNTA) | | | |
| homolog 1 | 1 | 109 | Charcot-Leyden | NM_002705 | 1548 | 1756 |
| (AKT1) | | | crystal protein; | | | |
| (GenBank ® | | | periplakin | | | |
| Accession No: | | | (PPL) | | | |
| M63167) | | | dystonin | AB018271 | 254 | 469 |
| | | | (KIAA0728) | | | |
| | 1 | 118 | golgi autoantigen, | NM_005113 | 609 | 731 |
| | | | golgin subfamily a, | | | |
| | | | 5 | | | |
| | | | (Golgin-84) | | | |
| | 1 | 150 | tetratricopeptide | D84294 | 1058 | 1189 |
| | | | repeat domain 3 | | | |
| | | | (TPRD) | | | |

TABLE 3
AKT2 and Its Interacting Partners

| Bait Protein | | | Prey Proteins | | | |
|---|---|---|---|---|---|---|
| Name and | Amino Acid | | | GenBank ® | Amino Acid | |
| GenBank ® | Coordinates | | | Accession | Coordinates | |
| Accession No | Start | Stop | Names | Nos. | Start | Stop |
| v-akt murine | 1 | 152 | tetratricopeptide | L07597 | 1058 | 1 |
| thymoma viral | | | repeat domain 3 | | | |
| oncogene | | | (TPRD) | | | |
| homolog 2 | 1 | 108 | aldo-keto reductase | AF026947 | 82 | 330 |
| (AKT2) | | | family 7, member | | | |
| (GenBank ® | | | A2 | | | |
| Accession No: | | | (AKR7A2) | | | |
| M95936) | | | intracellular | X87689 | 51 | 210 |
| | | | chloride channel | | 98 | 210 |
| | | | protein | | | |
| | | | (CLIC1) | | | |
| | | | tetratricopeptide | L07597 | 1058 | 1189 |
| | | | repeat domain 3 | | | |
| | | | (TPRD) | | | |

TABLE 4
p90RSK and Its Interacting Partners

| Bait Protein | | | Prey Proteins | | | |
|---|---|---|---|---|---|---|
| Name and | Amino Acid | | | GenBank ® | Amino Acid | |
| GenBank ® | Coordinates | | | Accession | Coordinates | |
| Accession No | Start | Stop | Names | Nos. | Start | Stop |
| ribosomal | 600 | 736 | dystonin | AB018271 | 45 | 469 |
| protein S6 | | | (KIAA0728) | | | |
| kinase, 90 kDa, | 418 | 467 | upstream of N-ras, | AB020692 | 110 | 528 |
| polypeptide 1 | | | alt. transcript (798) | | | |
| (p90RSK) | | | (UNR) | | | |
| (GenBank ® | | | | | | |
| Accession No: | | | | | | |
| L07597) | | | | | | |

TABLE 5
TSG101 and Its Interacting Partner

| Bait Protein | | | Prey Proteins | | | |
|---|---|---|---|---|---|---|
| Name and | Amino Acid | | | GenBank ® | Amino Acid | |
| GenBank ® | Coordinates | | | Accession | Coordinates | |
| Accession No. | Start | Stop | Names | Nos. | Start | Stop |
| Tumor | 231 | 391 | Golgin-67 | AF163441 | 68 | 228 |
| Susceptibility | 240 | 291 | | | 123 | 226 |
| Gene 101 | | | | | 135 | 226 |
| (TSG101) | | | | | 1 | 231 |
| (GenBank ® | 231 | 391 | Kinectin | Z22551 | 851 | 1110 |
| Accession No. | | | | | 854 | 1110 |
| U82130) | | | | | 851 | 1113 |
| | 231 | 391 | Cytoplasmic linker | NM_003388 | 607 | 947 |
| | | | 2 (CYLN2) | | | |
| | 240 | 391 | Restin | M97501 | 770 | 898 |
| | | | | | 660 | 903 |
| | 231 | 391 | Tropomyosin | X05276 | 79 | 142 |
| | | | TM30p1 (TPM4) | | 91 | 142 |
| | 231 | 391 | FK506-binding | AB014574 | 770 | 880 |
| | 12 | 326 | protein homolog | | | |
| | | | KIAA0674 | | | |
| | 12 | 326 | Plectin | U53204 | 1325 | 1504 |
| | | | (PLEC1(4574)) | | 1328 | 1504 |
| | 1 | 274 | Actinin (ACTN4) | NM_004924 | 425 | 884 |
| | 12 | 326 | PIBF1 | Y09631 | 392 | 758 |
| | 12 | 326 | Accessory proteins | NM_005745 | 184 | 246 |
| | | | BAP31/BAP29 | | | |
| | | | (BAP31) | | | |
| | 231 | 391 | Zink finger protein | AF052224 | 2308 | 2438 |
| | | | 231 (ZNF231) | | | |
| | 231 | 391 | Chromosome- | AF020043 | 208 | 300 |
| | 264 | 391 | associated | | 119 | 353 |
| | | | polypeptide HCAP | | | |
| | | | (HCAP) | | | |
| | 1 | 274 | p53-induced protein | AF010312 | 1 | 106 |
| | | | 7 (PIG7) | | | |
| | 12 | 326 | hypothetical protein | AA300702 | 9 | 108 |
| | | | AA300702 | | | |
| | 1 | 274 | AT-hook | AK024431 | 165 | 357 |
| | | | transcription factor | | | |
| | | | FLJ00020 (AKNA) | | | |
| | 1 | 157 | target of myb1 | AJ010071 | 155 | 476 |
| | | | (chicken) homolog- | | | |
| | | | like 1 (TOM1L1) | | | |
| | 12 | 326 | novel protein | (SEQ ID NO: 2) | 268 | 422 |
| | | | PN9667 | | | |
| | 12 | 326 | Synaptic nuclei | NM_015293 | 1093 | 1249 |
| | | | expressed gene 1, | | | |
| | | | alt. Transcript beta | | | |
| | | | (3321) | | | |
| | | | (SYNE-1) | | | |
| | 1 | 274 | A-kinase anchor | M90360 | 324 | 483 |
| | | | protein (AKAP13) | | 324 | 587 |
| | | | | | 324 | 589 |
| | 265 | 391 | P87/89 motor | D21094 | 152 | 335 |
| | | | protein | | | |
| | 317 | 391 | Amplified in | U41635 | 171 | 350 |
| | | | osteosarcoma-9 | | 213 | 503 |
| | | | (OS-9) | | | |
| | 265 | 391 | Death associated | X89713 | 16 | 157 |
| | | | protein 5 (DAP5) | | | |
| | 231 | 391 | Growth arrest- | NM_005890 | 69 | 249 |
| | | | specific 7 (GAS7B) | | 70 | 278 |
| | | | | | 66 | 301 |
| | 231 | 391 | GrpE-Like protein | XP_052625 | 55 | 79 |
| | | | cochaperone | | 40 | 89 |
| | | | (PN19062) | | | |
| | 231 | 391 | Rho-associated | U43195 | 462 | 617 |
| | | | (ROCK1) | | | |
| | 265 | 391 | Guanine nucleotide | U72206 | 667 | 895 |
| | | | regulatory factor | | | |
| | | | GEF-H1 (GEF-H1) | | | |
| | 265 | 391 | Protein kinase C | AF128536 | 174 | 367 |
| | | | and casein kinase | | | |
| | | | substrate | | | |
| | | | (PACSIN2) | | | |
| | 240 | 391 | Desmoplakin I | J05211 | 1501 | 1589 |
| | | | | | 1438 | 1609 |
| | 140 | 270 | Synexin | J04543 | 22 | 329 |
| | 240 | 391 | Golgin-95 | L06147 | 23 | 189 |
| | 1 | 157 | Keratin 5 | D50666 | 9 | 171 |
| | 240 | 391 | | | 325 | 446 |
| | | | | | 282 | 448 |
| | | | | | 379 | 452 |
| | | | | | 335 | 473 |
| | | | | | 349 | 475 |
| | | | | | 384 | 475 |
| | | | | | 347 | 485 |
| | 240 | 391 | Keratin 6C | L42601 | 373 | 444 |
| | 240 | 391 | Keratin 8 | X98614 | 293 | 394 |
| | | | | | 147 | 406 |
| | 240 | 391 | GTPase-activiating | D29640 | 1406 | 1547 |
| | | | protein 1 | | 1404 | 1553 |
| | | | | | 1299 | 1555 |
| | | | | | 1439 | 1565 |
| | | | | | 1413 | 1567 |
| | | | | | 1439 | 1567 |
| | | | | | 1463 | 1568 |
| | | | | | 1308 | 1606 |
| | | | | | 1392 | 1657 |
| | | | | | 1419 | 1657 |
| | 240 | 391 | Endosome- | X78998 | 872 | 1039 |
| | | | associated protein 1 | | | |
| | 240 | 391 | 88-kDa Golgi | AB020662 | 128 | 237 |
| | | | protein | | 186 | 273 |
| | | | | | 148 | 287 |
| | | | | | 98 | 402 |
| | | | | | 118 | 487 |
| | 240 | 391 | Centromere protein | U19769 | 104 | 332 |
| | | | F | | 190 | 420 |
| | 240 | 391 | Serum deprivation | NM_004657 | 75 | 258 |
| | | | response | | | |
| | 240 | 391 | Mitotic spindle | NM_006461 | 668 | 895 |
| | | | coiled-coil related | | 723 | 1012 |
| | | | protein | | 942 | 1021 |
| | | | | | 701 | 1082 |
| | 147 | 391 | Golgi autoantigen | NM_005113 | 198 | 501 |
| | 231 | 391 | (Golgin-84) | | 198 | 501 |
| | 12 | 326 | | | 198 | 497 |
| | | | | | 198 | 501 |
| | 50 | 391 | Hypothetical | NM_018131 | 1 | 231 |
| | | | protein FLJ10540 | | 1 | 110 |
| | | | | | 1 | 117 |
| | | | | | 115 | 231 |
| | 140 | 270 | | | 1 | 120 |
| | | | | | 2 | 132 |
| | | | | | 1 | 140 |
| | | | | | 1 | 115 |
| | | | | | 1 | 74 |
| | 147 | 391 | VPS28 protein | NM_016208 | 10 | 221 |
| | | | | | 27 | 221 |
| | 231 | 391 | | | 9 | 211 |
| | 265 | 391 | | | 10 | 221 |
| | 317 | 391 | | | 290 | 555 |
| | 240 | 391 | Hook2 protein | AF080470 | 201 | 559 |
| | 240 | 391 | Intersection 1 | NM_003024 | 436 | 547 |
| | | | | | 437 | 584 |
| | | | | | 387 | 611 |
| | | | | | 210 | 633 |
| | 240 | 391 | Pallid | AF08080470 | 21 | 172 |
| | 240 | 391 | Catenin | U96136 | 684 | 1148 |
| | 1 | 274 | Actinin (ACTN1) | M95178 | 719 | 892 |
| | 12 | 326 | Myosin (MYH9) | M31013 | 693 | 869 |
| | 12 | 326 | Kinesin Family | U06698 | 564 | 725 |
| | | | Member 5A | | | |
| | | | (KIF5A) | | | |
| | 12 | 326 | Actin binding | AB029290 | 2326 | 2487 |
| | | | Protein (ABP620) | | | |

TABLE 6
Binding Regions of Survivin and Its Interacting Partners

| Bait Protein | | | Prey Proteins | | | |
|---|---|---|---|---|---|---|
| Name and | Amino Acid | | | GenBank ® | Amino Acid | |
| GenBank ® | Coordinates | | | Accession | Coordinates | |
| Accession No. | Start | Stop | Names | Nos. | Start | Stop |
| Survivin | 89 | 143 | Cytoplasmic dynein | U32944 | 1 | 90 |
| (BIRC5) | | | light chain 1 | | | |
| (GenBank ® | | | (HDLC1) | | | |
| Accession No.: | 3 | 99 | Cytoplasmic dynein | U32944 | −20 | 89 |
| U75285) | | | light chain 1 | | | |
| | | | (HDLC1) | | | |
| | 47 | 143 | Cytoplasmic dynein | U32944 | −20 | 89 |
| | | | light chain 1 | | | |
| | | | (HDLC1) | | | |
| | 3 | 99 | beta-actin | K00790 | 3336 | 3735 |
| | | | (ACTB) | | | |
| | 3 | 99 | DNA helicase II, | S38729 | 1131 | 4404 |
| | | | ATP-dependent, | | | |
| | | | 70 kD subunit | | | |
| | | | (KU70) | | | |
| | 47 | 143 | Beta-prime subunit | X70476 | 7796 | 906 |
| | | | of coatomer | | | |
| | | | complex (COPP) | | | |
| | 3 | 99 | Osteopontin, | BC007016 | 1 | 56 |
| | | | alt. transcript 1 | | | |
| | | | (OSTP) | | | |
| | 52 | 143 | Na⁺/Ca²⁺-exchange | M91368 | 302 | 575 |
| | | | protein 1 | | | |
| | | | (SLC8A1) | | | |
| | 52 | 143 | Catenin, alpha 2 | M94151 | 1 | 166 |
| | | | (A2-CAT) | | | |
| | 52 | 143 | Catenin, alpha 2 | M94151 | 55 | 487 |
| | | | (A2-CAT) | | | |

Note:
The negative numbers in the start column of bait and/or prey protein amino acid coordinates refer to amino acid residues encoded within the 5′ untranslated region of native cellular mRNAs, i.e., residues encoded upstream of the normal translational start site.

TABLE 7
Binding Regions of Bcl-xL and Its Interacting Partner

| Bait Protein | | | Prey Protein | | |
|---|---|---|---|---|---|
| Name and | Amino Acid | | Name and | Amino Acid | |
| GenBank ® | Coordinates | | GenBank ® | Coordinates | |
| Accession No. | Start | Stop | Accession No. | Start | Stop |
| Apoptosis Regulator | 1 | 216 | translationally- | 5 | 172 |
| Bcl-X, long form | | | controlled tumor | | |
| (Bcl-xL) | | | protein 1 (TCTP) | | |
| (GenBank ® | | | (GenBank ® | | |
| Accession No.: | | | Accession | | |
| L20121) | | | No. X16064) | | |

TABLE 8
Binding Regions of Caspase-7 and Its Interacting Partner

| Bait Protein | | | Prey Proteins | | | |
|---|---|---|---|---|---|---|
| Name and | Amino Acid | | | GenBank ® | Amino Acid | |
| GenBank ® | Coordinates | | | Accession | Coordinates | |
| Accession No. | Start | Stop | Names | Nos. | Start | Stop |
| Caspase-7 | 1 | 254 | cholesteryl | M74775 | 33 | 215 |
| (GenBank ® | | | ester | | | |
| Accession No.: | | | hydrolase/ | | | |
| U37449) | | | lysosomal | | | |
| | | | acid lipase A | | | |
| | | | (LIPA) | | | |

TABLE 9
Binding Regions of APOA1 and Its Interacting Partners

| Bait Protein | | | Prey Proteins | | | |
|---|---|---|---|---|---|---|
| Name and | Amino Acid | | | GenBank ® | Amino Acid | |
| GenBank ® | Coordinates | | | Accession | Coordinates | |
| Accession No | Start | Stop | Names | Nos. | Start | Stop |
| Apolipoprotein | 106 | 267 | prenylated Rab | NM_006423 | 1 | 185* |
| A-I | | | acceptor 1 | | | |
| (APOA1) | | | (“PRA1”) | | | |
| (GenBank ® | 23 | 267 | golgi autoantigen, | NM_005113 | 534 | 731 |
| Accession No.: | 23 | 267 | 84 kD protein | | 563 | 731 |
| NM_000039) | 106 | 267 | (“Golgin-84”) | | 512 | 726 |
| | 106 | 267 | syntaxin 2 | D14582 | 156 | 261 |
| | | | (“STX2”) | | | |
| | 106 | 267 | apolipoprotein B- | X04506 | 4307 | 4522 |
| | | | 100 (“APOB”) | | | |
| | 106 | 267 | hypothetical protein | NM_017942 | 162 | 248 |
| | | | FLJ20724 | | | |
| | | | (“FLJ20724”) | | | |

TABLE 10
Binding Regions of Apolipoprotein A-II and Its Interacting Partners\

| Bait Protein | | | Prey Proteins | | | |
|---|---|---|---|---|---|---|
| Name and | Amino Acid | | | GenBank ® | Amino Acid | |
| GenBank ® | Coordinates | | | Accession | Coordinates | |
| Accession No. | Start | Stop | Names | Nos. | Start | Stop |
| apolipoprotein | 22 | 101 | apolipoprotein A-I | NM_000039 | 87 | 267 |
| A-II | | | (“APOA1”) | | 92 | 267 |
| (APOA2) | | | giantin | X75304 | 2975 | 3259 |
| (GenBank ® | | | (“gcp372”) | | | |
| Accession No.: | | | prenylated Rab | AJ133534 | 1 | 185 |
| X04898) | | | acceptor | | 3 | 185 |
| | | | (“PRA1”) | | 11 | 185 |
| | | | | | 15 | 185 |
| | | | | | 20 | 185 |
| | | | golgi autoantigen | NM_005113 | 534 | 731 |
| | | | (“Golgin-84”) | | | |
| | | | hypothetical protein | NM_017801 | 1 | 183 |
| | | | FLJ20396 | | 14 | 183 |
| | | | (“FLJ20396”) | | 21 | 183 |
| | | | hypothetical protein | NM_022373 | 47 | 234 |
| | | | FLJ22313 | | | |
| | | | (“FLJ22313”) | | | |

TABLE 11
Binding Regions of COX1 and Its Interacting Partner

| Bait Protein | | | Prey Protein | | |
|---|---|---|---|---|---|
| Name and | Amino Acid | | Name and | Amino Acid | |
| GenBank ® | Coordinates | | GenBank ® | Coordinates | |
| Accession No. | Start | Stop | Accession No. | Start | Stop |
| Cyclooxygenase 1 | 563 | 634 | Thyroid hormone | 30 | 146 |
| (COX1) | 563 | 634 | responsive Spot 14 | 34 | 146 |
| (GenBank ® | | | (“THR S14”) | | |
| Accession | | | (GenBank ® | | |
| No.: S36219) | | | Accession | | |
| | | | No. Y08409) | | |
| | 563 | 634 | protein KIAA0567 | 197 | 425 |
| | 563 | 634 | (“Opa 1”) | 180 | 439 |
| | | | (GenBank ® | | |
| | | | Accession No. | | |
| | | | AB011139) | | |

### 2.1. Biological Significance

We have demonstrated an interaction between FMS-related tyrosine kinase 4 (FLT4) and phospholipase C, gamma 1 (PLCG1) (Table 1). FLT4 is a receptor tyrosine kinase also known as vascular endothelial growth factor receptor 3 or VEGFR-3. It is related to two vascular endothelial growth factor receptors, FLT1 and FLK1/KDR. FLT4 contains seven immunoglobulin-like domains, a transmembrane domain, and a tyrosine kinase domain. It is expressed in most fetal tissues and in tumor cell lines such as a Wilms' tumor cell line, a retinoblastoma cell line, and a nondifferentiated teratocarcinoma cell line (Pajusola et al, Cancer Research 52:5738, (1992)). In the adult, FLT4 expression is more restricted, and is found in areas such as the lymphatic endothelia and endothelia of some high endothelial venules. The ligand for FLT4 is VEGF-C. (Fielder et al., Leukemia 11:1234 (1997)) analyzed samples from acute myeloid leukemia (AML) patients, and found that FLT4 and VEGF-C were both expressed in a subset of these patients, whereas FLT4 was not found in bone marrow samples of normal volunteer donors. In addition to AML, FLT4 has been implicated in numerous other cancers. Kajita et al., Br J Cancer 85:255 (2001), found a positive correlation between VEGF-C expression of cancer cells from non-small cell lung cancer and FLT4 in vascular endothelial cells. Based on expression studies employing methods of immuno-histochemistry, both FLT4 and VEGF-C are also associated with angiogenesis in breast cancer (Valtola et al., Am J Pathol 154:1381 (1999)).

PLCG1 is activated by phosphorylation by tyrosine kinases upon binding of ligand to various growth factor receptors and immune system receptors. The activation of PLCG1 results in the formation of two important second messenger molecules, inositol 1,4,5-trisphosphate and diacylglycerol. This reaction requires the presence of calcium. PLCG1 is expressed in most tissues. It was found by in situ hybridization to be localized on 20q12-q13.1. (Bristol et al., Cold Spring Harb. Symp. Quant. Biol., 53:915 (1988)). Interestingly, this region is often involved in interstitial deletions and breakpoints in myeloid malignancy. Recently, Ye et al., Nature 415:541 (2002) demonstrated that PLCG1 functions as a guanine nucleotide exchange factor for PIKE, a nuclear GTPase. PIKE involved in nerve growth factor-mediated activation of nuclear phosphatidylinositol-3-hydroxykinase activity, and PLCG1 may therefore regulate some aspects of mitogenesis. Mice deficient in PLCG1 are embryonic lethals, due to an absence of erythrogenesis and vasculogenesis (Liao et al., J Biol Chem 277:9335 (2002)).

The interaction between FLT4 and PLCG1 indicates that like the related receptors, VEGFR-1 and -2 (Knight et al., Oncogene 19:5398 (2000); Gille et al., J Biol Chem 276:3222 (2001)), FLT4 most likely also binds to, phosphorylates, and activates PLCG1. This interaction may be important in FLT4 signal transduction and may play an important role in angiogenesis, erythrogenesis, or mitogenesis. For example, by modulating (e.g., disrupting) the interaction between FLT4 and PLCG1, FLT4 signal transduction in cells may be modulated and excessive angiogenesis may be inhibited or prevented, leading to the treatment of the diseases associated with excessive angiogenesis. On the other hand, modulation (e.g., promotion) of the FLT4-PLGC1 interaction may also stimulate angiogenesis.

In addition, we have also demonstrated an interaction between FMS-related tyrosine kinase 4 (FLT4) and E6-AP ubiquitin-protein ligase (UBE3A) (Table 1). UBE3A functions as a ubiquitin ligase that interacts with and forms a complex with the E6 cancer-associated human papillomavirus (Huibregtse et al., Mol. Cell. Biol., 13:775 (1993)). This complex targets the p53 tumor suppressor protein for degradation via ubiquitin-mediated proteolysis, thus deregulating cell growth control. UBE3A can also target itself for degradation. Mutations in the UBE3A gene results in a neurological disorder called Angelman syndrome (also called happy puppet syndrome), which is characterized by mental retardation, seizures, abnormal gait and limb movements, hyperactivity, and frequent, uncontrolled smiling and laughter (Kishino et al., Nat. Genet. 15:70 (1997)). Ubiquitination-conjugation is involved in the internalization and degradation of receptors, transporters, and channels. (Qui et al. (J Biol Chem 275:35734 (2000)) showed that Itch, which is homologous to the carboxyl terminus of UBE3A, associates with the intracellular portion of the transmembrane protein, Notch. UBE3A has also been shown by (Nawaz et al., Mol Cell Biol 19:1182 (1999)) to have additional functions as a coactivator for nuclear hormone receptors. The interaction between FLT4 and UBE3A could represent a means by which FLT4 surface expression is modulated. UBE3A could target FLT4 for internalization and/or subsequent degradation in lysosomes.

Thus, by modulating the interaction between UBE3A and FLT4, the FLT4 signal transduction may be modulated which may lead to the modulation of cellular processes such as angiogenesis and cell proliferation and transformation.

Akt1 and Akt2 are serine/threonine protein kinases capable of phosphorylating a variety of known proteins. Akt1 and Akt2 are activated by platelet-derived growth factor (PDGF), a growth factor involved in the decision between cellular proliferation and apoptosis (Franke et al., Cell. 81:727-36 (1995)). Akt kinases are also activated by insulin-like growth factor (IGF1), and in this capacity are involved in survival of cerebellar neurons (Dudek, H. et al. Science. 275:661-5 (1997)). Furthermore, Akt1 is involved in the activation of NFkB by tumor necrosis factor (TNF) (Ozes, O, N. et al., Nature 401:82-5 (1999)). Akt2 has been shown to be associated with pancreatic carcinomas (Cheng, J. Q. et al., Proc Natl Acad Sci USA. 93:3636-41 (1996)). Akt kinases have been implicated in insulin-regulated glucose transport and the development of non-insulin dependent diabetes mellitus (Krook, A. et al., Diabetes. 47:1281-6 (1998)).

The p90/RSK kinase (also known as HU1) is also involved in intracellular signaling cascades relevant to human disease. p90/RSK activity is regulated by growth factors, and the phosphorylation of two p90/RSK substrates, BAD and CREB, suppresses apoptosis in neurons (Bonni, A. et al., Science. 286:1358-62 (1999)). p90/RSK is also implicated in cell cycle control in response to Mos-MEK1 signaling (Bhatt, R. R. and Ferrell, J. E., Jr. Science. 286:1362-5 (1999); Gross, S. D. et al., Science. 286:1365-7 (1999)).

Clearly, these kinases play varied and important roles in a number of intracellular signaling pathways, and are thus good starting points from which to identify novel protein interactions that define disease-related signal transduction pathways. Towards this end, Akt1 and Akt2 were used in yeast two-hybrid assays to identify Akt-interacting proteins that may be potential targets for drug intervention. Here, we describe new protein-protein interactions for Akt1, Akt2, and p90/RSK.

The first interactor for Akt1 is the alpha subunit of p21 (RAS) farnesyl transferase (FNTA) (Table 2). FNTA has been shown to bind to both the TGF-beta and activin receptors in the yeast two-hybrid assay (Ventura, F. et al., J Biol Chem. 271:13931-4 (1996); Wang, T. et al., Science. 271:1120-2 (1996)). Further, it has been shown that FNTA binds to the TGF-beta receptor in the absence of ligand, and that ligand binding causes the phosphorylation and release of FNTA. Presumably, FNTA is then free to interact with other cytoplasmic factors in the transmission of the TGF-beta signal. The finding that Akt1 interacts with FNTA suggests a direct connection between receptors at the cell surface and the intracellular signal transduction machinery involving Akt1.

The second interactor for Akt1 is the periplakin protein (PPL) (Table 2). The plakins are cytoskeletal coiled-coil proteins that bind to intermediate filaments as well as actin and microtubule networks. Periplakin has been shown to bind to the intracellular portion of collagen type XVII in a yeast two-hybrid assay (Aho, S. et al., Genomics 48:242-7 (1998)). Periplakin appears to be highly expressed in tissues that are rich in epithelial cells. The interaction of periplakin with Akt1 suggests it may be a substrate of this kinase, and that its function may be modulated by phosphorylation. Alternatively, the subcellular localization of Akt1 may be altered by its interaction with periplakin.

The hypothetical protein KIAA0728, now recognized as “dystonin,” was found as an interactor of both Akt1 and p90/RSK (Tables 2 & 4). KIAA0728 contains an EF hand calcium-binding motif, a nuclear localization sequence and six spectrin repeats. The Akt1- and p90/RSK-interacting regions of KIAA0728 overlap, suggesting these proteins may bind the same domain of KIAA0728. The interaction of KIAA0728 with both Akt1 and p90/RSK suggests that it may act as a substrate for both enzymes, or alternatively that KIAA0728, by virtue of its spectrin repeats, may serve as a scaffold to link these two kinases together.

Akt1 was also found to interact with the integral membrane protein Golgin-84 (Table 2). Golgin-84 is a coiled-coil containing protein that was originally isolated as an yeast two-hybrid interactor of the OCRL1 phosphatidylinositol(4,5)P2 5-phosphatase that is implicated in oculocerebrorenal syndrome (Bascom, R. A. et al., J Biol Chem. 274:2953-62 (1999)). In vitro studies indicate that most of the golgin-84 protein is predicted to be cytoplasmic with only the most extreme C-terminus of the protein extending to the extracellular/vesicular side of membranes. Importantly, the cytoplasmic portion of golgin-84 associates with Akt1.

The TPR domain protein TPRD was found to interact with both Akt1 and Akt2 (Tables 2 & 3). TPDR may play a major role in development since it is localized to the Down syndrome-critical region on human chromosome 21q22.2 (Ohira, M. et al., DNA Res. 3:9-16 (1996); Tsukahara, F. et al., J Biochem (Tokyo). 120:820-7 (1996)). Analysis of the amino acid sequence of TPRD reveals the presence of TPR repeats towards the N-terminus of the protein, a bipartite nuclear localization sequence, and a zinc finger. The region of TPRD that associates with the two Akts (amino acids 1058 to 1189) is located near the center of the protein and is distinct from any of the predicted structural domains.

Akt2 was also found to interact with the aldehyde reductase AKR7A2 (aflatoxin B1-dialdehyde reductase or AFAR) (Table 3). AKR7A2 is an aldoketoreductase that resides in the cytoplasm of many if not all tissues. AKR7A2 appears to be highly regulated at the transcriptional level. Studies using rats have demonstrated that AKR7A2 mRNA and protein levels increase dramatically in the liver following exposure to dietary antioxidants (Ellis, E. M. et al., Cancer Res. 56:2758-66 (1996)). The finding that AKR7A2 associates with Akt2 suggests that perhaps this enzyme is also regulated at the post-translational level by Akt2.

The intracellular chloride channel protein CLIC1 was also shown to interact with Akt2 (Table 3). CLIC1, also known as NCC27 (nuclear chloride channel-27), was first cloned from human U937 myelomonocytic cells and is the first member of the CLIC family of chloride channels (Valenzuela, S. M. et al., J Biol Chem. 272:12575-82 (1997)). CLIC1 primarily localizes to the nuclear membrane and likely plays a role in the transport of chloride into the nucleus. The finding that CLIC1 and Akt2 associate with one another is rather intriguing, and it suggests that Akt2 may play a role in regulating nuclear ion transport. Interestingly, another related CLIC family member that localizes to the nuclear membrane, CLIC3, has been demonstrated to interact with a signal transduction protein, ERK7 (Qian, Z. et al., J Biol Chem. 274:1621-7 (1999)). Taken together, these results suggest that intracellular chloride channels may be intimately linked to transduction of extracellular signals.

Finally, the UNR (upstream of N-ras) protein was shown to associate with p90/RSK (Table 4). UNR has no known function though it does contain several cold shock DNA-binding domains and two predicted peroxidase active sites. Transcription of UNR, which is located immediately upstream of the N-ras gene, interferes with transcription of N-ras (Boussadia, O. et al., Biochim Biophys Acta 1172:64-72 (1993)). Furthermore, the human and rat UNR genes appear to undergo exon skipping that is tissue-dependent (Boussadia, O. et al., Biochim Biophys Acta 1172:64-72 (1993)). Interestingly, one of the UNR protein products has been shown to interact with the protein product of the ALL-1 gene, which is involved in human chromosome translocations and other rearrangements in acute lymphocytic leukemia (Leshkowitz, D. et al., Oncogene. 13:2027-31 (1996)). ALL-1 is the human homolog of the Drosophila trithorax protein and plays a role in the regulation of homeotic genes involved in body segmentation. The finding that p90/RSK binds to UNR suggests that RSK may be capable of phosphorylating UNR, thereby affecting its function. Because UNR interacts with ALL-1, it seems likely that such regulation of UNR by p90/RSK might affect gene transcription.

As shown in Table 5 above, the inventors of the present invention identified a large number of protein interactors of tumor susceptibility gene 101 protein (TSG101), many of which are known to be involved in intracellular vesicle trafficking and vacuolar protein sorting.

Human TSG101 interacts with human vacuolar protein sorting 28 (VPS28): In accordance with the present invention, C-terminal fragments of TSG101 interacted with VPS28 in two different searches. One search of a hippocampal library utilized a TSG11 bait fragment consisting of residues 147-391, while the other search of a breast and prostate cancer library utilized a shorter C-terminal fragment consisting of amino acid residues 240-391. Both TSG11 fragments contain an alpha-helical region, and the longer fragment contained an overlapping coiled coil region as well. Both TSG101 fragments also interacted with VPS28 via residues 27-221. In addition, VPS28 residues 10-221 were also isolated as a prey using the TSG101 bait fragment amino acids. VPS28 is a class E protein involved in endocytosis. It consists of 221 amino acids and plays a role in the formation of multivesicular bodies and endosomal sorting. (Rieder et al., Mol. Biol. Cell, 7(6):985-99 (1996)). Mutations in VPS28 result in defects in endocytic traffic destined for the vacuole. Although TSG101 and VPS28 are predominantly cytosolic, both proteins are recruited to endosomal vacuoles when a dominant-negative mutant VPS4 is expressed. Thus, both TSG101 and VPS28 may be involved in endosomal sorting by functioning together in a multiprotein complex.

TSG101 interacts with a GTPase-activating protein (IQGAP1): A C-terminal fragment of TSG101 consisting of amino acid residues 240-391 was used in two different searches of a breast and prostate cancer library. This TSG101 fragment, which contains most of an alpha-helical region, interacted with an IQ motif-containing GTPase-activating protein (IQGAP). IQGAP, a protein of 1657 amino acids, is expressed in many tissues including placenta, lung, and kidney. It contains several motifs including a Ras-related GTPase-activating (RasGAP) domain, a calponin homology domain, and four IQ motifs (named for the presence of tandem isoleucine and glutamine residues), which are known to modulate binding with subsequently cloned its cDNA. Recombinant IQGAP bound to activated Cdc42 and Rac and inhibited their GTPase activity while the C-terminal domain IQGAP was shown to inhibit the GTPase activity of Cdc42. (Hart et al., EMBO J., 15(12):2997-3005 (1996)). IQGAP has also been shown to bind to actin, calmodulin, E-cadherin and beta-catenin. (Li et al., J. Biol. Chem. 274(53):37885-92 (1999); Fukata et al., J. Biol. Chem., 274(37):26044-50 (1999)). It may thus serve as a scaffolding protein and provide a link between calcium/calmodulin and Cdc42 signaling as well as with cell adhesion and the actin cytoskeleton. (Ho et al., J. Biol. Chem., 274(1):464-70 (1999)). Interestingly, the small GTPases Cdc42 and rac, both of which associate with Tsg101, appear to be involved in endocytosis. (Malecz et al, Curr. Biol., 10(21):1383-6 (2000)). With its multiple domains, its association with the actin cytoskeleton, and its RasGAP-like domain, IQGAP could be a good candidate for a regulator of endocytic trafficking.

TSG101 binds to hook2 protein: A C-terminal fragment of TSG101 consisting of amino acid residues 240-390 was used in searches of a breast and prostate cancer library. This TSG101 fragment, which contains most of an alpha-helical region, interacted with Hook2 (via amino acids 132-428). Hook was originally identified in Drosophila as a protein involved with endocytic trafficking. Kramer and Phistry, J. Cell Biol., 133(6):1205-15 (1996). The gene encoding Hook2 (719 amino acids) was identified from sequence-homology searches of EST databases as having significant homology to the Drosophila hook gene. Kramer and Phistry, Genetics, 151(2):675-84 (1999). The Hook2 protein can be alternatively spliced, yielding a protein lacking amino acids 173-522. All Hook proteins contain two coiled coil regions in the central portion of the protein and a conserved 125 amino acid N-terminal domain of unknown function. Immunohistochemical studies showed that Hook localizes to endocytic vesicles and large vacuoles, implicating Hook in late endocytic trafficking. In hook mutants, cells lack mature MVBs and have an overabundance of late endosomes or lysosomes, indicating that Hook may stabilize mature MVBs and negatively regulate transport to late endosomes perhaps by inhibiting the fusion of MVBs to late endosomes. (Sunio et al., Mol. Biol. Cell., 10(4):847-59 (1999)). The TSG101 and Hook proteins appear to be prime candidates for regulating fusion at the MVB and endosome stages. The fact that they interact lends further support to this theory.

TSG101 interacts with intersectin 1 (ITSN1): A C-terminal fragment of TSG101 consisting of amino acid residues 240-391 was used in two different searches of a breast and prostate cancer library. This TSG101 fragment, which contains most of an alpha-helical region, interacted with a number of different fragments of Intersectin 1 within the amino acids 201-633 region as indicated in the tables above. Northern analysis showed that intersectin mRNA is widely expressed, but most highly in brain, heart, and skeletal muscle. Intersectin 1 is a protein consisting of 1721 amino acids that contains two N-terminal EH domains, a central coiled coil domain and five C-terminal SH3 domains. The regions interacting with TSG11 correspond to more C-terminal EH domain and more N-terminal coiled coil domain. It has been found that Intersectin 1 binds in vivo to Eps15. (Sengar et al., EMBO J., 18(5):1159-71 (1999)). The EH domain of Intersectin 1 binds to Epsin whereas its SH3 domains bind to dynamin. Eps15 is an essential component of the early endocytic pathway that is localized to the neck of clathrin-coated pits. (Benmerah et al., J. Cell Biol., 140(5):1055-62 (1998)). Dynamin is a GTPase which presumably functions to sever forming vesicles from the plasma membrane and is essential for receptor-mediated endocytosis. Epsin binds to clathrin and regulates receptor-mediated endocytosis. The interaction between Intersectin 1 and Eps15 appears to function as a scaffold which links dynamin, epsin, and other endocytic pathway components. The interaction between TSG101 and Intersectin 1 suggests that TSG101 may play a role in budding of membrane particles in various stages of endocytosis.

TSG101 interacts with GEF-H1: A search of a brain library with the TSG101 identified GEF-H1 as an interactor. GEF-H1 is an 894 amino acid protein identified by homology to guanine nucleotide exchange factors (GEFs) in a screen of a HeLa cell cDNA library. (Ren et al., J Biol Chem, 273(52):34954-60 (1998)). GEF-H1 contains a Dbl-type GEF domain in tandem with a pleckstrin homology domain, a motif typically responsible for protein or lipid/membrane interaction. GEF-H1 binds Rac and Rho (known regulators of the cytoskeleton) and stimulates guanine nucleotide exchange of these GTPases, but GEF-H1 is inactive towards Cdc42, Ras, or other small GTPases. GEF-H1 also contains a C-terminal coiled-coil domain; immunofluorescence experiments reveal that this domain is responsible for colocalization of GEF-H1 with microtubules. Overexpression of GEF-H1 in COS-7 cells induces membrane ruffles. Together, these findings suggest that GEF-H1 may have a direct role in activating Rac and/or Rho and may localize these GTPases to microtubules, thereby coordinating cytoskeletal reorganization.

TSG101 interacts with the protein kinase ROCK1: A search of a macrophage library with the TSG101 identified the Rho-associated coiled coil-containing kinase ROCK1 as an interactor. ROCK1, also known as ROK or p160, is a 1354 amino acid Ser/Thr-kinase that is activated by the small GTPase Rho, a known cytoskeletal regulator. (Fujisawa et al., J Biol Chem 20; 271(38):23022-8 (1996); Leung et al., Mol. Cell Biol., 16(10):5313-27 (1996)). Activation of ROCK1 by Rho results in phosphorylation of LIM kinase, which in turn phosphorylates cofilin and inhibits its actin-depolymerizing activity. (Maekawa et al., Science 285(5429):895-8 (1999)). ROCK1 activity also results in phosphorylation of myosin light chain (MLC) and ERM (ezrin/radixin/moesin) proteins, which in turn mediate cytoskeletal responses. (Tran et al., EMBO J, 19(17):4565-76 (2000); Kosako et al., Oncogene, 19(52):6059-64 (2000); Takaishi et al., Genes Cells, 5(11):929-936 (2000)). The effect of ROCK1 on MLC phosphorylation appears to be both indirect (via inhibition of MLC phosphatase and/or activiation of MLC kinase) and direct. (Tatsukawa et al., J. Cell Biol., 150(4):797-806 (2000); Kosako et al., Oncogene, 19(52):6059-64 (2000)). Substantial evidence supports roles for ROCK1 in processes such as formation of stress fibers, axonal outgrowth, smooth muscle contraction, cell motility, tumor cell invasion, and cytokinesis. See references above; (Watanabe et al., Nat. Cell Biol., 1(2):E31-3 (1999); Bito et al., Neuron, 26(2):431-41 (2000)). ROCK1 has also been implicated in intracellular lysosome trafficking by controlling microtubule organization. (Nishimura et al., Cell Tissue Res., 301(3):341-51 (2000)). In these studies, ROCK1 activity was shown to be both necessary and sufficient for the formation of apoptotic membrane blebs (a process dependent on MLC phosphorylation) and for relocalization of fragmented genomic DNA to these blebs. Interestingly, a ROCK1-specific inhibitor has been identified; this compound, designated Y-27632 [(+)-(R)-trans-4-(1-aminoethyl)-N-(4-pyridyl)cyclohexanecarboxamide], is commercially-available from Tocris and is highly selective for ROCK1. This compound has been used in many of the studies cited above to inhibit ROCK1-dependent processes in various cell lines. The ROCK1 protein contains an N-terminal protein kinase domain, a large central coiled-coil domain, a leucine zipper (which mediates interaction with RhoA), and a C-terminal pleckstrin homology domain (protein and/or membrane/lipid interaction motif). Two prey constructs encoding amino acids 462-617 of ROCK1 were isolated according to the present invention; this region corresponds to part of the central coiled-coil motif. Analysis of homologous ESTs indicates that ROCK1 is expressed in a wide variety of tissues.

The known functions of ROCK1 in controlling the cytoskeleton, vesicular trafficking, and membrane blebbing are intriguing in light of the proposed roles for TSG101 in viral assembly. The interaction of TSG101 with ROCK1 suggests ROCK1 may be targeted to sites of viral budding, where it may recruit and activate proteins involved in the final stages of this process. Thus, inhibitors of ROCK1 may be useful in inhibiting viral budding and in treating viral infection such as HIV infection and AIDS. Thus, the present invention provides a method of treating viral infection, particularly HIV infection and AIDS using Y-27632 [(+)-(R)-trans-4-(1-aminoethyl)-N-(4-pyridyl)cyclohexanecarboxamide] by administering the compound to a patient in need of treatment.

TSG101 interacts with PACSIN2: A search of a macrophage library with the TSG101 identified PACSIN2 as an interactor. PACSIN2 (which stands for PKC and casein kinase substrate in neurons 2) is a 486 amino acid protein isolated by its similarity (primary sequence and domain organization) to PACSIN1, a protein that is upregulated during neuronal differentiation and is phosphorylated by both PKC and casein kinase II. (Ritter et al., FEBS Lett 454(3):356-62 (1999)). Immunofluorescence microscopy of transfected NIH3T3 fibroblasts reveals a broad, vesicle-like PACSIN2 distribution pattern, suggesting a role in vesicular trafficking and/or the regulation of the actin cytoskeleton. In support of this, PACSIN2 is closely related (˜90% amino acid identity) to rat syndapin II proteins, which are involved in receptor-mediated endocytosis and actin cytoskeleton reorganization. Qualmann and Kelly, J Cell Biol, 148(5):1047-62 (2000). PACSIN2 is a 486 amino acid protein that contains an N-terminal FCH domain, which is found in proteins such as CIP4, an intermediate protein between Cdc42 kinase and cytoskeletal proteins, and Cdc15, a protein kinase involved in regulating actin at mitosis. PACSIN2 also contains a C-terminal SH3 domain, suggesting interaction with certain signaling proteins. EST analysis suggests expression of PACSIN2 in a wide variety of tissues.

TSG101 interacts with the integral membrane protein Golgin-84: A search of a spleen library with the TSG101 identified Golgin-84 as an interactor. Golgin-84 is a 731 amino acid protein that was originally identified in a yeast two-hybrid search using the peripheral Golgi phosphatidylinositol phosphatase OCRL1 as bait. (Bascom et al., J. Biol. Chem., 274(5):2953-62 (1999)). Golgin-84 is an integral membrane protein with a single transmembrane domain located near its C-terminus. In addition, Golgin-84 contains a large central coiled-coil motif. In vitro, the protein inserts post-translationally into microsomal membranes with an N-cytoplasmic and C-lumen orientation. Crosslinking experiments indicate that Golgin-84 is able to form homodimers, presumably via the large coiled-coil motif. Interestingly, when fused to the RET tyrosine kinase domain, this coiled-coil motif of Golgin-84 activates RET and forms the RET-II oncogene. Structurally, Golgin-84 is similar to giantin, which is involved in tethering coatamer complex I vesicles to the Golgi, suggesting that Golgin-84 may perform a similar tethering function. Expression studies and analysis of homologous ESTs indicate ubiquitous expression of Golgin-84.

TSG101 interacts with the integral membrane protein Golgin-67: A search of a spleen library with the TSG101 identified golgin-67 as an interactor. Golgin-67 was fortuitously identified in searches of a T-cell expression library with antibodies against the mitotic target of Src, Sam68. (Jakymiw et al., J. Biol. Chem., 275(6):4137-44 (2000)). Golgin-67 was also identified as an autoimmune antigen in various systemic rheumatic diseases. (Eystathioy et al., J. Autoimmun., 14(2):179-87 (2000)). The 460 amino acid golgin-67 protein is structurally similar to golgin-84; both contain C-terminal transmembrane domains and large central coiled-coil regions. Cytological analysis demonstrates that golgin-67 is localized to the Golgi complex, and the transmembrane domain is necessary for localization to the Golgi.

TSG101 interacts with kinectin: A two-hybrid search of a brain library with the TSG101 was found to interact with kinectin as an interactor. Kinectin is a large (1,356 amino acid) integral ER membrane protein that contains an N-terminal transmembrane domain and C-terminal coiled-coil and leucine zipper motifs. (Futterer et al., Mol. Biol. Cell, 6(2): 161-70 (1995); Yu et al., Mol. Biol Cell, 6(2):171-83 (1995)). Antibodies against kinectin reveal a perinuclear, ER-like protein distribution. In vitro, kinectin is able to bind kinesin, a microtubule-associated ATP-dependent motor protein involved in vesicular transport along microtubules, and kinectin has been proposed to function as a receptor for kinesin on the surface of certain organelles. The C-terminal region of kinectin is responsible for interaction with kinesin. (Ong et al., J. Biol. Chem., 275(42):32854-60 (2000)). Interaction of these proteins enhances the microtubule-stimulated ATPase activity of kinesin, and overexpression of the kinesin-binding domain of kinectin inhibits kinesin-dependent organelle motility in vivo, supporting a role for kinectin in vesicular transport. Kinectin has been shown to be a proteolytic target of caspases during apoptosis (Machleidt et al., FEBS Lett., 436(1):51-4 (1998)), suggesting a role in mediating programmed cell death. Kinectin is also a translocation partner of the RET tyrosine kinase in certain thyroid carcinomas, resulting in a constitutively active form of RET. (Salassidis et al., Cancer Res., 60(11):2786-9 (2000)). This is potentially interesting, in light of the observation that fusions between RET and another protein thought to be involved in vesicular transport, Golgin-84, also result in activation of RET. (Bascom et al., J. Biol. Chem., 274(5):2953-62 (1999)). Finally, kinectin has been shown in the literature to interact with the GTP-bound forms (but not the GDP-bound forms) of various small Rho-family GTPases involved in cytoskeletal regulation, including RhoA, Rac1, and Cdc42. (Hotta et al., Biochem Biophys Res Commun 225(1):69-74 (1996)). This observation provides further links between TSG101 and proteins involved in regulating the cytoskeleton. Three prey clones corresponding to kinectin were isolated; these encode similar, but distinct, fragments of the protein that overlap the region of kinectin responsible for interaction with kinesin.

TSG101 interacts with CYLN2: A search of a brain library with the TSG101 identified the cytoplasmic linker protein CYLN2 (also known as CLIP-115, for cytoplasmic linker protein-115 kD) as an interactor. CYLN2 is a large (1,046 amino acid) protein that contains an N-terminal globular domain with two CAP-Gly (microtubule-binding) motifs, and a large central coiled-coil region. CAP-Gly domains are ˜42 amino acid motifs found in proteins such as Restin (also known as CLIP-170), which links endocytic vesicles to microtubules, and dynactin, which stimulates dynein-mediated vesicle transport. The presence of these motifs suggests that CYLN2 functions to control vesicular transport in association with the cytoskeleton, and indeed this is the case. CYLN2 is able to bind microtubules and is enriched in dendritic lamellar body (DLB), an organelle that is actively localized to dendritic appendages in a microtubule-dependent fashion. Recent analyses demonstrate that the association of CYLN2 with microtubules is sensitive to phosphorylation and is dependent not only on its CAP-Gly domains but also on the surrounding basic, Ser-rich regions, and furthermore that CYLN2 colocalizes with Restin at the distal ends of microtubules in transfected COS-1 cells. (Hoogenrad et al., J. Cell Sci., 113 (Pt 12):2285-97 (2000)). There is also evidence suggesting clinical relevance of CYLN2: the CYLN2 gene is localized to 7q11.23, a region commonly deleted in Williams syndrome, a multisystemic developmental disorder that includes infantile hypercalcemia, dysmorphic facies, and mental retardation. (Hoogenrad et al., Genomics, 53(3):348-58 (1998)). However, it has not yet been demonstrated whether deletion of CYLN2 is responsible for Williams syndrome. Although CYLN2 has been described by one group as a brain-specific protein, expression of homologous ESTs is observed in a wide variety of tissues. One clone encoding amino acids 607-947 of CYLN2 (corresponding to part of the central coiled-coil motif) was isolated according to the present invention.

In addition, we also identified an interaction between TSG101 and Restin. The similarity of both the domain structures and functions of Restin and CYLN2 strengthens the notion that the interaction of TSG101 with these proteins is physiologically relevant.

TSG101 interacts with the tropomyosin TPM4: A search of a macrophage library with the TSG101 identified the tropomyosin TPM4 as an interactor. Tropomyosins are small, acidic, coiled-coil proteins that bind as dimers along the length of actin filaments and coordinate the formation of contractile bundles (as opposed to a network of actin filaments). Binding of tropomyosin stabilizes and stiffens the actin filament, inhibits the binding of filamin, and facilitates the binding of myosin to actin filaments, thereby facilitating the formation of a contractile actin bundle. TPM4 was isolated from human fibroblasts based on homology to horse tropomyosin, and was described as one of five proteins in human fibroblasts similar to tropomyosins. (MacLeod et al., J. Mol. Biol., 194(1):1-10 (1987)). TPM4 is a non-muscle tropomyosin, but both muscle and non-muscle forms are produced by alternative splicing of the same four genes. The interaction of TSG11 with TPM4 provides yet another link between TSG101 and regulation of the cytoskeleton. Analysis of homologous ESTs suggests widespread expression of TPM4.

TSG101 interacts with KIAA0674: A search of a macrophage and spleen libraries with two different TSG101 baits identified the FK506-binding protein (FKBP) homolog KIAA0674 as an interactor. The available KIAA0674 sequence, which is incomplete, predicts a 1234 amino acid protein. KIAA0674 contains an FKBP-type peptidyl-prolyl cis-trans isomerase (PPIase) domain, which is likely involved in promoting protein folding by catalyzing the isomerization of proline imidic peptide bonds. FKBPs, which bind the immunosuppressive drug FK506, possess this domain and display PPIase activity. In addition, KIAA0674 contains an N-terminal WASp homology (WH) domain, found in the Wiskott-Aldrich syndrome protein (WASp) involved in the transmission of signals to the cytoskeleton. The WH motif is also found in Horner proteins (e.g. Horner-1 B), which are involved in neurotransmitter release, and there is evidence that the WH domain is responsible for binding polyproline-containing peptides in glutamate receptors and cytoskeletal components. In addition, KIAA0674 contains a central coiled-coil region that displays weak similarity to myosin heavy chain, plectin, and golgin-like proteins. The presence of these domains suggests a function for KIAA0674 in controlling the conformation of cytoskeletal or other proteins, perhaps in response to extracellular signals. Analysis of homologous ESTs suggests expression of KIAA0674 in a wide variety of tissues. Six prey clones encoding amino acids 770-880 of KIAA0674 were isolated according the present invention; this region corresponds to the central coiled-coil domain. The isolation of multiple KIAA0674 clones with independent TSG101 baits strengthens the notion that this may be a biologically relevant interaction.

Interestingly, the HIV GAG protein has been shown to interact with the PPIase-domain protein folding catalysts cyclophilin A and cyclophilin B. (Luban et al., Cell, 73(6): 1067-78 (1993)). Cyclophilin A (CypA) is incorporated into HIV virions (Colgan et al., J. Virol., 70(7):4299-310 (1996)), and there is evidence that CypA mediates attachment of the virus to the cell surface by binding to heparan. (Saphire et al., EMBO J., 18(23):6771-85 (1999)). Consistent with this, HIV-1 exhibits decreased replication in T cells in which the CypA gene has been deleted by homologous recombination, and viruses produced by CypA-deficient cells are less infectious than virions from wild type cells. While it seems that CypA plays a role in an early step in viral infection, it is also possible that CypA, and other PPIase proteins including KIAA0674, also function during viral assembly and budding; the functions of these proteins as catalysts of protein folding certainly raises the possibility that they assist in the assembly of virus particles.

TSG11 interacts with Plectin 1: A search of a spleen library with the TSG11 identified Plectin 1 (plectin) as an interactor. Plectin is an intermediate filament binding protein that crosslinks intermediate filaments, links intermediate filaments to microtubules and microfilaments, and anchors intermediate filaments to both the plasma and nuclear membranes. Plectin is able to self-associate, forming networks that stabilize the cytoskeleton. Plectin is one of the largest known proteins (4574 amino acids, 518 kD). (Liu et al., Proc. Natl. Acad. Sci., 93(9):4278-83 (1996)). Plectin contains an N-terminal globular domain with two calponin homology (CH) motifs (responsible for binding to actin), a central rod-like domain containing coiled-coil regions, and a repetitive C-terminal globular domain (plectin repeats). Mutations in plectin have been shown to cause muscular dystrophy with epidermolysis bullosa simplex (MD-EBS), a disorder characterized by epidermal blister formation associated with muscular dystrophy. (Gache et al., J. Clin. Invest., 97(10):2289-98 (1996); Smith et al., Nat. Genet., 13(4):450-7 (1996); MacLean et al., Genes Dev., 10(14):1724-35 (1996)). Plectin has been shown to be a major early substrate for caspase-8 during CD95- and TNF receptor-mediated apoptosis, and in primary fibroblasts from plectin-deficient mice, apoptosis-induced reorganization of the cytoskeleton was severely impaired. (Stegh et al., Mol. Cell Biol., 20(15):5665-79 (2000)). These results suggest an active role for plectin in controlling the cellular changes associated with apoptosis.

Immunocytological analysis of transfected HeLa cells demonstrates the localization of Vif protein to perinuclear aggregates, and the relocalization of cytoskeletal components including vimentin and plectin (but not tubulin) to these sites. In COS-7 cells, Vif does not form perinuclear aggregates, but rather is found throughout the cytoplasm; nonetheless, Vif expression in COS-7 cells is still able to induce perinuclear aggregation of vimentin and plectin. Although the redistribution of plectin upon Vif expression is certainly not proof of physical interaction, it is suggestive of at least a functional connection between these proteins. Two prey clones from plectin were isolated; these encode similar but distinct fragments corresponding to the central coiled-coil region of the protein.

The interaction of TSG101 with plectin, and the altered intracellular behavior of plectin upon expression of HIV-1 Vif protein, suggest that plectin may be involved in viral infection, particularly HIV-1 infection.

TSG101 interacts with the actin binding protein ACTN4: A search of a spleen library with the TSG101 identified ACTN4 as an interactor. ACTN4 was identified as an actin-bundling protein associated with cell motility and cancer invasiveness. (Honda et al., J. Cell Biol, 140(6):1383-93 (1998)). ACTN4 localizes to the cytoplasm where it links actin to membranes in non-muscle cell types and anchors myofibrillar actin filaments in skeletal, cardiac, and smooth muscle cells. ACTN4 is conspicuously absent from focal adhesion plaques and adherens junctions, where the classic isoform (ACTN4 1) is localized. Subsequent analysis (El-Husseini et al., Biochem. Biophys. Res. Commun., 267(3):906-11 (2000)) demonstrated that ACTN4 binds to and colocalizes with BERP, a member of the RING-B-box-coiled-coil (RBCC) subgroup of RING finger proteins. BERP is a specific partner for the tail domain of myosin V, a class of myosins which are involved in the targeted transport of organelles, suggesting that BERP, and by inference ACTN4, may be involved in intracellular cargo transport. (El-Husseini et al., J. Biol. Chem., 274(28):19771-7 (1999)). Mutations in ACTN4 are associated with focal and segmental glomerulosclerosis (FSGS), a common, non-specific renal lesion characterized by urinary protein secretion and decreasing kidney function. (Kaplan et al., Nat. Genet., 24(3):251-6 (2000)). Mutant forms of ACTN4 bind actin more strongly than does the wild type protein, resulting in misregulation of the actin cytoskeleton in glomerular cells of affected FSGS patients. ACTN4 is an 884 amino acid protein with a domain structure very similar to that of PLEC1: ACTN4 contains two N-terminal CH (actin-binding) motifs and a C-terminal repetitive region (spectrin repeats). In addition, ACTN4 contains two C-terminal EF-hand calcium binding motifs.

TSG101 interacts with PIBF1: A search of a spleen library with the TSG101 (amino acids 12-326) identified PIBF1 as an interactor. PIBF1 is a 758 amino acid protein that contains numerous coiled-coil motifs and a weak match to the Syntaxin N-terminal domain motif, which is involved in interaction of SNAREs during vesicular docking and fusion. In addition, PIBF1 displays weak homology to myosin heavy chain. The interaction between TSG101 and PIBF1, as well as the presence of these domains suggest that PIBF1 may be involved in regulating the cytoskeleton or in vesicular transport. Analysis of homologous ESTs suggests expression of PIBF1 in a variety of tissues. Two prey clones from PIBF1 have been isolated; these encode a region of PIBF1 (amino acids 392-758) that contains two of the coiled-coil motifs.

TSG101 interacts with BAP31: A search of a spleen library using amino acids 12-326 of the TSG101 revealed an interaction with the transmembrane ER protein BAP31. BAP31 was initially identified as a protein that binds membrane immunoglobulins (IgM, IgD). (Kim et al., EMBO J., 13(16):3793-800 (1994)). BAP31 is a small protein (246 amino acids) with three predicted TM domains at the N-terminus and a C-terminal coiled-coil region. The C-terminus ends in -KKXX, a motif implicated in vesicular transport. BAP31 localizes to the ER membrane with the C-terminus extending into the cytoplasm; truncation of this tail abolishes the export of certain proteins, such as cellubrevin, from the ER. (Annaert et al., J. Cell Biol., 139(6):1397-1410 (1997)).

Together, these observations suggest a role for BAP31 as a cargo transporter, mediating the transfer of specific proteins out of the ER. Interestingly, BAP31 has been shown to form a complex with Bcl-2/Bcl-XL and procaspase-8 in the ER (Ng et al., J. Cell Biol., 139(2):327-38 (1997); Ng and Shore, J. Biol. Chem., 273(6):3140-3 (1998)), and is proposed to act as a bridge between Bcl proteins and caspases, thereby regulating caspase activity with respect to Bcl protein status.

Furthermore, BAP31 is cleaved by caspase-1 and -8 activity, removing eight C-terminal amino acids including the -KKXX motif (Maatta et al., FEBS Lett., 484(3):202-6 (2000)). Expression of the BAP31 cleavage product in BHK-21 and NRK (kidney) cells induces subsequent apoptotic events such as the formation of membrane blebs. Expression of the BAP31 cleavage product also prevents ER to Golgi transport of Semliki Forest virus glycoproteins and the Golgi-resident protein mannosidase II, further demonstrating a role for BAP31 in protein export from the ER. The prey construct isolated herein encodes the C-terminus of BAP31, corresponding to most of the C-terminal coiled-coil motif.

TSG101 interacts with zinc finger protein 231: A search of a brain library with the TSG11 (amino acids 231-390) identified the zinc finger protein 231 as an interactor. Zinc finger protein 231 is a very large protein (3926 amino acids) that was first discovered by its elevated expression in brains from patients with multiple system atrophy (MSA), a neurodegenerative disease. (Hashida et al., Genomics, 54(1):50-8 (1998)). Though first found in brain, analysis of homologous EST expression suggests that zinc finger protein 231 is ubiquitously expressed. Analysis of the zinc finger protein 231 protein sequence reveals two nuclear localization signals, numerous proline-, glutamic acid-, and glutamine-rich regions, several small coiled-coil motifs, and several weak matches to the PHD-type zinc finger motif, the PHD finger is a C4HC3 zinc-finger-like motif found in nuclear proteins involved in chromatin-mediated transcriptional regulation. Much of the domain structure of zinc finger protein 231 suggests a possible role as a transcription factor. However, zinc finger protein 231 also contains several weak matches to the FYVE-type zinc finger domain, which is found in proteins such as EEA1 and is a Zn- and PI3P-binding domain likely involved in endosomal targeting, suggesting roles for zinc finger protein 231 in vesicular trafficking. Strong support for such a role comes from analysis of the homologous murine protein, Bassoon, which displays an extraordinary degree of sequence similarity to zinc finger protein 231 (89% amino acid identity over the entire protein). Bassoon is a cytoskeletal-associated protein found in the presynaptic compartment of mouse brain cells, and is thought to be involved in controlling cytomatrix organization at the site of neurotransmitter release. (Dieck et al., J. Cell Biol., 142(2):499-509 (1998)). Electron microscopy of a synapse active zone fraction showed Bassoon associated with vesicular structures, suggesting a role for Bassoon in regulating neurotransmitter release. (Sanmarti-Vila et al., J. Cell Biol., 142(2):499-509 (2000)). Given the interaction between TSG101 and zinc finger protein 231, and the degree of sequence identity between Bassoon and zinc finger protein 231, it is reasonable to hypothesize a role for zinc finger protein 231 in neurotransmitter-containing vesicle docking, fusion, and/or recycling, and to propose that the interaction of zinc finger protein 231 with TSG101 facilitates viral budding.

TSG11 interacts with HCAP: Searches of a macrophage and spleen libraries with amino acids 231-390 and 119-353 of the TSG11 identified interactions with HCAP, a human chromosome-associated polypeptide. HCAP is a 1,217 amino acid protein thought to regulate the assembly and structural maintenance of mitotic chromosomes. (Shimizu et al., J Biol Chem 273(12):6591-4 (1998)). Analysis of homologous EST expression suggests ubiquitous tissue expression. HCAP has four domains of interest: N-terminal and C-terminal structural maintenance of chromosome (SMC) domains, a myosin tail domain, and a weak match to the ABC transporter domain. The SMC domain contains a P-loop and a DA box motif that act cooperatively to bind ATP. (Ghiselli et al., J. Biol. Chem., 274(24):17384-93 (1999)). HCAP is 99% identical over 1200 amino acids to murine and rat bamacan, a basement membrane-chondroitin sulfate proteoglycan. Overexpression of bamacan in NIH and Balb/c 3T3 cells causes transformation, and the levels of expression detected in those transformed cells were the same as levels in spontaneously transformed human colon carcinoma cells. (Ghiselli and Iozzo, J. Biol. Chem., 275(27):20235-8 (2000)). Concentrations of HCAP have been found in the nucleus, giving credibility to an interaction found between HCAP and the small G protein GDP dissociation stimulator-associated protein SMAP, which is also present in the nucleus. SMAP is phosphorylated by Src tyrosine kinase and interacts with 5 mg GDS, a protein which regulates Rho and Ras activity. (Shimizu et al., J. Biol. Chem., 271(43):27013-7 (1996); Sasaki et al., Biochem. Biophys. Res. Commun., 194(3):1188-93 (1993)). HCAP, SMAP, and KIF3B, a kinesin family member that functions as a microtubule-based motor for organelle transport, can be extracted from the nuclear fraction as a ternary complex. (Shimizu et al., J. Biol. Chem., 273(12):6591-4 (1998)). The discovery of this complex has led to the hypothesis that SMAP serves as a link between chromosomes, bound by HCAP, and ATP-based motor proteins like KIF3B.

TSG101 interacts with PIG7, AA300702, AKNA and TOM1L1: Using a two-hybrid assay, it has also been discovered that TSG101 interacts with p53-induced protein 7 (“PIG7”). PIG7 is a nuclear protein that regulates TNF alpha gene transcription. It is induced by p53 and lipopolysaccharide. The two-hybrid search also identified hypothetical protein AA300702 was also identified as an interactor of TSG101. The function of the protein AA300702 is heretofore unknown. Another TSG101-interacting protein identified in accordance with the present invention is AT-hook transcription factor (FLJO0020) (“AKNA”), a transcription factor that binds the A/T-rich regulatory elements of the promoters of CD40 and CD40 ligand (CD40L). The target of myb1 (chicken) homolog-like 1 (TOM1L1) is yet another identified TSG101-interacting protein identified in the two-hybrid screen. TOM1L1 is similar to the endosomal proteins HGS and STAM and is believed to regulate trafficking to the lysosome.

TSG101 interacts with novel protein PN9667: An interaction was also discovered between TSG101 and PN9667—a novel protein heretofore unknown in the art. The amino acid sequences of PN9667 is provided as SEQ ID NO:2, and the nucleotide sequence encoding PN9667 is provided as SEQ ID NO:1. PN9667 is 88% identical to (at the amino acid sequence level) the murine Syne-1B, a protein associated with the nuclear envelope in muscle cells at neuromuscular junctions (GenBank® Accession No. AF281870). PN9667 contains a number of spectrin motifs. In addition, a two-hybrid search using alpha-2 catenin as bait also was identified PN9667 as a protein interactor of alpha-2 catenin.

Subsequent to the discovery of its interaction with TSG101, PN9667 was found to be a fragment of synaptic nuclei expressed gene 1, alt. transcript beta (3321), also known as SYNE1(3321). The nucleotide sequence encoding SYNE1(3321) corresponds to GenBank® Accession No. NM_{—}015293, which is provided herein as SEQ ID NO:3; and the amino acid sequence of the corresponding encoded SYNE1(3321) protein is provided herein as SEQ ID NO:4.

Expression of Syne-1, a dominant negative fragment of the spectrin family, causes an accumulation of binucleate cells, suggesting a role for this protein in cytokinesis. An association of this fragment with the C-terminal tail domain of the kinesin II subunit KIF3B was identified by two-hybrid and co-precipitation assays, suggesting that the role of Syne-1 in cytokinesis involves an interaction with kinesin II. In support of this Fan and Beck (J Cell Sci. 117(Pt 4):619-29 (2004)) found that (1) expression of KIF3B tail domain also gives rise to multinucleate cells, (2) both Syne-1 and KIF3B localize to the central spindle and midbody during cytokinesis in a detergent resistant and ATP sensitive manner and (3) Syne-1 localization is blocked by expression of KIF3B tail. Also, membrane vesicles containing syntaxin associate with the spindle midbody with identical properties. Fan and Beck (J Cell Sci. 117(Pt 4):619-29 (2004)) conclude that Syne-1 and KIF3B function together in cytokinesis by facilitating the accumulation of membrane vesicles at the spindle midbody.

A search of the spleen library using amino acids residues 1-274 of the TSG101 revealed an interaction with protein kinase A (PKA) anchor protein AKAP13. PKA anchor proteins (AKAPs; reviewed in Feliciello et al., 2001; Diviani and Scott, 2001) determine the subcellular localization of cAMP-dependent PKA, regulating its function by controlling its proximity to substrates. Once targeted by AKAP proteins to specific subcellular compartments, PKA has effects on a wide variety of processes, including Na⁺/K⁺ ATPase function, centrosome assembly, cardiac myocyte contraction, cellular proliferation, and insulin secretion. Furthermore, AKAPs interact with signaling molecules other than PKA including phosphatases and other kinases, promoting the assembly of multiprotein signaling machines. AKAP13 was identified by computer modeling as one of four proteins that contain a 14 amino acid region with a high probability of forming an amphipathic helix (Carr et al., J Biol Chem. 266(22):14188-92 (1991)). The region of AKAP13 that contains this motif, when expressed in bacteria, is able to interact with PKA, and site-directed mutations in this region abolish PKA interaction. The AKAP 13 sequence is incomplete; the available sequence predicts a 1015 amino acid protein fragment with no discernible structural motifs other than the amphipathic helical region. Three prey constructs were isolated, encoding similar but distinct overlapping fragments of AKAP13. Two of the prey constructs overlap the PKA interaction domain.

A search of the macrophage library with amino acid residues 265-390* of the TSG101 identified the motor protein P87/89 as an interactor. P87/89, also known as heart muscle protein (HMP) and mitofilin, was initially identified as a protein expressed primarily in heart (Icho et al., Gene 144(2):301-6 (1994)). Sequence analysis revealed the presence of a possible ATP-binding domain at the N-terminus and large central coiled-coil region; a similar domain organization is observed in myosin II and kinesin (actin- and microtubule-based motor proteins, respectively), suggesting P87/89 may also function as a cytoskeleton-based motor protein. Immunofluorescence reveals colocalization of P87/89 with mitochondria, but not with Golgi or endoplasmic reticulum, and P87/89 copurifies with mitochondria (Odgren et al., J Cell Sci. 109 (Pt 9):2253-64 (1996)). The presence of mitochondrial targeting and stop-transfer sequences, along with partial accessibility of the protein to proteolysis, suggests localization of P87/89 predominantly in the intermembrane space. This localization is confirmed by immuno-electron microscopy, which reveals P87/89 primarily at the mitochondrial periphery, and more recent analyses (Gieffers et al., Exp Cell Res. 232(2):395-9 (1997)) which demonstrate the presence of an N-terminal transmembrane domain anchored in the inner mitochondrial membrane with the remainder of the protein located in the intermembrane space. Interestingly, P87/89 was identified as one of three mRNAs overexpressed in response to IL-2 activation of cultured lymphocytes (Damiani et al., Exp Cell Res. 245(1):27-33 (1998)), suggesting possible roles for P87/89 in IL-2 stimulated signal transduction pathways. Expression analyses indicate that P87/89 is expressed in a wide variety of tissues. Two clones corresponding to P87/89 were isolated by ProNet; both encode amino acids 152-335, which contains part of the central coiled-coil region.

The significance of an interaction between TSG101 and a protein localized to the mitochondrial periphery is not clear; however, the putative function of P87/89 as a motor protein is certainly consistent with the functions of a number of other TSG101 interactors, including kinectin (described above), and not all P87/89 protein necessarily localizes to the mitochondrial membrane. P87/89 has been baited by General ProNet. Eight baits are being constructed and will be searched against the brain, spleen, and macrophage libraries.

A search of the brain library with amino acid residues 317-390* of the TSG101 identified the amplified in osteosarcoma protein, OS-9, as an interactor. OS-9 was isolated by a chromosome microdissection-based hybrid selection strategy to clone regions of human chromosomes frequently amplified in human cancers (Su et al., Mol. Carcinogen 15, 270-275 (1996)). Subsequent analysis confirmed that OS-9 is coamplified with CDK4 in three of five sarcoma tissues examined. OS-9 is an acidic 667 amino acid protein that contains a small coiled-coil region and several regions of low sequence complexity (e.g. Leu- and Glu-rich). Other potential functional domains include a nuclear localization signal and an importin-beta N-terminal homology domain, which is necessary for nuclear pore complex function and mediates binding of Ran GTPase by nuclear pore complex proteins. The presence of these domains suggests OS-9 may function as a nuclear protein, perhaps involved in nucleo-cytoplasmic transport. Expression analysis indicates that OS-9 is expressed ubiquitously in normal tissues. Two alternative splice forms of OS-9 have been described (Kimura et al., J Biochem (Tokyo)123(5):876-82 (1998)); expression of these isoforms is also observed in sarcomas. Two clones corresponding to OS-9 were isolated by ProNet; these encode amino acids 171-350 and 213-503.

A search of the macrophage library with amino acid residues 265-390* of the TSG11 identified the death-associated protein DAP5 as an interactor. DAP5 (which stands for death-associated protein 5) was originally identified in a functional screen in HeLa cells for protein fragments that confer resistance to interferon (IFN) gamma-induced apoptosis (Levy-Strumpf et al., Mol Cell Biol. 17(3):1615-25 (1997)). DAP5 was independently identified (as NAT1) in a screen for messenger RNAs that are edited by the apoliprotein B mRNA-editing enzyme APOBEC-1 (Yamanaka et al., Genes Dev. 11(3):321-33 (1997)). DAP5 (NAT1) mRNA was shown to be extensively edited at multiple sites in rabbit and mouse liver carcinomas induced by APOBEC-1 transgene expression, resulting in the creation of stop codons and a reduction in the level of DAP5 (NAT1) protein. Analysis of the DAP5 protein sequence reveals that it is a member of the eukaryotic translation initiation factor 4G (eIF4G), which along with eIF4A and eIF4E constitutes the cap-binding complex eIF4F. However, unlike other eIF4G family members, DAP5 lacks a domain responsible for interaction with the cap-binding protein eIF4E, and biochemical analysis demonstrates that DAP5 is able to interact with eIF4A but not eIF4E. Transient transfection experiment demonstrate that DAP5 inhibits both cap-independent and cap-dependent translation and reduces overall protein synthesis (Imataka et al., EMBO J. 16(4):817-25 (1997)). These observations, along with the ability of a fragment of DAP5 to inhibit TNF-induced programmed cell death, suggests DAP5 may function to control the changes in the cellular translational machinery during apoptosis. Recently, it has been demonstrated that DAP5 is the proteolytic target of caspases during apoptosis, resulting in cleavage of DAP5 at a single site, and although there is an overall reduction in the rate of protein synthesis in apoptotic cells, the translation rate of DAP5 is selectively maintained via internal ribosome entry (Henis-Korenblit et al., Mol Cell Biol. 20(2):496-506 (2000)). In addition, DAP5, which is highly evolutionarily conserved, has been recently knocked out in mice; the phenotype includes embryonic lethality with a marked absence of differentiated cell types (Yamanaka et al., EMBO J. 19(20):5533-41 (2000)). These observations provide further support for a role for DAP5 in controlling translation during apoptosis and cellular differentiation. Expression analyses indicate ubiquitous expression of DAP5.

A search of the spleen library with amino acid reisdues 231-390* of the TSG101 identified an interaction with the growth arrest-specific protein GAS7b, also known as growth arrest-specific 7, isoform b (412). GAS7b was previously to interact with the capsid region of the HIV GAG polyprotein. GAS7b is expressed preferentially in cells that are entering the quiescent state. Inhibition of GAS7b expression in terminally differentiating cultures of embryonic murine cerebellum impedes neurite outgrowth, while overexpression in undifferentiated neuroblastoma cell cultures dramatically promotes neurite-like outgrowth (Ju et al., Proc. Natl. Acad. Sci. (USA) 95(19):11423-8 (1998)); (Lazakovitch et al., Genomics 61(3):298-306 (1999)). These findings suggest a role for GAS7b in controlling terminal cellular differentiation, and the domain structure of GAS7b suggests it may do this by regulating the cytoskeleton. The interaction of GAS7b with the HIV capsid and with TSG101 (which in turn interacts with the HIV p6 protein) strongly suggests these proteins form a multimolecular complex involved in the late stages of viral assembly and budding.

Three clones encoding a similar region of GAS7b were isolated using TSG101 as bait. The interaction of GAS7b with two different HIV GAG proteins (directly with capsid and indirectly with p6) and with two different regulators of small GTPases that control the actin cytoskeleton provides further evidence of a role for GAS7b in mediating HIV production by controlling the cytoskeleton.

A search of the macrophage library with amino acid residues 231-390* of the TSG101 identified the novel protein PN19062 as an interactor. The nucleotide sequence encoding PN19062 is provided herein as SEQ ID NO:5, and the amino acid sequence of the encoded PN19062 protein is provided as SEQ ID NO:6. At the time of this discovery PN19062 was a 217 amino acid protein; however, an upstream stop codon had not yet been identified in the 5′UTR, suggesting that this protein may be initiated from an ATG located further upstream and may therefore be considerably larger. Recently, a protein nearly identical to PN19062 was described in the literature Choglay et al., Gene 267(1):125-34 (2001); however, the sequence of this protein, designated HMGE (GenBank® AAG31605), is truncated and lacks the N-terminal Met found in PN19062. Therefore, we continued to refer to PN19062 as a “novel” protein PN19062/HMGE. PN19062/HMGE was later conclusively identified as a fragment of HMGE, GenBank Accession No. NM_{—}025196.2 (included herein as SEQ ID NO:7, along with the amino acid sequence of the encoded full-length HMGE proteins, as SEQ ID NO:8). PN19062/HMGE is 88% identical at the amino acid level to the rat mitochondrial homolog of the bacterial GrpE protein, which functions as a nucleotide exchange factor (co-chaperone) for the chaperone proteins DnaJ and DnaK involved in the recovery of cellular proteins from stress-induced denaturation (Brehmer et al., Nat Struct Biol. 8(5):427-32 (2001)). Despite the relatively low level of sequence conservation between PN19062/HMGE and E. coli GrpE, the human protein could be efficiently modeled on the X-ray structure of the bacterial protein, suggesting significant functional conservation. Although immunocytochemical analysis suggests localization of PN19062/HMGE in mitochondria (which is also true for the rat GrpE homolog), PN19062/HMGE is able to bind the cytoplasmic form of Hsp70 suggesting localization in the cytosol as well. Two clones from PN19062 were isolated by ProNet; these encode amino acids 55-79 and 40-89, overlapping a centrally-located coiled-coil motif. More recently, a protein identical to PN19062 was described in a GenBank® RefSeq entry (GenBank® NM_{—}025196.2; GI:37059725), and named GrpE-like protein cochaperone 1, or GRPEL1.

The interaction of a human GrpE homolog with TSG101 is interesting in light of the observations that a variety of other chaperones (Hsp27, Hsp70, and Hsp78) form complexes with HIV-1 viral proteins intracellularly, and Hsp70 and Hsp56 are incorporated into HIV-1 virions (reviewed (Brenner et al., Infect Dis Obstet Gynecol 7(1-2):80-90 (1999)). The interaction of TSG101 with the GrpE homolog PN19062 is reminiscent of the interaction of TSG101 with KIAA0674 (described above) which, by virtue of its peptidyl-prolyl isomerase (PPIase) domain, may also promote protein folding. The interaction of both of these proteins with TSG101, and the interaction of the protein folding catalysts cyclophilin A and cyclophilin B with the HIV GAG protein, make it tempting to speculate that these interactors assist in the assembly of infectious virus particles by catalyzing protein folding and the assembly of multiprotein complexes.

Survivin has clearly been shown to function as an anti-apoptotic factor and also appears to play a role in cytokinesis. (Olie et al., Cancer Res., 60(11):2805-9 (2000); Chen et al., Neoplasia, 2(3):235-41 (2000)). Survivin has been shown to inhibit caspase function and to override the mitotic spindle checkpoint. (Suzuki et al., Oncogene, 19(10): 1346-53 (2000); Li et al., Nature, 396(6711):580-4 (1998)). In addition, it has been shown that survivin has (positive) cell cycle effects that coincide with its movement from the cytoplasm to the nucleus where it interacts with the Cdk4/p16(INK4a) complex. (Suzuki et al., Oncogene, 19(29):3225-34 (2000)). This is followed by phosphorylation of Rb (anti-apoptotic).

Survivin is one of few known proteins that integrate cell cycle progression and programmed cell death, and thus is involved in preserving homeostasis and developmental morphogenesis (reviewed in Altieri et al., Lab Invest., 79:1327-1333 (1999)). Survivin, also known as API4 or BIRC5, is a 142 amino acid protein that contains a single Zn finger of the type found in other IAP (inhibitor of apoptosis) family members that is necessary for apoptosis inhibition, and a C-terminal RING finger thought to mediate protein-protein interaction (Cahill et al., Nature, 392:300-303 (1998)). In addition, survivin contains numerous potential phosphorylation sites. Suppression of survivin expression in HeLa cells results in increased apoptosis and inhibition of proliferation (Ambrosini et al., J. Biol. Chem., 273:11177-11182 (1998)). Furthermore, expression of a phosphorylation-defective (T34A) survivin mutant protein in several human melanoma cell lines triggered apoptosis and enhanced sensitivity to the chemotherapeutic drug cisplatin, and either prevented tumor formation or retarded tumor growth in mice (Grossman et al., Proc. Natl. Acad. Sci. U.S.A., 98:635-640 (2001)).

Survivin is expressed during G2/M phase of the cell cycle and associates with microtubules of the mitotic spindle during mitosis. Disruption of the association of survivin with microtubules results in loss of anti-apoptotic activity and an increase in caspase-3 activity during mitosis (Li et al., Nature, 396:580-584 (1998). Survivin interacts directly with, and inhibits, both caspase-3 and caspase-7, and therefore it has been proposed that the anti-apoptotic effects of survivin are due to sequestration of these caspases in an inactive form on microtubules by survivin (Shin et al., Biochemistry, 40:1117-1123 (2001)). Together, these results suggest that survivin function counteracts a default apoptotic mechanism at the G2/M transition. Overexpression of survivin in a variety of cancers (e.g. adenocarcinoma and high-grade lymphomas; (Cahill et al., Nature, 392:300-303 (1998)) may overcome an apoptotic checkpoint and favor aberrant cell cycle progression.

Survivin interacts with dynein light chain 1: A search of the brain library by two-hybrid analysis using as bait the C-terminal fragment (amino acids 89 to 143) of survivin was found to interact with cytoplasmic dynein light chain 1 (HDLC1) (Table 6). The interacting region of survivin is the C-terminal to the BIR repeat (baculovirus inhibitor of apoptosis repeat), and it contains a coiled coil (aa 99-142), which is found in some structural proteins, such as myosins, and in some DNA-binding proteins as the so-called leucine-zipper. HDLC1 is an 89-amino acid protein, and the prey isolated here encodes the entire ORF as well as 20 “amino acids” of translated 5′-UTR. Dyneins are molecular motors that translocate along microtubules. Null mutations of Drosophila dlc1 were lethal and caused embryonic degeneration and widespread apoptotic cell death. Recently, the proapoptotic Bcl-2 family member Bim was shown to be sequestered by LC8 DLC in healthy cells, and this interaction was disrupted by certain apoptotic stimuli. This freed Bim to translocate together with LC8 to Bcl-2 and to neutralize its antiapoptotic activity. Furthermore, it has been found that the 10-kD human DLC1 protein physically interacts with and inhibits the activity of neuronal nitric oxide synthase. Jaffrey and Snyder, Science, 274:774-7 (1996). In the brain, nitric oxide is responsible for the glutamate-linked enhancement of 3-prime, 5-prime cyclic guanosine monophosphate levels and may be involved in apoptosis, synaptogenesis, and neuronal development. It is thus not surprising that survivin interacts with a protein having a central role in apoptosis.

Searches of the brain library with additional fragments of survivin (aa 3-99 and 47-142) confirmed cytoplasmic dynein light chain 1 (HDLC1) as an interactor. The prey is the full-length protein including 19 amino acids of translated 5′-UTR. Survivin is 142 amino acids long, and the previous survivin bait that isolated HDLC1 is amino acids 89-142. HDLC1 either interacts with both the N-terminus (aa 3-99) and C-terminus (aa 89-142) of survivin, or we have fortuitously identified a core HDLC1 binding site on survivin, namely aa 89-99. Survivin may be held in the cytoplasm in a complex with dynein light chain in the absence of an anti-apoptotic signal. This can occur either appropriately, such as during angiogenesis, or it can occur inappropriately, such as in many cancers. (Tran et al., Biochem. Biophys. Res. Commun., 264(3):781-8 (1999); O'Conner et al., Am. J. Pathol., 156(2):393-8 1 (2000)). Pathologically, survivin expression correlates with a bad prognosis for cancer survival. (Sarela et al., Gut, 46(5):645-50 (2000)). Survivin is an example of a more “cancer-specific” drug target, which may be useful in developing anticancer drugs that may have fewer cytotoxic side effects than do current chemotherapeutics. (Buolamwini, Curr. Opin. Chem. Biol., 3(4):500-9 (1999)).

Survivin functions as an inhibitor of apoptosis (IAP) because of its ability to interact with, and inhibit, both caspase-3 and caspase-7. Survivin is abundantly expressed in transformed cells of lymphoid and myeloid lineage, adenocarcinoma of the lung, pancreas, colon, breast, and prostate. Normally, survivin expression appears to be developmentally regulated; the protein is expressed in fetal tissues but is absent in adult, terminally differentiated tissues (Ambrosini et al., Nat Med 3:917, (1997)). Expression of survivin, which is known to be a microtubule-associated protein, is also specific to the G2/M phase of the cell cycle.

As noted above, survivin acts to suppress apoptosis through inhibition of caspases 3 and 7. Unlike most IAP proteins, survivin contains only a single BIR domain (baculovirus inhibitor of apoptosis repeat) and lacks a RING finger. Survivin has the highest sequence homology to the yeast Bir1 protein that functions in cell division control and chromosome segregation (Li F et al., J Biol Chem 275:6707, (2000); Li F et al., Nature 396:580, (1998)).

The C-terminal portion of survivin involved in the interaction with HDLC1 is downstream of the BIR domain and contains a coiled-coil domain (aa99-142) that appears to be responsible for the interaction of survivin with microtubules. A truncated survivin mutant (M1-G99) binds minimally to polymerized microtubules and is not cytoprotective against taxol-induced apoptosis (Li F et al Nature 396:580, (1998)). In addition, a point mutant (Cys84Ala) that alters a residue conserved throughout BIR domains, binds indistinguishably from wt survivin to microtubules but is not cytoprotective.

A functional BIR domain and localization to microtubules appear to be necessary for the inhibition of apoptosis by survivin. Inhibiting the interaction between HDLC1 and survivin may result in a loss of survivin from the microtubule and a negation of its function in inhibiting apoptosis. It has been demonstrated that gene targeting of survivin with an antisense mRNA derived from EPR-1 in HeLa cells increased apoptosis and inhibited the growth of these transformed cells (Ambrosini et al., J Biol Chem 273: 11177, (1998)).

In support of this approach are data derived with the Bcl-2 family member Bim. The pro-apoptotoc activity of Bim appears to be regulated by its interaction with the dynein motor complex through the LC8 cytoplasmic dynein light chain (Puthalakath H Mol Cell 3:287, 1999). Apoptotic stimuli disrupt the interaction of LC8 with the dynein complex and result in the translocation of LC8 and Bim away from microtubules. It has been hypothesized that this release allows Bim to move to Bcl-2 to inhibit its anti-apoptotic effect.

The search of the brain library with a portion of survivin comprising amino acid residues 3-99 also identified β-actin (ACTB) and ATP-dependent DNA helicase II, 70 kD subunit (KU70) as interactors (Table 6). The β-actin prey isolated here (aa 336-375) is C-terminal. β-actin is a non-muscle cytoskeletal actin involved in cell motility. Survivin and β-actin have not been linked in the literature, however, β-actin mRNA is differentially expressed in cells undergoing apoptosis, suggesting a link between β-actin and apoptosis, and therefore perhaps survivin. Naora and Naora, Biochem. Biophys. Res. Commun., 211(2):491-6 (1995). Actins in general have been linked to apoptosis as targets of caspases. (Rossiter et al., Neuropathol. Appl. Neurobiol., 26(4):342-6 (2000)). Actins have been shown to undergo rearrangement concomitant with apoptosis. (Mashima et al., Oncogene, 18(15):2423-30 (1999); Suarez-Huerta et al., J. Cell Physiol, 184(2):239-45 (2000)). Interestingly, it has been shown that p53 binds to filamentous actin in a calcium-dependent manner. (Metcalfe et al., Oncogene, 18(14):2351-5 (1999)). Thus, it has been speculated that this may play a role in the transient and reversible nuclear to cytoplasmic shuttling which p53 undergoes. (Metcalfe et al., Oncogene, 18(14):2351-5 (1999)). For instance, during DNA synthesis, when non-pathological DNA strand breaks are present, a p53 response should not be triggered.

The other prey found to interact with survivin comprised amino acid residues 131-403 of KU70 (Table 6). KU70 is a single-stranded DNA- and ATP-dependent helicase thought to have a role in DNA repair (DNA damage sensor) and chromosomal translocation (double-strand break repair protein). It has been proposed that the presence or absence of KU70 determines whether double-stranded breaks are repaired by nonhomologous end joining (DNA damage; KU70 present) or by homologous recombination (meiosis; KU70 absent). (Goedecke et al., Nat. Genet., 23(2):194-8 (1999); Li et al., Mol. Cell, 2(1):1-8 (1998)) suggested that KU70 is a candidate tumor suppressor gene since mice carrying a disruption of the KU70 gene showed a propensity for malignant transformation (T-cell lymphomas). Autoantibodies against Ku70 are common in cases of systemic lupus erythematosis and autoimmune thyroiditis (Grave's disease), and this is how Ku70 was first identified. Interestingly, (Takeda et al. J. Immunol., 163(11):6269-74 (1999)) mentioned that caspase-cleaved proteins can elicit the generation of autoantibodies, because cleavage of self antigens may enhance their immunogenicity. In the context of the survivin-KU70 interaction, it is possible that in the case of lupus, this represents a pathologic disorder of apoptosis in which KU70 is inappropriately cleaved. Physiologically, it is likely that survivin plays a role in targeting DNA metabolizing enzymes, such as the helicase KU70, for proteolysis as one facet of the orchestrated process of programmed cell death. It has been disclosed that an ionizing radiation-induced KU70-containing complex appears to regulate whether cells undergo apoptosis following a DNA insult. (Yang et al., Proc. Natl. Acad. Sci. USA, 97(11):5907-12 (2000)). In addition, it has been further suggested that KU70 up-regulation (following ionization) serves to determine either a course of DNA repair or an arresting response, such as cell death. (Brown et al., J. Biol. Chem., 275(9):6651-6 (2000)).

The interactions between survivin and proteins including HDLC1, beta-actin, DNA helicase II, COPP, OSTP, SLC8A1, A2-CAT (Table 6) suggest that these proteins are involved in common biological processes including, but not limited to, apoptosis, and disease pathways involving such cellular functions.

We have also discovered an interaction between apoptosis regulator Bcl-X, long transcript (Bcl-XL) and translationally-controlled tumor protein (TCTP) in a two-hybrid search of a brain library (Table 7). Bcl-XL is a Bcl2-related protein that functions as a regulator of programmed cell death (apoptosis). It is expressed in tissues containing post-mitotic cells, such as the adult brain. Bcl-XL is found localized in the mitochondria membrane, where it binds to and closes the mitochondrial voltage-dependent anion channel, VDAC, thus preventing the transport of cytochrome c, the caspase activator, from the mitochondrial lumen to the cytoplasm. (Shimizu et al., Nature, 399:483-487 (1999)). Interestingly, a shorter alternative splice variant, Bcl-XS, promotes apoptosis and is found in cells that have rapid turnover, such as developing lymphocytes. (Boise et al., Cell, 74:597 (1993)).

TCTP (also known as IgE-dependent histamine-releasing factor or HRF) functions in the release of histamine from basophils after the immediate reaction to allergan challenge. (MacDonald et al., Science, 269:688 (1995)). This cellular reaction is related to decreased airway function in such pathologic events as asthma. TCTP is also implicated in cell growth and it is a highly conserved and expressed protein in eukaryotes. TCTP appears to be a calcium-binding protein (Kim et al., Arch. Pharm. Res., 23:633 (2000)) as well as a member of the Mss4/Dss4 superfamily which functions as Rab guanine nucleotide-free chaperones. (Thaw et al., Nat. Struct. Biol., 8:701 (2001). Sanchez et al., Electrophoresis, 18:150 (1997) found that TCTP is expressed in normal and in tumor cells including erythrocytes, hepatocytes, macrophages, platelets, keratinocytes, erythroleukemia cells, gliomas, melanomas, hepatoblastomas, and lymphomas. It appears to be localized in the cytoplasm and contain heat stability. More recently, Baudet et al., Cell Death Differ 5:116 (1998) demonstrated TCTP is one of 61 differentially expressed genes in a rat brain cDNA library with exposure to 1,25-dihydroxyvitamin D3, a treatment that causes rat C6.9 glioma cells to undergo apoptosis.

The interaction between Bcl-XL and TCTP is highly interesting because of its potential role in regulating apoptosis. The biological function of TCTP is as yet unclear, but its differential expression under conditions that induce apoptosis and its interaction with Bcl-XL make it likely that TCTP functions in programmed cell death.

Caspase-7 is known to be involved in apoptosis. LIPA encodes lipase A, the lysosomal acid lipase (also known as cholesteyrl ester hydrolase). In lysosomes, LIPA functions to catalyze the hydrolysis of cholesteryl esters and triglycerides. In addition, mutations in LIPA have been found to be associated with Wolman disease and cholesteryl ester storage disease.

We have also discovered an interaction between caspase 7 (alt. transcript beta [253]) and cholesteryl ester hydrolase/lysosomal acid lipase A (LIPA) in a yeast two-hybrid search of a brain library (Table 8). Caspase 7 is a cytoplasmic cysteine protease that is involved in programmed cell death (apoptosis). Apoptosis involves a stepwise progression of cysteine protease activation; upstream or initiator caspases cleave and activate executioner caspases, which in turn cleave and activate proteins that produce the apoptotic phenotype. Caspase 7 is included in the executioner subtype. It is also known to activate sterol regulatory element binding proteins (SREBPs) and has been shown to cleave poly(ADP-ribose) polymerase (PARP).

Mutations in the LIPA gene appear to be the cause of two major disorders; the severe infantile-onset Wolman disease and the milder late-onset cholesteryl ester storage disease. LIPA is required for hydrolysis of cholesteryl esters that have been internalized by receptor-mediated endocytosis of lipoprotein particles, and the subsequent formation of fatty acids. It regulates the synthesis of endogenous cholesterol by the enzyme HMG-CoA reductase based on low density lipoprotein uptake.

Apoptosis, via caspase 3, induces the release and activation of sterol regulatory element-binding proteins (SREBPs) from the endoplasmic reticulum, enabling the activation of sterol-responsive genes. Higgins and Ioannou, J. Lipid Res., 42:1939 (2001). Although LIPA is normally localized in the lysosomal lumen, it has been shown that cholesterol oxidation products can cause lysosomal leakage, and subsequent apoptosis. (Yuan et al., Free Radic. Biol. Med., 28:208, (2000)). The interaction between caspase 7 and LIPA may be indicative of cellular damage by oxidized LDL. Caspase 7 may function to regulate intracellular cholesterol levels via its interaction with LIPA. In addition, the interaction may be involved in apoptosis.

We have also discovered an interaction between apolipoprotein A-I (APOA1) and a prenylated Rab acceptor 1 (PRA1) in a yeast two-hybrid search of a liver library (Table 9). APOA1 is synthesized and secreted by the liver and small intestine, and is the major protein component of HDL. It is required for HDL formation and reverse cholesterol transport (cholesterol efflux) from tissues to the liver. APOA1 functions as the acceptor of cholesterol from ABCA1-expressing cells as well as a cofactor for lecithin cholesterol acyltransferase (LCAT). A variant of APOA1 was found that fails to associate with HDL resulting in the absence of HDL, the accumulation of cholesteryl esters, and subsequent development of Tangier disease. (Schmitz et al., Proc. Nat. Acad. Sci., 80:6081 (1983)). Autosomal dominant non-neuropathic systemic amyloidosis is also the result of mutations in the APOA1 gene. (Soutar et al., Proc. Nat. Acad. Sci., 89:7389 (1992)).

PRA1 (Rab acceptor 1 or RABAC1) was previously identified as an interactor of activated (GTP-bound) prenylated Rab. (Bucci et al., Biochem. Biophys, Res. Commun., 58:657 (1999)). Rab proteins are members of the small GTPase Ras superfamily that are required in intracellular vesicle trafficking. They are believed to be required for the assembly of the SNARE fusion complex and to ensure proper docking and fusion of transport vesicles. (Søgaard et al., Cell, 78:937-948 (1994)).

Northern blot analysis indicates that PRA1 is expressed ubiquitously, although dot-blot analysis indicates that it may be most highly expressed in placenta, pituitary gland, kidney, and stomach. PRA1 contains several putative transmembrane domains.

Figueroa et al., J. Biol. Chem., 276: 28219-28225 (2001) suggested that PRA1 acts as an escort protein for small GTPases by binding to the hydrophobic isoprenoid moieties of the small GTPases and facilitates their trafficking through the endomembrane system. In addition, PRA1 also binds specifically to the synaptic vesicle protein VAMP2 (or synaptobrevin II) through the proline-rich domain of VAMP2. (Martincic et al., J. Biol. Chem., 272:26991-26998 (1997)).

The APOA1-PRA1 interaction may be relevant to the secretion of APOA1 or the interaction of APOA1 with membranes during cholesterol efflux. In particular, the interaction may be required for cholesterol efflux and thus, may contribute to the reduction of body cholesterol level. Thus, modulation of the interaction, in particular, enhancement of the interaction may prove to be beneficial to cholesterol-related diseases such as coronary artery diseases and dementia such as Alzheimer's disease.

In addition, we have also demonstrated an interaction between APOA1 and golgi autoantigen 84 KD protein (GOLGIN-84) (Table 9), which is another protein involved in intracellular vesicle trafficking. This interaction may also be involved in the secretion of APOA1 or the interaction of APOA1 with membranes during cholesterol efflux. Modulation of this interaction may also be effective in ameliorating coronary artery diseases and Alzheimer's disease.

With a fragment of APOA1 comprising the C-terminal half of the protein (residues 106-268) a fragment (residues 156-261) of syntaxin was isolated (Table 9). Syntaxin 2 belongs to the family of SNARE proteins involved in vesicle fusions. Syntaxin 2 has a cytosolic coiled coil domain and a C-terminal transmembrane domain. Several splice variants were identified that have different C-termini, one variant lacking the transmembrane domain. (Quinones et al., J. Cell Sci, 112:4291-4304 (1999)). The APOA1-syntaxin 2 interaction may be relevant to the secretion of APOA1 or the interaction of APOA1 with membranes during cholesterol efflux. Modulation of this interaction may also be effective in ameliorating coronary artery diseases and Alzheimer's disease.

With a fragment of APOA1 comprising the C-terminal half of the protein (residues 106-268) a C-terminal fragment (residues 4307-4522) of apolipoprotein B-100 (APOB) was isolated (Table 9). The 4563-residue APOB protein is the major protein component of chylomicrons, VLDL, and LDL and mediates interaction with the LDL receptor family. Defects in APOB can lead to coronary artery disease due to increased plasma levels of LDL cholesterol. A portion of esterified cholesterol is transferred from HDL to LDL particles via cholesteryl ester transfer protein (CETP). The underlying physical interaction between HDL and LDL particles may be mediated by direct protein interaction between their respective apolipoproteins, APOA1 and APOB. Disruption of the interaction may lead to the inhibition of the transfer of esterified cholesterol from HDL to LDL particles.

With a fragment of APOA1 comprising the C-terminal half of the protein (residues 106-268) a fragment (residues 162-248) of FLJ20724 was isolated (Table 9). The 482-residue FLJ20724 protein, also known as BTB domain-containing protein (BTBD1), contains a BTB/POZ domain which is found in some zinc finger proteins and in the kelch family of actin-associated proteins. (Carim-Todd et al., Gene 262:275-281 (2001)). The interaction between APOA1 and FLJ20724 suggests that FLJ20724 may also be involved in cholesterol transport and lipid metabolism.

Apolipoproteins are proteins in the blood that transport cholesterol, triglycerides, and other lipids to and from various tissues. When apolipoproteins bind lipids, the resulting complexes are termed lipoproteins. Lipoproteins form microparticles that are classified by their respective densities into two main categories, low density lipoproteins (LDL) and high density lipoproteins (HDL). The principal function of HDL appears to be the so-called “reverse transport” of cholesterol—carrying excess cholesterol (and probably other phospholipids and proteins) from a variety of tissues to the liver for “re-packaging” or excretion in the bile. Higher blood levels of HDL appear to be protective against coronary artery disease, thus HDL is sometimes referred to as “good” cholesterol. See Srivastava and Srivastava, Mol. Cell Biochem., 209:131-44 (2000).

Among the protein constituents of HDL, apolipoprotein A-II (APOA2) is second only to apolipoprotein A-I (APOA1) in abundance. Like APOA1, APOA2 is synthesized in the liver and small intestine and is secreted into the plasma. Individual polypeptide chains of APOA2, linked by a disulfide bond, form stable homodimers. Each monomer has a short N-terminal domain and a C-terminal domain composed of a variable number of lipid-binding amphipathic helices. Although the exact function of APOA2 remains to be established, it is thought to stabilize HDL structure by associating with lipids, thereby playing an important role in the regulation of cholesterol and lipid transport and metabolism.

A fragment of APOA2 consisting of amino acids 22 to 101 (lacking the amino-terminal signal sequence) was used as a bait to screen a liver activation domain library for interacting proteins in a yeast two-hybrid assay. The APOA2 fragment was found by the inventor to interact with six different prey proteins (see Table 10, above).

The first of these APOA2-interacting proteins were two overlapping fragments of APOA1. The fragments, residues 92 to 267 and 87 to 267, correspond to the C-terminal two-thirds of APOA1. As mentioned above, APOA1 is the major apolipoprotein of HDL, and, as such, it is a relatively abundant plasma protein with a concentration of 1.0-1.5 mg/ml. APOA1 serves as a cofactor for lecithin:cholesterol acetyltransferase, the enzyme responsible for the formation of most cholesteryl esters in plasma. Expression of APOA1 in mammals is known to promote the efflux of cholesterol from a variety of cells. In addition to its prominent role in the reverse transport of cholesterol, APOA1 is also found in chylomicrons, the structures involved in the transport of dietary lipids. Native APOA1 consists of two identical chains of 77 amino acids; an 18-amino acid signal peptide is removed co-translationally and a 6-amino acid propeptide is cleaved post-translationally.

Specific variant forms of APOA1 have been associated with specific human diseases. For example, unprocessed precursor APOA1 (pro-APOA1) is found in Tangiers disease patients. Pro-APOA1 fails to associate with HDL, so these patients lack plasma HDL and suffer from the subsequent accumulation of cholesteryl esters. Specific defects in APOA1 are also known to result in amyloidosis. Amyloidosis is characterized by extracellular deposits of protein fibrils with a high content of beta-sheets in secondary structure. The protein fibrils, together with proteoglycans, create amyloid fibrils that cause organ damage and serious morbidity. While wild-type APOA1 is not associated with amyloidosis, four naturally occurring mutant forms of the protein are known to result in the distinct forms of the disease. Although the details of disease etiology are still being worked out, these four mutant forms all end up liberating N-terminal fragments (residues 1-83 or 1-94) that accumulate in the amyloid deposits. (Genschel et al., FEBS Lett., 430:145-149 (1998)). The interaction between APOA2 and APOA1 probably contributes to the formation and stabilization of the HDL microparticles.

The second protein found to interact with APOA2 in the yeast two-hybrid screen, was a C-terminal fragment (residues 2975 to 3259) of giantin (gcp372). Giantin is a 3,259-amino acid (400 kDa) integral membrane protein localized to the Golgi complex and associated with the cytoskeleton. Linstedt and Hauri, Molec. Biol. Cell, 4:679-693 (1993). It has heptad repeats and coiled coil domains and is known to tether coatamer complex I vesicles to the Golgi apparatus. By virtue of its localization, conservation, and physical properties giantin likely participates in the formation of the intercisternal cross-bridges of the Golgi complex. Linstedt & Hauri, Molec. Biol. Cell, 4:679-693 (1993). The interaction between giantin and APOA2 is likely to be involved in the secretion of APOA2 and/or cholesterol and lipid transport and metabolism.

In addition, it has also been found that APOA2 interacts with prenylated Rab acceptor 1 (“PRA1”). PRA1 (also known as Rab acceptor 1 or RABAC1) was previously identified as an interactor of activated (GTP-bound) prenylated Rab. (Bucci et al., Biochem. Biophys, Res. Commun., 58:657 (1999)). Rab proteins are members of the small GTPase Ras superfamily that are required in intracellular vesicle trafficking. They are believed to be required for the assembly of the SNARE fusion complex and to ensure proper docking and fusion of transport vesicles. (Søgaard et al., Cell, 78:937-948 (1994)).

Northern blot analysis indicates that PRA1 is expressed ubiquitously, although dot-blot analysis indicates that it may be most highly expressed in placenta, pituitary gland, kidney, and stomach.

PRA1 contains several putative transmembrane domains. Figueroa et al., J. Biol. Chem., 276:28219-28225 (2001) suggested that PRA1 acts as an escort protein for small GTPases by binding to the hydrophobic isoprenoid moieties of the small GTPases and facilitates their trafficking through the endomembrane system. In addition, PRA1 also binds specifically to the synaptic vesicle protein VAMP2 (or synaptobrevin II) through the proline-rich domain of VAMP2. (Martincic et al., J. Biol. Chem., 272:26991-26998 (1997)).

The APOA2-PRA1 interaction may be relevant to the secretion of APOA2 or the interaction of APOA2 with membranes during cholesterol efflux. In particular, the interaction may be required for cholesterol and lipid transport and metabolism, and thus, may contribute to the reduction of body cholesterol and lipid level. Thus, modulation of the interaction, in particular, enhancement of the interaction may prove to be beneficial to cholesterol-related diseases such as coronary artery diseases and dementia such as Alzheimer's disease.

The fourth protein found to interact with APOA2 in the yeast two-hybrid screen, was a fragment (residues 534 to 731) of Golgi autoantigen, 84 kDa (golgin-84). Golgin-84 is a 731 amino acid, ubiquitously expressed, membrane protein that is abundant in testis. When expressed in vitro, golgin-84 inserts post-translationally into microsomal membranes in an N-cytoplasmic and C-lumen orientation. (Bascom et al., J. Biol. Chem., 274:2953-2962 (1999)). Golgin-84 is similar in structure and sequence to giantin, another APOA2 interactor described above. The interaction between APOA2 and golgin-84 may be important in intracellular trafficking, apolipoprotein A-II recycling, and/or assembly of HDL particles. Changes in any one of these processes could affect lipid transport and metabolism.

In addition, APOA2 has also been found to interact with the hypothetical proteins FLJ20396 and FLJ22313. The functions of these two proteins have been heretofore unknown. FLJ20396 contains three to four putative membrane-spanning regions, and appears to be a ubiquitously expressed protein. The middle portion of the protein has weak homology (29%) to a human BENE protein that is found in glycolipid and cholesterol-enriched membrane rafts and may be involved in cholesterol and caveolin-regulated processes. (de Marco et al., J. Biol. Chem., 276:23009-23017 (2001)). Thus, the interactions between APOA2 and these two proteins suggest that the two proteins and the interactions could play a role in APOA2 secretion and in lipid transport and metabolism.

Cyclooxygenases (Cox-1 and -2) catalyze the rate-limiting steps in prostanoid biosynthesis, and Cox-1 is the target of nonsteroidal anti-inflammatory drugs (NSAIDS) such as aspirin. Prostanoids produced by the COX pathway signal via plasma membrane-localized, G-protein-coupled receptors as well as via nuclear receptors. Physiologically, various extracellular stimuli such as growth factors, cytokines and tumor promoters regulate the expression of COX-1 and −2 genes. COX-2 is over-expressed in rheumatoid arthritis, colorectal and breast cancer. NSAIDS treat arthritis and reduce the relative risk of colorectal cancer in humans. So inhibition of cyclooxygenase activity continues to be explored both for anti-inflammatory purposes as well as anti-neoplastic effects. (Hla et al., Int. J. Biochem. Cell Biol., 31(5):551-7 (1999); DuBois, Aliment Pharmacol. Ther., 14 (Suppl. 1):64-7 (2000)). Studies using COX-1- and COX-2-deficient mice confirm that both isoforms can contribute to the inflammatory response and that both isoforms have significant roles in carcinogenesis. (Langenbach et al., Ann. N.Y. Acad. Sci., 889:52-61 (1999)).

A search of an adipose library with the cyclooxygenase 1 protein (COX1; aa 563-599) revealed an interaction with the thyroid hormone responsive Spot 14 protein (THR S14; aa 30-146 and 34-146) (Table 11). The Cox-1 bait used here contains the short fragment carboxy-terminal to the catalytic domain. The THR S14 prey fragments isolated here lack recognizable domains (as does the entire protein). “Spot 14” is a nuclear protein induced in liver by hormones, such as thyroid hormone (T3), insulin, and glucagon, and by dietary substrates, such as carbohydrates (glucose) and polyunsaturated fatty acids. It is implicated in the transduction of these hormonal and dietary signals for increased lipid metabolism (synthesis) in hepatocytes, and this includes regulation of genes required for long-chain fatty acid synthesis. (Kinlaw et al., J. Biol. Chem., 270(28):16615-8 (1995); Brown et al., J. Biol. Chem., 272(4):2163-6 (1997)). Spot 14 is abundant only in lipogenic tissues (liver, adipose, lactating mammary) and has been hypothesized to function as a homodimeric transcriptional activator that mediates the switch of hepatic metabolism from the fasted to the fed state. (Cunningham et al., Endocrinology, 138(12):5184-8 (1997)). S14 antisense oligonucleotides inhibit both the intracellular production of lipids and their export as very low-density lipoprotein particles. The S14 gene is located in a region that is amplified in a subset of aggressive breast cancers. S14 is expressed in most breast cancer-derived cell lines and most breast cancer specimens but not in normal nonlactating mammary glands. S14 is associated with enhanced tumor lipogenesis, an established marker of poor prognosis. (Cunningham et al., Thyroid, 8(9):815-25 (1998); Heemers et al., Biochem. Biophys. Res. Commun., 269(1):209-12 (2000)). The metabolism of lipids is central to cell (and tumor) biology. It has been suggested that arachidonic acid and other polyunsaturated fats and/or their metabolites may not only promote tumor cell proliferation but that they may also be anti-apoptotic. (Tang et al., Int. J. Cancer, 72(6):1078-87 (1997)). Therefore, it is conspicuous that Cox-1, which metabolizes arachidonic acid, associates with the S14 protein, which is involved in regulating long-chain fatty acid production. If the Cox-1-Spot 14 interaction functions to positively regulate lipid flow, then its disruption may counter tumor growth.

A search of the same adipose library with the same cyclooxygenase 1 bait protein also revealed an interaction with the hypothetical protein KIAA0567 protein (aa “180-439”; the KIAA0567 hypothetical protein is a fragment) (Table 11). The KIAA0567 prey fragment contains a coiled coil region (aa 225-272) and part of the dynamin GTPase catalytic domain. Dynamin GTPases are large GTPases that mediate vesicle trafficking. Dynamin participates in the endocytic uptake of receptors, associated ligands, and plasma membrane following an exocytic event. It has been shown that KIAA0567 appears to be the OPA1 gene, mutations in which give rise to the disease optic atrophy type 1. (Delettre et al., Nat. Genet., 26(2):207-10 (2000)). This autosomal dominant disease is the most prevalent hereditary optic neuropathy and results in progressive loss in visual acuity leading in many cases to legal blindness. Opa1 is a nuclear gene that is most abundantly expressed in retina, but it is also ubiquitously expressed, which is consistent with the finding that the Opa 1 protein is a component of the mitochondrial matrix. It has been hypothesized that dysfunction of the Opa1 protein affects mitochondrial integrity, resulting in an impairment of energy supply which is disastrous for optic nerve neurons which have a high energy demand. (Alexander et al., Nat. Genet., 26(2):211-5 (2000)). The two proteins are related by their involvement in lipid metabolism: Cox-1 by its metabolism of lipids to generate mediators of inflammation and Opa1 by its implicated role in mitochondrial energetics, central to which is the β-oxidation of fatty acids. The potential role of Opa1 in vesicle trafficking and membrane transport by virtue of its dynamin GTPase domain also makes it possible that Opa1 could play a role in the delivery of arachidonate to Cox-1.

It is somewhat more notable to view the Cox-1-Opa1 interaction in combination with the Cox-1-Spot14 interaction described above. It is conspicuous that Cox-1, a metabolizer of certain fatty acids, associates with a protein involved in regulating fatty acid production (Spot 14) as well as with a protein involved with fatty acid utilization (Opa 1).

The interactions between COX1 and the COX1-interacting proteins suggest that these proteins are involved in common biological processes including, but not limited to, lipid metabolism, cell proliferation, apoptosis, optic neuropathy, and inflammatory response, and disease pathways involving such cellular functions.

### 2.2. Protein Complexes

The present invention comprises complexes formed between full-length proteins, as well as fragments of these full-length proteins that interact in a manner analogous to the polypeptides disclosed in the tables above. Importantly, in most cases in the tables above, for each interacting protein pair, exemplary fragments of bait and prey proteins are provided that interact with each other to form a protein complex. It is widely accepted in the field of protein-protein interactions that interactions between fragments of full-length proteins are generally indicative of interactions formed between corresponding full-length proteins containing such fragments. Thus, in view of the disclosure provided herein, an ordinarily skilled person in the art, apprised of the interacting polypeptides disclosed in the tables above, would immediately envisage protein complexes comprising the corresponding full-length proteins, as well as numerous other complexes comprised of alternative fragments of the bait and prey proteins, interacting in the same manner. For example, such other fragments may include bait or prey protein fragments containing the relevant amino acid residues defined by the coordinates provided in the tables, but with additional amino acid residues flanking the defined amino acid residues at either or both ends.

Practically, one of skill in the art, using the exact species of interacting polypeptides disclosed in the tables above as a starting point, would understand that the genus of interacting polypeptides encompassed by the instant invention includes alternative polypeptide fragments, either shorter or longer, that include the necessary interaction domains required to form the protein-protein interactions, which result in the formation of the protein complexes of the present invention. Routine experimentation, optionally directed by analysis of multiple sequence alignments to identify conserved amino acid residues and stretches of contiguous identical amino acid residues, can be used to define the minimal interacting fragments of the polypeptides in the tables. The routine experimentation needed to define such minimal interacting fragments can be readily practiced by one of skill in the art of molecular biology using the polymerase chain reaction to selectively amplify nucleic acids encoding smaller and smaller portions of the polypeptides disclosed—portions of the interacting proteins disclosed in the tables. The amplified nucleic acids can then be incorporated into expression cassettes in expression vectors that can be used to direct the expression of shorter polypeptides. Such routine experimentation often involves systematically constructing expression vectors encoding progressively shorter portions of one of the two interacting polypeptides described in the tables. For example, a fixed number of nucleotides (i.e., about 15, 30, 45, or 60), encoding a fixed number of amino acid residues (i.e., about 5, 10, 15, or 20, respectively) from either end of the interacting fragment, can be excluded from the coding sequence that is inserted into the cassette that directs the expression of the recombinant polypeptide fragment. The terminally-shortened recombinant polypeptides so expressed are then tested for their ability to interact with the partner protein disclosed in the tables. Reiterative rounds of amplification and cloning of shorter and shorter nucleic acids, followed by the expression of shorter and shorter fragments of the interacting proteins, and subsequent testing to determine whether such truncated fragments still interact, will predictably lead to the elucidation of minimal fragments still capable of interacting with the interacting partner identified in the table.

Additionally, using the species of interacting polypeptides described in the tables above as a starting point, one of skill in the art would also understand that the genus of interacting polypeptides can include polypeptides that are longer than those disclosed, as long as they contain the necessary domain responsible for the interactions. Practically, one of skill of molecular biology, using routine experimentation, can extend the length of the polypeptides described in the tables, from either or both ends, to ultimately encompass full-length (e.g., native) proteins, or even fusion proteins, which interact to form a protein complex representing the naturally-occurring complex found in vivo.

Furthermore, one of skill in the art of molecular biology and protein-protein interactions, using routine experimentation, can introduce amino acid sequence variations in the polypeptides disclosed in the tables, or in polypeptides both longer or shorter than those disclosed. Optionally directed by analysis of multiple sequence alignments to identify variable amino acid residues, one of average skill in the art can use site-directed mutagenesis to introduce specific changes in the amino acid sequence of the interacting partner polypeptides, and thereby produce “synthetic homologues” of the bait or prey proteins, or any interacting fragments thereof. For example, one of average skill in the art could introduce changes in the codons encoding specific amino acid residues found to vary in orthologous proteins. Similarly, one of average skill in the art could introduce changes in the codons encoding specific amino acid residues that result in conservative substitutions of those amino acid residues. For example, using such methods, one of skill in the art could introduce a site-specific mutation that changes a “CTT” codon to an “ATT” codon, thereby causing a leucine residue in the native polypeptide to be replaced by an isoleucine residue in the synthetic homologue. To a first approximation, such a conservative substitution in the expressed polypeptide would not be expected to abrogate the ability of the synthetic homologue to interact with its partner protein, and it may in fact increase the affinity of the interaction (see Graversen et al., J. Biol. Chem. 275:37390-37396 (2000)).

As a typical example of the relative skill in the art of (a) identifying and cloning orthologous proteins from divergent species, (b) identifying conserved regions of such orthologous proteins, and (c) further identifying orthologous interactions made by such proteins in the formation of multiprotein complexes, the work of Queimado and colleagues (Queimado et al., Nucleic Acids Res. 29:1884-1891 (2001)) is incorporated herein by reference in its entirety. Queimado and coworkers used the amino acid sequence of the yeast Mms19 protein to screen public databases for orthologous human and murine proteins. Upon identifying likely candidate coding sequences in cDNA libraries, they rapidly amplified cDNAs corresponding to the mRNAs expressed from the human and murine MMS19 genes. The amplified cDNAs were cloned, sequenced, and used to express the respective Mms19 proteins, which were tested for their ability to functionally complement yeast cells with deleted MMS19 genes. The identification, cloning and sequencing of MMS19 genes from divergent species allowed Queimado and colleagues to identify conserved HEAT repeat domains, known to be responsible for the assembly of multiprotein complexes required for nucleotide excision repair and transcription by RNA Polymerase II. (See: Queimado et al., Nucleic Acids Res. 29:1884-1891 (2001).)

As a typical example of the relative skill in the art of using site-specific mutations to introduce amino acid substitutions of specific residues in a protein, and subsequently determining the effect of such substitutions on the ability of the “synthetic homologous proteins” (i.e., site-specific mutant proteins) to interact with a binding partner, the work of Graversen and colleagues (Graversen et al., J. Biol. Chem. 275:37390-37396 (2000)) is incorporated herein by reference in its entirety. Graversen and colleagues introduced over a dozen specific amino acid changes in the C-type lectin domain of tetranectin at four locations, and then determined the effect these single amino acid substitutions had on the affinity of binding of C-type lectin domain for plasminogen kringle 4—the natural binding partner of tetranectin—using surface plasmon resonance binding and isothermal titration calorimetry binding analyses.

Given the fact that routine experimentation can yield polypeptides that are either shorter or longer than those specific polypeptides disclosed in the tables above, or, alternatively, are synthetic homologues of the disclosed interacting polypetides; and given the fact that many such alternative fragments or synthetic homologues of these interacting polypeptides can still interact to form a protein complex—and can be readily confirmed as interacting with partner proteins—hundreds, if not thousands, of alternative species of protein complexes corresponding to each protein pair disclosed in the tables above are intrinsically disclosed herein to one of skill in the art apprised of the specific protein-protein interactions disclosed in the tables. Further, to a skilled artisan, such alternative species of protein complexes can not only be immediately envisaged and derived from the complexes identified in the tables above, but can be created and verified, using routine experimentation.

Fragments of the native full-length interacting proteins should contain interacting domains, and can interact to form a protein complex of the present invention. Such interacting fragments typically contain a contiguous span of anywhere from 5 to 10 amino acid residues up to several hundred amino acid residues. In most instances, however, the interacting fragments contain a contiguous span of about 20, 25, 30, 35, 40, 45, 50, 60, 70, 80, 90, 100, 120, 140, 160, 180, 200, or more, amino acid residues, from the native full-length interacting proteins. Interacting fragments can be identified by routine experimentation, as described herein. Representative fragments are shown in SEQ ID NOs:236-469.

The present invention also provides for protein complexes comprising homologues of the interacting proteins disclosed in the tables, and for fragments of such homologous proteins. Such protein complexes may contain any combination of homologous proteins, including naturally occurring homologues or, alternatively, “synthetic homologues” specifically engineered from naturally occurring proteins.

For example, the homologous proteins encompassed in the present invention include orthologous proteins, and fragments thereof, that can interact in a manner consistent with the interactions disclosed in the tables. Examples of such orthologous proteins include orthologous proteins from eukaryotic species, including plant, fungal, and animal orthologs, particularly mammalian orthologs. Especially useful orthologs are those orthologous proteins from species of ape, monkey, mouse, rat, rabbit, guinea pig, hamster, gerbil, cat, dog, pig, cow, sheep, goat, horse, chicken, duck, turkey, or fish, etc. The protein complexes encompassed by the present invention can comprise interacting proteins from the same species, or from different species.

When the protein complexes of the present invention comprise fragments of full-length proteins, the fragments of such homologous proteins can consist of about 5, 10, 15, 20, 25, 30, 35, 40, 45, 50, 55, 60, 65, 70, 75, 80, 85, 90, 95, 100, 110, 120, 130, 140, 150, 160, 170, 180, 190, 200, 220, 240, 260, 280, 300, or more, contiguous amino acid residues. Such fragments can be about 50%, 60%, or 70%, preferably about 80% or 85%, more preferably 90%, 91%, 92%, 93%, 94%, and even more preferably 95%, 96%, 97%, 98%, or 99% identical to a corresponding fragment of the human homologue. Corresponding fragments can be identified by pairwise alignment of the homologous fragment with the full-length human sequence using BLASTP program, as described above. Once a corresponding fragment is identified by alignment, the percent identity between the homologous fragment and corresponding fragment of a human protein can be calculated.

Importantly, when protein complexes of the present invention comprise homologous proteins, protein fragments, or fragments of homologous proteins, such proteins comprise an interaction domain that facilitates the interaction between the protein, homologous protein, protein fragment, or fragment of a homologous protein and its binding partner.

Thus, for example, one interacting partner in a protein complex can be a complete native APOA1, a APOA1 homologue capable of interacting with, e.g., PRA1, a APOA1 derivative, a derivative of the APOA1 homologue, a APOA1 fragment capable of interacting with PRA1 (APOA1 fragment(s) containing the coordinates shown in the tables above), a homologue or derivative of the APOA1 fragment, or a fusion protein containing (1) complete native APOA1, (2) a APOA1 homologue capable of interacting with PRA1 or (3) a APOA1 fragment capable of interacting with PRA1. Besides native PRA1, useful interacting partners for APOA1 or a homologue or derivative or fragment thereof also include homologues of PRA1 capable of interacting with APOA1, derivatives of the native or homologue PRA1 capable of interacting with APOA1, fragments of the PRA1 capable of interacting with APOA1 (e.g., a fragment containing the identified interacting regions shown in the tables above), derivatives of the PRA1 fragments, or fusion proteins containing (1) a complete PRA1, (2) a PRA1 homologue capable of interacting with APOA1 or (3) a PRA1 fragment capable of interacting with APOA1.

PRA1 fragments capable of interacting with APOA1 can be identified by the combination of molecular engineering of a PRA1-encoding nucleic acid and a method for testing protein-protein interaction. For example, the coordinates in the tables above can be used as starting points and various PRA1 fragments falling within the coordinates can be generated by deletions from either or both ends of the coordinates. The resulting fragments can be tested for their ability to interact with APOA1 using any methods known in the art for detecting protein-protein interactions (e.g., yeast two-hybrid method). Alternatively, various PRA1 fragments can be made by chemical synthesis. The PRA1 fragments can then be tested for their ability to interact with APOA1 using any method known in the art for detecting protein-protein interactions. Examples of such methods include protein affinity chromatography, affinity blotting, in vitro binding assays, yeast two-hybrid assays, surface plasmon resonance and isothermal titration calorimetry binding analyses, and the like. Likewise, APOA1 fragments capable of interacting with PRA1 can also be identified in a similar manner.

Other protein complexes can be formed in a similar manner based on other interactions provided in the tables.

In a specific embodiment of the protein complex of the present invention, two or more interacting partners are directly fused together, or covalently linked together through a peptide linker, forming a hybrid protein having a single unbranched polypeptide chain. Thus, the protein complex may be formed by “intramolecular” interactions between two portions of the hybrid protein. Again, one or both of the fused or linked interacting partners in this protein complex may be a native protein or a homologue, derivative or fragment of a native protein.

The protein complexes of the present invention can also be in a modified form. For example, an antibody selectively immunoreactive with the protein complex can be bound to the protein complex. In another example, a non-antibody modulator capable of enhancing the interaction between the interacting partners in the protein complex may be included. Alternatively, the protein members in the protein complex may be cross-linked for purposes of stabilization. Various crosslinking methods may be used. For example, a bifunctional reagent in the form of R—S—S—R′ may be used in which the R and R′ groups can react with certain amino acid side chains in the protein complex forming covalent linkages. See e.g., Traut et al., in Creighton ed., Protein Function: A Practical Approach, IRL Press, Oxford, 1989; Baird et al., J. Biol. Chem., 251:6953-6962 (1976). Other useful crosslinking agents include, e.g., Denny-Jaffee reagent, a heterbiofunctional photoactivable moiety cleavable through an azo linkage (See Denny et al., Proc. Natl. Acad. Sci. USA, 81:5286-5290 (1984)), and ¹²⁵I-{S—[N-(3-iodo-4-azidosalicyl)cysteaminyl]-2-thiopyridine}, a cysteine-specific photocrosslinking reagent (see Chen et al., Science, 265:90-92 (1994)).

The above-described protein complexes may further include any additional components, e.g., other proteins, nucleic acids, lipid molecules, monosaccharides or polysaccharides, ions, etc.

### 2.3. Methods of Preparing Protein Complexes

The protein complex of the present invention can be prepared by a variety of methods. Specifically, a protein complex can be isolated directly from an animal tissue sample, preferably a human tissue sample containing the protein complex. Alternatively, a protein complex can be purified from host cells that recombinantly express the members of the protein complex. As will be apparent to a skilled artisan, a protein complex can be prepared from a tissue sample or recombinant host cells by coimmunoprecipitation using an antibody immunoreactive with an interacting protein partner, or preferably an antibody selectively immunoreactive with the protein complex as will be discussed in detail below.

The antibodies can be monoclonal or polyclonal. Coimmunoprecipitation is a commonly used method in the art for isolating or detecting bound proteins. In this procedure, generally a serum sample or tissue or cell lysate is admixed with a suitable antibody. The protein complex bound to the antibody is precipitated and washed. The bound protein complexes are then eluted.

Alternatively, immunoaffinity chromatography and immunoblotting techniques may also be used in isolating the protein complexes from native tissue samples or recombinant host cells using an antibody immunoreactive with an interacting protein partner, or preferably an antibody selectively immunoreactive with the protein complex. For example, in protein immunoaffinity chromatography, the antibody is covalently or non-covalently coupled to a matrix (e.g., Sepharose), which is then packed into a column. Extract from a tissue sample, or lysate from recombinant cells is passed through the column where it contacts the antibodies attached to the matrix. The column is then washed with a low-salt solution to wash away the unbound or loosely (non-specifically) bound components. The protein complexes that are retained in the column can be then eluted from the column using a high-salt solution, a competitive antigen of the antibody, a chaotropic solvent, or sodium dodecyl sulfate (SDS), or the like. In immunoblotting, crude proteins samples from a tissue sample extract or recombinant host cell lysate are fractionated by polyacrylamide gel electrophoresis (PAGE) and then transferred to a membrane, e.g., nitrocellulose. Components of the protein complex can then be located on the membrane and identified by a variety of techniques, e.g., probing with specific antibodies.

In another embodiment, individual interacting protein partners may be isolated or purified independently from tissue samples or recombinant host cells using similar methods as described above. The individual interacting protein partners are then combined under conditions conducive to their interaction thereby forming a protein complex of the present invention. It is noted that different protein-protein interactions may require different conditions. As a starting point, for example, a buffer having 20 mM Tris-HCl, pH 7.0 and 500 mM NaCl may be used. Several different parameters may be varied, including temperature, pH, salt concentration, reducing agent, and the like. Some minor degree of experimentation may be required to determine the optimum incubation condition, this being well within the capability of one skilled in the art once apprised of the present disclosure.

In yet another embodiment, the protein complex of the present invention may be prepared from tissue samples or recombinant host cells or other suitable sources by protein affinity chromatography or affinity blotting. That is, one of the interacting protein partners is used to isolate the other interacting protein partner(s) by binding affinity thus forming protein complexes. Thus, an interacting protein partner prepared by purification from tissue samples or by recombinant expression or chemical synthesis may be bound covalently or non-covalently to a matrix, e.g., Sepharose, which is then packed into a chromatography column. The tissue sample extract or cell lysate from the recombinant cells can then be contacted with the bound protein on the matrix. A low-salt solution is used to wash off the unbound or loosely bound components, and a high-salt solution is then employed to elute the bound protein complexes in the column. In affinity blotting, crude protein samples from a tissue sample or recombinant host cell lysate can be fractionated by polyacrylamide gel electrophoresis (PAGE) and then transferred to a membrane, e.g., nitrocellulose. The purified interacting protein member is then bound to its interacting protein partner(s) on the membrane forming protein complexes, which are then isolated from the membrane.

It will be apparent to skilled artisans that any recombinant expression methods may be used in the present invention for purposes of expressing the protein complexes or individual interacting proteins. Generally, a nucleic acid encoding an interacting protein member can be introduced into a suitable host cell. For purposes of forming a recombinant protein complex within a host cell, nucleic acids encoding two or more interacting protein members should be introduced into the host cell.

Typically, the nucleic acids, preferably in the form of DNA, are incorporated into a vector to form expression vectors capable of directing the production of the interacting protein member(s) once introduced into a host cell. Many types of vectors can be used for the present invention. Methods for the construction of an expression vector for purposes of this invention should be apparent to skilled artisans apprised of the present disclosure. (See generally, Current Protocols in Molecular Biology, Vol. 2, Ed. Ausubel, et al., Greene Publish. Assoc. & Wiley Interscience, Ch. 13, 1988; Glover, DNA Cloning, Vol. II, IRL Press, Wash., D.C., Ch. 3, 1986; Bitter, et al., in Methods in Enzymology 153:516-544 (1987); The Molecular Biology of the Yeast Saccharomyces, Eds. Strathern et al., Cold Spring Harbor Press, Vols. I and II, 1982; and Sambrook et al., Molecular Cloning: A Laboratory Manual, Cold Spring Harbor Press, 1989.)

Generally, the expression vectors include an expression cassette having a promoter operably linked to a DNA encoding an interacting protein member. The promoter can be a native promoter, i.e., the promoter found in naturally occurring cells to be responsible for the expression of the interacting protein member in the cells. Alternatively, the expression cassette can be a chimeric one, i.e., having a heterologous promoter that is not the native promoter responsible for the expression of the interacting protein member in naturally occurring cells. The expression vector may further include an origin of DNA replication for the replication of the vectors in host cells. Preferably, the expression vectors also include a replication origin for the amplification of the vectors in, e.g., E. coli, and selection marker(s) for selecting and maintaining only those host cells harboring the expression vectors. Additionally, the expression cassettes preferably also contain inducible elements, which function to control the transcription from the DNA encoding an interacting protein member. Other regulatory sequences such as transcriptional enhancer sequences and translation regulation sequences (e.g., Shine-Dalgarno sequence) can also be operably included in the expression cassettes. Termination sequences such as the polyadenylation signals from bovine growth hormone, SV40, lacZ and AcMNPV polyhedral protein genes may also be operably linked to the DNA encoding an interacting protein member in the expression cassettes. An epitope tag coding sequence for detection and/or purification of the expressed protein can also be operably linked to the DNA encoding an interacting protein member such that a fusion protein is expressed. Examples of useful epitope tags include, but are not limited to, influenza virus hemagglutinin (HA), Simian Virus 5 (V5), polyhistidine (6×His), c-myc, lacZ, GST, and the like. Proteins with polyhistidine tags can be easily detected and/or purified with Ni affinity columns, while specific antibodies immunoreactive with many epitope tags are generally commercially available. The expression vectors may also contain components that direct the expressed protein extracellularly or to a particular intracellular compartment. Signal peptides, nuclear localization sequences, endoplasmic reticulum retention signals, mitochondrial localization sequences, myristoylation signals, palmitoylation signals, and transmembrane sequences are examples of optional vector components that can determine the destination of expressed proteins. When it is desirable to express two or more interacting protein members in a single host cell, the DNA fragments encoding the interacting protein members may be incorporated into a single vector or different vectors.

The thus constructed expression vectors can be introduced into the host cells by any techniques known in the art, e.g., by direct DNA transformation, microinjection, electroporation, viral infection, lipofection, gene gun, and the like. The expression of the interacting protein members may be transient or stable. The expression vectors can be maintained in host cells in an extrachromosomal state, i.e., as self-replicating plasmids or viruses. Alternatively, the expression vectors can be integrated into chromosomes of the host cells by conventional techniques such as selection of stable cell lines or site-specific recombination. In stable cell lines, at least the expression cassette portion of the expression vector is integrated into a chromosome of the host cells.

The vector construct can be designed to be suitable for expression in various host cells, including but not limited to bacteria, yeast cells, plant cells, insect cells, and mammalian and human cells. Methods for preparing expression vectors for expression in different host cells should be apparent to a skilled artisan.

Homologues and fragments of the native interacting protein members can also be easily expressed using the recombinant methods described above. For example, to express a protein fragment, the DNA fragment incorporated into the expression vector can be selected such that it only encodes the protein fragment. Likewise, a specific hybrid protein can be expressed using a recombinant DNA encoding the hybrid protein. Similarly, a homologue protein may be expressed from a DNA sequence encoding the homologue protein. A homologue-encoding DNA sequence may be obtained by manipulating the native protein-encoding sequence using recombinant DNA techniques. For this purpose, random or site-directed mutagenesis can be conducted using techniques generally known in the art. To make protein derivatives, for example, the amino acid sequence of a native interacting protein member may be changed in predetermined manners by site-directed DNA mutagenesis to create or remove consensus sequences for, e.g., phosphorylation by protein kinases, glycosylation, ribosylation, myristolation, palmytoylation, ubiquitination, and the like. Alternatively, non-natural amino acids can be incorporated into an interacting protein member during the synthesis of the protein in recombinant host cells. For example, photoreactive lysine derivatives can be incorporated into an interacting protein member during translation by using a modified lysyl-tRNA. (See, e.g., Wiedmann et al., Nature, 328:830-833 (1989); Musch et al., Cell, 69:343-352 (1992). Other photoreactive amino acid derivatives can also be incorporated in a similar manner. See, e.g., High et al., J. Biol. Chem., 368:28745-28751 (1993)). Indeed, the photoreactive amino acid derivatives thus incorporated into an interacting protein member can function to cross-link the protein to its interacting protein partner in a protein complex under predetermined conditions.

In addition, derivatives of the native interacting protein members of the present invention can also be prepared by chemically linking certain moieties to amino acid side chains of the native proteins.

If desired, the homologues and derivatives thus generated can be tested to determine whether they are capable of interacting with their intended partners to form protein complexes. Testing can be conducted by e.g., the yeast two-hybrid system or other methods known in the art for detecting protein-protein interaction.

A hybrid protein as described above having any interacting pair of the proteins described in the tables, or a homologue, derivative, or fragment thereof covalently linked together by a peptide bond or a peptide linker can be expressed recombinantly from a chimeric nucleic acid, e.g., a DNA or mRNA fragment encoding the fusion protein. Accordingly, the present invention also provides a nucleic acid encoding the hybrid protein of the present invention. In addition, an expression vector having incorporated therein a nucleic acid encoding the hybrid protein of the present invention is also provided. The methods for making such chimeric nucleic acids and expression vectors containing them will be apparent to skilled artisans apprised of the present disclosure.

### 2.4. Protein Microchip

In accordance with another embodiment of the present invention, a protein microchip or microarray is provided having one or more of the protein complexes and/or antibodies selectively immunoreactive with the protein complexes of the present invention. Protein microarrays are becoming increasingly important in both proteomics research and protein-based detection and diagnosis of diseases. The protein microarrays in accordance with this embodiment of the present invention will be useful in a variety of applications including, e.g., large-scale or high-throughput screening for compounds capable of binding to the protein complexes or modulating the interactions between the interacting protein members in the protein complexes.

The protein microarray of the present invention can be prepared in a number of methods known in the art. An example of a suitable method is that disclosed in MacBeath and Schreiber, Science, 289:1760-1763 (2000). Essentially, glass microscope slides are treated with an aldehyde-containing silane reagent (SuperAldehyde Substrates purchased from TeleChem International, Cupertino, Calif.). Nanoliter volumes of protein samples in a phosphate-buffered saline with 40% glycerol are then spotted onto the treated slides using a high-precision contact-printing robot. After incubation, the slides are immersed in a bovine serum albumin (BSA)-containing buffer to quench the unreacted aldehydes and to form a BSA layer that functions to prevent non-specific protein binding in subsequent applications of the microchip. Alternatively, as disclosed in MacBeath and Schreiber, proteins or protein complexes of the present invention can be attached to a BSA-NHS slide by covalent linkages. BSA-NHS slides are fabricated by first attaching a molecular layer of BSA to the surface of glass slides and then activating the BSA with N,N′-disuccinimidyl carbonate. As a result, the amino groups of the lysine, aspartate, and glutamate residues on the BSA are activated and can form covalent urea or amide linkages with protein samples spotted on the slides. See MacBeath and Schreiber, Science, 289:1760-1763 (2000).

Another example of a useful method for preparing the protein microchip of the present invention is that disclosed in PCT Publication Nos. WO 00/4389A2 and WO 00/04382, both of which are assigned to Zyomyx and are incorporated herein by reference. First, a substrate or chip base is covered with one or more layers of thin organic film to eliminate any surface defects, insulate proteins from the base materials, and to ensure uniform protein array. Next, a plurality of protein-capturing agents (e.g., antibodies, peptides, etc.) are arrayed and attached to the base that is covered with the thin film. Proteins or protein complexes can then be bound to the capturing agents forming a protein microarray. The protein microchips are kept in flow chambers with an aqueous solution.

The protein microarray of the present invention can also be made by the method disclosed in PCT Publication No. WO 99/36576 assigned to Packard Bioscience Company, which is incorporated herein by reference. For example, a three-dimensional hydrophilic polymer matrix, i.e., a gel, is first dispensed on a solid substrate such as a glass slide. The polymer matrix gel is capable of expanding or contracting and contains a coupling reagent that reacts with amine groups. Thus, proteins and protein complexes can be contacted with the matrix gel in an expanded aqueous and porous state to allow reactions between the amine groups on the protein or protein complexes with the coupling reagents thus immobilizing the proteins and protein complexes on the substrate. Thereafter, the gel is contracted to embed the attached proteins and protein complexes in the matrix gel.

Alternatively, the proteins and protein complexes of the present invention can be incorporated into a commercially available protein microchip, e.g., the ProteinChip System from Ciphergen Biosystems Inc., Palo Alto, Calif. The ProteinChip System comprises metal chips having a treated surface, which interact with proteins. Basically, a metal chip surface is coated with a silicon dioxide film. The molecules of interest such as proteins and protein complexes can then be attached covalently to the chip surface via a silane coupling agent.

The protein microchips of the present invention can also be prepared with other methods known in the art, e.g., those disclosed in U.S. Pat. Nos. 6,087,102, 6,139,831, 6,087,103; PCT Publication Nos. WO 99/60156, WO 99/39210, WO 00/54046, WO 00/53625, WO 99/51773, WO 99/35289, WO 97/42507, WO 01/01142, WO 00/63694, WO 00/61806, WO 99/61148, WO 99/40434, all of which are incorporated herein by reference.

### 3. Antibodies

In accordance with another aspect of the present invention, an antibody immunoreactive against a protein complex of the present invention is provided. In one embodiment, the antibody is selectively immunoreactive with a protein complex of the present invention. Specifically, the phrase “selectively immunoreactive with a protein complex” as used herein means that the immunoreactivity of the antibody of the present invention with the protein complex is substantially higher than that with the individual interacting members of the protein complex so that the binding of the antibody to the protein complex is readily distinguishable from the binding of the antibody to the individual interacting member proteins based on the strength of the binding affinities. Preferably, the binding constants differ by a magnitude of at least 2 fold, more preferably at least 5 fold, even more preferably at least 10 fold, and most preferably at least 100 fold. In a specific embodiment, the antibody is not substantially immunoreactive with the interacting protein members of the protein complex.

The antibodies of the present invention can be readily prepared using procedures generally known in the art. See, e.g., Harlow and Lane, Antibodies: A Laboratory Manual, Cold Spring Harbor Press, 1988. Typically, the protein complex against which an immunoreactive antibody is desired is used as the antigen for producing an immune response in a host animal. In one embodiment, the protein complex used consists of the native proteins. Preferably, the protein complex includes only protein fragments containing interacting regions provided in the tables. As a result, a greater portion of the total antibodies may be selectively immunoreactive with the protein complexes. The interaction domains can be selected from, e.g., those regions summarized in the tables above. In addition, various techniques known in the art for predicting epitopes may also be employed to design antigenic peptides based on the interacting protein members in a protein complex of the present invention to increase the possibility of producing an antibody selectively immunoreactive with the protein complex. Suitable epitope-prediction computer programs include, e.g., MacVector from International Biotechnologies, Inc. and Protean from DNAStar.

In a specific embodiment, a hybrid protein as described above in Section 2.1 is used as an antigen which has a first protein that is any one of the proteins described in the tables, or a homologue, derivative, or fragment thereof covalently linked by a peptide bond or a peptide linker to a second protein which is the interacting partner of the first protein, or a homologue, derivative, or fragment of the second protein. In a preferred embodiment, the hybrid protein consists of two interacting domains selected from the regions identified in a table above, or homologues or derivatives thereof, covalently linked together by a peptide bond or a linker molecule.

The antibody of the present invention can be a polyclonal antibody to a protein complex of the present invention. To produce the polyclonal antibody, various animal hosts can be employed, including, e.g., mice, rats, rabbits, goats, guinea pigs, hamsters, etc. A suitable antigen which is a protein complex of the present invention or a derivative thereof as described above can be administered directly to a host animal to illicit immune reactions. Alternatively, it can be administered together with a carrier such as keyhole limpet hemocyanin (KLH), bovine serum albumin (BSA), ovalbumin, and Tetanus toxoid. Optionally, the antigen is conjugated to a carrier by a coupling agent such as carbodiimide, glutaraldehyde, and MBS. Any conventional adjuvants may be used to boost the immune response of the host animal to the protein complex antigen. Suitable adjuvants known in the art include but are not limited to Complete Freund's Adjuvant (which contains killed mycobacterial cells and mineral oil), incomplete Freund's Adjuvant (which lacks the cellular components), aluminum salts, MF59 from Chiron (Emeryville, Calif.), monophospholipid, synthetic trehalose dicorynomycolate (TDM) and cell wall skeleton (CWS) both from Corixa Corp. (Seattle, Wash.), non-ionic surfactant vesicles (NISV) from Proteus International PLC (Cheshire, U.K.), and saponins. The antigen preparation can be administered to a host animal by subcutaneous, intramuscular, intravenous, intradermal, or intraperitoneal injection, or by injection into a lymphoid organ.

The antibodies of the present invention may also be monoclonal. Such monoclonal antibodies may be developed using any conventional techniques known in the art. For example, the popular hybridoma method disclosed in Kohler and Milstein, Nature, 256:495-497 (1975) is now a well-developed technique that can be used in the present invention. See U.S. Pat. No. 4,376,110, which is incorporated herein by reference. Essentially, B-lymphocytes producing a polyclonal antibody against a protein complex of the present invention can be fused with myeloma cells to generate a library of hybridoma clones. The hybridoma population is then screened for antigen binding specificity and also for immunoglobulin class (isotype). In this manner, pure hybridoma clones producing specific homogenous antibodies can be selected. See generally, Harlow and Lane, Antibodies: A Laboratory Manual, Cold Spring Harbor Press, 1988. Alternatively, other techniques known in the art may also be used to prepare monoclonal antibodies, which include but are not limited to the EBV hybridoma technique, the human N-cell hybridoma technique, and the trioma technique.

In addition, antibodies selectively immunoreactive with a protein complex of the present invention may also be recombinantly produced. For example, cDNAs prepared by PCR amplification from activated B-lymphocytes or hybridomas may be cloned into an expression vector to form a cDNA library, which is then introduced into a host cell for recombinant expression. The cDNA encoding a specific desired protein may then be isolated from the library. The isolated cDNA can be introduced into a suitable host cell for the expression of the protein. Thus, recombinant techniques can be used to produce specific native antibodies, hybrid antibodies capable of simultaneous reaction with more than one antigen, chimeric antibodies (e.g., the constant and variable regions are derived from different sources), univalent antibodies that comprise one heavy and light chain pair coupled with the Fc region of a third (heavy) chain, Fab proteins, and the like. (See U.S. Pat. No. 4,816,567; European Patent Publication No. 0088994; Munro, Nature, 312:597 (1984); Morrison, Science, 229:1202 (1985); Oi et al., BioTechniques, 4:214 (1986); and Wood et al., Nature, 314:446-449 (1985)), all of which are incorporated herein by reference. Antibody fragments such as Fv fragments, single-chain Fv fragments (scFv), Fab′ fragments, and F(ab′)₂ fragments can also be recombinantly produced by methods disclosed in, (e.g., U.S. Pat. No. 4,946,778; Skerra & Plückthun, Science, 240:1038-1041 (1988); Better et al., Science, 240:1041-1043 (1988); and Bird, et al., Science, 242:423-426 (1988)), all of which are incorporated herein by reference.

In a preferred embodiment, the antibodies provided in accordance with the present invention are partially or fully humanized antibodies. For this purpose, any methods known in the art may be used. For example, partially humanized chimeric antibodies having V regions derived from the tumor-specific mouse monoclonal antibody, but human C regions are disclosed in Morrison and Oi, Adv. Immunol., 44:65-92 (1989). In addition, fully humanized antibodies can be made using transgenic non-human animals. For example, transgenic non-human animals such as transgenic mice can be produced in which endogenous immunoglobulin genes are suppressed or deleted, while heterologous antibodies are encoded entirely by exogenous immunoglobulin genes, preferably human immunoglobulin genes, recombinantly introduced into the genome. (See e.g., U.S. Pat. Nos. 5,530,101; 5,545,806; 6,075,181; PCT Publication No. WO 94/02602; Green et. al., Nat. Genetics, 7: 13-21 (1994); and Lonberg et al., Nature 368: 856-859 (1994)), all of which are incorporated herein by reference. The transgenic non-human host animal may be immunized with suitable antigens such as a protein complex of the present invention or one or more of the interacting protein members thereof to illicit specific immune response thus producing humanized antibodies. In addition, cell lines producing specific humanized antibodies can also be derived from the immunized transgenic non-human animals. For example, mature B-lymphocytes obtained from a transgenic animal producing humanized antibodies can be fused to myeloma cells and the resulting hybridoma clones may be selected for specific humanized antibodies with desired binding specificities. Alternatively, cDNAs may be extracted from mature B-lymphocytes and used in establishing a library that is subsequently screened for clones encoding humanized antibodies with desired binding specificities.

In yet another embodiment, a bifunctional antibody is provided that has two different antigen binding sites, each being specific to a different interacting protein member in a protein complex of the present invention. The bifunctional antibody may be produced using a variety of methods known in the art. For example, two different monoclonal antibody-producing hybridomas can be fused together. One of the two hybridomas may produce a monoclonal antibody specific against an interacting protein member of a protein complex of the present invention, while the other hybridoma generates a monoclonal antibody immunoreactive with another interacting protein member of the protein complex. The thus formed new hybridoma produces different antibodies including a desired bifunctional antibody, i.e., an antibody immunoreactive with both of the interacting protein members. The bifunctional antibody can be readily purified. See Milstein and Cuello, Nature, 305:537-540 (1983).

Alternatively, a bifunctional antibody may also be produced using heterobifunctional crosslinkers to chemically link two different monoclonal antibodies, each being immunoreactive with a different interacting protein member of a protein complex. Therefore, the aggregate will bind to two interacting protein members of the protein complex. (See Staerz et al., Nature, 314:628-631 (1985); Perez et al., Nature, 316:354-356 (1985)).

In addition, bifunctional antibodies can also be produced by recombinantly expressing light and heavy chain genes in a hybridoma that itself produces a monoclonal antibody. As a result, a mixture of antibodies including a bifunctional antibody is produced. (See DeMonte et al., Proc. Natl. Acad. Sci., USA, 87:2941-2945 (1990); Lenz and Weidle, Gene, 87:213-218 (1990)).

Preferably, a bifunctional antibody in accordance with the present invention is produced by the method disclosed in U.S. Pat. No. 5,582,996, which is incorporated herein by reference. For example, two different Fabs can be provided and mixed together. The first Fab can bind to an interacting protein member of a protein complex, and has a heavy chain constant region having a first complementary domain not naturally present in the Fab but capable of binding a second complementary domain. The second Fab is capable of binding another interacting protein member of the protein complex, and has a heavy chain constant region comprising a second complementary domain not naturally present in the Fab but capable of binding to the first complementary domain. Each of the two complementary domains is capable of stably binding to the other but not to itself. For example, the leucine zipper regions of c-fos and c-jun oncogenes may be used as the first and second complementary domains. As a result, the first and second complementary domains interact with each other to form a leucine zipper thus associating the two different Fabs into a single antibody construct capable of binding to two antigenic sites.

Other suitable methods known in the art for producing bifunctional antibodies may also be used, which include those disclosed in (Holliger et al., Proc. Nat'l Acad. Sci. USA, 90:6444-6448 (1993); de Kruif et al., J. Biol. Chem., 271:7630-7634 (1996); Coloma and Morrison, Nat. Biotechnol., 15:159-163 (1997); Muller et al., FEBS Lett., 422:259-264 (1998); and Muller et al., FEBS Lett., 432:45-49 (1998)), all of which are incorporated herein by reference.

### 4. Methods of Detecting Protein Complexes

Another aspect of the present invention relates to methods for detecting the protein complexes of the present invention, particularly for determining the concentration of a specific protein complex in a patient sample.

In one embodiment, the concentration of a protein complex of the present invention is determined in cells, tissue, or an organ of a patient. For example, the protein complex can be isolated or purified from a patient sample obtained from cells, tissue, or an organ of the patient and the amount thereof is determined. As described above, the protein complex can be prepared from cells, tissue or organ samples by coimmunoprecipitation using an antibody immunoreactive with an interacting protein member, a bifunctional antibody that is immunoreactive with two or more interacting protein members of the protein complex, or preferably an antibody selectively immunoreactive with the protein complex. When bifunctional antibodies or antibodies immunoreactive with only free interacting protein members are used, individual interacting protein members not complexed with other proteins may also be isolated along with the protein complex containing such individual proteins. However, they can be readily separated from the protein complex using methods known in the art, e.g., size-based separation methods such as gel filtration, or by subtracting the protein complex from the mixture using an antibody specific against another individual interacting protein member. Additionally, proteins in a sample can be separated in a gel such as polyacrylamide gel and subsequently immunoblotted using an antibody immunoreactive with the protein complex.

Alternatively, the concentration of the protein complex can be determined in a sample without separation, isolation or purification. For this purpose, it is preferred that an antibody selectively immunoreactive with the specific protein complex is used in an immunoassay. For example, immunocytochemical methods can be used. Other well known antibody-based techniques can also be used including, e.g., enzyme-linked immunosorbent assay (ELISA), radioimmunoassay (RIA), immunoradiometric assays (IRMA), fluorescent immunoassays, protein A immunoassays, and immunoenzymatic assays (IEMA). See e.g., U.S. Pat. Nos. 4,376,110 and 4,486,530, both of which are incorporated herein by reference.

In addition, since a specific protein complex is formed from its interacting protein members, if one of the interacting protein members is at a relatively low concentration in a patient, it may be reasonably expected that the concentration of the protein complex in the patient may also be low. Therefore, the concentration of an individual interacting protein member of a specific protein complex can be determined in a patient sample which can then be used as a reasonably accurate indicator of the concentration of the protein complex in the sample. For this purpose, antibodies against an individual interacting protein member of a specific complex can be used in any one of the methods described above. In a preferred embodiment, the concentration of each of the interacting protein members of a protein complex is determined in a patient sample and the relative concentration of the protein complex is then deduced.

In addition, the relative protein complex concentration in a patient can also be determined by determining the concentration of the mRNA encoding an interacting protein member of the protein complex. Preferably, each interacting protein member's mRNA concentration in a patient sample is determined. For this purpose, methods for determining mRNA concentration generally known in the art may all be used. Examples of such methods include, e.g., Northern blot assay, dot blot assay, PCR assay (preferably quantitative PCR assay), in situ hybridization assay, and the like.

As discussed above, each interaction between members of an interacting protein pair of the present invention suggests that the proteins and/or the protein complexes formed by such proteins may be involved in common biological processes and disease pathways. In addition, the interactions under physiological conditions may lead to the formation of protein complexes in vivo. The protein complexes are expected to mediate the functions and biological activities of the interacting members of the protein complexes. Thus, aberrations in the protein complexes or the individual proteins and the degree of the aberration may be indicators for the diseases or disorders. These aberrations may be used as parameters for classifying and/or staging one of the above-described diseases. In addition, they may also be indicators for patients' response to a drug therapy.

Association between a physiological state (e.g., physiological disorder, predisposition to the disorder, a disease state, response to a drug therapy, or other physiological phenomena or phenotypes) and a specific aberration in a protein complex of the present invention or an individual interacting member thereof can be readily determined by comparative analysis of the protein complex and/or the interacting members thereof in a normal population and an abnormal or affected population. Thus, for example, one can study the concentration, localization and distribution of a particular protein complex, mutations in the interacting protein members of the protein complex, and/or the binding affinity between the interacting protein members in both a normal population and a population affected with a particular physiological disorder described above. The study results can be compared and analyzed by statistical means. Any detected statistically significant difference in the two populations would indicate an association. For example, if the concentration of the protein complex is statistically significantly higher in the affected population than in the normal population, then it can be reasonably concluded that higher concentration of the protein complex is associated with the physiological disorder.

Thus, once an association is established between a particular type of aberration in a particular protein complex of the present invention or in an interacting protein member thereof and a physiological disorder or disease or predisposition to the physiological disorder or disease, then the particular physiological disorder or disease or predisposition to the physiological disorder or disease can be diagnosed or detected by determining whether a patient has the particular aberration.

Accordingly, the present invention also provides a method for diagnosing in a patient a disease or physiological disorder, or a predisposition to the disease or disorder, such as abnormal cell proliferation (hyperproliferation or dysproliferation), keloid, liver cirrhosis, psoriasis, altered wound healing, cancer, especially cancers of the breast, ovary, colon, prostate, and lung, melanomas and cancers of hematopoietic origin, type 2 diabetes, obesity, hypertension, hypercholesterolemia, atherosclerosis, coronary artery disease, peripheral vascular disease, optic neuropathy, inflammatory response, amyloidosis, dementia, Alzheimer's disease, Tangier disease, Angelman syndrome, and specific viral infections; and by determining whether there is any aberration in the patient with respect to a protein complex identified according to the present invention. The same protein complex is analyzed in a normal individual and is compared with the results obtained in the patient. In this manner, any protein complex aberration in the patient can be detected. As used herein, the term “aberration” when used in the context of protein complexes of the present invention means any alterations of a protein complex including increased or decreased concentration of the protein complex in a particular cell or tissue or organ or the total body, altered localization of the protein complex in cellular compartments or in locations of a tissue or organ, changes in binding affinity of an interacting protein member of the protein complex, mutations in an interacting protein member or the gene encoding the protein, and the like. As will be apparent to a skilled artisan, the term “aberration” is used in a relative sense. That is, an aberration is relative to a normal condition.

As used herein, the term “diagnosis” means detecting a disease or disorder or determining the stage or degree of a disease or disorder. The term “diagnosis” also encompasses detecting a predisposition to a disease or disorder, determining the therapeutic effect of a drug therapy, or predicting the pattern of response to a drug therapy or xenobiotics. The diagnosis methods of the present invention may be used independently, or in combination with other diagnosing and/or staging methods known in the medical art for a particular disease or disorder.

Thus, in one embodiment, the method of diagnosis is conducted by detecting, in a patient, the concentrations of one or more protein complexes of the present invention using any one of the methods described above, and determining whether the patient has an aberrant concentration of the protein complexes.

The diagnosis may also be based on the determination of the concentrations of one or more interacting protein members (at the protein, cDNA or mRNA level) of a protein complex of the present invention. An aberrant concentration of an interacting protein member may indicate a physiological disorder or a predisposition to a physiological disorder.

In another embodiment, the method of diagnosis comprises determining, in a patient, the cellular localization, or tissue or organ distribution of a protein complex of the present invention and determining whether the patient has an aberrant localization or distribution of the protein complex. For example, immunocytochemical or immunohistochemical assays can be performed on a cell, tissue or organ sample from a patient using an antibody selectively immunoreactive with a protein complex of the present invention. Antibodies immunoreactive with both an individual interacting protein member and a protein complex containing the protein member may also be used, in which case it is preferred that antibodies immunoreactive with other interacting protein members are also used in the assay. In addition, nucleic acid probes may also be used in in situ hybridization assays to detect the localization or distribution of the mRNAs encoding the interacting protein members of a protein complex. Preferably, the mRNA encoding each interacting protein member of a protein complex is detected concurrently.

In yet another embodiment, the method of diagnosis of the present invention comprises detecting any mutations in one or more interacting protein members of a protein complex of the present invention. In particular, it is desirable to determine whether the interacting protein members have any mutations that will lead to, or are associated with, changes in the functional activity of the proteins or changes in their binding affinity to other interacting protein members in forming a protein complex of the present invention. Examples of such mutations include but are not limited to, e.g., deletions, insertions and rearrangements in the genes encoding the protein members, and nucleotide or amino acid substitutions and the like. In a preferred embodiment, the domains of the interacting protein members that are responsible for the protein-protein interactions, and lead to protein complex formation, are screened to detect any mutations therein. For example, genomic DNA or cDNA encoding an interacting protein member can be prepared from a patient sample, and sequenced. The thus obtained sequence may be compared with known wild-type sequences to identify any mutations. Alternatively, an interacting protein member may be purified from a patient sample and analyzed by protein sequencing or mass spectrometry to detect any amino acid sequence changes. Any methods known in the art for detecting mutations may be used, as will be apparent to skilled artisans apprised of the present disclosure.

In another embodiment, the method of diagnosis includes determining the binding constant of the interacting protein members of one or more protein complexes. For example, the interacting protein members can be obtained from a patient by direct purification or by recombinant expression from genomic DNAs or cDNAs prepared from a patient sample encoding the interacting protein members. Binding constants represent the strength of the protein-protein interaction between the interacting protein members in a protein complex. Thus, by measuring binding constants, subtle aberrations in binding affinity may be detected.

A number of methods known in the art for estimating and determining binding constants in protein-protein interactions are reviewed in (Phizicky and Fields, et al., Microbiol. Rev., 59:94-123 (1995)), which is incorporated herein by reference. For example, protein affinity chromatography may be used. First, columns are prepared with different concentrations of an interacting protein member, which is covalently bound to the columns. Then a preparation of an interacting protein partner is run through the column and washed with buffer. The interacting protein partner bound to the interacting protein member linked to the column is then eluted. A binding constant is then estimated based on the concentrations of the bound protein and the eluted protein. Alternatively, the method of sedimentation through gradients monitors the rate of sedimentation of a mixture of proteins through gradients of glycerol or sucrose. At concentrations above the binding constant, proteins can sediment as a protein complex. Thus, binding constant can be calculated based on the concentrations. Other suitable methods known in the art for estimating binding constant include but are not limited to gel filtration column such as nonequilibrium “small-zone” gel filtration columns (See e.g., Gill et al., J. Mol. Biol., 220:307-324 (1991)), the Hummel-Dreyer method of equilibrium gel filtration (See e.g., Hummel and Dreyer, Biochim. Biophys. Acta, 63:530-532 (1962)) and large-zone equilibrium gel filtration (See e.g., Gilbert and Kellett, J. Biol. Chem., 246:6079-6086 (1971)), sedimentation equilibrium (See e.g., Rivas and Minton, Trends Biochem., 18:284-287 (1993)), fluorescence methods such as fluorescence spectrum (See e.g., Otto-Bruc et al., Biochemistry, 32:8632-8645 (1993)) and fluorescence polarization or anisotropy with tagged molecules (See e.g., Weiel and Hershey, Biochemistry, 20:5859-5865 (1981)), solution equilibrium measured with immobilized binding protein (See e.g., Nelson and Long, Biochemistry, 30:2384-2390 (1991)), and surface plasmon resonance (See e.g., Panayotou et al., Mol. Cell. Biol., 13:3567-3576 (1993)).

In another embodiment, the diagnosis method of the present invention comprises detecting protein-protein interactions in functional assay systems such as the yeast two-hybrid system. Accordingly, to determine the protein-protein interaction between two interacting protein members that normally form a protein complex in normal individuals, cDNAs encoding the interacting protein members can be isolated from a patient to be diagnosed. The thus cloned cDNAs or fragments thereof can be subcloned into vectors for use in yeast two-hybrid systems. Preferably a reverse yeast two-hybrid system is used such that failure of interaction between the proteins may be positively detected. The use of yeast two-hybrid systems or other systems for detecting protein-protein interactions is known in the art and is described below in Section 5.3.1.

A kit may be used for conducting the diagnosis methods of the present invention. Typically, the kit should contain, in a carrier or compartmentalized container, reagents useful in any of the above-described embodiments of the diagnosis method. The carrier can be a container or support, in the form of, e.g., bag, box, tube, rack, and is optionally compartmentalized. The carrier may define an enclosed confinement for safety purposes during shipment and storage. In one embodiment, the kit includes an antibody selectively immunoreactive with a protein complex of the present invention. In addition, antibodies against individual interacting protein members of the protein complexes may also be included. The antibodies may be labeled with a detectable marker such as radioactive isotopes, or enzymatic or fluorescence markers. Alternatively secondary antibodies such as labeled anti-IgG and the like may be included for detection purposes. Optionally, the kit can include one or more of the protein complexes of the present invention prepared or purified from a normal individual or an individual afflicted with a physiological disorder associated with an aberration in the protein complexes or an interacting protein member thereof. In addition, the kit may further include one or more of the interacting protein members of the protein complexes of the present invention prepared or purified from a normal individual or an individual afflicted with a physiological disorder associated with an aberration in the protein complexes or an interacting protein member thereof. Suitable oligonucleotide primers useful in the amplification of the genes or cDNAs for the interacting protein members may also be provided in the kit. In particular, in a preferred embodiment, the kit includes a first oligonucleotide selectively hybridizable to the mRNA or cDNA encoding one member of an interacting pair of proteins and a second oligonucleotide selectively hybridizable to the mRNA or cDNA encoding the other of the interacting pair. Additional oligonucleotides hybridizing to a region of the genes encoding an interacting pair of proteins may also be included. Such oligonucleotides may be used as PCR primers for, e.g., quantitative PCR amplification of mRNAs encoding the interacting proteins, or as hybridizing probes for detecting the mRNAs. The oligonucleotides may have a length of from about 8 nucleotides to about 100 nucleotides, preferably from about 12 to about 50 nucleotides, and more preferably from about 15 to about 30 nucleotides. In addition, the kit may also contain oligonucleotides that can be used as hybridization probes for detecting the cDNAs or mRNAs encoding the interacting protein members. Preferably, instructions for using the kit or reagents contained therein are also included in the kit.

### 5. Use of Protein Complexes or Interacting Protein Members Thereof in Screening Assays for Modulators

The protein complexes of the present invention and interacting members thereof can also be used in screening assays to identify modulators of the protein complexes, and/or the interacting proteins. In addition, homologues, derivatives or fragments of the interacting proteins provided in this invention may also be used in such screening assays. As used herein, the term “modulator” encompasses any compounds that can cause any form of alteration of the biological activities or functions of the proteins or protein complexes, including, e.g., enhancing or reducing their biological activities, increasing or decreasing their stability, altering their affinity or specificity to certain other biological molecules, etc. In addition, the term “modulator” as used herein also includes any compounds that simply bind any of the proteins described in the tables, and/or the proteins complexes of the present invention. For example, a modulator can be an “interaction antagonist” capable of interfering with or disrupting or dissociating protein-protein interaction between an interacting pair of proteins identified in the tables, or homologues, fragments or derivatives thereof. A modulator can also be an “interaction agonist” that initiates or strengthens the interaction between the protein members of a protein complex of the present invention, or homologues, fragments or derivatives thereof.

In addition, the discovery of protein ligands of the present invention allows the use of screening assays to identify modulators of individual proteins of the protein complexes. Typical high-throughput screening assays involve measuring the modulation of the enzymatic activity of a protein. However, typical high-throughput screening assays are not applicable to proteins that exhibit little or no measurable enzymatic activity. The present discovery of novel ligands of proteins allows a screen to be setup that does not utilize enzymatic activity measurements. Consequently, the present invention enables a non-enzymatic high-throughput assay to be performed for modulators of individual proteins and/or protein complexes described in the tables.

Accordingly, the present invention provides screening methods for selecting modulators of any of the proteins described in the tables, or a mutant form thereof, or a protein-protein interaction between an interacting pair of proteins provided in the present invention, or homologues, fragments or derivatives thereof.

The selected compounds can be tested for their ability to modulate (interfere with or strengthen) the interaction between the interacting partners within the protein complexes of the present invention. In addition, the compounds can also be further tested for their ability to modulate (inhibit or enhance) cellular functions such as angiogenesis, cell proliferation and transformation, intracellular vesicle trafficking, vacuolar protein sorting, formation of multivesicular bodies and endocytosis, inhibiting viral budding, suppress tumorigenesis and cell transformation, and reduce autoimmune response. In addition, the methods may also be used in the treatment or prevention of diseases and disorders such as viral infection, cancer and autoimmune diseases, cancer, AIDS, asthma, ischemia, stroke, autoimmune diseases, neurodegenerative diseases, inflammatory disorders, sepsis, and osteoporosis. The methods may also be used in the treatment or prevention of diseases and disorders such as cholesterol transport and lipid metabolism such as dementia such as Alzheimer's disease and cardiovascular diseases such as coronary artery disease, atherosclerosis, hypercholesterolemia, Tangier disease and amyloidosis, formation and maintenance of high density lipoprotein (HDL) microparticles, cholesterol and lipid transport and metabolism in cells, hyperlipidemia, hypercholesterolemia, and cardiovascular disease.

The compounds can be tested for their ability to modulate the protein complexes of tables 1 through 11 in cells, as well as for their effectiveness in treating diseases such as abnormal cell proliferation (hyperproliferation or dysproliferation), keloid, liver cirrhosis, psoriasis, altered wound healing, cancer, especially cancers of the breast, ovary, colon, prostate, and lung, melanomas and cancers of hematopoietic origin, type 2 diabetes, obesity, hypertension, hypercholesterolemia, atherosclerosis, coronary artery disease, peripheral vascular disease, optic neuropathy, inflammatory response, amyloidosis, dementia, Alzheimer's disease, Tangier disease, Angelman syndrome, and specific viral infections.

The modulators selected in accordance with the screening methods of the present invention can be effective in modulating the functions or activities of individual interacting proteins, or the protein complexes of the present invention. For example, compounds capable of binding to the protein complexes may be capable of modulating the functions of the protein complexes. Additionally, compounds that interfere with, weaken, dissociate or disrupt, or alternatively, initiate, facilitate or stabilize the protein-protein interaction between the interacting protein members of the protein complexes can also be effective in modulating the functions or activities of the protein complexes. Thus, the compounds identified in the screening methods of the present invention can be made into therapeutically or prophylactically effective drugs for preventing or ameliorating diseases, disorders or symptoms caused by or associated with a protein complex or an interacting member thereof. Alternatively, they may be used as leads to aid the design and identification of therapeutically or prophylactically effective compounds for diseases, disorders or symptoms caused by or associated with the protein complex or interacting protein members thereof. The protein complexes and/or interacting protein members thereof in accordance with the present invention can be used in any of a variety of drug screening techniques. Drug screening can be performed as described herein or using well-known techniques, such as those described in U.S. Pat. Nos. 5,800,998 and 5,891,628, both of which are incorporated herein by reference.

### 5.1. Test Compounds

Any test compounds may be screened in the screening assays of the present invention to select modulators of the protein complexes or interacting members thereof. By the term “selecting” or “select” compounds it is intended to encompass both (a) choosing compounds from a group previously unknown to be modulators of a protein complex or interacting protein members thereof; and (b) testing compounds that are known to be capable of binding, or modulating the functions and activities of, a protein complex or interacting protein members thereof. Both types of compounds are generally referred to herein as “test compounds.” The test compounds may include, by way of example, proteins (e.g., antibodies, small peptides, artificial or natural proteins), nucleic acids, and derivatives, mimetics and analogs thereof, and small organic molecules having a molecular weight of no greater than 10,000 daltons, more preferably less than 5,000 daltons. Preferably, the test compounds are provided in library formats known in the art, e.g., in chemically synthesized libraries, recombinantly expressed libraries (e.g., phage display libraries), and in vitro translation-based libraries (e.g., ribosome display libraries).

For example, the screening assays of the present invention can be used in the antibody production processes described in Section 3 to select antibodies with desirable specificities. Various forms of antibodies or derivatives thereof may be screened, including but not limited to, polyclonal antibodies, monoclonal antibodies, bifunctional antibodies, chimeric antibodies, single chain antibodies, antibody fragments such as Fv fragments, single-chain Fv fragments (scFv), Fab′ fragments, and F(ab′)₂ fragments, and various modified forms of antibodies such as catalytic antibodies, and antibodies conjugated to toxins or drugs, and the like. The antibodies can be of any types such as IgG, IgE, IgA, or IgM. Humanized antibodies are particularly preferred. Preferably, the various antibodies and antibody fragments may be provided in libraries to allow large-scale high throughput screening. For example, expression libraries expressing antibodies or antibody fragments may be constructed by a method disclosed, e.g., in (Huse et al., Science, 246:1275-1281 (1989)), which is incorporated herein by reference. Single-chain Fv (scFv) antibodies are of particular interest in diagnostic and therapeutic applications. Methods for providing antibody libraries are also provided in U.S. Pat. Nos. 6,096,551; 5,844,093; 5,837,460; 5,789,208; and 5,667,988, all of which are incorporated herein by reference.

Peptidic test compounds may be peptides having L-amino acids and/or D-amino acids, phosphopeptides, and other types of peptides. The screened peptides can be of any size, but preferably have less than about 50 amino acids. Smaller peptides are easier to deliver into a patient's body. Various forms of modified peptides may also be screened. Like antibodies, peptides can also be provided in, e.g., combinatorial libraries. (See generally, Gallop et al., J. Med. Chem., 37:1233-1251 (1994)). Methods for making random peptide libraries are disclosed in, (e.g., Devlin et al., Science, 249:404-406 (1990)). Other suitable methods for constructing peptide libraries and screening peptides therefrom are disclosed in, (e.g., Scott and Smith, Science, 249:386-390 (1990); Moran et al., J. Am. Chem. Soc., 117:10787-10788 (1995) (a library of electronically tagged synthetic peptides); Stachelhaus et al., Science, 269:69-72 (1995); U.S. Pat. Nos. 6,156,511; 6,107,059; 6,015,561; 5,750,344; 5,834,318; 5,750,344), all of which are incorporated herein by reference. For example, random-sequence peptide phage display libraries may be generated by cloning synthetic oligonucleotides into the gene III or gene VIII of an E. coli filamentous phage. The thus generated phage can propagate in E. coli. and express peptides encoded by the oligonucleotides as fusion proteins on the surface of the phage. (Scott and Smith, Science, 249:368-390 (1990)). Alternatively, the “peptides on plasmids” method may also be used to form peptide libraries. In this method, random peptides may be fused to the C-terminus of the E. coli. Lac repressor by recombinant technologies and expressed from a plasmid that also contains Lac repressor-binding sites. As a result, the peptide fusions bind to the same plasmid that encodes them.

Small organic or inorganic non-peptide non-nucleotide compounds are preferred test compounds for the screening assays of the present invention. They too can be provided in a library format. (See generally, Gordan et al. J. Med. Chem., 37:1385-1401 (1994). For example, benzodiazepine libraries are provided in Bunin and Ellman, J. Am. Chem. Soc., 114:10997-10998 (1992)), which is incorporated herein by reference. Methods for constructing and screening peptoid libraries are disclosed in (Simon et al., Proc. Natl. Acad. Sci. USA, 89:9367-9371 (1992)). Methods for the biosynthesis of novel polyketides in a library format are described in (McDaniel, Science, 262:1546-1550 (1993) and Kao et al., Science, 265:509-512 (1994)). Various libraries of small organic molecules and methods of construction thereof are disclosed in U.S. Pat. Nos. 6,162,926 (multiply-substituted fullerene derivatives); 6,093,798 (hydroxamic acid derivatives); 5,962,337 (combinatorial 1,4-benzodiazepin-2,5-dione library); 5,877,278 (Synthesis of N-substituted oligomers); 5,866,341 (compositions and methods for screening drug libraries); 5,792,821 (polymerizable cyclodextrin derivatives); 5,766,963 (hydroxypropylamine library); and 5,698,685 (morpholino-subunit combinatorial library), all of which are incorporated herein by reference.

Other compounds such as oligonucleotides and peptide nucleic acids (PNA), and analogs and derivatives thereof may also be screened to identify clinically useful compounds. Combinatorial libraries of oligonucleotides are also known in the art. (See Gold et al., J. Biol. Chem., 270:13581-13584 (1995)).

### 5.2. In Vitro Screening Assays

The test compounds may be screened in an in vitro assay to identify compounds capable of binding the protein complexes or interacting protein members thereof in accordance with the present invention. For this purpose, a test compound is contacted with a protein complex or an interacting protein member thereof under conditions and for a time sufficient to allow specific interaction between the test compound and the target components to occur, thereby resulting in the binding of the compound to the target, and the formation of a complex. Subsequently, the binding event is detected.

Various screening techniques known in the art may be used in the present invention. The protein complexes and the interacting protein members thereof may be prepared by any suitable methods, e.g., by recombinant expression and purification. The protein complexes and/or interacting protein members thereof (both are referred to as “target” hereinafter in this section) may be free in solution. A test compound may be mixed with a target forming a liquid mixture. The compound may be labeled with a detectable marker. Upon mixing under suitable conditions, the binding complex having the compound and the target may be co-immunoprecipitated and washed. The compound in the precipitated complex may be detected based on the marker on the compound.

In a preferred embodiment, the target is immobilized on a solid support or on a cell surface. Preferably, the target can be arrayed into a protein microchip in a method described in Section 2.3. For example, a target may be immobilized directly onto a microchip substrate such as glass slides or onto multi-well plates using non-neutralizing antibodies, i.e., antibodies capable of binding to the target but do not substantially affect its biological activities. To affect the screening, test compounds can be contacted with the immobilized target to allow binding to occur to form complexes under standard binding assay conditions. Either the targets or test compounds are labeled with a detectable marker using well-known labeling techniques. For example, U.S. Pat. No. 5,741,713 discloses combinatorial libraries of biochemical compounds labeled with NMR active isotopes. To identify binding compounds, one may measure the formation of the target-test compound complexes or kinetics for the formation thereof. When combinatorial libraries of organic non-peptide non-nucleic acid compounds are screened, it is preferred that labeled or encoded (or “tagged”) combinatorial libraries are used to allow rapid decoding of lead structures. This is especially important because, unlike biological libraries, individual compounds found in chemical libraries cannot be amplified by self-amplification. Tagged combinatorial libraries are provided in, e.g., Borchardt and Still, J. Am. Chem. Soc., 116:373-374 (1994) and Moran et al., J. Am. Chem. Soc., 117:10787-10788 (1995), both of which are incorporated herein by reference.

Alternatively, the test compounds can be immobilized on a solid support, e.g., forming a microarray of test compounds. The target protein or protein complex is then contacted with the test compounds. The target may be labeled with any suitable detection marker. For example, the target may be labeled with radioactive isotopes or fluorescence marker before binding reaction occurs. Alternatively, after the binding reactions, antibodies that are immunoreactive with the target and are labeled with radioactive materials, fluorescence markers, enzymes, or labeled secondary anti-Ig antibodies may be used to detect any bound target thus identifying the binding compound. One example of this embodiment is the protein probing method. That is, the target provided in accordance with the present invention is used as a probe to screen expression libraries of proteins or random peptides. The expression libraries can be phage display libraries, in vitro translation-based libraries, or ordinary expression cDNA libraries. The libraries may be immobilized on a solid support such as nitrocellulose filters. See e.g., Sikela and Hahn, Proc. Natl. Acad. Sci. USA, 84:3038-3042 (1987). The probe may be labeled with a radioactive isotope or a fluorescence marker. Alternatively, the probe can be biotinylated and detected with a streptavidin-alkaline phosphatase conjugate. More conveniently, the bound probe may be detected with an antibody.

In one embodiment, the proteins identified in the tables are used as targets in an assay to select modulators of the proteins in the tables. In a specific embodiment, a screening assay for modulators of APOA1 is performed by using PRA1 as a ligand for APOA1. For example, in this screen, APOA1 can be immobilized on a solid support and is contacted with test compounds. PRA1 can be labeled with a detectable marker such as radioactive materials or fluorescence markers using label techniques known in the art. The labeled PRA1 is allowed to contact the immobilized APOA1 and levels of APOA1-PRA1 protein complex formed are detected by washing away unbound PRA1. The ability of the test compounds to modulate APOA1 is determined by comparing the level of APOA1-PRA1 complex formed when APOA1 is contacted with test compounds to the level formed in the absence of test compounds. Alternatively, as will be apparent to skilled artisans, the PRA1 protein can be detected with labeled antibody against PRA1, or by an antibody specific to a polypeptide that is fused to PRA1.

In yet another embodiment, the protein complexes identified in the tables are used as a target in the assay. In a specific embodiment, a protein complex used in the screening assay includes a hybrid protein as described in Section 2.1, which is formed by fusion of two interacting protein members or fragments or interaction domains thereof. The hybrid protein may also be designed such that it contains a detectable epitope tag fused thereto. Suitable examples of such epitope tags include sequences derived from, e.g., influenza virus hemagglutinin (HA), Simian Virus 5 (V5), polyhistidine (6×His), c-myc, lacZ, GST, and the like.

In addition, a known ligand capable of binding to the target can be used in competitive binding assays. Complexes between the known ligand and the target can be formed and then contacted with test compounds. The ability of a test compound to interfere with the interaction between the target and the known ligand is measured. One exemplary ligand is an antibody capable of specifically binding the target. Particularly, such an antibody is especially useful for identifying peptides that share one or more antigenic determinants of the target protein complex or interacting protein members thereof.

In a specific preferred embodiment, the target is one member of an interacting pair of proteins disclosed according the present invention, or a homologue, derivative, or fragment thereof, and the competitive ligand is the other member of the interacting pair of proteins, or a homologue, derivative, or fragment thereof. Preferably, either the target or the ligand or both are labeled with or detectable marker. Alternatively, either the target or the ligand or both are fusion proteins that contain a detectable epitope tag having one or more sequences derived from, e.g., influenza virus hemagglutinin (HA), Simian Virus 5 (V5), polyhistidine (6×His), c-myc, lacZ, GST, and the like.

Thus, for example, the target can be immobilized to a solid support. The ligand can be a fusion protein having a fragment of an interactor of the target protein fused to an epitope tag, e.g., c-myc. The ligand can be contacted with the target in the presence or absence of one or more test compounds. Both ligand molecules associated with the immobilized target and ligand molecules not associated with the target can be detected with, e.g., an antibody against the c-myc tag. As a result, test compounds capable of binding the target or ligand, or disrupting the protein-protein interaction between the target and ligand can be identified or selected.

Test compounds may also be screened in an in vitro assay to identify compounds capable of dissociating the protein complexes identified in the tables above. Thus, for example, any one of the interacting pairs of proteins described in the tables above can be contacted with a test compound and the integrity of the protein complex can be assessed. Conversely, test compounds may also be screened to identify compounds capable of enhancing the interactions between the constituent members of the protein complexes formed by the interactions described in the tables. The assays can be conducted in a manner similar to the binding assays described above. For example, the presence or absence of a particular pair of interacting proteins can be detected by an antibody selectively immunoreactive with the protein complex formed by those two proteins. Thus, after incubation of the protein complex with a test compound, an immunoprecipitation assay can be conducted with the antibody. If the test compound disrupts the protein complex, then the amount of immunoprecipitated protein complex in this assay will be significantly less than that in a control assay in which the same protein complex is not contacted with the test compound. Similarly, two proteins—the interaction between which is to be enhanced—may be incubated together with a test compound. Thereafter, a protein complex formed by the two interacting proteins may be detected by the selectively immunoreactive antibody. The amount of protein complex may be compared to that formed in the absence of the test compound. Various other detection methods may be suitable in the dissociation assay, as will be apparent to a skilled artisan apprised of the present disclosure.

In another embodiment, fluorescent resonance energy transfer (FRET) is used to screen for modulators of interacting proteins of the protein complexes of the present invention. FRET assays measure the energy transfer of a fluorescent label to another fluorescent label. Fluorescent labels absorb light preferentially at one wavelength and emit light preferentially at a second wavelength. FRET assays utilize this characteristic by selecting a fluorescent label, called a donor fluorophore, that emits light preferentially at the wavelength a second label, called the acceptor fluorophore, preferentially absorbs light. The proximity of the donor and acceptor fluorophore can be determined by measuring the energy transfer from the donor fluorophore to the acceptor fluorophore. Measuring the energy transfer is performed by shining light on a solution containing acceptor and donor fluorophores at the wavelength the donor fluorophore absorbs light and measuring fluorescence at the wavelength the acceptor fluorophore emits light. The amount of fluorescence of the acceptor fluorophore indicates the proximity of the donor and acceptor fluorophores.

For example, FRET assays can be used to screen for modulators of APOA1 by labeling APOA1 or an antibody to APOA1 with an acceptor fluorophore and labeling a APOA1 substrate or interactor (e.g., PRA1) or an antibody to a APOA1 substrate/interactor with an acceptor fluorophore. If the test compound is a APOA1 modulator it will decrease the fluorescence of the acceptor fluorophore because the acceptor and donor fluorphore will not be as close to each other.

In a specific embodiment of a FRET assay, TP³⁺ is attached to an antibody to APOA1, and BODIPY-TMR is attached to an antibody to an interactor (e.g., PRA1). The fluorescently labeled antibodies, APOA1, and APOA1 substrates are put in solution together. Light at the wavelength that TP³⁺ preferentially absorbs light is shined on the solution and the fluorescence of the solution is measured at the wavelength that BODIPY-TMR preferentially emits light. A test compound is then added to the solution and light at the wavelength that TP³⁺ preferentially absorbs light is shined on the solution and the fluorescence of the solution is measured at the wavelength that BODIPY-TMR preferentially emits light. If the fluorescence of the solution with the test compound decreases compared to the fluorescence of the solution without the test compound then the test compound is an APOA1 modulator.

### 5.3. In vivo Screening Assays

Test compounds can also be screened in any in vivo assays to select modulators of the protein complexes or interacting protein members thereof in accordance with the present invention. For example, any in vivo assays known in the art to be useful in identifying compounds capable of strengthening or interfering with the stability of the protein complexes of the present invention may be used.

In a specific example, a screening assay for modulators of a APOA1 is performed by using PRA1 as a ligand for APOA1. In this screen, APOA1 is contacted with test compounds in the presence of PRA1 and the levels of APOA1-PRA1 protein complex formed when APOA1 is contacted with the test compound in the presence of PRA1 is detected. The ability of the test compounds to modulate APOA1 is determined by comparing the level of APOA1-PRA1 complex formed when APOA1 is contacted with test compounds to the level formed in the absence of test compounds. If the level of APOA1-PRA1 protein complex formed when APOA1 is contacted with the test compound then the test compound is a modulator of APOA1.

To screen peptidic compounds for modulators of APOA1, the two-hybrid systems described in Section 4 may be used in the screening assays in which the APOA1 protein is expressed in, e.g., a bait fusion protein and the peptidic test compounds are expressed in, e.g., prey fusion proteins. Screening peptidic compounds for modulators of the proteins identified in the tables can also be performed using the two-hybrid systems described in Section 4 by expressing the proteins identified in the tables in, e.g., a bait fusion protein and expressing the peptidic test compounds in e.g., prey fusion proteins.

To screen for modulators of the protein-protein interaction between APOA1 and an APOA1-interacting protein, the methods of the present invention typically comprise contacting the APOA1 protein with the APOA1-interacting protein in the presence of a test compound, and determining the interaction between the APOA1 protein and the APOA1-interacting protein. In a preferred embodiment, a two-hybrid system, e.g., a yeast two-hybrid system as described in detail in Section 4 is employed.

### 5.3.1. Two-Hybrid Assays

In a preferred embodiment, one of the yeast two-hybrid systems or their analogous or derivative forms is used. Examples of suitable two-hybrid systems known in the art include, but are not limited to, those disclosed in U.S. Pat. Nos. 5,283,173; 5,525,490; 5,585,245; 5,637,463; 5,695,941; 5,733,726; 5,776,689; 5,885,779; 5,905,025; 6,037,136; 6,057,101; 6,114,111; and Bartel and Fields, eds., The Yeast Two-Hybrid System, Oxford University Press, New York, N.Y., 1997, all of which are incorporated herein by reference.

Typically, in a classic transcription-based two-hybrid assay, two chimeric genes are prepared encoding two fusion proteins: one contains a transcription activation domain fused to an interacting protein member of a protein complex of the present invention or an interaction domain or fragment of the interacting protein member, while the other fusion protein includes a DNA binding domain fused to another interacting protein member of the protein complex or a fragment or interaction domain thereof. For the purpose of convenience, the two interacting protein members, fragments or interaction domains thereof are referred to as “bait fusion protein” and “prey fusion protein,” respectively. The chimeric genes encoding the fusion proteins are termed “bait chimeric gene” and “prey chimeric gene,” respectively. Typically, a “bait vector” and a “prey vector” are provided for the expression of a bait chimeric gene and a prey chimeric gene, respectively.

### 5.3.1.1. Vectors

Many types of vectors can be used in a transcription-based two-hybrid assay. Methods for the construction of bait vectors and prey vectors should be apparent to skilled artisans in the art apprised of the present disclosure. See generally, Current Protocols in Molecular Biology, Vol. 2, Ed. Ausubel, et al., Greene Publish. Assoc. & Wiley Interscience, Ch. 13, 1988; Glover, DNA Cloning, Vol. II, IRL Press, Wash., D.C., Ch. 3, 1986; Bitter, et al., in Methods in Enzymology 153:516-544 (1987); The Molecular Biology of the Yeast Saccharomyces, Eds. Strathern et al., Cold Spring Harbor Press, Vols. I and II, 1982; and Rothstein in DNA Cloning: A Practical Approach, Vol. 11, Ed. DM Glover, IRL Press, Wash., D.C., 1986.

Generally, the bait and prey vectors include an expression cassette having a promoter operably linked to a chimeric gene for the transcription of the chimeric gene. The vectors may also include an origin of DNA replication for the replication of the vectors in host cells and a replication origin for the amplification of the vectors in, e.g., E. coli, and selection marker(s) for selecting and maintaining only those host cells harboring the vectors. Additionally, the expression cassette preferably also contains inducible elements, which function to control the expression of a chimeric gene. Making the expression of the chimeric genes inducible and controllable is especially important in the event that the fusion proteins or components thereof are toxic to the host cells. Other regulatory sequences such as transcriptional enhancer sequences and translation regulation sequences (e.g., Shine-Dalgarno sequence) can also be included in the expression cassette. Termination sequences such as the bovine growth hormone, SV40, lacZ and AcMNPV polyhedral polyadenylation signals may also be operably linked to a chimeric gene in the expression cassette. An epitope tag coding sequence for detection and/or purification of the fusion proteins can also be operably linked to the chimeric gene in the expression cassette. Examples of useful epitope tags include, but are not limited to, influenza virus hemagglutinin (HA), Simian Virus 5 (V5), polyhistidine (6×His), c-myc, lacZ, GST, and the like. Proteins with polyhistidine tags can be easily detected and/or purified with Ni affinity columns, while specific antibodies to many epitope tags are generally commercially available. The vectors can be introduced into the host cells by any techniques known in the art, e.g., by direct DNA transformation, microinjection, electroporation, viral infection, lipofection, gene gun, and the like. The bait and prey vectors can be maintained in host cells in an extrachromosomal state, i.e., as self-replicating plasmids or viruses. Alternatively, one or both vectors can be integrated into chromosomes of the host cells by conventional techniques such as selection of stable cell lines or site-specific recombination.

The in vivo assays of the present invention can be conducted in many different host cells, including but not limited to bacteria, yeast cells, plant cells, insect cells, and mammalian cells. A skilled artisan will recognize that the designs of the vectors can vary with the host cells used. In one embodiment, the assay is conducted in prokaryotic cells such as Escherichia coli, Salmonella, Klebsiella, Pseudomonas, Caulobacter, and Rhizobium. Suitable origins of replication for the expression vectors useful in this embodiment of the present invention include, e.g., the ColE1, pSC101, and M13 origins of replication. Examples of suitable promoters include, for example, the T7 promoter, the lacZ promoter, and the like. In addition, inducible promoters are also useful in modulating the expression of the chimeric genes. For example, the lac operon from bacteriophage lambda plac5 is well known in the art and is inducible by the addition of IPTG to the growth medium. Other known inducible promoters useful in a bacteria expression system include pL of bacteriophage λ, the trp promoter, and hybrid promoters such as the tac promoter, and the like.

In addition, selection marker sequences for selecting and maintaining only those prokaryotic cells expressing the desirable fusion proteins should also be incorporated into the expression vectors. Numerous selection markers including auxotrophic markers and antibiotic resistance markers are known in the art and can all be useful for purposes of this invention. For example, the bla gene, which confers ampicillin resistance, is the most commonly used selection marker in prokaryotic expression vectors. Other suitable markers include genes that confer neomycin, kanamycin, or hygromycin resistance to the host cells. In fact, many vectors are commercially available from vendors such as Invitrogen Corp. of Carlsbad, Calif., Clontech Corp. of Palo Alto, Calif., and Stratagene Corp. of La Jolla, Calif., and Promega Corp. of Madison, Wis. These commercially available vectors, e.g., pBR322, pSPORT, pBluescriptIISK, pcDNAI, and pcDNAII all have a multiple cloning site into which the chimeric genes of the present invention can be conveniently inserted using conventional recombinant techniques. The constructed expression vectors can be introduced into host cells by various transformation or transfection techniques generally known in the art.

In another embodiment, mammalian cells are used as host cells for the expression of the fusion proteins and detection of protein-protein interactions. For this purpose, virtually any mammalian cells can be used including normal tissue cells, stable cell lines, and transformed tumor cells. Conveniently, mammalian cell lines such as CHO cells, Jurkat T cells, NIH 3T3 cells, HEK-293 cells, CV-1 cells, COS-1 cells, HeLa cells, VERO cells, MDCK cells, WI38 cells, and the like are used. Mammalian expression vectors are well known in the art and many are commercially available. Examples of suitable promoters for the transcription of the chimeric genes in mammalian cells include viral transcription promoters derived from adenovirus, simian virus 40 (SV40) (e.g., the early and late promoters of SV40), Rous sarcoma virus (RSV), and cytomegalovirus (CMV) (e.g., CMV immediate-early promoter), human immunodeficiency virus (HIV) (e.g., long terminal repeat (LTR)), vaccinia virus (e.g., 7.5K promoter), and herpes simplex virus (HSV) (e.g., thymidine kinase promoter). Inducible promoters can also be used. Suitable inducible promoters include, for example, the tetracycline responsive element (TRE) (See Gossen et al., Proc. Natl. Acad. Sci. USA, 89:5547-5551 (1992)), metallothionein IIA promoter, ecdysone-responsive promoter, and heat shock promoters. Suitable origins of replication for the replication and maintenance of the expression vectors in mammalian cells include, e.g., the Epstein Barr origin of replication in the presence of the Epstein Barr nuclear antigen (see Sugden et al., Mole. Cell. Biol., 5:410-413 (1985)) and the SV40 origin of replication in the presence of the SV40 T antigen (which is present in COS-1 and COS-7 cells) (see Margolskee et al., Mole. Cell. Biol., 8:2837 (1988)). Suitable selection markers include, but are not limited to, genes conferring resistance to neomycin, hygromycin, zeocin, and the like. Many commercially available mammalian expression vectors may be useful for the present invention, including, e.g., pCEP4, pcDNAI, pIND, pSecTag2, pVAX1, pcDNA3.1, and pBI-EGFP, and pDisplay. The vectors can be introduced into mammalian cells using any known techniques such as calcium phosphate precipitation, lipofection, electroporation, and the like. The bait vector and prey vector can be co-transformed into the same cell or, alternatively, introduced into two different cells which are subsequently fused together by cell fusion or other suitable techniques.

Viral expression vectors, which permit introduction of recombinant genes into cells by viral infection, can also be used for the expression of the fusion proteins. Viral expression vectors generally known in the art include viral vectors based on adenovirus, bovine papilloma virus, murine stem cell virus (MSCV), MFG virus, and retrovirus. See Sarver, et al., Mol. Cell. Biol., 1: 486 (1981); Logan & Shenk, Proc. Natl. Acad. Sci. USA, 81:3655-3659 (1984); Mackett, et al., Proc. Natl. Acad. Sci. USA, 79:7415-7419 (1982); Mackett, et al., J. Virol, 49:857-864 (1984); Panicali, et al., Proc. Natl. Acad. Sci. USA, 79:4927-4931 (1982); Cone & Mulligan, Proc. Natl. Acad. Sci. USA, 81:6349-6353 (1984); Mann et al., Cell, 33:153-159 (1993); Pear et al., Proc. Natl. Acad. Sci. USA, 90:8392-8396 (1993); Kitamura et al., Proc. Natl. Acad. Sci. USA, 92:9146-9150 (1995); Kinsella et al., Human Gene Therapy, 7:1405-1413 (1996); Hofmann et al., Proc. Natl. Acad. Sci. USA, 93:5185-5190 (1996); Choate et al., Human Gene Therapy, 7:2247 (1996); WO 94/19478; Hawley et al., Gene Therapy, 1:136 (1994) and Rivere et al., Genetics, 92:6733 (1995), all of which are incorporated by reference.

Generally, to construct a viral vector, a chimeric gene according to the present invention can be operably linked to a suitable promoter. The promoter-chimeric gene construct is then inserted into a non-essential region of the viral vector, typically a modified viral genome. This results in a viable recombinant virus capable of expressing the fusion protein encoded by the chimeric gene in infected host cells. Once in the host cell, the recombinant virus typically is integrated into the genome of the host cell. However, recombinant bovine papilloma viruses typically replicate and remain as extrachromosomal elements.

In another embodiment, the detection assays of the present invention are conducted in plant cell systems. Methods for expressing exogenous proteins in plant cells are well known in the art. See generally, Weissbach & Weissbach, Methods for Plant Molecular Biology, Academic Press, NY, 1988; Grierson & Corey, Plant Molecular Biology, 2d Ed., Blackie, London, 1988. Recombinant virus expression vectors based on, e.g., cauliflower mosaic virus (CaMV) or tobacco mosaic virus (TMV) can all be used. Alternatively, recombinant plasmid expression vectors such as Ti plasmid vectors and Ri plasmid vectors are also useful. The chimeric genes encoding the fusion proteins of the present invention can be conveniently cloned into the expression vectors and placed under control of a viral promoter such as the 35S RNA and 19S RNA promoters of CaMV or the coat protein promoter of TMV, or of a plant promoter, e.g., the promoter of the small subunit of RUBISCO and heat shock promoters (e.g., soybean hsp17.5-E or hsp17.3-B promoters).

In addition, the in vivo assay of the present invention can also be conducted in insect cells, e.g., Spodoptera frugiperda cells, using a baculovirus expression system. Expression vectors and host cells useful in this system are well known in the art and are generally available from various commercial vendors. For example, the chimeric genes of the present invention can be conveniently cloned into a non-essential region (e.g., the polyhedrin gene) of an Autographa californica nuclear polyhedrosis virus (AcNPV) vector and placed under control of an AcNPV promoter (e.g., the polyhedrin promoter). The non-occluded recombinant viruses thus generated can be used to infect host cells such as Spodoptera frugiperda cells in which the chimeric genes are expressed. See U.S. Pat. No. 4,215,051.

In a preferred embodiment of the present invention, the fusion proteins are expressed in a yeast expression system using yeasts such as Saccharomyces cerevisiae, Hansenula polymorpha, Pichia pastoris, and Schizosaccharomyces pombe as host cells. The expression of recombinant proteins in yeasts is a well-developed field, and the techniques useful in this respect are disclosed in detail in The Molecular Biology of the Yeast Saccharomyces, Eds. Strathern et al., Vols. I and II, Cold Spring Harbor Press, 1982; Ausubel et al., Current Protocols in Molecular Biology, New York, Wiley, 1994; and Guthrie and Fink, Guide to Yeast Genetics and Molecular Biology, in Methods in Enzymology, Vol. 194, 1991, all of which are incorporated herein by reference. Sudbery, Curr. Opin. Biotech., 7:517-524 (1996) reviews the successes in the art of expressing recombinant proteins in various yeast species; the entire content and references cited therein are incorporated herein by reference. In addition, Bartel and Fields, eds., The Yeast Two-Hybrid System, Oxford University Press, New York, N.Y., 1997 contains extensive discussions of recombinant expression of fusion proteins in yeasts in the context of various yeast two-hybrid systems, and cites numerous relevant references. These and other methods known in the art can all be used for purposes of the present invention. The application of such methods to the present invention should be apparent to a skilled artisan apprised of the present disclosure.

Generally, each of the two chimeric genes is included in a separate expression vector (bait vector and prey vector). Both vectors can be co-transformed into a single yeast host cell. As will be apparent to a skilled artisan, it is also possible to express both chimeric genes from a single vector. In a preferred embodiment, the bait vector and prey vector are introduced into two haploid yeast cells of opposite mating types, e.g., a-type and a-type, respectively. The two haploid cells can be mated at a desired time to form a diploid cell expressing both chimeric genes.

Generally, the bait and prey vectors for recombinant expression in yeast include a yeast replication origin such as the 2μ origin or the ARSH4 sequence for the replication and maintenance of the vectors in yeast cells. Preferably, the vectors also have a bacteria origin of replication (e.g., ColE1) and a bacteria selection marker (e.g., amp^{R} marker, i.e., bla gene). Optionally, the CEN6 centromeric sequence is included to control the replication of the vectors in yeast cells. Any constitutive or inducible promoters capable of driving gene transcription in yeast cells may be employed to control the expression of the chimeric genes. Such promoters are operably linked to the chimeric genes. Examples of suitable constitutive promoters include but are not limited to the yeast ADH1, PGK1, TEF2, GPD1, HIS3, and CYC1 promoters. Examples of suitable inducible promoters include but are not limited to the yeast GAL1 (inducible by galactose), CUP1 (inducible by Cu⁺⁺), and FUS1 (inducible by pheromone) promoters; the AOX/MOX promoter from H. polymorpha and P. pastoris (repressed by glucose or ethanol and induced by methanol); chimeric promoters such as those that contain LexA operators (inducible by LexA-containing transcription factors); and the like. Inducible promoters are preferred when the fusion proteins encoded by the chimeric genes are toxic to the host cells. If it is desirable, certain transcription repressing sequences such as the upstream repressing sequence (URS) from SPO13 promoter can be operably linked to the promoter sequence, e.g., to the 5′ end of the promoter region. Such upstream repressing sequences function to fine-tune the expression level of the chimeric genes.

Preferably, a transcriptional termination signal is operably linked to the chimeric genes in the vectors. Generally, transcriptional termination signal sequences derived from, e.g., the CYC1 and ADH1 genes can be used.

Additionally, it is preferred that the bait vector and prey vector contain one or more selectable markers for the selection and maintenance of only those yeast cells that harbor one or both chimeric genes. Any selectable markers known in the art can be used for purposes of this invention so long as yeast cells expressing the chimeric gene(s) can be positively identified or negatively selected. Examples of markers that can be positively identified are those based on color assays, including the lacZ gene (which encodes β-galactosidase), the firefly luciferase gene, secreted alkaline phosphatase, horseradish peroxidase, the blue fluorescent protein (BFP), and the green fluorescent protein (GFP) gene (see Cubitt et al., Trends Biochem. Sci., 20:448-455 (1995)). Other markers allowing detection by fluorescence, chemiluminescence, UV absorption, infrared radiation, and the like can also be used. Among the markers that can be selected are auxotrophic markers including, but not limited to, URA3, HIS3, TRP1, LEU2, LYS2, ADE2, and the like. Typically, for purposes of auxotrophic selection, the yeast host cells transformed with bait vector and/or prey vector are cultured in a medium lacking a particular nutrient. Other selectable markers are not based on auxotrophies, but rather on resistance or sensitivity to an antibiotic or other xenobiotic. Examples of such markers include but are not limited to chloramphenicol acetyl transferase (CAT) gene, which confers resistance to chloramphenicol; CAN1 gene, which encodes an arginine permease and thereby renders cells sensitive to canavanine (see Sikorski et al., Meth. Enzymol., 194:302-318 (1991)); the bacterial kanamycin resistance gene (kan^{R}), which renders eukaryotic cells resistant to the aminoglycoside G418 (see Wach et al., Yeast, 10:1793-1808 (1994)); and CYH2 gene, which confers sensitivity to cycloheximide (see Sikorski et al., Meth. Enzymol., 194:302-318 (1991)). In addition, the CUP1 gene, which encodes metallothionein and thereby confers resistance to copper, is also a suitable selection marker. Each of the above selection markers may be used alone or in combination. One or more selection markers can be included in a particular bait or prey vector. The bait vector and prey vector may have the same or different selection markers. In addition, the selection pressure can be placed on the transformed host cells either before or after mating the haploid yeast cells.

As will be apparent, the selection markers used should complement the host strains in which the bait and/or prey vectors are expressed. In other words, when a gene is used as a selection marker gene, a yeast strain lacking the selection marker gene (or having mutation in the corresponding gene) should be used as host cells. Numerous yeast strains or derivative strains corresponding to various selection markers are known in the art. Many of them have been developed specifically for certain yeast two-hybrid systems. The application and optional modification of such strains with respect to the present invention will be apparent to a skilled artisan apprised of the present disclosure. Methods for genetically manipulating yeast strains using genetic crossing or recombinant mutagenesis are well known in the art. See e.g., Rothstein, Meth. Enzymol., 101:202-211 (1983). By way of example, the following yeast strains are well known in the art, and can be used in the present invention upon necessary modifications and adjustment:

L40 strain which has the genotype MATa his3Δ200 trp1-901 leu2-3,112 ade2 LYS2::(lexAop)4-HIS3 URA3::(lexAop)8-lacZ;

EGY48 strain which has the genotype MATa trp1 his3 ura3 6ops-LEU2; and

MaV103 strain which has the genotype MATa ura3-52 leu2-3,112 trp1-901 his3A200 ade2-101 gal4Δ gal80Δ SPAL10::URA3 GAL1::HIS3::lys2 (see Kumar et al., J. Biol. Chem. 272:13548-13554 (1997); Vidal et al., Proc. Natl. Acad. Sci. USA, 93:10315-10320 (1996)). Such strains are generally available in the research community, and can also be obtained by simple yeast genetic manipulation. See, e.g., The Yeast Two-Hybrid System, Bartel and Fields, eds., pages 173-182, Oxford University Press, New York, N.Y., 1997.

In addition, the following yeast strains are commercially available:

Y190 strain which is available from Clontech, Palo Alto, Calif. and has the genotype MATa gal4 gal80 his3Δ200 trp1-901 ade2-101 ura3-52 leu2-3, 112 URA3::GAL1-lacZ LYS2::GAL1-HIS3 cyh^{r}; and

YRG-2 Strain which is available from Stratagene, La Jolla, Calif. and has the genotype MATα ura3-52 his3-200 ade2-101 lys2-801 trp1-901 leu2-3, 112 gal4-542 gal80-538 LYS2::GAL1-HIS3 URA3::GAL1/CYC1-lacZ.

In fact, different versions of vectors and host strains specially designed for yeast two-hybrid system analysis are available in kits from commercial vendors such as Clontech, Palo Alto, Calif. and Stratagene, La Jolla, Calif., all of which can be modified for use in the present invention.

### 5.3.1.2. Reporters

Generally, in a transcription-based two-hybrid assay, the interaction between a bait fusion protein and a prey fusion protein brings the DNA-binding domain and the transcription-activation domain into proximity forming a functional transcriptional factor that acts on a specific promoter to drive the expression of a reporter protein. The transcription activation domain and the DNA-binding domain may be selected from various known transcriptional activators, e.g., GAL4, GCN4, ARD1, the human estrogen receptor, E. coli LexA protein, herpes simplex virus VP16 (Triezenberg et al., Genes Dev. 2:718-729 (1988)), the E. coli B42 protein (acid blob, see Gyuris et al., Cell, 75:791-803 (1993)), NF-kB p65, and the like. The reporter gene and the promoter driving its transcription typically are incorporated into a separate reporter vector. Alternatively, the host cells are engineered to contain such a promoter-reporter gene sequence in their chromosomes. Thus, the interaction or lack of interaction between two interacting protein members of a protein complex can be determined by detecting or measuring changes in the assay system's reporter. Although the reporters and selection markers can be of similar types and used in a similar manner in the present invention, the reporters and selection markers should be carefully selected in a particular detection assay such that they are distinguishable from each other and do not interfere with each other's function.

Many different types of reporters are useful in the screening assays. For example, a reporter protein may be a fusion protein having an epitope tag fused to a protein. Commonly used and commercially available epitope tags include sequences derived from, e.g., influenza virus hemagglutinin (HA), Simian Virus 5 (V5), polyhistidine (6×His), c-myc, lacZ, GST, and the like. Antibodies specific to these epitope tags are generally commercially available. Thus, the expressed reporter can be detected using an epitope-specific antibody in an immunoassay.

In another embodiment, the reporter is selected such that it can be detected by a color-based assay. Examples of such reporters include, e.g., the lacZ protein (β-galactosidase), the green fluorescent protein (GFP), which can be detected by fluorescence assay and sorted by flow-activated cell sorting (FACS) (See Cubitt et al., Trends Biochem. Sci., 20:448-455 (1995)), secreted alkaline phosphatase, horseradish peroxidase, the blue fluorescent protein (BFP), and luciferase photoproteins such as aequorin, obelin, mnemiopsin, and berovin (See U.S. Pat. No. 6,087,476, which is incorporated herein by reference).

Alternatively, an auxotrophic factor is used as a reporter in a host strain deficient in the auxotrophic factor. Thus, suitable auxotrophic reporter genes include, but are not limited to, URA3, HIS3, TRP1, LEU2, LYS2, ADE2, and the like. For example, yeast cells containing a mutant URA3 gene can be used as host cells (Ura⁻phenotype). Such cells lack URA3-encoded functional orotidine-5′-phosphate decarboxylase, an enzyme required by yeast cells for the biosynthesis of uracil. As a result, the cells are unable to grow on a medium lacking uracil. However, wild-type orotidine-5′-phosphate decarboxylase catalyzes the conversion of a non-toxic compound 5-fluoroorotic acid (5-FOA) to a toxic product, 5-fluorouracil. Thus, yeast cells containing a wild-type URA3 gene are sensitive to 5-FOA and cannot grow on a medium containing 5-FOA. Therefore, when the interaction between the interacting protein members in the fusion proteins results in the expression of active orotidine-5′-phosphate decarboxylase, the Ura⁻ (Foa^{R}) yeast cells will be able to grow on a uracil deficient medium (SC-Ura plates). However, such cells will not survive on a medium containing 5-FOA. Thus, protein-protein interactions can be detected based on cell growth.

Additionally, antibiotic resistance reporters can also be employed in a similar manner. In this respect, host cells sensitive to a particular antibiotic are used. Antibiotic resistance reporters include, for example, the chloramphenicol acetyl transferase (CAT) gene and the kan^{R} gene, which confer resistance to G418 in eukaryotes, and kanamycin in prokaryotes, respectively.

### 5.3.1.3. Screening Assays for Interaction Antagonists

The screening assays of the present invention are useful for identifying compounds capable of interfering with, disrupting, or dissociating the protein-protein interactions formed between members of the interacting protein pairs disclosed in the tables above, or between mutant and wild type, or mutant and mutant forms of these proteins. Since the protein complexes of the present invention are associated with abnormal cell proliferation (hyperproliferation or dysproliferation), keloid, liver cirrhosis, psoriasis, altered wound healing, cancer, especially cancers of the breast, ovary, colon, prostate, and lung, melanomas and cancers of hematopoietic origin, type 2 diabetes, obesity, hypertension, hypercholesterolemia, atherosclerosis, coronary artery disease, peripheral vascular disease, optic neuropathy, inflammatory response, amyloidosis, dementia, Alzheimer's disease, Tangier disease, Angelman syndrome, and specific viral infections (either directly through their known cellular roles or functions or through the association of mutant forms of these proteins with the disease, or indirectly—through their interactions with other proteins known to be linked to abnormal cell proliferation (hyperproliferation or dysproliferation), keloid, liver cirrhosis, psoriasis, altered wound healing, cancer, especially cancers of the breast, ovary, colon, prostate, and lung, melanomas and cancers of hematopoietic origin, type 2 diabetes, obesity, hypertension, hypercholesterolemia, atherosclerosis, coronary artery disease, peripheral vascular disease, optic neuropathy, inflammatory response, amyloidosis, dementia, Alzheimer's disease, Tangier disease, Angelman syndrome, and specific viral infections), disruption or dissociation of particular protein-protein interactions may be desirable to ameliorate the disease condition, or to alleviate disease symptoms. Alternatively, if the disease or disorder is associated with increased expression of any of the proteins presented in the tables, or with expression of a mutant form, or forms, of these proteins, then the disease or disorder may be ameliorated, or symptoms reduced, by weakening or dissociating the interaction between the interacting proteins in patients. Also, if a disease or disorder is associated with a mutant form of an interacting protein that form stronger protein-protein interactions with its protein partner than its wild type counterpart, then the disease or disorder may be treated with a compound that weakens, disrupts or interferes with the interaction between the mutant protein and its interacting partner.

In a screening assay for an interaction antagonist, a first protein, which is a protein selected from any of the protein pairs described in the tables (or a homologue, fragment or derivative thereof), or a mutant form of the first protein (or a homologue, fragment or derivative thereof), and a second protein, which is the interacting partner of the first protein identified in the tables above (or a homologue, fragment or derivative thereof), or a mutant form of the second protein (or a homologue, fragment or derivative thereof), are used as test proteins expressed in the form of fusion proteins as described above for purposes of a two-hybrid assay. The fusion proteins are expressed in a host cell and allowed to interact with each other in the presence of one or more test compounds.

In a preferred embodiment, a counterselectable marker is used as a reporter such that a detectable signal (e.g., appearance of color or fluorescence, or cell survival) is present only when the test compound is capable of interfering with the interaction between the two test proteins. In this respect, the reporters used in various “reverse two-hybrid systems” known in the art may be employed. Reverse two-hybrid systems are disclosed in, e.g., U.S. Pat. Nos. 5,525,490; 5,733,726; 5,885,779; Vidal et al., Proc. Natl. Acad. Sci. USA, 93:10315-10320 (1996); and Vidal et al., Proc. Natl. Acad. Sci. USA, 93:10321-10326 (1996), all of which are incorporated herein by reference.

Examples of suitable counterselectable reporters useful in a yeast system include the URA3 gene (encoding orotidine-5′-decarboxylase, which converts 5-fluoroorotic acid (5-FOA) to the toxic metabolite 5-fluorouracil), the CAN1 gene (encoding arginine permease, which transports the toxic arginine analog canavanine into yeast cells), the GAL1 gene (encoding galactokinase, which catalyzes the conversion of 2-deoxygalactose to toxic 2-deoxygalactose-1-phosphate), the LYS2 gene (encoding α-aminoadipate reductase, which renders yeast cells unable to grow on a medium containing α-aminoadipate as the sole nitrogen source), the MET15 gene (encoding O-acetylhomoserine sulfhydrylase, which confers on yeast cells sensitivity to methyl mercury), and the CYH2 gene (encoding L29 ribosomal protein, which confers sensitivity to cycloheximide). In addition, any known cytotoxic agents including cytotoxic proteins such as the diphtheria toxin (DTA) catalytic domain can also be used as counterselectable reporters. See U.S. Pat. No. 5,733,726. DTA causes the ADP-ribosylation of elongation factor-2 and thus inhibits protein synthesis and causes cell death. Other examples of cytotoxic agents include ricin, Shiga toxin, and exotoxin A of Pseudomonas aeruginosa.

For example, when the URA3 gene is used as a counterselectable reporter gene, yeast cells containing a mutant URA3 gene can be used as host cells (Ura⁻Foa^{R} phenotype) for the in vivo assay. Such cells lack URA3-encoded functional orotidine-5′-phosphate decarboxylase, an enzyme required for the biosynthesis of uracil. As a result, the cells are unable to grow on media lacking uracil. However, because of the absence of a wild-type orotidine-5′-phosphate decarboxylase, the yeast cells cannot convert non-toxic 5-fluoroorotic acid (5-FOA) to a toxic product, 5-fluorouracil. Thus, such yeast cells are resistant to 5-FOA and can grow on a medium containing 5-FOA. Therefore, for example, to screen for a compound capable of disrupting interactions between APOA1 (or a homologue, fragment or derivative thereof), or a mutant form of APOA1 (or a homologue, fragment or derivative thereof), and PRA1 (or a homologue, fragment or derivative thereof), or a mutant form of PRA1 (or a homologue, fragment or derivative thereof), APOA1 (or a homologue, fragment or derivative thereof) is expressed as a fusion protein with a DNA-binding domain of a suitable transcription activator while PRA1 (or a homologue, fragment or derivative thereof) is expressed as a fusion protein with a transcription activation domain of a suitable transcription activator. In the host strain, the reporter URA3 gene may be operably linked to a promoter specifically responsive to the association of the transcription activation domain and the DNA-binding domain. After the fusion proteins are expressed in the Ura⁻Foa^{R} yeast cells, an in vivo screening assay can be conducted in the presence of a test compound with the yeast cells being cultured on a medium containing uracil and 5-FOA. If the test compound does not disrupt the interaction between APOA1 and PRA1, active URA3 gene product, i.e., orotidine-5′-decarboxylase, which converts 5-FOA to toxic 5-fluorouracil, is expressed. As a result, the yeast cells cannot grow. On the other hand, when the test compound disrupts the interaction between APOA1 and PRA1, no active orotidine-5′-decarboxylase is produced in the host yeast cells. Consequently, the yeast cells will survive and grow on the 5-FOA-containing medium. Therefore, compounds capable of interfering with or dissociating the interaction between APOA1 and PRA1 can thus be identified based on colony formation.

As will be apparent, the screening assay of the present invention can be applied in a format appropriate for large-scale screening. For example, combinatorial technologies can be employed to construct combinatorial libraries of small organic molecules or small peptides. See generally, e.g., Kenan et al., Trends Biochem. Sc., 19:57-64 (1994); Gallop et al., J. Med. Chem., 37:1233-1251 (1994); Gordon et al., J. Med. Chem., 37:1385-1401 (1994); Ecker et al., Biotechnology, 13:351-360 (1995). Such combinatorial libraries of compounds can be applied to the screening assay of the present invention to isolate specific modulators of particular protein-protein interactions. In the case of random peptide libraries, the random peptides can be co-expressed with the fusion proteins of the present invention in host cells and assayed in vivo. See e.g., Yang et al., Nucl. Acids Res., 23:1152-1156 (1995). Alternatively, they can be added to the culture medium for uptake by the host cells.

Conveniently, yeast mating is used in an in vivo screening assay. For example, haploid cells of a-mating type expressing one fusion protein as described above are mated with haploid cells of α-mating type expressing the other fusion protein. Upon mating, the diploid cells are spread on a suitable medium to form a lawn. Drops of test compounds can be deposited onto different areas of the lawn. After culturing the lawn for an appropriate period of time, drops containing a compound capable of modulating the interaction between the particular test proteins in the fusion proteins can be identified by stimulation or inhibition of growth in the vicinity of the drops.

The screening assays of the present invention for identifying compounds capable of modulating protein-protein interactions can also be fine-tuned by various techniques to adjust the thresholds or sensitivity of the positive and negative selections. Mutations can be introduced into the reporter proteins to adjust their activities. The uptake of test compounds by the host cells can also be adjusted. For example, yeast high uptake mutants such as the erg6 mutant strains can facilitate yeast uptake of the test compounds. See Gaber et al., Mol. Cell. Biol., 9:3447-3456 (1989). Likewise, the uptake of the selection compounds such as 5-FOA, 2-deoxygalactose, cycloheximide, α-aminoadipate, and the like can also be fine-tuned.

Generally, a control assay is performed in which the above screening assay is conducted in the absence of the test compound. The result of this assay is then compared with that obtained in the presence of the test compound.

### 5.3.1.4. Screening Assays for Interaction Agonists

The screening assays of the present invention can also be used to identify compounds that trigger or initiate, enhance or stabilize the protein-protein interactions formed between members of the interacting protein pairs disclosed in the tables above, or between combinations of mutant and wild type forms of such proteins, or pairs of mutant proteins. For example, if a disease or disorder is associated with the decreased expression of any one of the individual proteins, or one of the protein pairs selected from the tables, then the disease or disorder may be treated by strengthening or stabilizing the interactions between the interacting partner proteins in patients. Alternatively, if a disease or disorder is associated with a mutant form, or forms, of the interacting proteins that exhibit weakened or abolished interactions with their binding partner(s), then the disease or disorder may be treated with a compound that initiates or stabilizes the interaction between the mutant form, or forms, of the interacting proteins.

Thus, a screening assay can be performed in the same manner as described above, except that a positively selectable marker is used. For example, a first protein, which is any protein selected from the proteins described in the tables (or a homologue, fragment, or derivative thereof), or a mutant form of the first protein (or a homologue, fragment, or derivative thereof), and a second protein, which is an interacting partner of the first protein (or a homologue, fragment, or derivative thereof), or a mutant form of the second protein (or a homologue, fragment, or derivative thereof), are used as test proteins expressed in the form of fusion proteins as described above for purposes of a two-hybrid assay. The fusion proteins are expressed in host cells and are allowed to interact with each other in the presence of one or more test compounds.

A gene encoding a positively selectable marker such as β-galatosidase may be used as a reporter gene such that when a test compound enables, enhances or strengthens the interaction between a first protein, (or a homologue, fragment, or derivative thereof), or a mutant form of the first protein (or a homologue, fragment, or derivative thereof), and a second protein (or a homologue, fragment, or derivative thereof), or a mutant form of the second (or a homologue, fragment, or derivative thereof), β-galatosidase is expressed. As a result, the compound may be identified based on the appearance of a blue color when the host cells are cultured in a medium containing X-Gal.

Generally, a control assay is performed in which the above screening assay is conducted in the absence of the test compound. The result of this assay is then compared with that obtained in the presence of the test compound.

### 5.4. Optimization of the Identified Compounds

Once test compounds are selected that are capable of modulating the interaction between the interacting protein pairs of proteins described in the tables, or modulating the activity or intracellular levels of their constituent proteins, a secondary assay can be performed to confirm the specificity and effect of the compounds selected in the primary screens. Exemplary secondary assays are cell-based assays or animal based assays.

In addition, once test compounds are selected that are capable of modulating the proteins in the tables or the interaction between the interacting protein pairs of proteins described in the tables, or modulating the activity or intracellular levels of their constituent proteins, a data set including data defining the identity or characteristics of the test compounds can be generated. The data set may include information relating to the properties of a selected test compound, e.g., chemical structure, chirality, molecular weight, melting point, etc. Alternatively, the data set may simply include assigned identification numbers understood by the researchers conducting the screening assay and/or researchers receiving the data set as representing specific test compounds. The data or information can be cast in a transmittable form that can be communicated or transmitted to other researchers, particularly researchers in a different country. Such a transmittable form can vary and can be tangible or intangible. For example, the data set defining one or more selected test compounds can be embodied in texts, tables, diagrams, molecular structures, photographs, charts, images or any other visual forms. The data or information can be recorded on a tangible media such as paper or embodied in computer-readable forms (e.g., electronic, electromagnetic, optical or other signals). The data in a computer-readable form can be stored in a computer usable storage medium (e.g., floppy disks, magnetic tapes, optical disks, and the like) or transmitted directly through a communication infrastructure. In particular, the data embodied in electronic signals can be transmitted in the form of email or posted on a website on the Internet or Intranet. In addition, the information or data on a selected test compound can also be recorded in an audio form and transmitted through any suitable media, e.g., analog or digital cable lines, fiber optic cables, etc., via telephone, facsimile, wireless mobile phone, Internet phone and the like.

Thus, the information and data on a test compound selected in a screening assay described above or by virtual screening as discussed below can be produced anywhere in the world and transmitted to a different location. For example, when a screening assay is conducted offshore, the information and data on a selected test compound can be generated and cast in a transmittable form as described above. The data and information in a transmittable form thus can be imported into the U.S. or transmitted to any other countries, where the data and information may be used in further testing the selected test compound and/or in modifying and optimizing the selected test compound to develop lead compounds for testing in clinical trials.

Compounds can also be selected based on structural models of the target protein or protein complex and/or test compounds. In addition, once an effective compound is identified, structural analogs or mimetics thereof can be produced based on rational drug design with the aim of improving drug efficacy and stability, and reducing side effects. Methods known in the art for rational drug design can be used in the present invention. See, e.g., Hodgson et al., Bio/Technology, 9:19-21 (1991); U.S. Pat. Nos. 5,800,998 and 5,891,628, all of which are incorporated herein by reference. An example of rational drug design is the development of HIV protease inhibitors. See Erickson et al., Science, 249:527-533 (1990).

In this respect, structural information on the target protein or protein complex is obtained. Preferably, atomic coordinates defining a three-dimensional structure of the target protein or protein complex can be obtained. For example, each of the interacting pairs can be expressed and purified. The purified interacting protein pairs are then allowed to interact with each other in vitro under appropriate conditions. Optionally, the interacting protein complex can be stabilized by crosslinking or other techniques. The interacting complex can be studied using various biophysical techniques including, e.g., X-ray crystallography, NMR, computer modeling, mass spectrometry, and the like. Likewise, structural information can also be obtained from protein complexes formed by interacting proteins and a compound that initiates or stabilizes the interaction of the proteins. Methods for obtaining such atomic coordinates by X-ray crystallography, NMR, and the like are known in the art and the application thereof to the target protein or protein complex of the present invention should be apparent to skilled persons in the art of structural biology. See Smyth and Martin, Mol. Pathol., 53:8-14 (2000); Oakley and Wilce, Clin. Exp. Pharmacol. Physiol., 27(3):145-151 (2000); Ferentz and Wagner, Q. Rev. Biophys., 33:29-65 (2000); Hicks, Curr. Med. Chem., 8(6):627-650 (2001); and Roberts, Curr. Opin. Biotechnol., 10:42-47 (1999).

In addition, understanding of the interaction between the proteins of interest in the presence or absence of a modulator can also be derived by mutagenic analysis using a yeast two-hybrid system or other methods for detecting protein-protein interactions. In this respect, various mutations can be introduced into the interacting proteins and the effect of the mutations on protein-protein interaction examined by a suitable method such as the yeast two-hybrid system.

Various mutations including amino acid substitutions, deletions and insertions can be introduced into a protein sequence using conventional recombinant DNA technologies. Generally, it is particularly desirable to decipher the protein binding sites. Thus, it is important that the mutations introduced only affect protein-protein interactions and cause minimal structural disturbances. Mutations are preferably designed based on knowledge of the three-dimensional structure of the interacting proteins. Preferably, mutations are introduced to alter charged amino acids or hydrophobic amino acids exposed on the surface of the proteins, since ionic interactions and hydrophobic interactions are often involved in protein-protein interactions. Alternatively, the “alanine scanning mutagenesis” technique is used. See Wells, et al., Methods Enzymol., 202:301-306 (1991); Bass et al., Proc. Natl. Acad. Sci. USA, 88:4498-4502 (1991); Bennet et al., J. Biol. Chem., 266:5191-5201 (1991); Diamond et al., J. Virol., 68:863-876 (1994). Using this technique, charged or hydrophobic amino acid residues of the interacting proteins are replaced by alanine, and the effect on the interaction between the proteins is analyzed using e.g., the yeast two-hybrid system. For example, the entire protein sequence can be scanned in a window of five amino acids. When two or more charged or hydrophobic amino acids appear in a window, the charged or hydrophobic amino acids are changed to alanine using standard recombinant DNA techniques. The thus-mutated proteins are used as “test proteins” in the above-described two-hybrid assays to examine the effect of the mutations on protein-protein interaction. Preferably, the mutational analyses are conducted both in the presence and in the absence of an identified modulator compound. In this manner, the domains or residues of the proteins important to protein-protein interaction and/or the interaction between the modulator compound and the interacting proteins can be identified.

Based on the information obtained, structural relationships between the interacting proteins, as well as between the identified modulators and the interacting proteins are elucidated. For example, for the identified modulators (i.e., lead compounds), the three-dimensional structure and chemical moieties critical to their modulating effect on the interacting proteins are revealed. Using this information and various techniques known in the art of molecular modeling (i.e., simulated annealing), medicinal chemists can then design analog compounds that might be more effective modulators of the protein-protein interactions of the present invention. For example, the analog compounds might show more specific or tighter binding to their targets, and thereby might exhibit fewer side effects, or might have more desirable pharmacological characteristics (e.g., greater solubility).

In addition, if the lead compound is a peptide, it can also be analyzed by the alanine scanning technique and/or the two-hybrid assay to determine the domains or residues of the peptide important to its modulating effect on particular protein-protein interactions. The peptide compound can be used as a lead molecule for rational design of small organic molecules or peptide mimetics. See Huber et al., Curr. Med. Chem., 1:13-34 (1994).

The domains, residues or moieties critical to the modulating effect of the identified compound constitute the active region of the compound known as its “pharmacophore.” Once the pharmacophore has been elucidated, a structural model can be established by a modeling process that may incorporate data from NMR analysis, X-ray diffraction data, alanine scanning, spectroscopic techniques and the like. Various techniques including computational analysis (e.g., molecular modeling and simulated annealing), similarity mapping and the like can all be used in this modeling process. See e.g., Perry et al., in OSAR: Quantitative Structure-Activity Relationships in Drug Design, pp. 189-193, Alan R. Liss, Inc., 1989; Rotivinen et al., Acta Pharmaceutical Fennica, 97:159-166 (1988); Lewis et al., Proc. R. Soc. Lond., 236:125-140 (1989); McKinaly et al., Annu. Rev. Pharmacol. Toxiciol., 29:111-122 (1989). Commercial molecular modeling systems available from Polygen Corporation, Waltham, Mass., include the CHARMm program, which performs energy minimization and molecular dynamics functions, and QUANTA program, which performs construction, graphic modeling and analysis of molecular structure. Such programs allow interactive construction, modification, and visualization of molecules. Other computer modeling programs are also available from BioDesign, Inc. (Pasadena, Calif.), Hypercube, Inc. (Cambridge, Ontario), and Allelix, Inc. (Mississauga, Ontario, Canada).

A template can be formed based on the established model. Various compounds can then be designed by linking various chemical groups or moieties to the template. Various moieties of the template can also be replaced. In addition, in the case of a peptide lead compound, the peptide or mimetics thereof can be cyclized, e.g., by linking the N-terminus and C-terminus together, to increase its stability. These rationally designed compounds are further tested. In this manner, pharmacologically acceptable and stable compounds with improved efficacy and reduced side effects can be developed. The compounds identified in accordance with the present invention can be incorporated into a pharmaceutical formulation suitable for administration to an individual.

In addition, the structural models or atomic coordinates defining a three-dimensional structure of the target protein or protein complex can also be used in virtual screen to select compounds capable of modulating the target protein or protein complex. Various methods of computer-based virtual screen using atomic coordinates are generally known in the art. For example, U.S. Pat. No. 5,798,247 (which is incorporated herein by reference) discloses a method of identifying a compound (specifically, an interleukin converting enzyme inhibitor) by determining binding interactions between an organic compound and binding sites of a binding cavity within the target protein. The binding sites are defined by atomic coordinates.

The compounds designed or selected based on rational drug design or virtual screen can be tested for their ability to modulate (interfere with or strengthen) the interaction between the interacting partners within the protein complexes of the present invention. In addition, the compounds can also be further tested for their ability to modulate (inhibit or enhance) cellular functions such as angiogenesis, cell proliferation and transformation, intracellular vesicle trafficking, vacuolar protein sorting, formation of multivesicular bodies and endocytosis, inhibiting viral budding, suppress tumorigenesis and cell transformation, and reduce autoimmune response. In addition, the methods may also be used in the treatment or prevention of diseases and disorders such as viral infection, cancer and autoimmune diseases, cancer, AIDS, asthma, ischemia, stroke, autoimmune diseases, neurodegenerative diseases, inflammatory disorders, sepsis, and osteoporosis. The methods may also be used in the treatment or prevention of diseases and disorders such as cholesterol transport and lipid metabolism such as dementia such as Alzheimer's disease and cardiovascular diseases such as coronary artery disease, atherosclerosis, hypercholesterolemia, Tangier disease and amyloidosis; formation and maintenance of high density lipoprotein (HDL) microparticles, cholesterol and lipid transport and metabolism in cells, hyperlipidemia, hypercholesterolemia, and cardiovascular disease.

The compounds can be tested for their ability to modulate in cells as well as their effectiveness in treating diseases such as abnormal cell proliferation (hyperproliferation or dysproliferation), keloid, liver cirrhosis, psoriasis, altered wound healing, cancer, especially cancers of the breast, ovary, colon, prostate, and lung, melanomas and cancers of hematopoietic origin, type 2 diabetes, obesity, hypertension, hypercholesterolemia, atherosclerosis, coronary artery disease, peripheral vascular disease, optic neuropathy, inflammatory response, amyloidosis, dementia, Alzheimer's disease, Tangier disease, Angelman syndrome, and specific viral infections.

Following the selection of desirable compounds according to the methods disclosed above, the methods of the present invention further provide for the manufacture of the selected compounds. Compounds found to desirably modulate the interaction between the interacting protein pairs of proteins of the present invention, or to desirably modulate the activity or intracellular levels of their constituent proteins, can be manufactured for further experimental studies, or for therapeutic use.

### 6. Therapeutic Applications

As described above, the interactions between the interacting pairs of proteins of the present invention suggest that these proteins and/or the protein complexes formed by them may be involved in common biological processes and disease pathways. The protein complexes may mediate the functions of the individual proteins of each interacting protein pair, or of the interacting pairs themselves, in the biological processes or disease pathways. Thus, one may modulate such biological processes or treat diseases by modulating the functions and activities of any of the individual proteins described in the tables, and/or a protein complex comprising some combination of these proteins. As used herein, modulating a protein selected from the tables, or a protein complex comprising some combination of these proteins means altering (enhancing or reducing) the intracellular concentrations or activities of the proteins or protein complexes, e.g., increasing the concentrations of a particular protein described in the tables, or a protein complex comprising some combination of these proteins, enhancing or reducing their biological activities, increasing or decreasing their stability, altering their affinity or specificity to certain other biological molecules, etc. For example, a pair of interacting proteins listed in the tables may be involved in angiogenesis, cell proliferation and transformation, intracellular vesicle trafficking, vacuolar protein sorting, formation of multivesicular bodies and endocytosis, inhibiting viral budding, suppress tumorigenesis and cell transformation, and reduce autoimmune response. In addition, the methods may also be used in the treatment or prevention of diseases and disorders such as viral infection, cancer and autoimmune diseases, cancer, AIDS, asthma, ischemia, stroke, autoimmune diseases, neurodegenerative diseases, inflammatory disorders, sepsis, and osteoporosis. The methods may also be used in the treatment or prevention of diseases and disorders such as cholesterol transport and lipid metabolism such as dementia such as Alzheimer's disease and cardiovascular diseases such as coronary artery disease, atherosclerosis, hypercholesterolemia, Tangier disease and amyloidosis, formation and maintenance of high density lipoprotein (HDL) microparticles, cholesterol and lipid transport and metabolism in cells, hyperlipidemia, hypercholesterolemia, and cardiovascular disease

Thus, assays such as those described in Section 4 may be used in determining the effect of an aberration in a particular protein complex or an interacting member thereof on angiogenesis, cell proliferation and transformation, intracellular vesicle trafficking, vacuolar protein sorting, formation of multivesicular bodies and endocytosis, inhibiting viral budding, suppress tumorigenesis and cell transformation, and reduce autoimmune response. In addition, the methods may also be used in the treatment or prevention of diseases and disorders such as viral infection, cancer and autoimmune diseases, cancer, AIDS, asthma, ischemia, stroke, autoimmune diseases, neurodegenerative diseases, inflammatory disorders, sepsis, and osteoporosis. The methods may also be used in the treatment or prevention of diseases and disorders such as cholesterol transport and lipid metabolism such as dementia such as Alzheimer's disease and cardiovascular diseases such as coronary artery disease, atherosclerosis, hypercholesterolemia, Tangier disease and amyloidosis, formation and maintenance of high density lipoprotein (HDL) microparticles, cholesterol and lipid transport and metabolism in cells, hyperlipidemia, hypercholesterolemia, and cardiovascular disease.

In addition, it is also possible to determine, using the same assay methods, the presence or absence of an association between a protein complex of the present invention or an interacting member thereof and a physiological disorder or disease such as abnormal cell proliferation (hyperproliferation or dysproliferation), keloid, liver cirrhosis, psoriasis, altered wound healing, cancer, especially cancers of the breast, ovary, colon, prostate, and lung, melanomas and cancers of hematopoietic origin, type 2 diabetes, obesity, hypertension, hypercholesterolemia, atherosclerosis, coronary artery disease, peripheral vascular disease, optic neuropathy, inflammatory response, amyloidosis, dementia, Alzheimer's disease, Tangier disease, Angelman syndrome, and specific viral infections; or predisposition to a physiological disorder or disease.

Once such associations are established, the diagnostic methods as described in Section 4 can be used in diagnosing the disease or disorder, or a patient's predisposition to it. In addition, various in vitro and in vivo assays may be employed to test the therapeutic or prophylactic efficacies of the various therapeutic approaches described in Sections 6.2 and 6.3 that are aimed at modulating the functions and activities of a particular protein complex of the present invention, or an interacting member thereof. Similar assays can also be used to test whether the therapeutic approaches described in Sections 6.2 and 6.3 result in the modulation of angiogenesis, cell proliferation and transformation, intracellular vesicle trafficking, vacuolar protein sorting, formation of multivesicular bodies and endocytosis, inhibiting viral budding, suppress tumorigenesis and cell transformation, and reduce autoimmune response. In addition, the methods may also be used in the treatment or prevention of diseases and disorders such as viral infection, cancer and autoimmune diseases, cancer, AIDS, asthma, ischemia, stroke, autoimmune diseases, neurodegenerative diseases, inflammatory disorders, sepsis, and osteoporosis. The methods may also be used in the treatment or prevention of diseases and disorders such as cholesterol transport and lipid metabolism such as dementia such as Alzheimer's disease and cardiovascular diseases such as coronary artery disease, atherosclerosis, hypercholesterolemia, Tangier disease and amyloidosis, formation and maintenance of high density lipoprotein (HDL) microparticles, cholesterol and lipid transport and metabolism in cells, hyperlipidemia, hypercholesterolemia, and cardiovascular disease.

The cell model or transgenic animal model described in Section 7 may be employed in the in vitro and in vivo assays.

In accordance with this aspect of the present invention, methods are provided for modulating (promoting or inhibiting) a protein complex of the present invention formed by the interactions described in the tables. The human cells can be in in vitro cell or tissue cultures. The methods are also applicable to human cells in a patient.

In one embodiment, the concentration of a protein complex formed by the interactions described in the tables is reduced in the cells. Various methods can be employed to reduce the concentration of the protein complex. For example, the protein complex concentration can be reduced by interfering with the interactions between the interacting protein partners. Hence, compounds capable of interfering with interactions between interacting pairs of proteins identified in the tables can be administered to the cells in vitro or in vivo in a patient. Such compounds can be compounds capable of binding specific proteins listed in the tables. They can also be antibodies immunoreactive with specific proteins identified in the tables. Also, the compounds can be small peptides derived from a first interacting protein of the present invention, or a mimetic thereof, that are capable of binding a second protein of the present invention, the second protein being a binding partner of the first protein as shown in the tables above.

In another embodiment, the method of modulating the protein complex includes inhibiting the expression of any of the individual proteins described in the tables. The inhibition can be at the transcriptional, translational, or post-translational level. For example, antisense compounds and ribozyme compounds can be administered to human cells in cultures or in human bodies. In addition, RNA interference technologies may also be employed to administer to cells double-stranded RNA or RNA hairpins capable of “knocking down” the expression of any of the interacting proteins of the present invention.

In the various embodiments described above, preferably the concentrations or activities of both partners in an interacting pair of proteins of the present invention are reduced or inhibited, or the concentration or activity of a single constituent protein of a protein complex formed by the interactions described in the tables is reduced or inhibited.

In yet another embodiment, an antibody selectively immunoreactive with a pair of interacting proteins identified in the tables is administered to cells in vitro or in human bodies to inhibit the protein complex activities and/or reduce the concentration of the protein complex in the cells or patient.

Further provided by the present invention is a method of treatment of a disease or disorder comprising identifying a patient that has a particular disease or disorder, shows symptoms of having a particular disease or disorder, is predisposed to, or at risk of developing a particular disease or disorder, and treating the disease or disorder by modulating a protein or protein-protein interaction according to the present invention.

### 6.1. Applicable Diseases

Pathological angiogenesis including abnormal capillary growth, abnormal vascular formation and remodelling is a hallmark of various diseases such as cancer, ischaemic and inflammatory diseases. See generally, Carmeliet and Jain, Nature, 407:249-257 (2000). Particularly, tumors require new blood vessels for metastasis. Thus, in tumors, the balance between pro-angiogenic molecules and anti-angiogenic molecules is derailed, and significant tumor vessels develop during tumor formation and metastasis. Abnormal excessive angiogenesis is also involved in many other diseases. For example, hypoxia in diabetes, Alzheimer's disease, asthma, atherosclerosis, infectious diseases (e.g., hepatitis and pneumonia), and hypertension typically stimulates excessive angiogenesis. Prolonged and excessive angiogenesis is also associated with inflammatory disorders. In addition, it has been shown that the obesity mediator leptin and insulin-induced VEGF and bFGF are mediators of angiogenesis in adipose tissue. See Sierra-Honigmann et al., Science, 281:1683-1686 (1998). Thus, angiogenesis may also contribute to obesity. Additionally, insufficient angiogenesis is also involved in various diseases and disorders including tissue ischemia (e.g. coronary artery disease), peripheral vascular disease, various neurodegenerative disorders, and impaired wound healing. In addition, appropriate angiogenesis is also critical to successful transplantation of organs, tissues and cells. See, Carmeliet and Jain, Nature, 407:249-257 (2000).

Particular examples of neoplasms and other diseases known to involve abnormal angiogenesis include, but are not limited to, athersclerosis, haemangioma, haemangioendothelioma, vascular malformations in blood vessels, skin warts, pyogenic granulomas, abnormal hair growth, Kaposi's sarcoma, scar keloids, allergic oedema, psoriasis, decubitus or stasis ulcers, gastrointestinal ulcers, dysfunctional uterine bleeding, follicular cysts, endometriosis, ascites, peritoneal sclerosis, adhesion formation, ischaemic heart and limb disease, obesity, rheumatoid arthritis, synovitis, bone and cartilage destruction, osteomyelitis, pannus growth, osteophyte formation, aseptic necrosis, impaired healing of fractures, hepatitis, pneumonia, glomerulonephritis, asthma, nasal polyps, liver regeneration, pulmonary hypertension, diabetes, retinopathy of prematurity, diabetic retinopathy, choroidal and other intraocular disorders, leukomalacia, stroke, vascular dementia, Alzheimer's disease, CADASIL, thyroiditis, thyroid enlargement, thyroid pseudocyst, lymphoproliferative disorders, lymphoedema, AIDS (Kaposi), etc.

The methods for modulating the functions and activities of protein complex of the present invention, or an interacting member thereof, may be employed to modulate angiogenesis, cell proliferation and transformation. In addition, the methods may also be used in the treatment or prevention of diseases and disorders such as cancer, diabetes, Alzheimer's disease, asthma, atherosclerosis, infectious diseases (e.g., hepatitis and pneumonia), hypertension, inflammatory disorders, obesity, tissue ischemia (e.g. coronary artery disease), peripheral vascular disease, various neurodegenerative disorders, impaired wound healing, lymphedema and Angelman syndrome.

Therefore, the methods can be applicable to a variety of tumors, i.e., abnormal growth, whether cancerous (malignant) or noncancerous (benign), and whether primary tumors or secondary tumors. Such disorders include but are not limited to lung cancers such as bronchogenic carcinoma (e.g., squamous cell carcinoma, small cell carcinoma, large cell carcinoma, and adenocarcinoma), alveolar cell carcinoma, bronchial adenoma, chondromatous hamartoma (noncancerous), and sarcoma (cancerous); heart tumors such as myxoma, fibromas and rhabdomyomas; bone tumors such as osteochondromas, condromas, chondroblastomas, chondromyxoid fibromas, osteoid osteomas, giant cell tumors, chondrosarcoma, multiple myeloma, osteosarcoma, fibrosarcomas, malignant fibrous histiocytomas, Ewing's tumor (Ewing's sarcoma), and reticulum cell sarcoma; brain tumors such as gliomas (e.g., glioblastoma multiforme), anaplastic astrocytomas, astrocytomas, and oligodendrogliomas, medulloblastomas, chordoma, Schwannomas, ependymomas, meningiomas, pituitary adenoma, pinealoma, osteomas, and hemangioblastomas, craniopharyngiomas, chordomas, germinomas, teratomas, dermoid cysts, and angiomas; various oral cancers; tumors in digestive system such as leiomyoma, epidermoid carcinoma, adenocarcinoma, leiomyosarcoma, stomach adenocarcinomas, intestinal lipomas, intestinal neurofibromas, intestinal fibromas, polyps in large intestine, familial polyposis such as Gardner's syndrome and Peutz-Jeghers syndrome, colorectal cancers (including colon cancer and rectal cancer); liver cancers such as hepatocellular adenomas, hemangioma, hepatocellular carcinoma, fibrolamellar carcinoma, cholangiocarcinoma, hepatoblastoma, and angiosarcoma; kidney tumors such as kidney adenocarcinoma, renal cell carcinoma, hypernephroma, and transitional cell carcinoma of the renal pelvis; bladder cancers; tumors in blood system including acute lymphocytic (lymphoblastic) leukemia, acute myeloid (myelocytic, myelogenous, myeloblastic, myelomonocytic) leukemia, chronic lymphocytic leukemia (e.g., Sezary syndrome and hairy cell leukemia), chronic myelocytic (myeloid, myelogenous, granulocytic) leukemia, Hodgkin's lymphoma, non-Hodgkin's lymphoma, mycosis fungoides, and myeloproliferative disorders (including myeloproliferative disorders are polycythemia vera, myelofibrosis, thrombocythemia, and chronic myelocytic leukemia); skin cancers such as basal cell carcinoma, squamous cell carcinoma, melanoma, Kaposi's sarcoma, and Paget's disease; head and neck cancers; eye-related cancers such as retinoblastoma and intraocular melanocarcinoma; male reproductive system cancers such as benign prostatic hyperplasia, prostate cancer, and testicular cancers (e.g., seminoma, teratoma, embryonal carcinoma, and choriocarcinoma); breast cancer; female reproductive system cancers such as uterus cancer (endometrial carcinoma), cervical cancer (cervical carcinoma), cancer of the ovaries (ovarian carcinoma), vulvar carcinoma, vaginal carcinoma, fallopian tube cancer, and hydatidiform mole; thyroid cancer (including papillary, follicular, anaplastic, or medullary cancer); pheochromocytomas (adrenal gland); noncancerous growths of the parathyroid glands; cancerous or noncancerous growths of the pancreas; etc.

In addition, the methods are potentially also applicable to premalignant conditions to prevent or stop the progression of such conditions towards malignancy, or cause regression of the premalignant conditions. Examples of premalignant conditions include hyperplasia, dysplasia, and metaplasia.

Thus, the term “treating cancer” as used herein, specifically refers to administering therapeutic agents to a patient diagnosed of cancer, i.e., having established cancer in the patient, to inhibit the further growth or spread of the malignant cells in the cancerous tissue, and/or to cause the death of the malignant cells. The term “treating cancer” also encompasses treating a patient having premalignant conditions to stop the progression of, or cause regression of, the premalignant conditions.

The methods of the present invention may also be useful in treating or preventing other diseases and disorders caused by abnormal cell proliferation (hyperproliferation or dysproliferation), e.g., keloid, liver cirrhosis, psoriasis, etc. In addition, the methods may also find applications in promoting wound healing, and other cell and tissue growth-related conditions.

Additionally, the methods of the present invention may also be applied to treatment of benign proliferative conditions including, but not limited to, diabetic proliferative retinopathy, idiopathic fibrotic diseases such as fibrosing alveolitis, and vascular smooth muscle proliferation following balloon antioplasty that can lead to re-stenosis.

In addition, the methods for modulating the functions of protein complexes disclosed herein may be used in treating or preventing diabetes, Alzheimer's disease, asthma, atherosclerosis, infectious diseases (e.g., hepatitis and pneumonia), hypertension, inflammatory disorders, obesity, tissue ischemia (e.g. coronary artery disease), peripheral vascular disease, various neurodegenerative disorders, and impaired wound healing.

The methods for modulating functions and activities of complexes of the present invention, or an interacting member thereof, may be employed to modulate intracellular vesicle trafficking, vacuolar protein sorting, formation of multivesicular bodies and endocytosis, inhibiting viral budding, suppress tumorigenesis and cell transformation, and reduce autoimmune response. In addition, the methods may also be used in the treatment or prevention of diseases and disorders such as viral infection, cancer and autoimmune diseases.

In one aspect, the methods of the present invention may be useful in treating or preventing diseases or disorders associated with viral infection in animals, particularly humans. Such viral infection can be caused by viruses including, but not limited to, lentiviruses such as HIV, SIV, VMV, BIV, FIV, CAEV and EIAV, hepatitis A, hepatitis B, hepatitis C, hepatitis D virus, hepatitis E virus, hepatitis G virus, human foamy virus, human herpes viruses (e.g., human herpes virus 1, human herpes virus 2, human herpes virus 4/Epstein Barr virus, human herpes virus 5, human herpes virus 7), human papilloma virus, human parechovirus 2, human T-cell lymphotropic virus, mumps virus, Measles virus, Rubella virus, Semliki Forest virus, West Nile virus, Colorado tick fever virus, foot-and-mouth disease virus, Marburg virus, polyomavirus, TT virus, Lassa virus, lymphocytic choriomeningitis virus, vesicular stomatitis virus, influenza viruses, human parainfluenza viruses, respiratory syncytial virus, rotavirus, herpes simplex virus, herpes zoster virus, varicella virus, parvovirus, vaccinia virus, Ebola virus, cytomegalovirus, variola virus, encephalitis viruses, adenovirus, echovirus, rhinoviruses, filoviruses, coxachievirus, coronavirus, HTLV-I, HTLV-II, Dengue viruses, yellow fever virus, regional hemorrhagic fever viruses, molluscum virus, poliovirus, rabiesvirus, etc. In preferred embodiments, the methods can be used in treating or preventing infection by viruses that utiliz cellular machineries of membrane/vescicle trafficking and cellular MVB sorthing pathway. In more preferred embodiments, the methods are used in treating or preventing enveloped viruses. In specific embodiments, various human retroviruses are treated by the methods of the present invention.

As used herein, the term “HIV infection” generally encompasses infection of a host animal, particularly a human host, by the human immunodeficiency virus (HIV) family of retroviruses including, but not limited to, HIV I, HIV II, HIV III (a.k.a. HTLV-III, LAV-1, LAV-2), and the like. “HIV” can be used herein to refer to any strains, forms, subtypes, clades and variations in the HIV family. Thus, treating HIV infection will encompass the treatment of a person who is a carrier of any of the HIV family of retroviruses or a person who is diagnosed of active AIDS, as well as the treatment or prophylaxis of the AIDS-related conditions in such persons. A carrier of HIV may be identified by any methods known in the art. For example, a person can be identified as HIV carrier on the basis that the person is anti-HIV antibody positive, or is HIV-positive, or has symptoms of AIDS. That is, “treating HIV infection” should be understood as treating a patient who is at any one of the several stages of HIV infection progression, which, for example, include acute primary infection syndrome (which can be asymptomatic or associated with an influenza-like illness with fevers, malaise, diarrhea and neurologic symptoms such as headache), asymptomatic infection (which is the long latent period with a gradual decline in the number of circulating CD⁴⁺ T cells), and AIDS (which is defined by more serious AIDS-defining illnesses and/or a decline in the circulating CD4 cell count to below a level that is compatible with effective immune function). In addition, “treating or preventing HIV infection” will also encompass treating suspected infection by HIV after suspected past exposure to HIV by e.g., contact with HIV-contaminated blood, blood transfusion, exchange of body fluids, “unsafe” sex with an infected person, accidental needle stick, receiving a tattoo or acupuncture with contaminated instruments, or transmission of the virus from a mother to a baby during pregnancy, delivery or shortly thereafter. The term “treating HIV infection” may also encompass treating a person who is free of HIV infection but is believed to be at risk of infection by HIV.

The term “treating AIDS” means treating a patient who exhibits more serious AIDS-defining illnesses and/or a decline in the circulating CD4 cell count to below a level that is compatible with effective immune function. The term “treating AIDS” also encompasses treating AIDS-related conditions, which means disorders and diseases incidental to or associated with AIDS or HIV infection such as AIDS-related complex (ARC), progressive generalized lymphadenopathy (PGL), anti-HIV antibody positive conditions, and HIV-positive conditions, AIDS-related neurological conditions (such as dementia or tropical paraparesis), Kaposi's sarcoma, thrombocytopenia purpurea and associated opportunistic infections such as Pneumocystis carinii pneumonia, Mycobacterial tuberculosis, esophageal candidiasis, toxoplasmosis of the brain, CMV retinitis, HIV-related encephalopathy, HIV-related wasting syndrome, etc.

Thus, the term “preventing AIDS” as used herein means preventing in a patient who has HIV infection or is suspected to have HIV infection or is at risk of HIV infection from developing AIDS (which is characterized by more serious AIDS-defining illnesses and/or a decline in the circulating CD4 cell count to below a level that is compatible with effective immune function) and/or AIDS-related conditions.

In another specific embodiment, the present invention provides methods for treating or preventing HBV infection and hepatitis B by interfering with the interaction between TSG101 and an interacting partner thereof according to the present invention, or by inhibiting a protein complex of the present invention or an interacting member thereof. As used herein, the term “HBV infection” generally encompasses infection of a human by any strain or serotype of hepatitis B virus, including acute hepatitis B infection and chronic hepatitis B infection. Thus, treating HBV infection means the treatment of a person who is a carrier of any strain or serotype of hepatitis B virus or a person who is diagnosed of active hepatitis B to reduce the HBV viral load in the person or to alleviate one or more symptoms associated with HBV infection and/or hepatitis B, including, e.g., nausea and vomiting, loss of appetite, fatigue, muscle and joint aches, elevated transaminase blood levels, increased prothrombin time, jaundice (yellow discoloration of the eyes and body) and dark urine. A carrier of HBV may be identified by any methods known in the art. For example, a person can be identified as HBV carrier on the basis that the person is anti-HBV antibody positive (e.g., based on hepatitis B core antibody or hepatitis B surface antibody), or is HBV-positive (e.g., based on hepatitis B surface antigens (HBeAg or HbsAg) or HBV RNA or DNA) or has symptoms of hepatitis B infection or hepatitis B. That is, “treating HBV infection” should be understood as treating a patient who is at any one of the several stages of HBV infection progression. In addition, the term “treating HBV infection” will also encompass treating suspected infection by HBV after suspected past exposure to HBV by, e.g., contact with HBV-contaminated blood, blood transfusion, exchange of body fluids, “unsafe” sex with an infected person, accidental needle stick, receiving a tattoo or acupuncture with contaminated instruments, or transmission of the virus from a mother to a baby during pregnancy, delivery or shortly thereafter. The term “treating HBV infection” will also encompass treating a person who is free of HBV infection but is believed to be at risk of infection by HBV.

The term “preventing hepatitis B” as used herein means preventing in a patient who has HBV infection or is suspected to have HBV infection or is at risk of HBV infection from developing hepatitis B (which are characterized by more serious hepatitis-defining symptoms), cirrhosis, or hepatocellular carcinoma.

In another specific embodiment, the present invention provides methods for treating or preventing HCV infection and hepatitis C by interfering with the interaction between TSG101 and an interacting partner thereof according to the present invention, or by inhibiting a protein complex of the present invention or an interacting member thereof.

As used herein, the term “HCV infection” generally encompasses infection of a human by any types or subtypes of hepatitis C virus, including acute hepatitis C infection and chronic hepatitis C infection. Thus, treating HCV infection means the treatment of a person who is a carrier of any types or subtypes of hepatitis C virus or a person who is diagnosed of active hepatitis C to reduce the HCV viral load in the person or to alleviate one or more symptoms associated with HCV infection and/or hepatitis C. A carrier of HCV may be identified by any methods known in the art. For example, a person can be identified as HCV carrier on the basis that the person is anti-HCV antibody positive, or is HCV-positive (e.g., based on HCV RNA or DNA) or has symptoms of hepatitis C infection or hepatitis C (e.g., elevated serum transaminases). That is, “treating HCV infection” should be understood as treating a patient who is at any one of the several stages of HCV infection progression. In addition, the term “treating HCV infection” will also encompass treating suspected infection by HCV after suspected past exposure to HCV by, e.g., contact with HCV-contaminated blood, blood transfusion, exchange of body fluids, “unsafe” sex with an infected person, accidental needle stick, receiving a tattoo or acupuncture with contaminated instruments, or transmission of the virus from a mother to a baby during pregnancy, delivery or shortly thereafter. The term “treating HCV infection” will also encompass treating a person who is free of HCV infection but is believed to be at risk of infection by HCV. The term of “preventing HCV” as used herein means preventing in a patient who has HCV infection or is suspected to have HCV infection or is at risk of HCV infection from developing hepatitis C (which is characterized by more serious hepatitis-defining symptoms), cirrhosis, or hepatocellular carcinoma.

Specifically, breast cancers, colon cancers, prostate cancers, lung cancers and skin cancers may be amenable to the treatment by the methods of the present invention. In addition, premalignant conditions may also be treated by the methods of the present invention to prevent or stop the progression of such conditions towards malignancy, or cause regression of the premalignant conditions. Examples of premalignant conditions include hyperplasia, dysplasia, and metaplasia.

The methods of the present invention may also be useful in treating or preventing other diseases and disorders caused by abnormal cell proliferation (hyperproliferation or dysproliferation), e.g., keloid, liver cirrhosis, psoriasis, etc. In addition, the methods may also find applications in promoting wound healing, and other cell and tissue growth-related conditions.

In accordance with yet another aspect of the present invention, the methods for modulating the functions and activities of protein complexes or the interacting protein members thereof may be used in treating or preventing autoimmune diseases and disorders including, but not limited to, rheumatoid arthritis, systemic lupus erythematosus (SLE), Sjogren's syndrome, Canale-Smith syndrome, psoriasis, scleroderma, dermatomyositis, polymyositis, Behcet's syndrome, skin-related autoimmue diseases such as bullus pemphigoid, IgA dermatosis, pemphigus vulgaris, pemphigus foliaceus, dermatitis herpetiformis, contact dermatitis, autoimmune allopecia, erythema nodosa, and epidermolysis bullous aquisita, drug-induced hemotologic autoimmune disorders, autoimmue thrombocytopenic purpura, autoimmune neutropenia, systemic sclerosis, multiple sclerosis, imflammatory demyelinating, diabetes mellitus, autoimmune polyglandular syndromes, vasculitides, Wegener's granulomatosis, Hashimoto's disease, multinodular goitre, Grave's disease, autoimmune encephalomyelitis (EAE), demyelinating diseases, etc.

In another embodiments, the therapeutic and prophylactic methods of the present invention are employed to treat or prevent breast cancer, colon cancer, prostate cancer, lung cancer and skin cancer.

The therapeutic and prophylactic methods of the present invention can also be applied to premalignant conditions to prevent or stop the progression of such conditions towards malignancy, or cause regression of the premalignant conditions. Examples of premalignant conditions include hyperplasia, dysplasia, and metaplasia.

The methods for modulating the functions and activities of a protein complex of the present invention, or an interacting member thereof, may be employed to modulate angiogenesis, cell proliferation and transformation, intracellular vesicle trafficking, vacuolar protein sorting, formation of multivesicular bodies and endocytosis, inhibiting viral budding, suppress tumorigenesis and cell transformation, and reduce autoimmune response. In addition, the methods may also be used in the treatment or prevention of diseases and disorders such as viral infection, cancer and autoimmune diseases, cancer, AIDS, asthma, ischemia, stroke, autoimmune diseases, neurodegenerative diseases, inflammatory disorders, sepsis, and osteoporosis. The methods may also be used in the treatment or prevention of diseases and disorders such as cholesterol transport and lipid metabolism such as dementia such as Alzheimer's disease and cardiovascular diseases such as coronary artery disease, atherosclerosis, hypercholesterolemia, Tangier disease and amyloidosis, formation and maintenance of high density lipoprotein (HDL) microparticles, cholesterol and lipid transport and metabolism in cells, hyperlipidemia, hypercholesterolemia, and cardiovascular disease.

In addition, the methods may also be used in the treatment or prevention of diseases and disorders such as abnormal cell proliferation (hyperproliferation or dysproliferation), keloid, liver cirrhosis, psoriasis, altered wound healing, cancer, especially cancers of the breast, ovary, colon, prostate, and lung, melanomas and cancers of hematopoietic origin, type 2 diabetes, obesity, hypertension, hypercholesterolemia, atherosclerosis, coronary artery disease, peripheral vascular disease, optic neuropathy, inflammatory response, amyloidosis, dementia, Alzheimer's disease, Tangier disease, Angelman syndrome, and specific viral infections.

In accordance with yet another aspect of the present invention, the methods for modulating the functions and activities of the complexes or the interacting protein members thereof may be used in treating or preventing autoimmune diseases and disorders including, but not limited to, rheumatoid arthritis, systemic lupus erythematosus (SLE), Sjogren's syndrome, Canale-Smith syndrome, psoriasis, scleroderma, dermatomyositis, polymyositis, Behcet's syndrome, skin-related autoimmue diseases such as bullus pemphigoid, IgA dermatosis, pemphigus vulgaris, pemphigus foliaceus, dermatitis herpetiformis, contact dermatitis, autoimmune allopecia, erythema nodosa, and epidermolysis bullous aquisita, drug-induced hemotologic autoimmune disorders, autoimmue thrombocytopenic purpura, autoimmune neutropenia, systemic sclerosis, multiple sclerosis, imflammatory demyelinating, diabetes mellitus, autoimmune polyglandular syndromes, vasculitides, Wegener's granulomatosis, Hashimoto's disease, multinodular goitre, Grave's disease, autoimmune encephalomyelitis (EAE), demyelinating diseases, etc.

The term “treating AIDS” means treating a patient who exhibits more serious AIDS-defining illnesses and/or a decline in the circulating CD4 cell count to below a level that is compatible with effective immune function. The term “treating AIDS” also encompasses treating AIDS-related conditions, which means disorders and diseases incidental to or associated with AIDS or HIV infection such as AIDS-related complex (ARC), progressive generalized lymphadenopathy (PGL), anti-HIV antibody positive conditions, and HIV-positive conditions, AIDS-related neurological conditions (such as dementia or tropical paraparesis), Kaposi's sarcoma, thrombocytopenia purpurea and associated opportunistic infections such as Pneumocystis carinii pneumonia, Mycobacterial tuberculosis, esophageal candidiasis, toxoplasmosis of the brain, CMV retinitis, HIV-related encephalopathy, HIV-related wasting syndrome, etc.

Thus, the term “preventing AIDS” as used herein means preventing in a patient who has HIV infection or is suspected to have HIV infection or is at risk of HIV infection from developing AIDS (which is characterized by more serious AIDS-defining illnesses and/or a decline in the circulating CD4 cell count to below a level that is compatible with effective immune function) and/or AIDS-related conditions.

In addition, the term “treating HCV infection” will also encompass treating suspected infection by HCV after suspected past exposure to HCV by, e.g., contact with HCV-contaminated blood, blood transfusion, exchange of body fluids, “unsafe” sex with an infected person, accidental needle stick, receiving a tattoo or acupuncture with contaminated instruments, or transmission of the virus from a mother to a baby during pregnancy, delivery or shortly thereafter. The term “treating HCV infection” will also encompass treating a person who is free of HCV infection but is believed to be at risk of infection by HCV. The term of “preventing HCV” as used herein means preventing in a patient who has HCV infection or is suspected to have HCV infection or is at risk of HCV infection from developing hepatitis C (which is characterized by more serious hepatitis-defining symptoms), cirrhosis, or hepatocellular carcinoma.

### 6.2. Inhibiting Protein Complex or Interacting Protein Members Thereof

In one aspect of the present invention, methods are provided for reducing in cells or tissue the concentration and/or activity of a protein complex identified in accordance with the present invention that comprises one or more of the interacting pairs of proteins described in the tables. In addition, methods are also provided for reducing in cells or tissue the concentration and/or activity of any of the individual proteins identified in the tables. By reducing the concentration of a protein complex and/or one or more of the protein constituents of the protein complex and/or inhibiting the functional activities of the protein complex and/or one or more of the protein constituents of the protein complex, the diseases involving such a protein complex or protein constituents of the protein complex may be treated or prevented.

### 6.2.1. Antibody Therapy

In one embodiment, an antibody may be administered to cells or tissue in vitro or to patients. The antibody administered may be immunoreactive with any of the individual proteins described in the tables, or with one of the protein complexes of the present invention. Suitable antibodies may be monoclonal or polyclonal that fall within any antibody class, e.g., IgG, IgM, IgA, IgE, etc. The antibody suitable for this invention may also take a form of various antibody fragments including, but not limited to, Fab and F(ab′)₂, single-chain fragments (scFv), and the like. In another embodiment, an antibody selectively immunoreactive with the protein complex formed from at least one of the interacting pairs of proteins described in the tables, is administered to cells or tissue in vitro or in to patient. In yet another embodiment, an antibody specific to an individual protein selected from any of the tables is administered to cells or tissue in vitro or in a patient. Methods for making the antibodies of the present invention should be apparent to a person of skill in the art, especially in view of the discussions in Section 3 above. The antibodies can be administered in any suitable form via any suitable route as described in Section 8 below. Preferably, the antibodies are administered in a pharmaceutical composition together with a pharmaceutically acceptable carrier.

Alternatively, the antibodies may be delivered by a gene-therapy approach. That is, nucleic acids encoding the antibodies, particularly single-chain fragments (scFv), may be introduced into cells or tissue in vitro or in a patient such that desirable antibodies may be produced recombinantly in vivo from the nucleic acids. For this purpose, the nucleic acids with appropriate transcriptional and translation regulatory sequences can be directly administered into the patient. Alternatively, the nucleic acids can be incorporated into a suitable vector as described in Sections 2.2 and 5.3.1.1 and delivered into cells or tissue in vitro or in a patient along with the vector. The expression vector containing the nucleic acids can be administered directly to cells or tissue in vitro or in a patient. It can also be introduced into cells, preferably cells derived from a patient to be treated, and subsequently delivered into the patient by cell transplantation. See Section 6.3.2 below.

### 6.2.2. siRNA Therapy

In another embodiment, double-stranded small interfering RNA (siRNA) compounds specific to nucleic acids encoding one or more interacting protein members of a protein complex identified in the present invention are administered to cells or tissue in vitro or in a patient to be therapeutically or prophylactically treated.

As is generally known in the art now, siRNA compounds are RNA duplexes comprising two complementary single-stranded RNAs of 21 nucleotides that form 19 base pairs and possess 3′ overhangs of two nucleotides. See Elbashir et al., Nature 411:494-498 (2001); and PCT Publication Nos. WO 00/44895; WO 01/36646; WO 99/32619; WO 00/01846; WO 01/29058; WO 99/07409; and WO 00/44914. When appropriately targeted via its nucleotide sequence to a specific mRNA in cells, an siRNA can specifically suppress gene expression through a process known as RNA interference (RNAi). See e.g., Zamore & Aronin, Nature Medicine, 9:266-267 (2003). siRNAs can reduce the cellular level of specific mRNAs, and decrease the level of proteins coded by such mRNAs. siRNAs utilize sequence complementarity to target an mRNA for destruction, and are sequence-specific. Thus, they can be highly target-specific, and in mammals have been shown to target mRNAs encoded by different alleles of the same gene. Because of this precision, side effects typically associated with traditional drugs can be reduced or eliminated. In addition, they are relatively stable, and like antisense and ribozyme molecules, they can also be modified to achieve improved pharmaceutical characteristics, such as increased stability, deliverability, and ease of manufacture. Moreover, because siRNA molecules take advantage of a natural cellular pathway, i.e., RNA interference, they are highly efficient in destroying targeted mRNA molecules. As a result, it is relatively easy to achieve a therapeutically effective concentration of an siRNA compound in patients. Thus, siRNAs are a promising new class of drugs being actively developed by pharmaceutical companies.

Indeed, in vivo inhibition of specific gene expression by RNAi has been achieved in various organisms including mammals. For example, Song et al., Nature Medicine, 9:347-351 (2003) discloses that intravenous injection of Fas siRNA compounds into laboratory mice with autoimmune hepatitis specifically reduced Fas mRNA levels and expression of Fas protein in mouse liver cells. The gene silencing effect persisted without diminution for 10 days after the intravenous injection. The injected siRNA was effective in protecting the mice from liver failure and fibrosis. Song et al., Nature Medicine, 9:347-351 (2003). Several other approaches for delivery of siRNA into animals have also proved to be successful. See e.g., McCaffery et al., Nature, 418:38-39 (2002); Lewis et al., Nature Genetics, 32:107-108 (2002); and Xia et al., Nature Biotech., 20:1006-1010 (2002).

The siRNA compounds provided according to the present invention can be synthesized using conventional RNA synthesis methods. For example, they can be chemically synthesized using appropriately protected ribonucleoside phosphoramidites and a conventional DNA/RNA synthesizer. Various applicable methods for RNA synthesis are disclosed in, e.g., Usman et al., J. Am. Chem. Soc., 109:7845-7854 (1987) and Scaringe et al., Nucleic Acids Res., 18:5433-5441 (1990). Custom siRNA synthesis services are available from commercial vendors such as Ambion (Austin, Tex., USA), Dharmacon Research (Lafayette, Colo., USA), Pierce Chemical (Rockford, Ill., USA), ChemGenes (Ashland, Mass., USA), Proligo (Hamburg, Germany), and Cruachem (Glasgow, UK).

The siRNA compounds can also be various modified equivalents of the siRNA structures. As used herein, “modified equivalent” means a modified form of a particular siRNA compound having the same target-specificity (i.e., recognizing the same mRNA molecules that complement the unmodified particular siRNA compound). Thus, a modified equivalent of an unmodified siRNA compound can have modified ribonucleotides, that is, ribonucleotides that contain a modification in the chemical structure of an unmodified nucleotide base, sugar and/or phosphate (or phosphodiester linkage). As is known in the art, an “unmodified ribonucleotide” has one of the bases adenine, cytosine, guanine, and uracil joined to the 1′ carbon of beta-D-ribo-furanose.

Preferably, modified siRNA compounds contain modified backbones or non-natural internucleoside linkages, e.g., modified phosphorous-containing backbones and non-phosphorous backbones such as morpholino backbones; siloxane, sulfide, sulfoxide, sulfone, sulfonate, sulfonamide, and sulfamate backbones; formacetyl and thioformacetyl backbones; alkene-containing backbones; methyleneimino and methylenehydrazino backbones; amide backbones, and the like.

Examples of modified phosphorous-containing backbones include, but are not limited to phosphorothioates, phosphorodithioates, chiral phosphorothioates, phosphotriesters, aminoalkylphosphotriesters, alkyl phosphonates, thionoalkylphosphonates, phosphinates, phosphoramidates, thionophosphoramidates, thionoalkylphosphotriesters, and boranophosphates and various salt forms thereof. See e.g., U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050, each of which is herein incorporated by reference.

Examples of the non-phosphorous containing backbones described above are disclosed in, e.g., U.S. Pat. Nos. 5,034,506; 5,185,444; 5,214,134; 5,216,141; 5,235,033; 5,264,562; 5,264,564; 5,405,938; 5,434,257; 5,470,967; 5,489,677; 5,541,307; 5,561,225; 5,596,086; 5,610,289; 5,602,240; 5,608,046; 5,610,289; 5,618,704; 5,623,070; 5,663,312; 5,677,437; and 5,677,439, each of which is herein incorporated by reference.

Modified forms of siRNA compounds can also contain modified nucleosides (nucleoside analogs), i.e., modified purine or pyrimidine bases, e.g., 5-substituted pyrimidines, 6-Page 137 of 180 azapyrimidines, pyridin-4-one, pyridin-2-one, phenyl, pseudouracil, 2,4,6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine), 2-thiouridine, 4-thiouridine, 5-(carboxyhydroxymethyl)uridine, 5′-carboxymethylaminomethyl-2-thiouridine, 5-carboxymethylaminomethyluridine, 5-methoxyaminomethyl-2-thiouridine, 5-methylaminomethyluridine, 5-methylcarbonylmethyluridine, 5-methyloxyuridine, 5-methyl-2-thiouridine, 4-acetylcytidine, 3-methylcytidine, propyne, quesosine, wybutosine, wybutoxosine, beta-D-galactosylqueosine, N-2, N-6 and O-substituted purines, inosine, 1-methyladenosine, 1-methylinosine, 2,2-dimethylguanosine, 2-methyladenosine, 2-methylguanosine, N6-methyladenosine, 7-methylguanosine, 2-methylthio-N-6-isopentenyladenosine, beta-D-mannosylqueosine, uridine-5-oxyacetic acid, 2-thiocytidine, threonine derivatives, and the like. See e.g., U.S. Pat. Nos. 3,687,808; 4,845,205; 5,130,302; 5,175,273; 5,367,066; 5,432,272; 5,459,255; 5,484,908; 5,502,177; 5,525,711; 5,587,469; 5,594,121; 5,596,091; 5,681,941; and 5,750,692, PCT Publication No. WO 92/07065; PCT Publication No. WO 93/15187; and Limbach et al., Nucleic Acids Res., 22:2183 (1994), each of which is incorporated herein by reference in its entirety.

In addition, modified siRNA compounds can also have substituted or modified sugar moieties, e.g., 2′-O-methoxyethyl sugar moieties. See e.g., U.S. Pat. Nos. 4,981,957; 5,118,800; 5,319,080; 5,393,878; 5,446,137; 5,466,786; 5,514,785; 5,567,811; 5,576,427; 5,591,722; 5,610,300; 5,627,0531 5,639,873; 5,646,265; 5,658,873; 5,670,633; and 5,700,920, each of which is herein incorporated by reference.

Modified siRNA compounds may be synthesized by the methods disclosed in, e.g., U.S. Pat. No. 5,652,094; International Publication Nos. WO 91/03162; WO 92/07065 and WO 93/15187; European Patent Application No. 92110298.4; Perrault et al., Nature, 344:565 (1990); Pieken et al., Science, 253:314 (1991); and Usman and Cedergren, Trends in Biochem. Sci., 17:334 (1992).

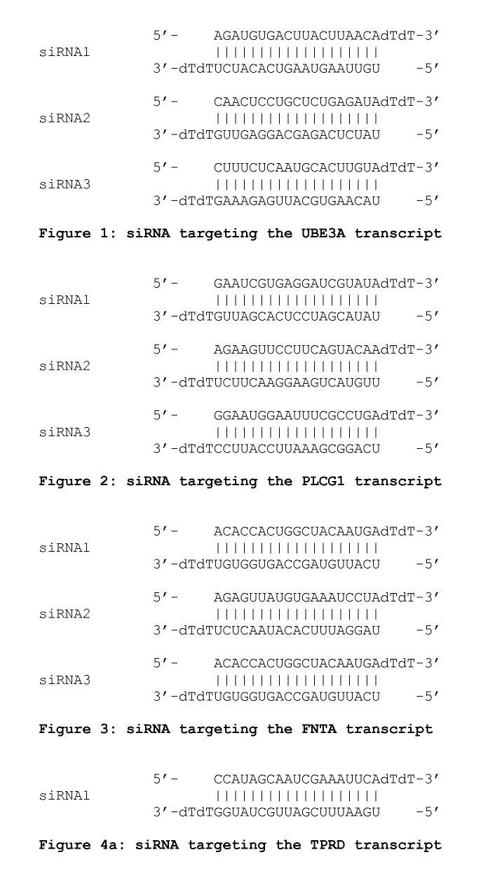

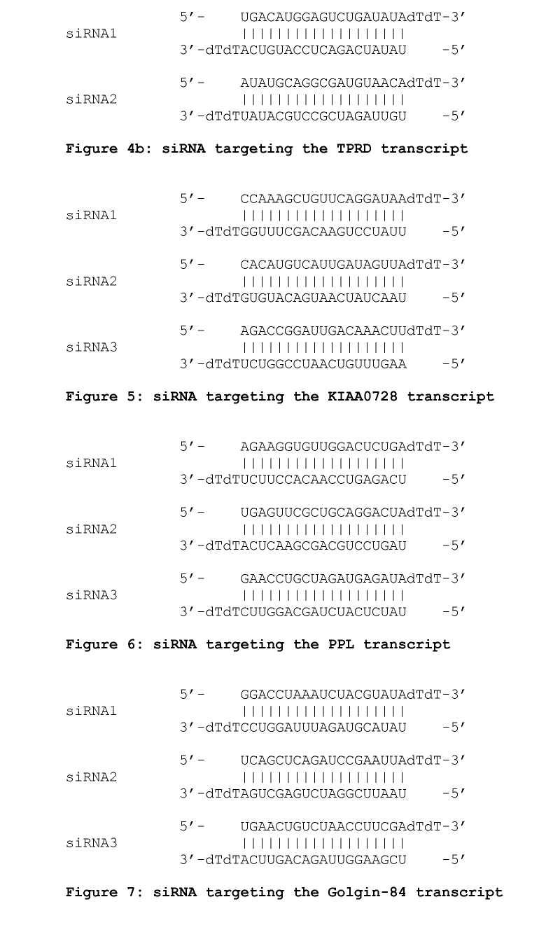

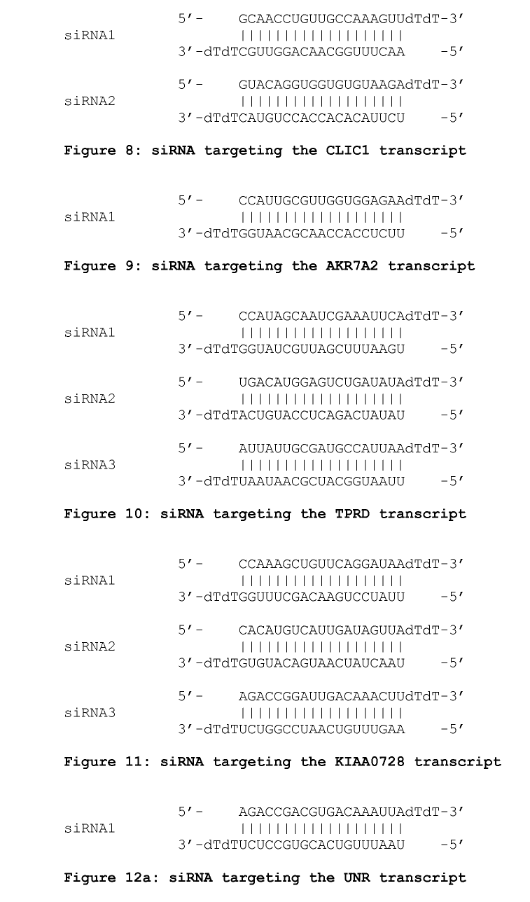

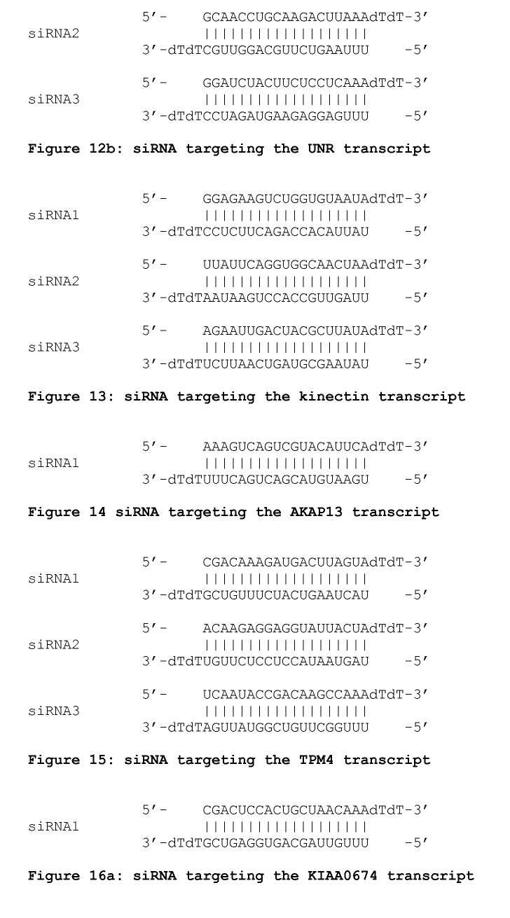

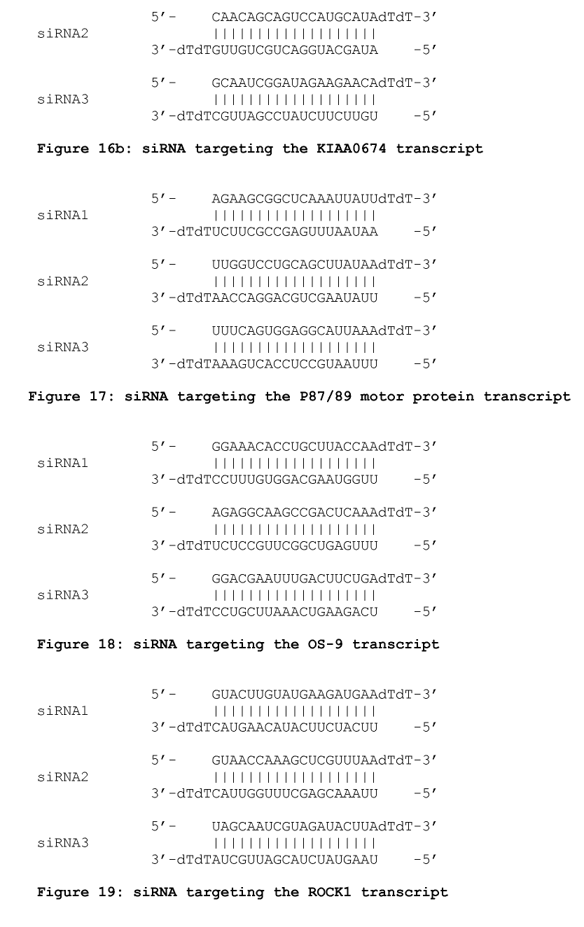

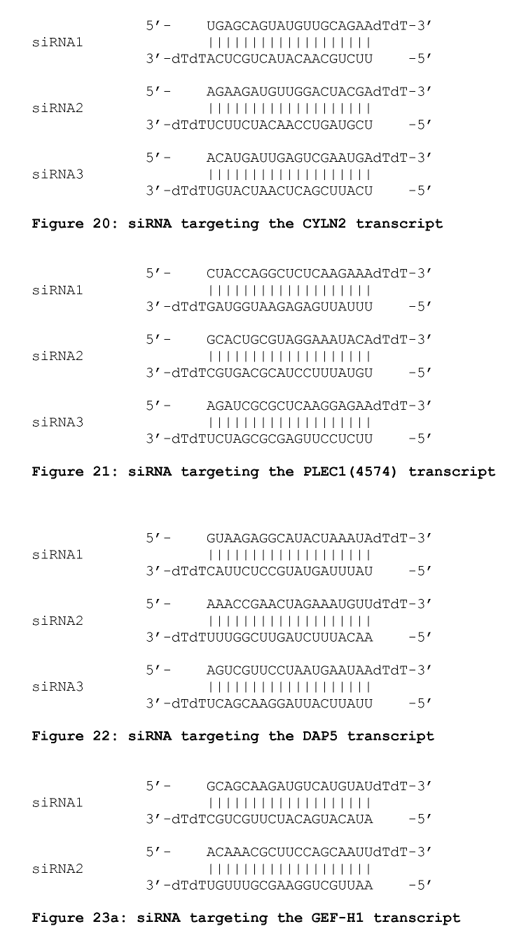

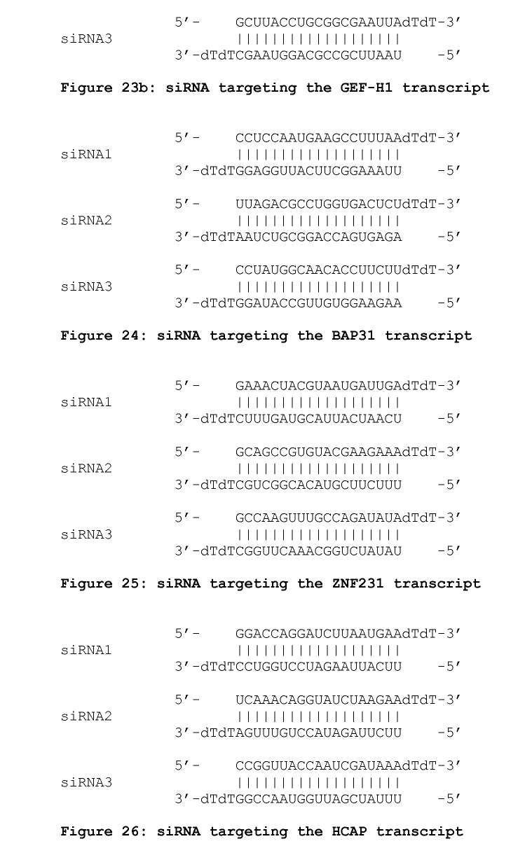

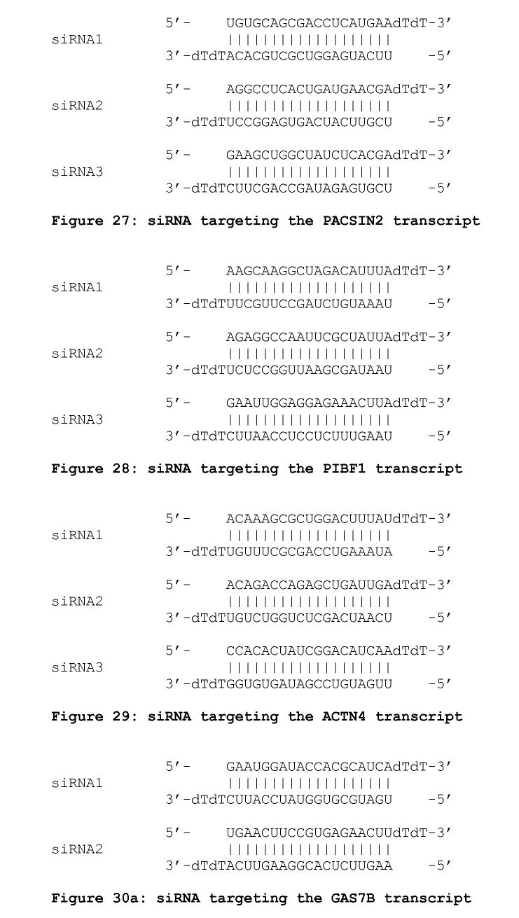

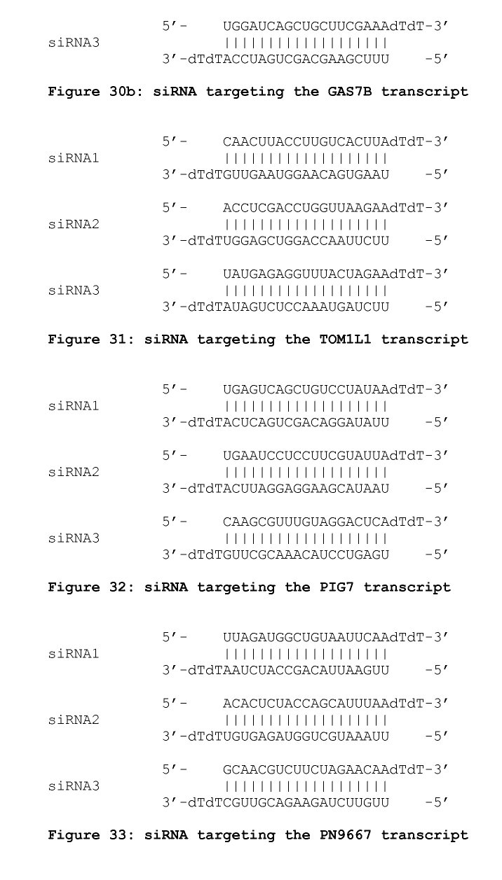

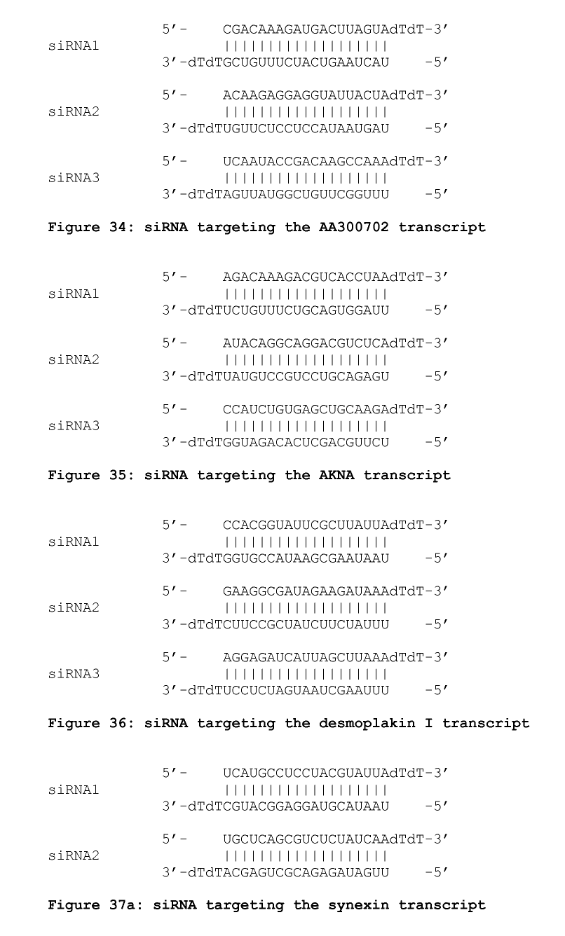

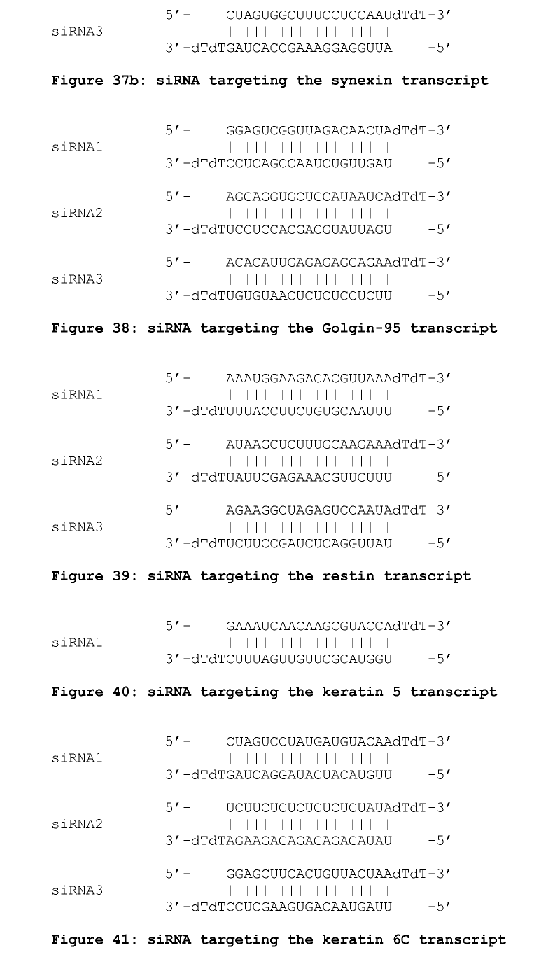

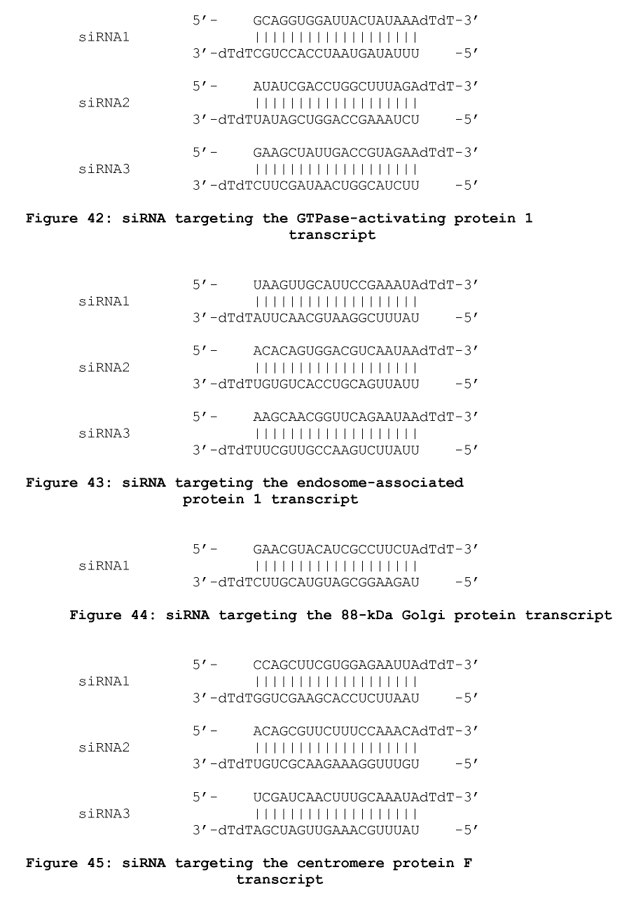

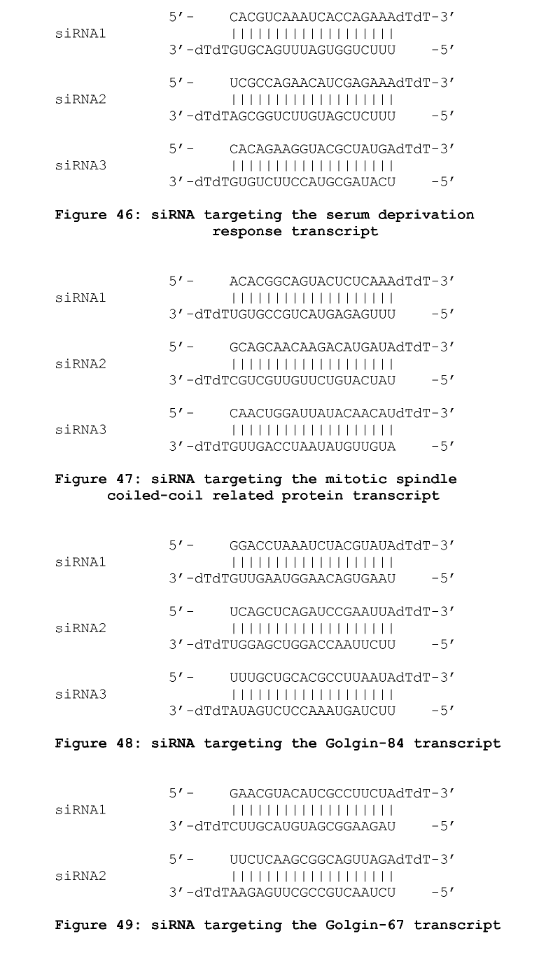

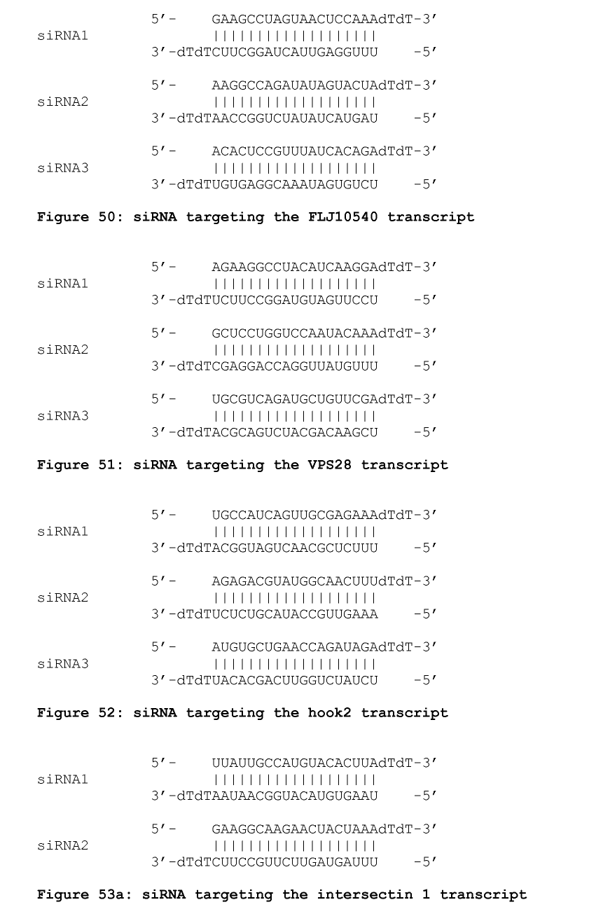

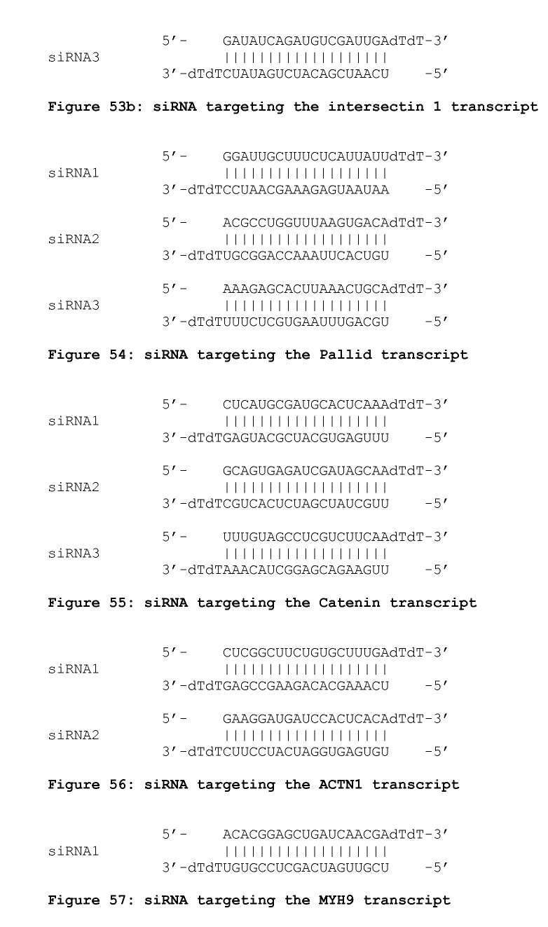

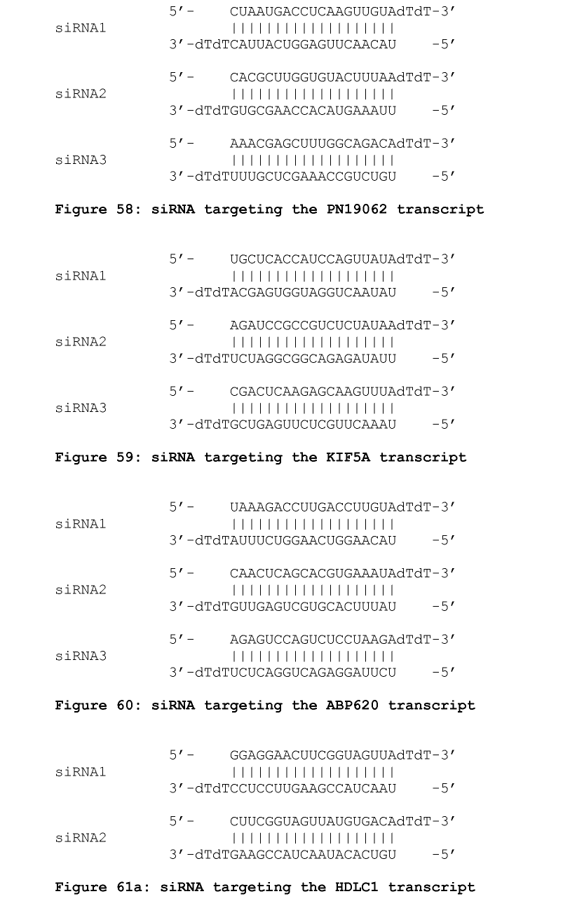

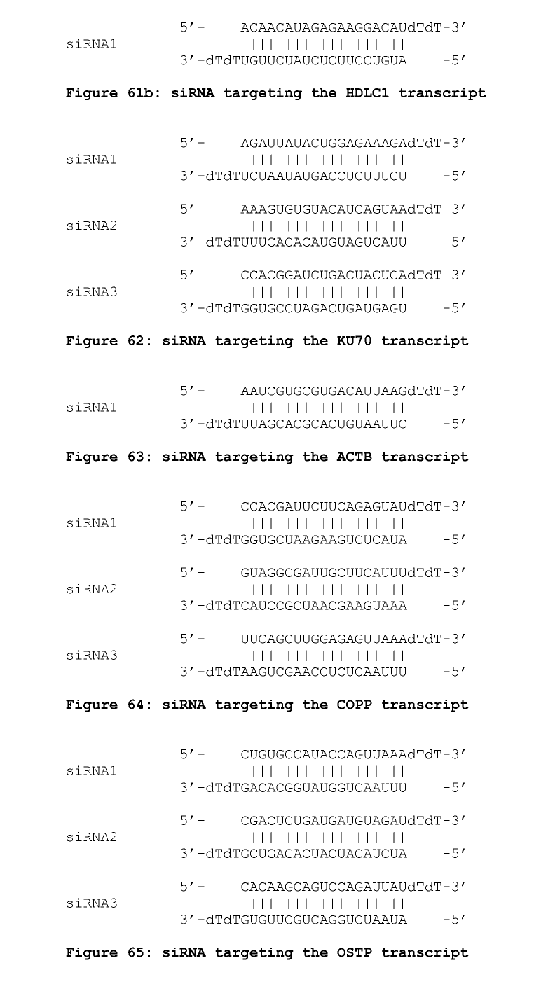

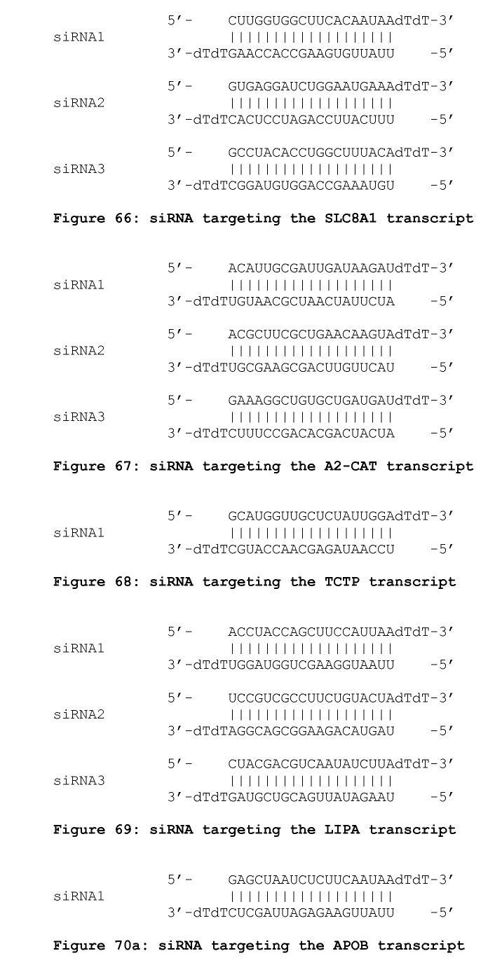

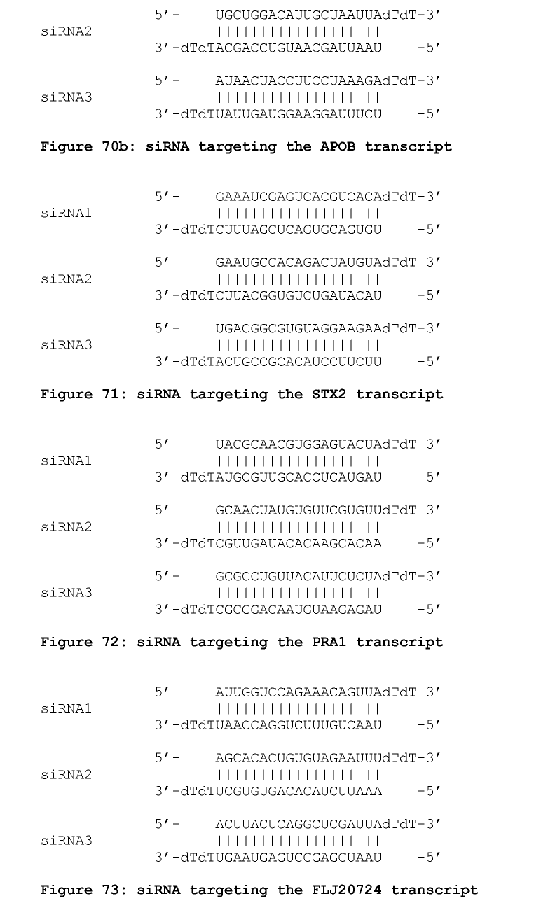

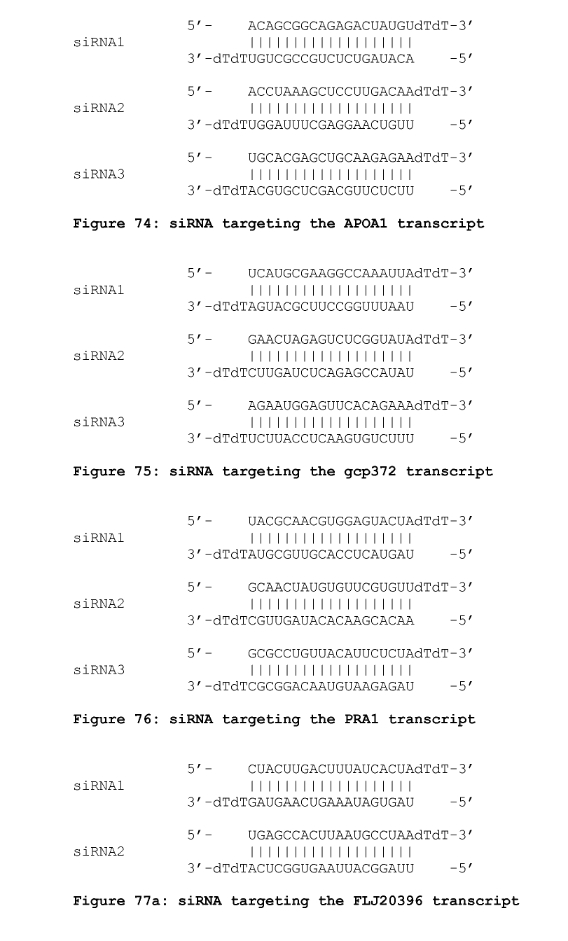

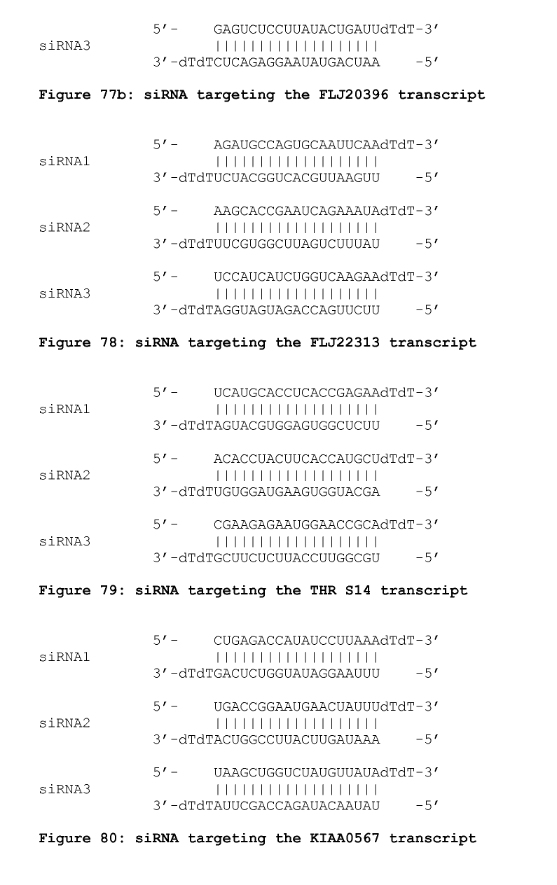

Preferably, the 3′ overhangs of the siRNAs of the present invention are modified to provide resistance to cellular nucleases. In one embodiment the 3′ overhangs comprise 2′-deoxyribonucleotides. In a preferred embodiment (depicted in FIGS. 1-80) these 3′ overhangs comprise a dinucleotide made of two 2′-deoxythymine residues (i.e., dTdT) linked by a 5′-3′ phosphodiester linkage.

siRNA compounds may be administered to mammals by various methods through different routes. For example, they can be administered by intravenous injection. See Song et al., Nature Medicine, 9:347-351 (2003). They can also be delivered directly to a particular organ or tissue by any suitable localized administration methods. Several other approaches for delivery of siRNA into animals have also proved to be successful. See e.g., McCaffery et al., Nature, 418:38-39 (2002); Lewis et al., Nature Genetics, 32:107-108 (2002); and Xia et al., Nature Biotech., 20:1006-1010 (2002). Alternatively, they may be delivered encapsulated in liposomes, by iontophoresis, or by incorporation into other vehicles such as hydrogels, cyclodextrins, biodegradable nanocapsules, and bioadhesive microspheres.

In addition, they may also be delivered by a gene therapy approach, using a DNA vector from which siRNA compounds in, e.g., small hairpin form (shRNA), can be transcribed directly. Recent studies have demonstrated that while double-stranded siRNAs are very effective at mediating RNAi, short, single-stranded, hairpin-shaped RNAs can also mediate RNAi, presumably because they fold into intramolecular duplexes that are processed into double-stranded siRNAs by cellular enzymes. Sui et al., Proc. Natl. Acad. Sci. U.S.A., 99:5515-5520 (2002); Yu et al., Proc. Natl. Acad. Sci. U.S.A., 99:6047-6052 (2002); and Paul et al., Nature Biotech., 20:505-508 (2002)). This discovery has significant and far-reaching implications, since the production of such shRNAs can be readily achieved in vivo by transfecting cells or tissues with DNA vectors bearing short inverted repeats separated by a small number of (e.g., 3 to 9) nucleotides that direct the transcription of such small hairpin RNAs. Additionally, if mechanisms are included to direct the integration of the transcription cassette into the host cell genome, or to ensure the stability of the transcription vector, the RNAi caused by the encoded shRNAs, can be made stable and heritable. Not only have such techniques been used to “knock down” the expression of specific genes in mammalian cells, but they have now been successfully employed to knock down the expression of exogenously expressed transgenes, as well as endogenous genes in the brain and liver of living mice. See generally Hannon, Nature. 418:244-251 (2002) and Shi, Trends Genet., 19:9-12 (2003); see also Xia et al., Nature Biotech., 20:1006-1010 (2002).

Additional siRNA compounds targeted at different sites of the nucleic acids encoding one or more interacting protein members of a protein complex identified in the present invention may also be designed and synthesized according to general guidelines provided herein and generally known to skilled artisans. See e.g., Elbashir, et al. (Nature 411: 494-498 (2001). For example, guidelines have been compiled into “The siRNA User Guide” which is available at the website of The Rockefeller University, New York, N.Y.

Additionally, to assist in the design of siRNAs for the efficient RNAi-mediated silencing of any target gene, several siRNA supply companies maintain web-based design tools that utilize these general guidelines for “picking” siRNAs when presented with the mRNA or coding DNA sequence of the target gene. Examples of such tools can be found at the web sites of Dharmacon, Inc. (Lafayette, Colo.), Ambion, Inc. (Austin, Tex.), and Qiagen, Inc. (Valencia, Calif.), among others. Generally speaking, when provided with an mRNA or coding DNA sequence, these design tools scan the sequence for potential siRNA targets, using several distinct criteria. For example, the design tools may scan for an open reading frame and limit further scanning to that region of sequence. They may then scan for a particular dinucleotide, the most desirable of which being AA, or alternatively CA, GA or TA. Upon finding one of these dinucleotides, they will then examine the dinucleotide and the 19 nucleotides immediately 3′ of it for G/C content, nucleotide triplets (esp. GGG & CCC), and, using a BLAST algorithm search, for whether or not the 19 nucleotide sequence is unique to a specific target gene in the human genome. The features that make for an “ideal” target sequence are: (1) a 5′-most dinucleotide sequence of AA, or, less preferably, CA, GA or TA; (2) a G/C content of approximately 30-50%; (3) lack of trinucleotide repeats, especially GGG and CCC, and (4) being unique to the target gene (i.e., sequences that share no significant homology with genes other than the one being targeted), so that other genes are not inadvertently targeted by the same siRNA designed for this particular target sequence. Another criteria to be considered is whether or not the target sequence includes a known polymorphic site. If so, siRNAs designed to target one particular allele may not effectively target another allele, since single base mismatches between the target sequence and its complementary strand in a given siRNA can greatly reduce the effectiveness of RNAi induced by that siRNA. Given that target sequence and such design tools and design criteria, an ordinarily skilled artisan apprised of the present disclosure should be able to design and synthesized additional siRNA compounds useful in reducing the mRNA level and therefore protein level of one or more interacting protein members of a protein complex identified in the present invention.

### 6.2.3. Antisense Therapy

In another embodiment, antisense compounds specific to nucleic acids encoding one or more interacting protein members of a protein complex identified in the present invention are administered to cells or tissue in vitro or in a patient to be therapeutically or prophylactically treated. The antisense compounds should specifically inhibit the expression of the one or more interacting protein members. Examples of antisense compounds specific to nucleic acids encoding individual proteins in the tables above are provided in SEQ ID NOs:9-235.

As is known in the art, antisense drugs generally act by hybridizing to a particular target nucleic acid thus blocking gene expression. Methods for designing antisense compounds and using such compounds in treating diseases are well known and well developed in the art. For example, the antisense drug Vitravene® (fomivirsen), a 21-base long oligonucleotide, has been successfully developed and marketed by Isis Pharmaceuticals, Inc. for treating cytomegalovirus (CMV)-induced retinitis.

Any methods for designing and making antisense compounds may be used for the purpose of the present invention. See generally, Sanghvi et al., eds., Antisense Research and Applications, CRC Press, Boca Raton, 1993. Typically, antisense compounds are oligonucleotides designed based on the nucleotide sequence of the mRNA or gene of one or more target proteins, e.g., the interacting protein members of a particular protein complex of the present invention. In particular, antisense compounds can be designed to specifically hybridize to a particular region of the gene sequence or mRNA of one or more of the interacting protein members to modulate (increase or decrease) replication, transcription, or translation. As used herein, the term “specifically hybridize” or paraphrases thereof means a sufficient degree of complementarity or pairing between an antisense oligo and a target DNA or mRNA such that stable and specific binding occurs therebetween. In particular, 100% complementary or pairing is not required. Specific hybridization takes place when sufficient hybridization occurs between the antisense compound and its intended target nucleic acids in the substantial absence of non-specific binding of the antisense compound to non-target sequences under predetermined conditions, e.g., for purposes of in vivo treatment, preferably under physiological conditions. Preferably, specific hybridization results in the interference with normal expression of the target DNA or mRNA.

For example, antisense oligonucleotides can be designed to specifically hybridize to target genes, in regions critical for regulation of transcription; to pre-mRNAs, in regions critical for correct splicing of nascent transcripts; and to mature mRNAs, in regions critical for translation initiation or mRNA stability and localization.

As is generally known in the art, commonly used oligonucleotides are oligomers or polymers of ribonucleotides or deoxyribonucleotides, that are composed of a naturally-occurring nitrogenous base, a sugar (ribose or deoxyribose) and a phosphate group. In nature, the nucleotides are linked together by phosphodiester bonds between the 3′ and 5′ positions of neighboring sugar moieties. However, it is noted that the term “oligonucleotides” also encompasses various non-naturally occurring mimetics and derivatives, i.e., modified forms, of naturally occurring oligonucleotides as described below. Typically an antisense compound of the present invention is an oligonucleotide having from about 6 to about 200, and preferably from about 8 to about 30 nucleoside bases.

The antisense compounds preferably contain modified backbones or non-natural internucleoside linkages, including but not limited to, modified phosphorous-containing backbones and non-phosphorous backbones such as morpholino backbones; siloxane, sulfide, sulfoxide, sulfone, sulfonate, sulfonamide, and sulfamate backbones; formacetyl and thioformacetyl backbones; alkene-containing backbones; methyleneimino and methylenehydrazino backbones; amide backbones, and the like.

Examples of modified phosphorous-containing backbones include, but are not limited to phosphorothioates, phosphorodithioates, chiral phosphorothioates, phosphotriesters, aminoalkylphosphotriesters, alkyl phosphonates, thionoalkylphosphonates, phosphinates, phosphoramidates, thionophosphoramidates, thionoalkylphosphotriesters, and boranophosphates and various salt forms thereof. See e.g., U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050, each of which is herein incorporated by reference.

Examples of the non-phosphorous containing backbones described above are disclosed in, e.g., U.S. Pat. Nos. 5,034,506; 5,185,444; 5,214,134; 5,216,141; 5,235,033; 5,264,562; 5,264,564; 5,405,938; 5,434,257; 5,470,967; 5,489,677; 5,541,307; 5,561,225; 5,596,086; 5,610,289; 5,602,240; 5,608,046; 5,610,289; 5,618,704; 5,623,070; 5,663,312; 5,677,437; and 5,677,439, each of which is herein incorporated by reference.

Another useful modified oligonucleotide is peptide nucleic acid (PNA), in which the sugar-backbone of an oligonucleotide is replaced with an amide containing backbone, e.g., an aminoethylglycine backbone. See U.S. Pat. Nos. 5,539,082 and 5,714,331; and Nielsen et al., Science, 254, 1497-1500 (1991), all of which are incorporated herein by reference. PNA antisense compounds are resistant to RNase H digestion and thus exhibit longer half-life. In addition, various modifications may be made in PNA backbones to impart desirable drug profiles such as better stability, increased drug uptake, higher affinity to target nucleic acid, etc.

Alternatively, the antisense compounds are oligonucleotides containing modified nucleosides, i.e., modified purine or pyrimidine bases, e.g., 5-substituted pyrimidines, 6-azapyrimidines, and N-2, N-6 and O-substituted purines, and the like. See e.g., U.S. Pat. Nos. 3,687,808; 4,845,205; 5,130,302; 5,175,273; 5,367,066; 5,432,272; 5,459,255; 5,484,908; 5,502,177; 5,525,711; 5,587,469; 5,594,121; 5,596,091; 5,681,941; and 5,750,692, each of which is incorporated herein by reference in its entirety.

In addition, oligonucleotides with substituted or modified sugar moieties may also be used. For example, an antisense compound may have one or more 2′-O-methoxyethyl sugar moieties. See e.g., U.S. Pat. Nos. 4,981,957; 5,118,800; 5,319,080; 5,393,878; 5,446,137; 5,466,786; 5,514,785; 5,567,811; 5,576,427; 5,591,722; 5,610,300; 5,627,0531 5,639,873; 5,646,265; 5,658,873; 5,670,633; and 5,700,920, each of which is herein incorporated by reference.

Other types of oligonucleotide modifications are also useful including linking an oligonucleotide to a lipid, phospholipid or cholesterol moiety, cholic acid, thioether, aliphatic chain, polyamine, polyethylene glycol (PEG), or a protein or peptide. The modified oligonucleotides may exhibit increased uptake into cells, and improved stability, i.e., resistance to nuclease digestion and other biodegradations. See e.g., U.S. Pat. No. 4,522,811; Burnham, Am. J. Hosp. Pharm., 15:210-218 (1994).

Antisense compounds can be synthesized using any suitable methods known in the art. In fact, antisense compounds may be custom made by commercial suppliers. Alternatively, antisense compounds may be prepared using DNA synthesizers available commercially from various vendors, e.g., Applied Biosystems Group of Norwalk, Conn.

The antisense compounds can be formulated into a pharmaceutical composition with suitable carriers and administered into cells or tissue in vitro or in a patient using any suitable route of administration. Alternatively, the antisense compounds may also be used in a “gene-therapy” approach. That is, the oligonucleotide is subcloned into a suitable vector and transformed into human cells. The antisense oligonucleotide is then produced in vivo through transcription. Methods for gene therapy are disclosed in Section 6.3.2 below.

### 6.2.4. Ribozyme Therapy

In another embodiment, an enzymatic RNA or ribozyme is designed to target the nucleic acids encoding one or more of the interacting protein members of the protein complexes of the present invention. Ribozymes are RNA molecules possessing enzymatic activity. One class of ribozymes is capable of repeatedly cleaving other separate RNA molecules into two or more pieces in a nucleotide base sequence specific manner. See Kim et al., Proc. Natl. Acad. of Sci. USA, 84:8788 (1987); Haseloff and Gerlach, Nature, 334:585 (1988); and Jefferies et al., Nucleic Acid Res., 17:1371 (1989). Such ribozymes typically have two functional domains: a catalytic domain and a binding sequence that guides the binding of ribozymes to a target RNA through complementary base-pairing. Once a specifically-designed ribozyme is bound to a target mRNA, it enzymatically cleaves the target mRNA, typically reducing its stability and destroying its ability to direct translation of an encoded protein. After a ribozyme has cleaved its RNA target, it is released from that target RNA and thereafter can bind and cleave another target. That is, a single ribozyme molecule can repeatedly bind and cleave new targets. Therefore, one advantage of ribozyme treatment is that a lower amount of exogenous RNA is required as compared to conventional antisense therapies. In addition, ribozymes exhibit less affinity to mRNA targets than DNA-based antisense oligonucleotides, and therefore are less prone to bind to unintended targets.

In accordance with the present invention, a ribozyme may target any portion of the mRNA encoding one or more interacting protein members of the protein complexes formed by the interactions described in the tables. Methods for selecting a ribozyme target sequence and designing and making ribozymes are generally known in the art. See e.g., U.S. Pat. Nos. 4,987,071; 5,496,698; 5,525,468; 5,631,359; 5,646,020; 5,672,511; and 6,140,491, each of which is incorporated herein by reference in its entirety. For example, suitable ribozymes may be designed in various configurations such as hammerhead motifs, hairpin motifs, hepatitis delta virus motifs, group I intron motifs, or RNase P RNA motifs. See e.g., U.S. Pat. Nos. 4,987,071; 5,496,698; 5,525,468; 5,631,359; 5,646,020; 5,672,511; and 6,140,491; Rossi et al., AIDS Res. Human Retroviruses 8:183 (1992); Hampel and Tritz, Biochemistry 28:4929 (1989); Hampel et al., Nucleic Acids Res., 18:299 (1990); Perrotta and Been, Biochemistry 31:16 (1992); and Guerrier-Takada et al., Cell, 35:849 (1983).

Ribozymes can be synthesized by the same methods used for normal RNA synthesis. For example, such methods are disclosed in Usman et al., J. Am. Chem. Soc., 109:7845-7854 (1987) and Scaringe et al., Nucleic Acids Res., 18:5433-5441 (1990). Modified ribozymes may be synthesized by the methods disclosed in, e.g., U.S. Pat. No. 5,652,094; International Publication Nos. WO 91/03162; WO 92/07065 and WO 93/15187; European Patent Application No. 92110298.4; Perrault et al., Nature, 344:565 (1990); Pieken et al., Science, 253:314 (1991); and Usman and Cedergren, Trends in Biochem. Sci., 17:334 (1992).

Ribozymes of the present invention may be administered to cells by any known methods, e.g., disclosed in International Publication No. WO 94/02595. For example, they can be administered directly to cells or tissue in vitro or in a patient through any suitable route, e.g., intravenous injection. Alternatively, they may be delivered encapsulated in liposomes, by iontophoresis, or by incorporation into other vehicles such as hydrogels, cyclodextrins, biodegradable nanocapsules, and bioadhesive microspheres. In addition, they may also be delivered by a gene therapy approach, using a DNA vector from which the ribozyme RNA can be transcribed directly. Gene therapy methods are disclosed in detail below in Section 6.3.2.

### 6.2.5. Other Methods

The in-patient concentrations and activities of the protein complexes and interacting proteins of the present invention may also be altered by other methods. For example, compounds identified in accordance with the methods described in Section 5 that are capable of interfering with or dissociating protein-protein interactions between the interacting protein members of a protein complex may be administered to cells or tissue in vitro or in a patient. Compounds identified in in vitro binding assays described in Section 5.2 that bind to the protein complexes of the present invention, or the interacting members thereof, may also be used in the treatment. Compounds identified in in vitro binding assays described in Section 5.2 that bind to the protein complexes of the present invention, or the interacting members thereof, may also be used in the treatment.

In addition, potentially useful agents also include incomplete proteins, i.e., fragments of the interacting protein members that are capable of binding to their respective binding partners in a protein complex but are defective with respect to their normal cellular functions. For example, binding domains of the interacting member proteins of a protein complex may be used as competitive inhibitors of the activities of the protein complex. As will be apparent to skilled artisans, derivatives or homologues of the binding domains may also be used. Binding domains can be easily identified using molecular biology techniques, e.g., mutagenesis in combination with yeast two-hybrid assays. Preferably, the protein fragment used is a fragment of an interacting protein member having a length of less than 90%, 80%, more preferably less than 75%, 65%, 50%, or less than 40% of the full length of the protein member. Examples of protein fragments of the proteins in the tables above that are potentially useful agents are provided by SEQ ID NOs:236-469.

In one embodiment, a fragment of a protein identified in the tables above is administered. In a specific embodiment, one or more of the interaction domains of a protein identified in the tables, within the regions listed in the tables, is administered to cells or tissue in vitro, or are administered to a patient in need of such treatment. For example, suitable protein fragments can include polypeptides having a contiguous span of 4, 5, 6, 7, 8, 9, 10, 11, 12, 13, 14, 15, 18, 20 or 25, preferably from 4 to 30, 40 or 50 amino acids or more of the sequence of a first protein identified in the tables, that are capable of interacting with a second protein described in the tables. Also, suitable protein fragments can include peptides capable of binding one or more of the proteins described in the tables, and having an amino acid sequence of from 4 to 30 amino acids that is at least 75%, 80%, 82%, 85%, 87%, 90%, 95% or more identical to a contiguous span of amino acids of a protein described in the tables. Alternatively, a polypeptide capable of interacting with a first protein of an interacting pair of proteins of the present invention, and having a contiguous span of 4, 5, 6, 7, 8, 9, 10, 11, 12, 13, 14, 15, 18, 20 or 25, preferably from 4 to 30, 40 or 50 or more amino acids of the amino acid sequence of a second protein of the same interacting pair of proteins, may be administered. Also, other examples of suitable compounds include a peptide capable of binding a first interacting partner of a pair of interacting proteins of the present invention and having an amino acid sequence of from 4 to 30, 40, 50 or more amino acids that is at least 75%, 80%, 82%, 85%, 87%, 90%, 92%, 95% or more identical to a contiguous span of amino acids from a second interacting partner of a pair of interacting proteins of the present invention. In addition, the administered compounds can be an antibody or antibody fragment, preferably a single-chain antibody immunoreactive with any of the proteins listed in the tables, or a protein complex of the present invention.

The protein fragments suitable as competitive inhibitors can be delivered into cells by direct cell internalization, receptor mediated endocytosis, or via a “transporter.” It is noted that when the target proteins or protein complexes to be modulated reside inside cells, the compound administered to cells in vitro or in vivo in the method of the present invention preferably is delivered into the cells in order to achieve optimal results. Thus, preferably, the compound to be delivered is associated with a transporter capable of increasing the uptake of the compound by cells harboring the target protein or protein complex. As used herein, the term “transporter” refers to an entity (e.g., a compound or a composition or a physical structure formed from multiple copies of a compound or multiple different compounds) that is capable of facilitating the uptake of a compound of the present invention by animal cells, particularly human cells. That is, the cell uptake of a compound of the present invention in the presence of a “transporter” is at least 50% higher than the cell uptake of the compound in the absence of the “transporter.” Preferably, a “transporter” is selected such that the cell uptake of a compound of the present invention in the presence of a “transporter” is at least 75% higher, preferably at least 100% or 200% higher, and more preferably at least 300%, 400% or 500% higher than the cell uptake of the compound in the absence of the “transporter.” Methods of assaying cell uptake of a compound should be apparent to skilled artisans. For example, the compound to be delivered can be labeled with a radioactive isotope or another detectable marker (e.g., a fluorescence marker), and added to cultured cells in the presence or absence of a transporter, and incubated for a time period sufficient to allow maximal uptake. Cells can then be separated from the culture medium and the detectable signal (e.g., radioactivity) caused by the compound inside the cells can be measured. The result obtained in the presence of a transporter can be compared to that obtained in the absence of a transporter.

Many molecules and structures known in the art can be used as “transporters.” In one embodiment, a penetratin is used as a transporter. For example, the homeodomain of Antennapedia, a Drosophila transcription factor, can be used as a transporter to deliver a compound of the present invention. Indeed, any suitable member of the penetratin class of peptides can be used to carry a compound of the present invention into cells. Penetratins are disclosed in, e.g., Derossi et al., Trends Cell Biol., 8:84-87 (1998), which is incorporated herein by reference. Penetratins transport molecules attached thereto across cytoplasmic membranes or nuclear membranes efficiently, in a receptor-independent, energy-independent, and cell type-independent manner. Methods for using a penetratin as a carrier to deliver oligonucleotides and polypeptides are also disclosed in U.S. Pat. No. 6,080,724; Pooga et al., Nat. Biotech., 16:857 (1998); and Schutze et al., J. Immunol., 157:650 (1996), all of which are incorporated herein by reference. U.S. Pat. No. 6,080,724 defines the minimal requirements for a penetratin peptide as a peptide of 16 amino acids with 6 to 10 of which being hydrophobic. The amino acid at position 6 counting from either the N- or C-terminus is tryptophan, while the amino acids at positions 3 and 5 counting from either the N- or C-terminus are not both valine. Preferably, the helix 3 of the homeodomain of Drosophila Antennapedia is used as a transporter. More preferably, a peptide having a sequence of amino acid residues 43-58 of the homeodomain Antp is employed as a transporter. In addition, other naturally occurring homologs of the helix 3 of the homeodomain of Drosophila Antennapedia can be used. For example, homeodomains of Fushi-tarazu and Engrailed have been shown to be capable of transporting peptides into cells. See Han et al., Mol. Cells, 10:728-32 (2000). As used herein, the term “penetratin” also encompasses peptoid analogs of the penetratin peptides. Typically, the penetratin peptides and peptoid analogs thereof are covalently linked to a compound to be delivered into cells thus increasing the cellular uptake of the compound.

In another embodiment, the HIV-1 tat protein or a derivative thereof is used as a “transporter” covalently linked to a compound according to the present invention. The use of HIV-1 tat protein and derivatives thereof to deliver macromolecules into cells has been known in the art. See Green and Loewenstein, Cell, 55:1179 (1988); Frankel and Pabo, Cell, 55:1189 (1988); Vives et al., J. Biol. Chem., 272:16010-16017 (1997); Schwarze et al., Science, 285:1569-1572 (1999). It is known that the sequence responsible for cellular uptake consists of the highly basic region, amino acid residues 49-57. See e.g., Vives et al., J. Biol. Chem., 272:16010-16017 (1997); Wender et al., Proc. Nat'l Acad. Sci. USA, 97:13003-13008 (2000). The basic domain is believed to target the lipid bilayer component of cell membranes. It causes a covalently linked protein or nucleic acid to cross cell membrane rapidly in a cell type-independent manner. Proteins ranging in size from 15 to 120 kD have been delivered with this technology into a variety of cell types both in vitro and in vivo. See Schwarze et al., Science, 285:1569-1572 (1999). Any HIV tat-derived peptides or peptoid analogs thereof capable of transporting macromolecules such as peptides can be used for purposes of the present invention. For example, any native tat peptides having the highly basic region, amino acid residues 49-57 can be used as a transporter by covalently linking it to the compound to be delivered. In addition, various analogs of the tat peptide of amino acid residues 49-57 can also be useful transporters for purposes of this invention. Examples of various such analogs are disclosed in Wender et al., Proc. Nat'l Acad. Sci. USA, 97:13003-13008 (2000) (which is incorporated herein by reference) including, e.g., d-Tat₄₉₋₅₇, retro-inverso isomers of l- or d-Tat₄₉₋₅₇ (i.e., l-Tat₅₇₋₄₉ and d-Tat₅₇₋₄₉), L-arginine oligomers, D-arginine oligomers, L-lysine oligomers, D-lysine oligomers, L-histine oligomers, D-histine oligomers, L-ornithine oligomers, D-ornithine oligomers, and various homologues, derivatives (e.g., modified forms with conjugates linked to the small peptides) and peptoid analogs thereof. Preferably, arginine oligomers are preferred to the other oligomers, since arginine oligomers are much more efficient in promoting cellular uptake. As used herein, the term “oligomer” means a molecule that includes a covalently linked chain of amino acid residues of the same amino acids having a large enough number of such amino acid residues to confer transporter activities on the molecule. Typically, an oligomer contains at least 6, preferably at least 7, 8, or 9 such amino acid residues. In one embodiment, the transporter is a peptide that includes at least six contiguous amino acid residues that are a combination of two or more of L-arginine, D-arginine, L-lysine, D-lysine, L-histidine, D-histine, L-ornithine, and D-ornithine.

Other useful transporters known in the art include, but are not limited to, short peptide sequences derived from fibroblast growth factor (See Lin et al., J. Biol. Chem., 270:14255-14258 (1998)), Galparan (See Pooga et al., FASEB J. 12:67-77 (1998)), and HSV-1 structural protein VP22 (See Elliott and O'Hare, Cell, 88:223-233 (1997)).

As the above-described various transporters are generally peptides, fusion proteins can be conveniently made by recombinant expression to contain a transporter peptide covalently linked by a peptide bond to a competitive protein fragment. Alternatively, conventional methods can be used to chemically synthesize a transporter peptide or a peptide of the present invention or both.

The hybrid peptide can be administered to cells or tissue in vitro or to a patient in a suitable pharmaceutical composition as provided in Section 8.

In addition to peptide-based transporters, various other types of transporters can also be used, including but not limited to cationic liposomes (see Rui et al., J. Am. Chem. Soc., 120:11213-11218 (1998)), dendrimers (Kono et al., Bioconjugate Chem., 10:1115-1121 (1999)), siderophores (Ghosh et al., Chem. Biol., 3:1011-1019 (1996)), etc. In a specific embodiment, the compound according to the present invention is encapsulated into liposomes for delivery into cells.

Additionally, when a compound according to the present invention is a peptide, it can be administered to cells by a gene therapy method. That is, a nucleic acid encoding the peptide can be administered to in vitro cells or to cells in vivo in a human or animal body. Any suitable gene therapy methods may be used for purposes of the present invention. Various gene therapy methods are well known in the art and are described in Section 6.3.2. below. Successes in gene therapy have been reported recently. See e.g., Kay et al., Nature Genet., 24:257-61 (2000); Cavazzana-Calvo et al., Science, 288:669 (2000); and Blaese et al., Science, 270: 475 (1995); Kantoff, et al., J. Exp. Med., 166:219 (1987).

In yet another embodiment, the gene therapy methods discussed in Section 6.3.2 below are used to “knock out” the gene encoding an interacting protein member of a protein complex, or to reduce the gene expression level. For example, the gene may be replaced with a different gene sequence or a non-functional sequence or simply deleted by homologous recombination. In another gene therapy embodiment, the method disclosed in U.S. Pat. No. 5,641,670, which is incorporated herein by reference, may be used to reduce the expression of the genes for the interacting protein members. Essentially, an exogenous DNA having at least a regulatory sequence, an exon and a splice donor site can be introduced into an endogenous gene encoding an interacting protein member by homologous recombination such that the regulatory sequence, the exon and the splice donor site present in the DNA construct become operatively linked to the endogenous gene. As a result, the expression of the endogenous gene is controlled by the newly introduced exogenous regulatory sequence. Therefore, when the exogenous regulatory sequence is a strong gene expression repressor, the expression of the endogenous gene encoding the interacting protein member is reduced or blocked. See U.S. Pat. No. 5,641,670.

### 6.3. Activation of Protein Complex or Interacting Protein Members Thereof

The present invention also provides methods for increasing in cells or tissue in vitro or in a patient the concentration and/or activity of a protein complex, or of an individual protein member thereof, identified in accordance with the present invention. Such methods can be particularly useful in instances where a reduced concentration and/or activity of a protein complex, or a protein member thereof, is associated with a particular disease or disorder to be treated, or where an increased concentration and/or activity of a protein complex, or a protein member thereof, would be beneficial to the improvement of a cellular function or disease state. By increasing the concentration of the protein complex, or a protein member thereof, and/or stimulating the functional activities of the protein complex or a protein member thereof, the disease or disorder may be treated or prevented.

### 6.3.1. Administration of Protein Complex or Protein Members Thereof

Where the concentration or activity of a particular protein complex of the present invention, or any individual protein constituent of a protein complex in cells or tissue in vitro or in a patient is determined to be low or is desired to be increased, the protein complex, or an individual constituent protein of the protein complex may be administered directly to the patient to increase the concentration and/or activity of the protein complex, or the individual constituent protein. For this purpose, protein complexes prepared by any one of the methods described in Section 2.2 may be administered to the patient, preferably in a pharmaceutical composition as described below. Alternatively, one or more individual interacting protein members of the protein complex may also be administered to the patient in need of treatment. For example, one or more of the individual proteins or the interacting pairs of proteins described in the tables may be given to cells or tissue in vitro or to a patient. Proteins isolated or purified from normal individuals or recombinantly produced can all be used in this respect. Preferably, two or more interacting protein members of a protein complex are administered. The proteins or protein complexes may be administered to a patient needing treatment using any of the methods described in Section 8.

### 6.3.2. Gene Therapy

In another embodiment, the concentration and/or activity of a particular protein complex comprising one or more of the interacting pairs of proteins described in the tables or an individual constituent protein of a protein complex of the present invention is increased or restored in patients, tissue or cells by a gene therapy approach. For example, nucleic acids encoding one or more protein members of a protein complex of the present invention, or portions or fragments thereof are introduced into patients, tissue, or cells such that the protein(s) are expressed from the introduced nucleic acids. For these purposes, nucleic acids encoding one or more of the proteins described in the tables, or fragments, homologues or derivatives thereof can be used in the gene therapy in accordance with the present invention. For example, if a disease-causing mutation exists in one of the protein members in cells or tissue in vitro or in a patient, then a nucleic acid encoding a wild-type protein can be introduced into tissue cells of the patient. The exogenous nucleic acid can be used to replace the corresponding endogenous defective gene by, e.g., homologous recombination. See U.S. Pat. No. 6,010,908, which is incorporated herein by reference. Alternatively, if the disease-causing mutation is a recessive mutation, the exogenous nucleic acid is simply used to express a wild-type protein in addition to the endogenous mutant protein. In another approach, the method disclosed in U.S. Pat. No. 6,077,705 may be employed in gene therapy. That is, the patient is administered both a nucleic acid construct encoding a ribozyme and a nucleic acid construct comprising a ribozyme resistant gene encoding a wild type form of the gene product. As a result, undesirable expression of the endogenous gene is inhibited and a desirable wild-type exogenous gene is introduced. In yet another embodiment, if the endogenous gene is of wild-type and the level of expression of the protein encoded thereby is desired to be increased, additional copies of wild-type exogenous genes may be introduced into the patient by gene therapy, or alternatively, a gene activation method such as that disclosed in U.S. Pat. No. 5,641,670 may be used.

Various gene therapy methods are well known in the art. Successes in gene therapy have been reported recently. See e.g., Kay et al., Nature Genet., 24:257-61 (2000); Cavazzana-Calvo et al., Science, 288:669 (2000); and Blaese et al., Science, 270: 475 (1995); Kantoff, et al., J. Exp. Med. 166:219 (1987).

Any suitable gene therapy methods may be used for the purposes of the present invention. Generally, a nucleic acid encoding a desirable protein (e.g., one selected from any of the tables) is incorporated into a suitable expression vector and is operably linked to a promoter in the vector. Suitable promoters include but are not limited to viral transcription promoters derived from adenovirus, simian virus 40 (SV40) (e.g., the early and late promoters of SV40), Rous sarcoma virus (RSV), and cytomegalovirus (CMV) (e.g., CMV immediate-early promoter), human immunodeficiency virus (HIV) (e.g., long terminal repeat (LTR)), vaccinia virus (e.g., 7.5K promoter), and herpes simplex virus (HSV) (e.g., thymidine kinase promoter). Where tissue-specific expression of the exogenous gene is desirable, tissue-specific promoters may be operably linked to the exogenous gene. In addition, selection markers may also be included in the vector for purposes of selecting, in vitro, those cells that contain the exogenous gene. Various selection markers known in the art may be used including, but not limited to, e.g., genes conferring resistance to neomycin, hygromycin, zeocin, and the like.

In one embodiment, the exogenous nucleic acid (gene) is incorporated into a plasmid DNA vector. Many commercially available expression vectors may be useful for the present invention, including, e.g., pCEP4, pcDNAI, pIND, pSecTag2, pVAX1, pcDNA3.1, and pBI-EGFP, and pDisplay.

Various viral vectors may also be used. Typically, in a viral vector, the viral genome is engineered to eliminate the disease-causing capability of the virus, e.g., the ability to replicate in the host cells. The exogenous nucleic acid to be introduced into cells or tissue in vitro or in a patient may be incorporated into the engineered viral genome, e.g., by inserting it into a viral gene that is non-essential to the viral infectivity. Viral vectors are convenient to use as they can be easily introduced into cells, tissues and patients by way of infection. Once in the host cell, the recombinant virus typically is integrated into the genome of the host cell. In rare instances, the recombinant virus may also replicate and remain as extrachromosomal elements.

A large number of retroviral vectors have been developed for gene therapy. These include vectors derived from oncoretroviruses (e.g., MLV), lentiviruses (e.g., HIV and SIV) and other retroviruses. For example, gene therapy vectors have been developed based on murine leukemia virus (See, Cepko, et al., Cell, 37:1053-1062 (1984), Cone and Mulligan, Proc. Natl. Acad. Sci. U.S.A., 81:6349-6353 (1984)), mouse mammary tumor virus (See, Salmons et al., Biochem. Biophys. Res. Commun., 159:1191-1198 (1984)), gibbon ape leukemia virus (See, Miller et al., J. Virology, 65:2220-2224 (1991)), HIV, (See Shimada et al., J. Clin. Invest., 88:1043-1047 (1991)), and avian retroviruses (See Cosset et al., J. Virology, 64:1070-1078 (1990)). In addition, various retroviral vectors are also described in U.S. Pat. Nos. 6,168,916; 6,140,111; 6,096,534; 5,985,655; 5,911,983; 4,980,286; and 4,868,116, all of which are incorporated herein by reference.

Adeno-associated virus (AAV) vectors have been successfully tested in clinical trials. See e.g., Kay et al., Nature Genet. 24:257-61 (2000). AAV is a naturally occurring defective virus that requires other viruses such as adenoviruses or herpes viruses as helper viruses. See Muzyczka, Curr. Top. Microbiol. Immun., 158:97 (1992). A recombinant AAV virus useful as a gene therapy vector is disclosed in U.S. Pat. No. 6,153,436, which is incorporated herein by reference.

Adenoviral vectors can also be useful for purposes of gene therapy in accordance with the present invention. For example, U.S. Pat. No. 6,001,816 discloses an adenoviral vector, which is used to deliver a leptin gene intravenously to a mammal to treat obesity. Other recombinant adenoviral vectors may also be used, which include those disclosed in U.S. Pat. Nos. 6,171,855; 6,140,087; 6,063,622; 6,033,908; and 5,932,210, and Rosenfeld et al., Science, 252:431-434 (1991); and Rosenfeld et al., Cell, 68:143-155 (1992).

Other useful viral vectors include recombinant hepatitis viral vectors (See, e.g., U.S. Pat. No. 5,981,274), and recombinant entomopox vectors (See, e.g., U.S. Pat. Nos. 5,721,352 and 5,753,258).

Other non-traditional vectors may also be used for purposes of this invention. For example, International Publication No. WO 94/18834 discloses a method of delivering DNA into mammalian cells by conjugating the DNA to be delivered with a polyelectrolyte to form a complex. The complex may be microinjected into or taken up by cells.

The exogenous gene fragment or plasmid DNA vector containing the exogenous gene may also be introduced into cells by way of receptor-mediated endocytosis. See e.g., U.S. Pat. No. 6,090,619; Wu and Wu, J. Biol. Chem., 263:14621 (1988); Curiel et al., Proc. Natl. Acad. Sci. USA, 88:8850 (1991). For example, U.S. Pat. No. 6,083,741 discloses introducing an exogenous nucleic acid into mammalian cells by associating the nucleic acid to a polycation moiety (e.g., poly-L-lysine having 3-100 lysine residues), which is itself coupled to an integrin receptor binding moiety (e.g., a cyclic peptide having the sequence Arg-Gly-Asp).

Alternatively, the exogenous nucleic acid or vectors containing it can also be delivered into cells via amphiphiles. See e.g., U.S. Pat. No. 6,071,890. Typically, the exogenous nucleic acid or a vector containing the nucleic acid forms a complex with the cationic amphiphile. Mammalian cells contacted with the complex can readily take it up.

The exogenous gene can be introduced into cells or tissue in vitro or in a patient for purposes of gene therapy by various methods known in the art. For example, the exogenous gene sequences alone or in a conjugated or complex form described above, or incorporated into viral or DNA vectors, may be administered directly by injection into an appropriate tissue or organ of a patient. Alternatively, catheters or like devices may be used to deliver exogenous gene sequences, complexes, or vectors into a target organ or tissue. Suitable catheters are disclosed in, e.g., U.S. Pat. Nos. 4,186,745; 5,397,307; 5,547,472; 5,674,192; and 6,129,705, all of which are incorporated herein by reference.

In addition, the exogenous gene or vectors containing the gene can be introduced into isolated cells using any known techniques such as calcium phosphate precipitation, microinjection, lipofection, electroporation, biolystics, receptor-mediated endocytosis, and the like. Cells expressing the exogenous gene may be selected and redelivered back to the patient by, e.g., injection or cell transplantation. The appropriate amount of cells delivered to a patient will vary with patient conditions, and desired effect, which can be determined by a skilled artisan. See e.g., U.S. Pat. Nos. 6,054,288; 6,048,524; and 6,048,729. Preferably, the cells used are autologous, i.e., cells obtained from the patient being treated.

### 6.4. Small Organic Compounds

Diseases or disorders in cells or tissue in vitro, or in a patient, associated with the decreased concentration or activity of a protein complex of the present invention, or an individual protein constituent of a protein complex identified in accordance with the present invention, can also be ameliorated by administering to the patient a compound identified by the methods described in Sections 5.3.1.4, 5.2, and Section 5.4, which is capable of modulating the functions or intracellular levels of the protein complex or a constituent protein, e.g., by triggering or initiating, enhancing or stabilizing protein-protein interaction between the interacting protein members of the protein complex, or the mutant forms of such interacting protein members found in the patient.

### 7. Cell and Animal Models

In another aspect of the present invention, cell and animal models are provided in which one or more of the constituent proteins of the interacting pairs of proteins described in the tables, exhibit aberrant function, activity, or concentration when compared with wild type cells and animals (e.g., increased or decreased concentration, altered interactions between protein complex constituents due to mutations in interaction domains, and/or altered distribution or localization of the proteins in organs, tissues, cells, or cellular compartments). Such cell and animal models are useful tools for studying cellular functions and biological processes associated with the proteins identified in the tables. Such cell and animal models are also useful tools for studying disorders and diseases associated with the proteins identified in the tables, and for testing various methods for modulating the cellular functions, and for treating the diseases and disorders, associated with aberrations in these proteins. For example, a cell or animal model may be used to determine if APOA1 exhibits aberrant function, activity, or concentration when compared with wild type cells or animals. In another example, a cell or animal model may be used to determine if PRA1 exhibits aberrant function, activity, or concentration when compared with wild type cells or animals.

### 7.1. Cell Models

Cell models having an aberrant form of one or more of the proteins or protein complexes identified in the tables are provided in accordance with the present invention.

The cell models may be established by isolating, from a patient, cells having an aberrant form of one or more of the protein complexes of the present invention. The isolated cells may be cultured in vitro as a primary cell culture. Alternatively, the cells obtained from the primary cell culture or directly from the patient may be immortalized to establish a human cell line. Any methods for constructing immortalized human cell lines may be used in this respect. See generally Yeager and Reddel, Curr. Opini. Biotech., 10:465-469 (1999). For example, the human cells may be immortalized by transfection of plasmids expressing the SV40 early region genes (See e.g., Jha et al., Exp. Cell Res., 245:1-7 (1998)), introduction of the HPV E6 and E7 oncogenes (See e.g., Reznikoff et al., Genes Dev., 8:2227-2240 (1994)), and infection with Epstein-Barr virus (See e.g., Tahara et al., Oncogene, 15:1911-1920 (1997)). Alternatively, the human cells may be immortalized by recombinantly expressing the gene for the human telomerase catalytic subunit hTERT in the human cells. See Bodnar et al., Science, 279:349-352 (1998).

In alternative embodiments, cell models are provided by recombinantly manipulating appropriate host cells. The host cells may be bacteria cells, yeast cells, insect cells, plant cells, animal cells, and the like. Preferably, the cells are derived from mammals, most preferably humans. The host cells may be obtained directly from an individual, or a primary cell culture, or preferably an immortal stable human cell line. In a preferred embodiment, human embryonic stem cells or pluripotent cell lines derived from human stem cells are used as host cells. Methods for obtaining such cells are disclosed in, e.g., Shamblott, et al., Proc. Natl. Acad. Sci. USA, 95:13726-13731 (1998) and Thomson et al., Science, 282:1145-1147 (1998).

In one embodiment, a cell model is provided by recombinantly expressing one or more of the proteins or protein complexes identified in the tables in cells that do not normally express such protein complexes. For example, cells that do not contain a particular protein or protein complex may be engineered to express the protein or protein complex. In a specific embodiment, a particular human protein complex is expressed in non-human cells. The cell model may be prepared by introducing into host cells nucleic acids encoding all interacting protein members required for the formation of a particular protein complex, and expressing the protein members in the host cells. For this purpose, the recombinant expression methods described in Section 2.2 may be used. In addition, the methods for introducing nucleic acids into host cells disclosed in the context of gene therapy in Section 6.3.2 may also be used.

In another embodiment, a cell model over-expressing one or more of the proteins or protein complexes identified in the tables. The cell model may be established by increasing the expression level of one or more of the interacting protein members of the protein complexes. In a specific embodiment, all interacting protein members of a particular protein complex are over-expressed. The over-expression may be achieved by introducing into host cells exogenous nucleic acids encoding the proteins to be over-expressed, and selecting those cells that over-express the proteins. The expression of the exogenous nucleic acids may be transient or, preferably stable. The recombinant expression methods described in Section 2.2, and the methods for introducing nucleic acids into host cells disclosed in the context of gene therapy in Section 6.3.2 may be used. Alternatively, the gene activation method disclosed in U.S. Pat. No. 5,641,670 can be used. Any host cells may be employed for establishing the cell model. Preferably, human cells lacking a protein or protein complex to be over-expressed, or having a normal concentration of the protein or protein complex, are used as host cells. The host cells may be obtained directly from an individual, or a primary cell culture, or preferably a stable immortal human cell line. In a preferred embodiment, human embryonic stem cells or pluripotent cell lines derived from human stem cells are used as host cells. Methods for obtaining such cells are disclosed in, e.g., Shamblott, et al., Proc. Natl. Acad. Sci. USA, 95:13726-13731 (1998), and Thomson et al., Science, 282:1145-1147 (1998).

In yet another embodiment, a cell model expressing an abnormally low level of one or more of the proteins or protein complexes identified in the tables is provided. Typically, the cell model is established by genetically manipulating cells that express a normal and detectable level of a protein or protein complex identified in the tables. Generally the expression level of one or more of the interacting protein members of the protein complex is reduced by recombinant methods. In a specific embodiment, the expression of all interacting protein members of a particular protein complex is reduced. The reduced expression may be achieved by “knocking out” the genes encoding one or more interacting protein members. Alternatively, mutations that can cause reduced expression level (e.g., reduced transcription and/or translation efficiency, and decreased mRNA stability) may also be introduced into the gene by homologous recombination. A gene encoding a ribozyme, antisense, or siRNA compound specific to the mRNA encoding an interacting protein member may also be introduced into the host cells, preferably stably integrated into the genome of the host cells. In addition, a gene encoding an antibody or fragment thereof specific to an interacting protein member may also be introduced into the host cells. The recombinant expression methods described in Sections 2.2, 6.1 and 6.2 can all be used for purposes of manipulating the host cells.

In a specific embodiment, an siRNA compound specific to the mRNA encoding APOA1 is introduced into a host cell in order to decrease the expression level of APOA1. In another specific embodiment, an siRNA compound specific to the mRNA encoding PRA1 is introduced into a host cell in order to decrease the expression level of PRA1.

The present invention also contemplates a cell model provided by recombinant DNA techniques that exhibits aberrant interactions between the interacting protein members of a protein complex identified in the present invention. For example, variants of the interacting protein members of a particular protein complex exhibiting altered protein-protein interaction properties and the nucleic acid variants encoding such variant proteins may be obtained by random or site-directed mutagenesis in combination with a protein-protein interaction assay system, particularly the yeast two-hybrid system described in Section 5.3.1. Essentially, the genes encoding one or more interacting protein members of a particular protein complex may be subject to random or site-specific mutagenesis and the mutated gene sequences are used in yeast two-hybrid system to test the protein-protein interaction characteristics of the protein variants encoded by the gene variants. In this manner, variants of the interacting protein members of the protein complex may be identified that exhibit altered protein-protein interaction properties in forming the protein complex, e.g., increased or decreased binding affinity, and the like. The nucleic acid variants encoding such protein variants may be introduced into host cells by the methods described above, preferably into host cells that normally do not express the interacting proteins.

### 7.2. Cell-Based Assays

The cell models of the present invention containing an aberrant form of a protein or protein complex identified in the tables are useful in screening assays for identifying compounds useful in treating diseases and disorders involving angiogenesis, cell proliferation and transformation, intracellular vesicle trafficking, vacuolar protein sorting, formation of multivesicular bodies and endocytosis, inhibiting viral budding, suppress tumorigenesis and cell transformation, and reduce autoimmune response. In addition, the methods may also be used in the treatment or prevention of diseases and disorders such as viral infection, cancer and autoimmune diseases, cancer, AIDS, asthma, ischemia, stroke, autoimmune diseases, neurodegenerative diseases, inflammatory disorders, sepsis, and osteoporosis. The methods may also be used in the treatment or prevention of diseases and disorders such as cholesterol transport and lipid metabolism such as dementia such as Alzheimer's disease and cardiovascular diseases such as coronary artery disease, atherosclerosis, hypercholesterolemia, Tangier disease and amyloidosis, formation and maintenance of high density lipoprotein (HDL) microparticles, cholesterol and lipid transport and metabolism in cells, hyperlipidemia, hypercholesterolemia, and cardiovascular disease, abnormal cell proliferation (hyperproliferation or dysproliferation), keloid, liver cirrhosis, psoriasis, altered wound healing, cancer, especially cancers of the breast, ovary, colon, prostate, and lung, melanomas and cancers of hematopoietic origin, type 2 diabetes, obesity, hypertension, hypercholesterolemia, atherosclerosis, coronary artery disease, peripheral vascular disease, optic neuropathy, inflammatory response, amyloidosis, dementia, Alzheimer's disease, Tangier disease, Angelman syndrome, and specific viral infections. In addition, they may also be used in in vitro pre-clinical assays for testing compounds, such as those identified in the screening assays of the present invention.

For example, cells may be treated with compounds to be tested and assayed for the compound's activity. A variety of parameters relevant to particularly physiological disorders or diseases may be analyzed.

### 7.3. Transgenic Animals

In another aspect of the present invention, transgenic non-human animals are created expressing an aberrant form of one or more of the protein complexes of the present invention. Animals of any species may be used to generate the transgenic animal models, including but not limited to, mice, rats, hamsters, sheep, pigs, rabbits, guinea pigs, preferably non-human primates such as monkeys, chimpanzees, baboons, and the like.

In one embodiment, transgenic animals are made to over-express one or more protein complexes formed from a first protein, which is any one of the proteins described in the tables, or a derivative, fragment or homologue thereof (including the animal counterpart of the first protein, i.e., an orthologue) and a second protein, which is any of the proteins described in the tables that interacts with the first protein, or derivatives, fragments or homologues thereof (including orthologues). Over-expression may be directed in a tissue or cell type that normally expresses animal counterparts of such protein complexes. Consequently, the concentration of the protein complex(es) will be elevated to higher levels than normal. Alternatively, the one or more protein complexes are expressed in tissues or cells that do not normally express such proteins and hence do not normally contain the protein complexes of the present invention. In a specific embodiment, a first protein, which is any one of the proteins described in the tables which is a human protein and a second protein, which is any of the proteins described in the tables that interacts with the first protein and is a human protein, are expressed in the transgenic animals.

To achieve over-expression in transgenic animals, the transgenic animals are made such that they contain and express exogenous, orthologous genes encoding a first protein, which is any of the proteins identified in the tables or a homologue, derivative or mutant form thereof, and one or more second proteins, which are any of the proteins described in the tables that interact with the first protein, or homologues, derivatives or mutant forms thereof. Preferably, the exogenous genes are human genes. Such exogenous genes may be operably linked to a native or non-native promoter, preferably a non-native promoter. For example, an exogenous gene encoding one of the proteins described in the tables may be operably linked to a promoter that is not the native promoter of that protein. If the expression of the exogenous gene is desired to be limited to a particular tissue, an appropriate tissue-specific promoter may be used.

Over-expression may also be achieved by manipulating the native promoter to create mutations that lead to gene over-expression, or by a gene activation method such as that disclosed in U.S. Pat. No. 5,641,670 as described above.

In another embodiment, the transgenic animal expresses an abnormally low concentration of the complex comprising at least one of the interacting pairs of proteins described in the tables. In a specific embodiment, the transgenic animal is a “knockout” animal wherein the endogenous gene encoding the animal orthologue of a first protein, which is any of the proteins described in the tables, and/or an endogenous gene encoding an animal orthologue of a second protein, which is any of the proteins identified in the tables that interacts with the first protein, are knocked out. In a specific embodiment, the expression of the animal orthologues of both the first and second proteins are reduced or knocked out. The reduced expression may be achieved by knocking out the genes encoding one or both interacting protein members, typically by homologous recombination. Alternatively, mutations that can cause reduced expression (e.g., reduced transcription and/or translation efficiency, or decreased mRNA stability) may also be introduced into the endogenous genes by homologous recombination. Genes encoding ribozymes or antisense compounds specific to the mRNAs encoding the interacting protein members may also be introduced into the transgenic animal. In addition, genes encoding antibodies or fragments thereof specific to the interacting protein members may also be introduced into the transgenic animal.

In an alternate embodiment, transgenic animals are made in which the endogenous genes encoding the animal orthologues of any of the proteins described in the tables are replaced with orthologous human genes.

In yet another embodiment, the transgenic animal of this invention expresses specific mutant forms of any of the proteins described in the tables that exhibit aberrant interactions. For this purpose, variants of any of the proteins described in the tables exhibiting altered protein-protein interaction properties, and the nucleic acid variants encoding such variant proteins, may be obtained by random or site-specific mutagenesis in combination with a protein-protein interaction assay system, particularly the yeast two-hybrid system described in Section 5.3.1. For example, variants of APOA1 and PRA1 exhibiting increased, decreased or abolished binding affinity to each other may be identified and isolated. The transgenic animal of the present invention may be made to express such protein variants by modifying the endogenous genes. Alternatively, the nucleic acid variants may be introduced exogenously into the transgenic animal genome to express the protein variants therein. In a specific embodiment, the exogenous nucleic acid variants are derived from orthologous human genes and the corresponding endogenous genes are knocked out.

Any techniques known in the art for making transgenic animals may be used for purposes of the present invention. For example, the transgenic animals of the present invention may be provided by methods described in, e.g., Jaenisch, Science, 240:1468-1474 (1988); Capecchi, et al., Science, 244:1288-1291 (1989); Hasty et al., Nature, 350:243 (1991); Shinkai et al., Cell, 68:855 (1992); Mombaerts et al., Cell, 68:869 (1992); Philpott et al., Science, 256:1448 (1992); Snouwaert et al., Science, 257:1083 (1992); Donehower et al., Nature, 356:215 (1992); Hogan et al., Manipulating the Mouse Embryo; A Laboratory Manual, 2^{nd} edition, Cold Spring Harbor Laboratory Press, 1994; and U.S. Pat. Nos. 4,873,191; 5,800,998; 5,891,628, all of which are incorporated herein by reference.

Generally, the founder lines may be established by introducing appropriate exogenous nucleic acids into, or modifying an endogenous gene in, germ lines, embryonic stem cells, embryos, or sperm which are then used in producing a transgenic animal. The gene introduction may be conducted by various methods including those described in Sections 2.2, 6.1 and 6.2. See also, Van der Putten et al., Proc. Natl. Acad. Sci. USA, 82:6148-6152 (1985); Thompson et al., Cell, 56:313-321 (1989); Lo, Mol. Cell. Biol., 3:1803-1814 (1983); Gordon, Trangenic Animals, Intl. Rev. Cytol. 115:171-229 (1989); and Lavitrano et al., Cell, 57:717-723 (1989). In a specific embodiment, the exogenous gene is incorporated into an appropriate vector, such as those described in Sections 2.2 and 6.2, and is transformed into embryonic stem (ES) cells. The transformed ES cells are then injected into a blastocyst. The blastocyst with the transformed ES cells is then implanted into a surrogate mother animal. In this manner, a chimeric founder line animal containing the exogenous nucleic acid (transgene) may be produced.

Preferably, site-specific recombination is employed to integrate the exogenous gene into a specific predetermined site in the animal genome, or to replace an endogenous gene or a portion thereof with the exogenous sequence. Various site-specific recombination systems may be used including those disclosed in Sauer, Curr. Opin. Biotechnol., 5:521-527 (1994); Capecchi, et al., Science, 244:1288-1291 (1989); and Gu et al., Science, 265:103-106 (1994). Specifically, the Cre/lox site-specific recombination system known in the art may be conveniently used which employs the bacteriophage P1 protein Cre recombinase and its recognition sequence loxP. See Rajewsky et al., J. Clin. Invest., 98:600-603 (1996); Sauer, Methods, 14:381-392 (1998); Gu et al., Cell, 73:1155-1164 (1993); Araki et al., Proc. Natl. Acad. Sci. USA, 92:160-164 (1995); Lakso et al., Proc. Natl. Acad. Sci. USA, 89:6232-6236 (1992); and Orban et al., Proc. Natl. Acad. Sci. USA, 89:6861-6865 (1992).

The transgenic animals of the present invention may be transgenic animals that carry a transgene in all cells or mosaic transgenic animals carrying a transgene only in certain cells, e.g., somatic cells. The transgenic animals may have a single copy or multiple copies of a particular transgene.

The founder transgenic animals thus produced may be bred to produce various offsprings. For example, they can be inbred, outbred, and crossbred to establish homozygous lines, heterozygous lines, and compound homozygous or heterozygous lines.

### 8. Pharmaceutical Compositions and Formulations

In another aspect of the present invention, pharmaceutical compositions are also provided containing one or more of the therapeutic agents provided in the present invention as described in Section 6. The compositions are prepared as a pharmaceutical formulation suitable for administration into a patient. Accordingly, the present invention also extends to pharmaceutical compositions, medicaments, drugs or other compositions containing one or more of the therapeutic agent in accordance with the present invention.

For example, such therapeutic agents include, but are not limited to, (1) small organic compounds selected based on the screening methods of the present invention capable of interfering with the interaction between a first protein which is any of the interacting proteins described in the tables and a second protein which is any of the proteins identified in the tables that interacts with the first protein, (2) antisense compounds specifically hybridizable to nucleic acids (gene or mRNA) encoding the first protein (3) antisense compounds specific to the gene or mRNA encoding the second protein, (4) ribozyme compounds specific to nucleic acids (gene or mRNA) encoding the first protein, (5) ribozyme compounds specific to the gene or mRNA encoding the second protein, (6) antibodies immunoreactive with the first protein or the second protein, (7) antibodies selectively immunoreactive with a protein complex of the present invention, (8) small organic compounds capable of binding a protein complex of the present invention, (9) small peptide compounds as described above (optionally linked to a transporter) capable of interacting with the first protein or the second protein, (10) nucleic acids encoding the antibodies or peptides, (11) siRNA compounds specific to the gene or mRNA encoding the first protein, (12) siRNA compounds specific to the gene or mRNA encoding the second protein, etc.

The compositions are prepared as a pharmaceutical formulation suitable for administration into a patient. Accordingly, the present invention also extends to pharmaceutical compositions, medicaments, drugs or other compositions containing one or more of the therapeutic agent in accordance with the present invention.

In the pharmaceutical composition, an active compound identified in accordance

with the present invention can be in any pharmaceutically acceptable salt form. As used herein, the term “pharmaceutically acceptable salts” refers to the relatively non-toxic, organic or inorganic salts of the compounds of the present invention, including inorganic or organic acid addition salts of the compound. Examples of such salts include, but are not limited to, hydrochloride salts, sulfate salts, bisulfate salts, borate salts, nitrate salts, acetate salts, phosphate salts, hydrobromide salts, laurylsulfonate salts, glucoheptonate salts, oxalate salts, oleate salts, laurate salts, stearate salts, palmitate salts, valerate salts, benzoate salts, naphthylate salts, mesylate salts, tosylate salts, citrate salts, lactate salts, maleate salts, succinate salts, tartrate salts, fumarate salts, and the like. See, e.g., Berge, et al., J. Pharm. Sci., 66:1-19 (1977).

For oral delivery, the active compounds can be incorporated into a formulation that includes pharmaceutically acceptable carriers such as binders (e.g., gelatin, cellulose, gum tragacanth), excipients (e.g., starch, lactose), lubricants (e.g., magnesium stearate, silicon dioxide), disintegrating agents (e.g., alginate, Primogel, and corn starch), and sweetening or flavoring agents (e.g., glucose, sucrose, saccharin, methyl salicylate, and peppermint). The formulation can be orally delivered in the form of enclosed gelatin capsules or compressed tablets. Capsules and tablets can be prepared in any conventional techniques. The capsules and tablets can also be coated with various coatings known in the art to modify the flavors, tastes, colors, and shapes of the capsules and tablets. In addition, liquid carriers such as fatty oil can also be included in capsules.

Suitable oral formulations can also be in the form of suspension, syrup, chewing gum, wafer, elixir, and the like. If desired, conventional agents for modifying flavors, tastes, colors, and shapes of the special forms can also be included. In addition, for convenient administration by enteral feeding tube in patients unable to swallow, the active compounds can be dissolved in an acceptable lipophilic vegetable oil vehicle such as olive oil, corn oil and safflower oil.

The active compounds can also be administered parenterally in the form of solution or suspension, or in lyophilized form capable of conversion into a solution or suspension form before use. In such formulations, diluents or pharmaceutically acceptable carriers such as sterile water and physiological saline buffer can be used. Other conventional solvents, pH buffers, stabilizers, anti-bacterial agents, surfactants, and antioxidants can all be included. For example, useful components include sodium chloride, acetate, citrate or phosphate buffers, glycerin, dextrose, fixed oils, methyl parabens, polyethylene glycol, propylene glycol, sodium bisulfate, benzyl alcohol, ascorbic acid, and the like. The parenteral formulations can be stored in any conventional containers such as vials and ampoules.

Routes of topical administration include nasal, bucal, mucosal, rectal, or vaginal applications. For topical administration, the active compounds can be formulated into lotions, creams, ointments, gels, powders, pastes, sprays, suspensions, drops and aerosols. Thus, one or more thickening agents, humectants, and stabilizing agents can be included in the formulations. Examples of such agents include, but are not limited to, polyethylene glycol, sorbitol, xanthan gum, petrolatum, beeswax, or mineral oil, lanolin, squalene, and the like. A special form of topical administration is delivery by a transdermal patch. Methods for preparing transdermal patches are disclosed, e.g., in Brown, et al., Annual Review of Medicine, 39:221-229 (1988), which is incorporated herein by reference.

Subcutaneous implantation for sustained release of the active compounds may also be a suitable route of administration. This entails surgical procedures for implanting an active compound in any suitable formulation into a subcutaneous space, e.g., beneath the anterior abdominal wall. See, e.g., Wilson et al., J. Clin. Psych. 45:242-247 (1984). Hydrogels can be used as a carrier for the sustained release of the active compounds. Hydrogels are generally known in the art. They are typically made by crosslinking high molecular weight biocompatible polymers into a network that swells in water to form a gel like material. Preferably, hydrogels is biodegradable or biosorbable. For purposes of this invention, hydrogels made of polyethylene glycols, collagen, or poly(glycolic-co-L-lactic acid) may be useful. See, e.g., Phillips et al., J. Pharmaceut. Sci. 73:1718-1720 (1984).

The active compounds can also be conjugated, to a water soluble non-immunogenic non-peptidic high molecular weight polymer to form a polymer conjugate. For example, an active compound is covalently linked to polyethylene glycol to form a conjugate. Typically, such a conjugate exhibits improved solubility, stability, and reduced toxicity and immunogenicity. Thus, when administered to a patient, the active compound in the conjugate can have a longer half-life in the body, and exhibit better efficacy. See generally, Burnham, Am. J. Hosp. Pharm., 15:210-218 (1994). PEGylated proteins are currently being used in protein replacement therapies and for other therapeutic uses. For example, PEGylated interferon (PEG-INTRON A®) is clinically used for treating Hepatitis B. PEGylated adenosine deaminase (ADAGEN®) is being used to treat severe combined immunodeficiency disease (SCIDS). PEGylated L-asparaginase (ONCAPSPAR®) is being used to treat acute lymphoblastic leukemia (ALL). It is preferred that the covalent linkage between the polymer and the active compound and/or the polymer itself is hydrolytically degradable under physiological conditions. Such conjugates known as “prodrugs” can readily release the active compound inside the body. Controlled release of an active compound can also be achieved by incorporating the active ingredient into microcapsules, nanocapsules, or hydrogels generally known in the art.

Liposomes can also be used as carriers for the active compounds of the present invention. Liposomes are micelles made of various lipids such as cholesterol, phospholipids, fatty acids, and derivatives thereof. Various modified lipids can also be used. Liposomes can reduce the toxicity of the active compounds, and increase their stability. Methods for preparing liposomal suspensions containing active ingredients therein are generally known in the art. See, e.g., U.S. Pat. No. 4,522,811; Prescott, Ed., Methods in Cell Biology, Volume XIV, Academic Press, New York, N.Y. (1976).

The active compounds can also be administered in combination with another active agent that synergistically treats or prevents the same symptoms or is effective for another disease or symptom in the patient treated so long as the other active agent does not interfere with or adversely affect the effects of the active compounds of this invention. Such other active agents include but are not limited to anti-inflammation agents, antiviral agents, antibiotics, antifungal agents, antithrombotic agents, cardiovascular drugs, cholesterol lowering agents, anti-cancer drugs, hypertension drugs, and the like.

Generally, the toxicity profile and therapeutic efficacy of the therapeutic agents can be determined by standard pharmaceutical procedures in cell models or animal models, e.g., those provided in Section 7. As is known in the art, the LD₅₀ represents the dose lethal to about 50% of a tested population. The ED₅₀ is a parameter indicating the dose therapeutically effective in about 50% of a tested population. Both LD₅₀ and ED₅₀ can be determined in cell models and animal models. In addition, the IC₅₀ may also be obtained in cell models and animal models, which stands for the circulating plasma concentration that is effective in achieving about 50% of the maximal inhibition of the symptoms of a disease or disorder. Such data may be used in designing a dosage range for clinical trials in humans. Typically, as will be apparent to skilled artisans, the dosage range for human use should be designed such that the range centers around the ED₅₀ and/or IC₅₀, but significantly below the LD₅₀ obtained from cell or animal models.

It will be apparent to skilled artisans that therapeutically effective amount for each active compound to be included in a pharmaceutical composition of the present invention can vary with factors including but not limited to the activity of the compound used, stability of the active compound in the patient's body, the severity of the conditions to be alleviated, the total weight of the patient treated, the route of administration, the ease of absorption, distribution, and excretion of the active compound by the body, the age and sensitivity of the patient to be treated, and the like. The amount of administration can also be adjusted as the various factors change over time.

### EXAMPLES

### 1. Yeast Two-Hybrid System

The principles and methods of the yeast two-hybrid system have been described in detail in The Yeast Two-Hybrid System, Bartel and Fields, eds., pages 183-196, Oxford University Press, New York, N.Y., 1997. The following is thus a description of the particular procedure that we used to identify the interactions of the present invention.

The cDNA encoding the bait protein was generated by PCR from cDNA prepared from a desired tissue. The cDNA product was then introduced by recombination into the yeast expression vector pGBT.Q, which is a close derivative of pGBT.C (See Bartel et al., Nat Genet., 12:72-77 (1996)) in which the polylinker site has been modified to include M13 sequencing sites. The new construct was selected directly in the yeast strain PNY200 for its ability to drive tryptophane synthesis (genotype of this strain: MATαtrp1-901 leu2-3,112 ura3-52 his3-200 ade2 gal4Δgal80). In these yeast cells, the bait was produced as a C-terminal fusion protein with the DNA binding domain of the transcription factor Gal4 (amino acids 1 to 147). Prey libraries were transformed into the yeast strain BK100 (genotype of this strain: MATa trp1-901 leu2-3,112 ura3-52 his3-200 gal4Δgal80LYS2::GAL-HIS3 GAL2-ADE2 met2::GAL7-lacZ), and selected for the ability to drive leucine synthesis. In these yeast cells, each cDNA was expressed as a fusion protein with the transcription activation domain of the transcription factor Gal4 (amino acids 768 to 881) and a 9 amino acid hemagglutinin epitope tag. PNY200 cells (MATα mating type), expressing the bait, were then mated with BK100 cells (MATa mating type), expressing prey proteins from a prey library. The resulting diploid yeast cells expressing proteins interacting with the bait protein were selected for the ability to synthesize tryptophan, leucine, histidine, and adenine. DNA was prepared from each clone, transformed by electroporation into E. coli strain KC8 (Clontech KC8 electrocompetent cells, Catalog No. C2023-1), and the cells were selected on ampicillin-containing plates in the absence of either tryptophane (selection for the bait plasmid) or leucine (selection for the library plasmid). DNA for both plasmids was prepared and sequenced by the dideoxynucleotide chain termination method. The identity of the bait cDNA insert was confirmed and the cDNA insert from the prey library plasmid was identified using the BLAST program to search against public nucleotide and protein databases. Plasmids from the prey library were then individually transformed into yeast cells together with a plasmid driving the synthesis of lamin and 5 other test proteins, respectively, fused to the Gal4 DNA binding domain. Clones that gave a positive signal in the β-galactosidase assay were considered false-positives and discarded. Plasmids for the remaining clones were transformed into yeast cells together with the original bait plasmid. Clones that gave a positive signal in the β-galactosidase assay were considered true positives.

Bait sequences indicated in the tables were used in the yeast two-hybrid system described above. The isolated prey sequences are summarized in the tables. The GenBank® Accession Nos. for the bait and prey proteins are also provided in the tables, upon which the bait and prey sequences are aligned.

### 2. Production of Antibodies Selectively Immunoreactive with Protein Complex

The APOA1-interacting region of PRA1 and the PRA1-interacting region of APOA1 are indicated in the tables above. Both regions, or fragments thereof, are recombinantly-expressed in E. coli. and isolated and purified. Mixing the two purified interacting regions forms a protein complex. A protein complex is also formed by mixing recombinantly expressed intact complete APOA1 and PRA1. The two protein complexes are used as antigens in immunizing a mouse. mRNA is isolated from the immunized mouse spleen cells, and first-strand cDNA is synthesized using the mRNA as a template. The V_{H} and V_{K} genes are amplified from the thus synthesized cDNAs by PCR using appropriate primers.

The amplified V_{H} and V_{K} genes are ligated together and subcloned into a phagemid vector for the construction of a phage display library. E. coli. cells are transformed with the ligation mixtures, and thus a phage display library is established. Alternatively, the ligated V_{H} and Vₖ genes are subcloned into a vector suitable for ribosome display in which the V_{H}-Vₖ sequence is under the control of a T7 promoter. See Schaffitzel et al., J. Immun. Meth., 231:119-135 (1999).

The libraries are screened for their ability to bind APOA1-PRA1 complex and APOA1 or PRA1, alone. Several rounds of screening are generally performed. Clones corresponding to scFv fragments that bind the APOA1-PRA1 complex, but not isolated APOA1 or PRA1 are selected and purified. A single purified clone is used to prepare an antibody selectively immunoreactive with the complex comprising APOA1 and PRA1. The antibody is then verified by an immunochemistry method such as RIA and ELISA.

In addition, the clones corresponding to scFv fragments that bind the complex comprising APOA1 and PRA1, and also bind isolated APOA1 and/or PRA1 may be selected. The scFv genes in the clones are diversified by mutagenesis methods such as oligonucleotide-directed mutagenesis, error-prone PCR (See Lin-Goerke et al., Biotechniques, 23:409 (1997)), dNTP analogues (See Zaccolo et al., J. Mol. Biol., 255:589 (1996)), and other methods. The diversified clones are further screened in phage display or ribosome display libraries. In this manner, scFv fragments selectively immunoreactive with the complex comprising APOA1 and PRA1 may be obtained.

### 3. Yeast Screen to Identify Small Molecule Inhibitors of the Interaction Between APOA1 and PRA1

Beta-galactosidase is used as a reporter enzyme to signal the interaction between yeast two-hybrid protein pairs expressed from plasmids in Saccharomyces cerevisiae. Yeast strain MY209 (ade2 his3 leu2 trp1 cyh2 ura3::GAL1p-lacZgal4 gal80 lys2::GAL1p-HIS3) bearing one plasmid with the genotype of LEU2 CEN4 ARS1 ADH1p-SV40NLS-GAL4 (768-881)—PRA1-PGK1t AmpR ColE1_ori, and another plasmid having a genotype of TRP1 CEN4 ARS ADH1p-GAL4(1-147)-APOA1-ADH1t AmpR ColE1_ori is cultured in synthetic complete media lacking leucine and tryptophan (SC-Leu-Trp) overnight at 30° C. The APOA1 and PRA1 nucleic acids in the plasmids can code for the full-length APOA1 and PRA1 proteins, respectively, or fragments thereof. This culture is diluted to 0.01 OD₆₃₀ units/ml using SC-Leu-Trp media. The diluted MY209 culture is dispensed into 96-well microplates. Compounds from a library of small molecules are added to the microplates; the final concentration of test compounds is approximately 60 μM. The assay plates are incubated at 30° C. overnight.

The following day an aliquot of concentrated substrate/lysis buffer is added to each well and the plates incubated at 37° C. for 1-2 hours. At an appropriate time an aliquot of stop solution is added to each well to halt the beta-galactosidase reaction. For all microplates an absorbance reading is obtained to assay the generation of product from the enzyme substrate. The presence of putative inhibitors of the interaction between APOA1 and PRA 1 results in inhibition of the beta-galactosidase signal generated by MY209. Additional testing eliminates compounds that decreased expression of beta-galactosidase by affecting yeast cell growth and non-specific inhibitors that affected the beta-galactosidase signal generated by the interaction of an unrelated protein pair.

Once a hit, i.e., a compound which inhibits the interaction between the interacting proteins, is obtained, the compound is identified and subjected to further testing wherein the compounds are assayed at several concentrations to determine an IC₅₀ value, this being the concentration of the compound at which the signal seen in the two-hybrid assay described in this Example is 50% of the signal seen in the absence of the inhibitor.

### 4. Enzyme-Linked Immunosorbent Assay (ELISA)

pGEX5X-2 (Amersham Biosciences; Uppsala, Sweden) is used for the expression of a GST-PRA1 fusion protein. The pGEX5X-2-PRA1 construct is transfected into Escherichia coli strain DH5a (Invitrogen; Carlsbad, Calif.) and fusion protein is prepared by inducing log phase cells (O.D. 595=0.4) with 0.2 mM isopropyl-β-D-thiogalactopyranoside (IPTG). Cultures are harvested after approximately 4 hours of induction, and cells pelleted by centrifugation. Cell pellets are resuspended in lysis buffer (1% nonidet P-40 [NP-40], 150 mM NaCl, 10 mM Tris pH 7.4, 1 mM ABESF [4-(2-aminoethyl) benzenesulfonyl fluoride]), lysed by sonication and the lysate cleared of insoluble materials by centrifugation. Cleared lysate is incubated with Glutathione Sepharose beads (Amersham Biosciences; Uppsala, Sweden) followed by thorough washing with lysis buffer. The GST-PRA1 fusion protein is then eluted from the beads with 5 mM reduced glutathione. Eluted protein is dialyzed against phosphate buffer saline (PBS) to remove the reduced glutathione.

A stable Drosophila Schneider 2 (S2) myc-APOA1 expression cell line is generated by transfecting S2 cells with pCoHygro (Invitrogen; Carlsbad, Calif.) and an expression vector that directs the expression of the myc-APOA1 fusion protein. Briefly, S2 cells are washed and re-suspended in serum free Express Five media (Invitrogen; Carlsbad, Calif.). Plasmid/liposome complexes are then added (NovaFECTOR, Venn Nova; Pompano Beach, Fla.) and allowed to incubate with cells for 12 hours under standard growth conditions (room temperature, no CO₂ buffering). Following this incubation period fetal bovine serum is added to a final concentration of 20% and cells are allowed to recover for 24 hours. The media is replaced and cells are grown for an additional 24 hours. Transfected cells are then selected in 350 μg/ml hygromycin for three weeks. Expression of myc-APOA1 is confirmed by Western blotting. This cell line is referred to as S2-myc-APOA1.

GST-PRA1 fusion protein is immobilized to wells of an ELISA plate as follows: Nunc Maxisorb 96 well ELISA plates (Nalge Nunc International; Rochester, N.Y.) are incubated with 100 μl of 10 μg/ml of GST-PRA1 in 50 mM carbonate buffer (pH 9.6) and stored overnight at 40 Celsius. This plate is referred to as the ELISA plate.

A compound dilution plate is generated in the following manner. In a 96 well polypropylene plate (Greiner, Germany) 50 μl of DMSO is pipetted into columns 2-12. In the same polypropylene plate pipette, 10 μl of each compound being tested for its ability to modulate protein-protein interactions is plated in the wells of column 1 followed by 90 μl of DMSO (final volume of 1001). Compounds selected from primary screens or from virtual screening, or designed based on the primary screen hits are then serially diluted by removing 50 μl from column 1 and transferring it to column 2 (50:50 dilution). Serial dilutions are continued until column 10. This plate is termed the compound dilution plate.

Next, 12 μl from each well of the compound dilution plate is transferred into its corresponding well in a new polypropylene plate. 108 μl of S2-myc-APOA1-containing lysate (1×10⁶ cell equivalents/ml) in phosphate buffered saline is added to all wells of columns 1-11. 108 μl of phosphate buffered saline without lysate is added into all wells of column 12. The plate is then mixed on a shaker for 15 minutes. This plate is referred to as the compound preincubation plate.

The ELISA plate is emptied of its contents and 400 μl of Superblock (Pierce Endogen; Rockford, Ill.) is added to all the wells and allowed to sit for 1 hour at room temperature. 100 μl from all columns of the compound preincubation plate are transferred into the corresponding wells of the ELISA binding plate. The plate is then covered and allowed to incubate for 1.5 hours room temperature.

The interaction of the myc-tagged APOA1 with the immobilized GST-PRA1 is detected by washing the ELISA plate followed by an incubation with 100 μl/well of 1 μg/ml of mouse anti-myc IgG (clone 9E10; Roche Applied Science; Indianapolis, Ind.) in phosphate buffered saline. After 1 hour at room temperature, the plates are washed with phosphate buffered saline and incubated with 100 μl/well of 250 ng/ml of goat anti-mouse IgG conjugated to horseradish peroxidase in phosphate buffer saline. Plates are then washed again with phosphate buffered saline and incubated with the fluorescent substrate solution Quantiblu (Pierce Endogen; Rockford, Ill.). Horseradish peroxidase activity is then measured by reading the plates in a fluorescent plate reader (325 nm excitation, 420 nm emission).

### 5. Effects of Antisense Inhibitors on Protein Expression

The effects of antisense inhibitors on protein expression can be measured by a variety of methods known in the art. A preferred method is to measure mRNA levels using real-time quantitative polymerase chain reaction (PCR) methods. Real-time PCR can be performed using the ABI PRISM™ 7700 Sequence Detection System according to the manufacturer's instructions. The ABI PRISM™ 7700 Sequence Detection System is available from PE-APPLIED Biosystems, Foster City, Calif.

Other methods of measuring mRNA levels may also be used to determine the effects of anitisense inhibitors on proteins. For example competitive PCR and Northern blot analysis are well known in the art and may be performed to determine mRNA levels. Specifically, methods of RNA isolation and Northern blot analysis may be performed according to Ausubel, F. M. et al., Current Protocols in Molecular Biology, Volume 1, pp. 4.1.1-4.2.9 and 4.5.1-4.5.3, John Wiley & Sons, Inc., 1993.

The effects of antisense inhibitors on protein expression may also be determined by measuring protein levels of the proteins of interest. Various methods known in the art may be used, such as immunoprecipitation, Western blot analysis, ELISA, or fluorescence-activated cell sorting (FACS). Antibodies to the proteins of interest are often commercially available, and may be found by such sources as the MSRS catalogue of antibodies (Aerie Corporation, Birmingham, Mich.). Antibodies can also be prepared through conventional antibody generation methods, such as found in Ausubel, F. M. et al., Current Protocols in Molecular Biology, Volume 2, pp. 11.12.1-11.12.9 and 11.4.1-11.11.5 John Wiley & Sons, Inc., 1997. Furthermore, immunoprecipitation analysis can be performed according to Ausubel, F. M. et al., Current Protocols in Molecular Biology, Volume 2, pp. 10.16.1-10.16.11, John Wiley & Sons, Inc., 1997, and ELISA can be performed according to Ausubel, F. M. et al., Current Protocols in Molecular Biology, Volume 2, pp. 11.1.1-11.2.22, John Wiley & Sons, Inc., 1997 or as described in Example 4, above.

Although the foregoing invention has been described in some detail by way of illustration and example for purposes of clarity of understanding, it will be obvious that certain changes and modifications may be practiced within the scope of the appended claims.

In various parts of this disclosure, certain publications or patents are discussed or cited. The mere discussion of, or reference to, such publications or patents is not intended as admission that they are prior art to the present invention.

What is claimed is:

1. An isolated protein complex comprising a first protein interacting with a second protein, said first protein being:
(a) a bait protein identified in any one of Tables 1 through 11,
(b) a homologue of (a) that interacts with said second protein and has an amino acid sequence at least 85% identical to that of (a),
(c) a fragment of (a) or (b) that interacts with said second protein, or
(d) a fusion protein comprising (a), (b), or (c); and
said second protein being:
(i) an interacting prey protein identified in the same table as said bait protein,
(ii) a homologue of (i) that interacts with said first protein and has an amino acid sequence at least 85% identical to that of (i),
(iii) a fragment of (i) or (ii) that interacts with said first protein, or
(iv) a fusion protein comprising (i), (ii), or (iii).

2. The isolated protein complex of claim 1 wherein said first protein and said second protein are both fusion proteins.

3. A method of making the isolated protein complex of claim 1 comprising:
providing said first protein and said second protein, and
contacting said first and second proteins under conditions that allow said first and second proteins to interact to form said isolated protein complex.

4. A method for selecting modulators of a protein complex of claim 1, comprising:
providing said first protein and said second protein;
contacting said first protein and said second protein in the presence and absence of a test compound; and
detecting the protein complex formed by the interaction between said first protein and said second protein in the presence and absence of said test compound.

5. The method of claim 4, wherein said detecting step comprises measuring the amount of the protein complex formed.

6. The method of claim 4, further comprising a step of generating a data set defining one or more selected test compounds, said data set being embodied in a transmittable form.

7. The method of claim 4, wherein at least one of said first and second proteins is a fusion protein having a detectable tag.

8. The method of claim 4, wherein said contacting step is conducted in a substantially cell free environment.

9. The method of claim 4, wherein the interaction between said first protein and said second protein occurs within a host cell.

10. The method of claim 9, wherein said host cell is a yeast cell.

11. A method for identifying modulators of an isolated protein complex of claim 1, comprising:
providing said isolated protein complex;
contacting said isolated protein complex with a test compound; and
determining whether the amount of said isolated protein complex is changed in the presence of said test compound, relative to the absence of said test compound.

12. A method for modulating, in a host cell, a protein complex of claim 1, comprising:
administering to said cell an siRNA or antisense oligonucleotide that causes the reduction of expression of said bait protein or said interacting prey protein.

13. A protein microarray comprising an isolated protein complex according to claim 1.

14. A method of treating abnormal cell proliferation, keloid, liver cirrhosis, psoriasis, altered wound healing, cancers of the breast, ovary, colon, prostate, and lung, melanomas and cancers of hematopoietic origin, type 2 diabetes, obesity, hypertension, hypercholesterolemia, atherosclerosis, coronary artery disease, peripheral vascular disease, optic neuropathy, inflammatory response, amyloidosis, dementia, Alzheimer's disease, Tangier disease, Angelman syndrome, or viral infections, comprising:
identifying a patient in need of treatment; and
administering to a patient in need of such treatment a compound that modulates the interaction between a first protein, which is a bait protein of one of Tables 1 through 11, and a second protein, which is the corresponding prey protein from said one of Tables 1 through 11.

15. The method of claim 14 wherein said compound that modulates the interaction between said first and second proteins binds to said first protein or to said second protein.

16. The method of claim 15 wherein said compound that modulates the interaction between said first and second proteins disrupts or interferes with the interaction of said first protein with said second protein.

17. A method of treating abnormal cell proliferation, keloid, liver cirrhosis, psoriasis, altered wound healing, cancers of the breast, ovary, colon, prostate, and lung, melanomas and cancers of hematopoietic origin, type 2 diabetes, obesity, hypertension, hypercholesterolemia, atherosclerosis, coronary artery disease, peripheral vascular disease, optic neuropathy, inflammatory response, amyloidosis, dementia, Alzheimer's disease, Tangier disease, Angelman syndrome, or viral infections, comprising:
identifying a patient in need of treatment; and
administering to a patient in need of such treatment an siRNA or antisense oligonucleotide that reduces the concentration of one of the protein complexes of Tables 1 through 11, by causing the reduction of expression of the bait protein or the interacting prey protein that interact to form said one of the protein complexes of Tables 1 through 11.

18. A method of detecting an alteration associated with a disease or disorder selected from abnormal cell proliferation, keloid, liver cirrhosis, psoriasis, altered wound healing, cancers of the breast, ovary, colon, prostate, and lung, melanomas and cancers of hematopoietic origin, type 2 diabetes, obesity, hypertension, hypercholesterolemia, atherosclerosis, coronary artery disease, peripheral vascular disease, optic neuropathy, inflammatory response, amyloidosis, dementia, Alzheimer's disease, Tangier disease, Angelman syndrome, or viral infections, comprising:
identifying a patient with said disease or disorder,
obtaining a sample from said patient, and
assaying said sample for an alteration in the nucleotide sequence of the gene or genes encoding a bait protein, an interacting prey protein, or both, identified in one of Tables 1 through 11, or an alteration in the level of expression of the gene or genes encoding a bait protein, an interacting prey protein, or both, identified in one of the Tables 1 through 11, as compared to patients without said disease or disorder;
wherein the detection of said alteration in said sample identifies said alteration as being associated with said disease or disorder.

19. The method of claim 18, wherein said alteration is an alteration in the nucleotide sequence of the gene or genes encoding a bait protein, an interacting prey protein, or both, identified in one of the Tables 1 through 11.

20. The method of claim 18, wherein said alteration is an alteration in the level of expression of a bait protein, an interacting prey protein, or both, identified in one of the Tables 1 through 11.

21. The method of claim 18 further comprising the step of determining if said alteration has been inherited.

22. A method of genotyping an individual with a disease or disorder selected from abnormal cell proliferation, keloid, liver cirrhosis, psoriasis, altered wound healing, cancers of the breast, ovary, colon, prostate, and lung, melanomas and cancers of hematopoietic origin, type 2 diabetes, obesity, hypertension, hypercholesterolemia, atherosclerosis, coronary artery disease, peripheral vascular disease, optic neuropathy, inflammatory response, amyloidosis, dementia, Alzheimer's disease, Tangier disease, Angelman syndrome, or viral infections, comprising:
identifying an individual with said disease or disorder, and
determining if said individual has alteration in the nucleotide sequence of the gene or genes encoding a bait protein, an interacting prey protein, or both, identified in one of the Tables 1 through 11, or an alteration in the level of expression of the gene or genes encoding a bait protein, an interacting prey protein, or both, identified in one of the Tables 1 through 11, as compared to patients without said disease or disorder.

23. The method of claim 22, wherein said alteration is an alteration in the nucleotide sequence of the gene or genes encoding a bait protein, an interacting prey protein, or both, identified in one of the Tables 1 through 11.

24. The method of claim 22, wherein said alteration is an alteration in the level of expression of a bait protein, an interacting prey protein, or both, identified in one of the Tables 1 through 11.

25. The method of claim 21 further comprising the step of determining if said alteration has been inherited.
